(19) United States
(12) Patent Application Publication
(10) Pub. No.: US 2025/0255909 A1
(43) Pub. Date: Aug. 14, 2025

(54) PREVOTELLA COPRI FORMULATIONS AND METHODS OF USE

(71) Applicants: Washington University, St. Louis, MO (US); International Centre For Diarrhoeal Disease Research, Bangladesh, Mohakhali, Daka (BD)

(72) Inventors: Jeffrey GORDON, St. Louis, MO (US); Yi WANG, St. Louis, MO (US); Hao-Wei CHANG, St. Louis, MO (US); Michael BARRATT, St. Louis, MO (US); Daniel WEBBER, St. Louis, MO (US); Matthew HIBBERD, St. Louis, MO (US); Tahmeed AHMED, Mohakhali, Dhaka City (BD)

(21) Appl. No.: 18/856,819

(22) PCT Filed: Apr. 14, 2023
(86) PCT No.: PCT/US2023/018738

§ 371 Date: Oct. 14, 2024

Related U.S. Application Data
(60) Provisional application No. 63/330,837, filed on Apr. 14, 2022.

(51) Int. Cl. A61K 35/741; A61K 9/00; A61K 35/00; A61K 35/745; A61K 47/46; C12N 1/20; C12R 1/01

(52) U.S. Cl. CPC A61K 35/741; A61K 9/0056; A61K 35/745; A61K 47/46; C12N 1/205; A61K 2035/115; C12R 2001/01

(57) ABSTRACT

Provided are probiotic compositions and methods of using such compositions for treatment of a spectrum of diseases like malnutrition. The probiotic compositions provided herein have Prevotella copri or engineered strains with genes from Prevotella copri.

### CROSS REFERENCE TO RELATED APPLICATIONS

This application claims the benefit of U.S. Provisional Application No. 63/330,837 filed Apr. 14, 2022 the disclosure of which are incorporated herein by reference in its entirety for all purposes.

### ACKNOWLEDGEMENT OF GOVERNMENT SUPPORT

This invention was made with government support under Grant No. DK30292 awarded by the National Institutes of Health (NIH). The government has certain rights in the invention.

### BACKGROUND

### 1. Field

The current invention relates to the field of treatment of malnutrition using the compositions and methods provided herein.

### 2. Background

The gut microbiome is a complex ecosystem with diverse microorganisms including bacteria, archaea, viruses, and fungi. More than a 100 trillion microorganisms live within a human body at any given point in time. The gut metagenome carries approximately 150 times more genes than are found in the human genome. The microbiome has a huge impact on health and well-being. Mechanisms by which these gut microorganisms impact health are manifold and include enhanced nutrient uptake, appetite signaling, competitive protection against harmful microorganisms, production of antimicrobials, role in development of the intestinal mucosa and immune system of the host, to a list a few. Imbalances in the microbiome are linked to development and progression of major human diseases including gastrointestinal diseases, infectious diseases, liver diseases, gastrointestinal cancers, metabolic diseases, respiratory diseases, mental or psychological diseases, and autoimmune diseases.

Childhood undernutrition is a vexing, pressing, and in many respects overwhelming global health issue. Undernutrition contributes to more than 40% of deaths worldwide among children under 5 years old. Acute undernutrition affects more than 50 million children and is defined by a low weight-for-height Z (WHZ) score [the number of standard deviations from the median value for a reference, multinational World Health Organization (WHO) cohort of children with healthy growth phenotypes]. Preschool children with severe wasting (WHZ<−3) have a 10-fold higher mortality rate than that of their well-nourished counterparts. In 2014, chronic undernutrition, which manifests as stunting [low height-for-age Z score (HAZ)], affected 159 million children, with almost all living in low-income countries. Despite these categorical distinctions, deficits in ponderal and linear growth frequently coexist and increase the risk that children will experience persistent stunting, defective immune responses, and impaired neurocognitive function into adulthood. Current approaches to treatment have only modest effects in correcting these long-term sequelae, suggesting that certain features of host biology are not being adequately repaired. This has led to the hypothesis that healthy growth is dependent, in part, on normal postnatal development of the gut microbiota and that perturbations in its development are causally related to undernutrition.

Addressing microbiome imbalances using probiotic formulations is becoming an important part of treatment plans for relevant disease for childhood undernutrition. The microbiome is however not static but evolves with dietary intake, and environmental factors. The microbiota also varies greatly between individuals from different geographical and socioeconomical backgrounds. Therefore, therapies are not a one-size-fits all approach. The effectiveness of any intervention to address microbiome imbalances is contingent on the various factors that impact the microbiome.

There is therefore a need to understand and tailor probiotic formulations to specific populations and diet contexts.

### SUMMARY OF THE INVENTION

In some aspects, the current disclosure encompasses a composition comprising a probiotic strain and at least a carrier, wherein the probiotic bacterial strain is operable to enhance utilization of xylooligosaccharides, fructooligosaccharides, oligogalacturonate, galactooligosaccharides, galactose, glucuronate, galacturonate and arabinooligosaccharides, or combinations thereof, when administered to a subject in need thereof compared to a subject lacking the probiotic strain. In some aspects, the probiotic bacterial strain comprises a genome sequence at least about 90% identical to any one of the sequences deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2.

In some aspects, the current disclosure also encompasses a composition comprising a probiotic strain and a carrier, wherein the probiotic bacterial strain comprises at least two, at least three, at least 4, at least 5, at least 6, at least 7, at least 8, at least 9, at least 10 or at least 20, at least 30 or more of a polynucleotide sequence encoding a protein from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of a genome sequence deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2.

In some aspects, the probiotic bacterial strain as provided herein comprises at least two, at least three, at least 4, at least 5, at least 6, at least 7, at least 8, at least 9, at least 10 or at least 20, at least 30 or more of polynucleotide sequences from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of P. copri strain NRRL deposit no. xxxxx or yyyyy or zzzzz. In some aspects, the probiotic bacterial strain is P. copri.

In some aspects, the probiotic bacterial strain as provided herein has a genome at least about 90% identical to the genome of any one of P. copri strain NRRL deposit no. xxxxx or yyyyy or zzzzz. In some aspects, the probiotic bacterial strain is any one of P. copri strain NRRL deposit no. xxxxx or yyyyy or zzzzz.

In some aspects the compositions as disclosed herein may further comprise a microbiome-directed therapeutic food (MDF). In some aspects, the MDF comprises chickpea flour, peanut flour, soy flour, green banana, sugar, at least one oil, optionally an amino acid mix, a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 6-24 months. In some aspects, the MDF contains no milk, powdered milk or milk product. In some aspects, the MDF has about 400 to about 600 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%. Non-limiting examples of MDF include MDCF-1, MDCF-2, MDCF-3, MDCF-2SS, MDSF, or MD-RUTF.

In some aspects, the compositions may further comprise an additional probiotic bacterial strain. In some aspects, the additional probiotic bacterial strain is a strain of Bifidobacterium longum subspecies infantis. In some aspects, the additional probiotic bacterial strain is Bifidobacterium longum subspecies infantis Bg_2D9. In some aspects, the additional probiotic bacterial strain is Bifidobacterium longum subsp. infantis with NRRL deposit #NRRL B-68253.

In some aspects the compositions as disclosed herein may be administered to a subject, wherein the subject is an undernourished child 0-5 years of age. In some aspects, the subject is a child is on a limited breast milk diet. In some aspects, the child is on a no breast milk diet. In some aspects, the subject may be a prospective mother. In some aspects, the composition may be administered before, during or after pregnancy and combinations thereof. In some aspects, the subject may be additionally administered a second composition comprising an MDF, at least one additional probiotic bacterial strain or both. In some aspects, the second composition is administered before, simultaneously or after the administration of the composition. In some aspects, the probiotic bacterial strain is an engineered probiotic bacterial strain.

In some aspects, the engineered probiotic bacterial strain comprises at least two, at least three, at least 4, at least 5, at least 6, at least 7, at least 8, at least 9, at least 10 or at least 20, at least 30 or more of a polynucleotide sequence encoding a protein from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of a genome sequence deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2. In some aspects, the engineered probiotic bacterial strain comprises a polynucleotide sequence at least about 60% identical to a polynucleotide sequence in any one of the polysaccharide utilization loci PUL3a, PUL3b, PUL9, PUL10, PUL15, PUL16, PUL17, PUL18, PUL 19, PUL20, PUL22, or PUL30 or any combination thereof, of P. copri strain NRRL deposit no. xxxxx or yyyyy or zzzzz, within its genome or as an extrachromosomal element.

In some aspects, the probiotic bacterial strain is present in an amount of more than 10² cfu per gram of the composition. In some aspects, the compositions as disclosed herein comprise at least a viable cell of the probiotic bacterial strain. In some aspects, the composition is formulated for oral administration. In some aspects, the composition is formulated for orogastric or nasogastric administration. In some aspects, the composition is in the form of a powder, a capsule, a tablet, a sachet, a liquid, an emulsion, or a suspension. In some aspects, the composition comprises an ingestible carrier. In some aspects, the ingestible carrier comprises a milk component. In some aspects, the ingestible carrier comprises baby formula or baby food. In some aspects, the ingestible carrier comprises F-75 or F-100 formulas. In some aspects, the ingestible carrier comprises a beverage.

In some aspects, the compositions further comprise one or more prebiotic, adjuvant, stabilizer, biological compound, dietary supplement, drug or combination thereof. In some aspects, the compositions as disclosed herein modify the gut microbiota of a subject in need thereof.

In some aspects, the current disclosure also encompasses an isolated bacterial strain comprising a genome sequence at least 95%, or at least 96%, or at least 97%, or at least 98%, or at least 99% identical to the genome sequence of P. copri strain NRRL deposit no. xxxxx or yyyyy or zzzzz. In some aspects, the isolated strain comprises a genome sequence more that 99% identical to the genome sequence of any one of the P. copri strain NRRL deposit no. xxxxx or yyyyy or zzzzz.

In some aspects the current disclosure also encompasses a method of treatment, the method comprising administering to a subject in need thereof, a therapeutically effective quantity of any of the compositions disclosed herein. In some aspects, the subject is a child 0-5 years of age. In some aspects, the subject exhibits symptoms of or is diagnosed with undernutrition, Moderate Acute Malnutrition (MAM), Severe Acute Malnutrition (SAM) or stunting. In some aspects, the subject is an infant with a limited to no breastmilk diet. In some aspects, the subject is exhibiting symptoms of or diagnosed with necrotizing enterocolitis, nosocomial infections, or enteric inflammation. In some aspects, the child is on a limited breast milk diet. In some aspects, the child is on a no breast milk diet. In some aspects, the subject is administered a second composition comprising an MDF, at least one additional probiotic bacterial strain or both. In some aspects, the second composition is administered before, simultaneously or after the administration of the composition. In some aspects, the MDF comprises chickpea flour, peanut flour, soy flour, green banana, sugar, at least one oil, optionally an amino acid mix, a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 6-24 months. In some aspects, the MDF contains no milk, powdered milk or milk product. In some aspects, the MDF has about 400 to about 600 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%. In some aspects, the MDF is selected from MDCF-1, MDCF-2, MDCF-3, MDCF-2SS, MDSF, or MD-RUTF. In some aspects, the method comprises administration of additional probiotic bacterial strain, wherein the strain is a strain of Bifidobacterium longum subspecies infantis. In some aspects, the additional probiotic bacterial strain is Bifidobacterium longum subspecies infantis Bg_2D9.

In some aspects, the current disclosure also encompasses use of the compositions as disclosed herein for modifying the gut microbiota of a subject in need thereof. In some aspects, the current disclosure also encompasses use of the compositions as disclosed herein for enhancing the utilization of one or more of xylooligosaccharides, fructooligosaccharides, oligogalacturonate, galactooligosaccharides, galactose, glucuronate, galacturonate and arabinooligosaccharides, or combinations thereof.

In some aspects, the current disclosure also encompasses a synbiotic formulation comprising at least a probiotic bacterial strain comprising a polynucleotide sequence at least about 90% identical to any one of the sequences deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2 and an MDF. In some aspects, the probiotic bacterial strain comprises at least two, at least three, at least 4, at least 5, at least 6, at least 7, at least 8, at least 9, at least 10 or at least 20, at least 30 or more of a polynucleotide sequence encoding a protein from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of a genome sequence deposited at the European Nucleotide Archive with accession numbers ERZ17359655a corresponding to P. copri Bg131, ERZ17359674 corresponding to P. copri BgF5_2 and ERZ17359677 corresponding to P. copri BgD5_2. In some aspects, the probiotic bacterial strain comprises at least two, at least three, at least 4, at least 5, at least 6, at least 7, at least 8, at least 9, at least 10 or at least 20, at least 30 or more of polynucleotide sequences from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of P. copri strain NRRL deposit no. xxxxx or yyyyy or zzzzz. In some aspects the probiotic bacterial strain is P. copri. In some aspects, the probiotic bacterial strain has a genome at least about 90% identical to the genome of any one of P. copri strain NRRL deposit no. xxxxx or yyyyy or zzzzz. In some aspects, probiotic bacterial strain is any one of P. copri strain NRRL deposit no. xxxxx or yyyyy or zzzzz. In some aspects of the symbiotic formulation, the MDF comprises chickpea flour, peanut flour, soy flour, green banana, sugar, at least one oil, optionally an amino acid mix, a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 6-24 months. In some aspects, the MDF contains no milk, powdered milk or milk product. In some aspects, the MDF has about 400 to about 600 kcal per 100 g of the composition, about 20 g to about 36 g of fat per 100 g of the composition, about 11 g to about 16 g of protein per 100 g of the composition, a protein energy ratio (PER) of about 8% to about 12%, and a fat energy ratio (FER) of about 45% to about 60%. In some aspects, the MDF is selected from MDCF-1, MDCF-2, MDCF-3, MDCF-2SS, MDSF, or MD-RUTF.

In some aspects, the current disclosure also encompasses a food formulation for example MDCF-1, MDCF-2, MDCF-3, MDCF-2SS, MDSF, or MD-RUTF or variants thereof, for treatment of MAM, SAM or stunting. In some aspects, the food formulation may be administered to augment the benefits of P. copri in the gut microbiome. In some aspects, the P. copri is administered as a composition as disclosed herein. In some aspects, the P. copri is not externally administered but exists in the subject's gut microbiome.

### BRIEF DESCRIPTION OF THE DRAWINGS

Embodiments of the present inventive concept are illustrated by way of example in which like reference numerals indicate similar elements and in which:

FIG. 1A shows photographs of the various food formulations developed for the trial.

FIG. 1B is a schematic of the timeline and phases of the study.

FIG. 2A shows a schematic of the study design.

FIG. 2B shows the Bioinformatic workflow for MAG assembly, refinement and quantitation. Pipeline for MAG assembly from short-read only or short-read plus long-read shotgun sequencing data. Steps are indicated on the left while the bioinformatic tools employed to accomplish each step are described within each box.

FIG. 2C shows comparison of MAG assembly summary statistics derived from CheckM (completeness, contamination) or Quast (contigs, length, N50) for 82 high-quality MAGs obtained from short-plus long-read hybrid assemblies versus 918 high-quality MAGs from short-read only assembly methods. Boxplots show the median, first and third quartiles; whiskers extend to the largest value no further than 1.5× the interquartile range. ***, P<0.001 (Wilcoxon test).

FIG. 2D shows volcano plot indicating the results of linear mixed-effects modeling of the relationship between MAG abundance and WLZ scores for all trial participants, irrespective of treatment. Bacterial genera that are abundant in the list of MAGs significantly associated with WLZ are colored by their taxonomic classification.

FIG. 2E shows the distribution of WLZ-associated MAGs across taxonomic groups. Left subpanel, density plot showing WLZ-associated MAGs tabulated based on their genus-level classification. β₁ refers to the coefficient in the mixed linear effects model presented at the bottom of the figure. Genera containing >3 significantly WLZ-associated MAGs are shown. Right subpanel, number of significant WLZ-associated MAGs assigned to each genus depicted in the left subpanel.

FIG. 2F shows results of gene set enrichment analysis (GSEA) of WLZ-associated MAGs ranked by the magnitude of their difference in abundance in response to MDCF-2 versus RUSF treatment. Plotted values indicate the mean log₂-fold difference (±SEM) in each model coefficient between the two treatment groups. The statistical significance of enrichment (q-value, GSEA) of MAGs that are positively or negatively associated with WLZ is shown.

FIG. 2G shows results of gene set enrichment analysis (GSEA) of WLZ-associated MAGs ranked by the magnitude of their change in ‘abundance over time’ in response to MDCF-2 versus RUSF treatment. Plotted values indicate the mean log₂-fold difference (±SEM) in each model coefficient between the two treatment groups. The statistical significance of enrichment (q-value, GSEA) of MAGs that are positively or negatively associated with WLZ is shown.

FIG. 2H shows enrichment of metabolic pathways in WLZ- and treatment-associated MAGs. MAGs were ranked by their WLZ association (negative to positive) or treatment association (RUSF-associated to MDCF-2 associated) and GSEA was employed to determine overrepresentation of pathways in MAGs at the extremes of each ranked list. The results (Normalized Enrichment Score, NES) only include pathways that display a statistically significant enrichment (q<0.05, GSEA) in both the WLZ-associated MAG and treatment-associated MAG analyses. For carbohydrate utilization pathways, disaccharides and oligosaccharides are indicated with an asterisk.

FIG. 3A provides LC-MS analysis of glycans for monosaccharides present in MDCF-2 and RUSF, and in the food ingredients used to formulate them. Mean±SD are plotted. *, P<0.05, **, P<0.01 (t-test).

FIG. 3B provides LC-MS analysis of glycans for glycosidic linkages present in MDCF-2 and RUSF, and in the food ingredients used to formulate them. Mean±SD are plotted. *, P<0.05, **, P<0.01 (t-test).

FIG. 3C shows polysaccharide structures of glycans enriched in components of MDCF-2 or RUSF.

FIG. 3D depicts the principal polysaccharides in MDCF-2, RUSF and their component ingredients. Mean values±SD are plotted. *, P<0.05; ***, P<0.001 (t-test).

FIG. 3E shows the structure of the galactans.

FIG. 3F shows the structure of the mannans.

FIG. 4A shows the principal taxonomic features and expressed functions of MDCF-2 and RUSF-treated fecal microbiomes. Significant enrichment of taxa (q<0.1; GSEA) along the first principal component (PC1) of MAG abundance or transcript abundance is shown.

FIG. 4B shows percent variance explained by top 10 principal components of a PCA analysis including abundance of MAGs.

FIG. 4C shows percent variance explained by top 10 principal components of a PCA analysis including transcripts across all available timepoints and study participants.

FIG. 4D shows significant enrichment of taxa (q<0.05, GSEA) along the first three principal components (PC1-PC3) of the fecal microbiome or meta-transcriptome.

FIG. 4E shows carbohydrate utilization pathways significantly enriched (q<0.1; GSEA) by treatment group (β₁, circles) or the interaction of treatment group and study week (β₃, squares). Right subpanel: Each point represents a MAG transcript assigned to each of the indicated functional pathways (rows), ranked by the direction and statistical significance of their differential expression in MDCF-2 versus RUSF treated participants (defined as the direction of the fold-change×−log 10 (P-value)). Transcripts are colored by their MAGs of origin. Larger, black outlined circles indicate leading edge transcripts assigned to the pathway described at the left of the panel.

FIG. 4F shows carbohydrate utilization pathways significantly enriched (q<0.1; GSEA) in upper- vs lower-WLZ quartile responders (β₁, diamonds) or the interaction of WLZ-response quartile and study week (β₃, triangles) (see linear mixed effects model). Right subpanel: Transcripts assigned to each functional pathway. Coloring and outlined circles have identical meaning as in panel b. The enrichment of glucuronate and galacturonate pathways was driven by the same transcripts, hence these pathways were considered as a single unit.

FIG. 5A shows unrooted, marker gene-based phylogenetic tree of 51 Prevotella MAGs from this study, plus 1,049 P. copri genomes and MAGs previously assigned to four clades. Pink stars denote the two WLZ-associated P. copri MAGs. The nine remaining P. copri MAGs from this study are highlighted by the green pentagons. The 40 Prevotella MAGs not classified as P. copri based on their having an average branch length >0.5 from all 1,049 reference P. copri isolates are grouped together and depicted as a yellow triangle

FIG. 5B shows mcSEED carbohydrate utilization pathways in 51 Prevotella MAGs from the current study. MAGs are hierarchically clustered based on the predicted presence (red) or absence (white) of these pathways.

FIG. 6 shows phylogenetic tree and inferred carbohydrate utilization phenotypes of Bifidobacterium MAGs. The phylogenetic tree indicates the relatedness of 34 Bifidobacterium MAGs and 14 reference genomes, as determined by sequence similarity among 142 core genes. The size of the pink circles in the dendrogram correspond to bootstrap support for the nodes (out of 100 bootstraps). Type stains used for taxonomic assignments and phenotypic comparisons are bolded. The matrix describes the presence (orange) or absence (white) of 25 predicted carbohydrate utilization phenotypes encompassing host- and plant-derived glycans. LNT, lacto-N-tetraose; LNnT, lacto-N-neotetraose; FL, 2′- and 3′-fucosyllactose; SL, 3′- and 6′-sialyllactose; Nglyc, N-glycans; Nglyc_core, N-glycan core (Fucα1-6GlcNAcβ1-Asn); GNB, galacto-N-biose; GlcNAc6S, N-acetylglucosamine-6-sulfate; Muc, mucin O-glycans; IMO, isomaltooligosaccharides and panose; MIz, melezitose; AXOS, arabinoxylooligosaccharides; XGIOS, xyloglucan oligosaccharides; ST, starch and glycogen; RST, resistant starch; GALA_I, type I galactan and arabinogalactan; AGII, type II galactan and arabinogalactan; GA, gum arabic; AR, arabinan; XL, xylan; AX, arabinoxylan; bMAN, β-mannan; XGL, xyloglucan; Gin, ginsenosides; Rgl, rhamnoglycosides.

FIG. 7A is a representation of seven highly conserved PULs, present in Bg0018 and Bg0019, among the nine other P. copri MAGs identified in study participants and six P. copri isolates obtained from Bangladeshi children. The phylogenetic tree (left) indicates the relatedness of P. copri MAGs and isolates as determined by a marker gene-based phylogenetic analysis. Tree tips are colored by their P. copri clade designation. The β₁(WLZ) coefficient for each P. copri MAG is indicated on the right of the figure; significant associations (q<0.05) are bolded. The color-coded matrix in the center indicates the extent of conservation of PULs in Bg0019 and Bg0018 versus the other P. copri MAGs identified in the fecal microbiomes of study participants. The known or predicted polysaccharide targets of these PULs are noted. The number of differentially expressed PUL transcripts in MAG Bg0018 and Bg0019 are shown in the colored cells; they were identified based on analysis of MDCF-2 versus RUSF treated participants and/or from upper versus lower WLZ-response quartile participants who all received MDCF-2.

FIG. 7B shows the relationship between PUL conservation in the 11 P. copri MAGs identified in study participants and the strength of each MAG's association with WLZ.

FIG. 7C shows the CAZyme components of select P. copri PULs.

FIG. 7D shows the locus structure of PUL7 in MAG Bg0019. Abbreviations: GH, CAZy glycoside hydrolase family assignment; CE, carbohydrate esterase.

FIG. 8A shows significant changes in fecal glycosidic linkage levels (q<0.05) over time in upper-compared to lower-WLZ quartile responders. Likely polysaccharide sources for each of the 14 glycosidic linkages are noted in the middle column. PULs present in P. copri MAGs Bg0018 and Bg0019 with known or predicted cleavage activity for the listed polysaccharide sources are noted on the right subpanel.

FIG. 8B is a boxplot of changes in the levels of fecal glycosidic linkages relative to initiation of treatment among upper- and lower-WLZ quartile responders. Levels of these 14 linkages increased to a significantly greater extent over time in the comparison of upper- vs lower WLZ-quartile (Model: linkage abundance˜WLZ-response quartile×study week+(1|PID)). Note that boxplots indicate the median, first and third quartiles; whiskers extend to the largest value no further than 1.5× the interquartile range.

FIG. 8C shows the β₃ coefficient for the interaction of WLZ-response quartile and study week is shown for CAZymes in PULs in Bg0018 and Bg0019. Predicted PUL substrates and potential glycosidic linkages in each of these substrates are shown at right. Glycosidic linkages with significant differences in fecal levels in upper versus lower WLZ-quartile responders are highlighted in bold font

FIG. 8D shows the polysaccharide structures, cleavage sites, and predicted products of CAZyme activity. Glycosidic linkages highlighted with arrows are those predicted as sites of cleavage by CAZymes expressed by the set of PULs, that are present in P. copri MAG Bg0019 and/or Bg0018. Consensus PUL numbers are listed except in the case of Bg0019 PUL3, which is not represented in Bg0018. The size of the arrows (large versus small) denotes the relative likelihood (high versus low, respectively) of cleavage of glycosidic linkages by P. copri CAZymes when considering steric hinderance at branch points.

FIG. 8E shows MDCF-2 polysaccharide substrates (left subpanels) and glycosidic linkage cleavage products predicted to be liberated by conserved P. copri MAGs Bg0019 and Bg0018 PULs. Linkages highlighted with arrows are putative sites of cleavage by the P. copri CAZymes based on their known or predicted enzyme activities; enzymes are labeled by their CAZyme module or modules predicted to perform the cleavage. The size of these arrows (large versus small) denotes the relative likelihood (high versus low, respectively) of glycosidic linkage cleavage by these CAZymes, considering steric hindrance at glycan branch points.

FIG. 8F shows the expression of PUL genes in MDCF-2 treated, upper- vs lower-WLZ quartile responders (only PUL genes with mcSEED or CAZy annotations are shown).

FIG. 8G shows predicted activity of PUL17b CAZymes, including cleavage of α-1,2- and α-1,3-linked arabinofuranose (Araf) side chains by GH51 (blue) and the α-1,5-Araf-linked backbone of branched arabinan by GH43 (brown, includes GH43_4 and GH43_5 subfamilies), respectively. Preferential cleavage of linear, unbranched regions of this glycan would be expected to yield oligosaccharide fragments containing t-Araf, 2-Araf, 5-Araf, and 2,3-Araf linkages, which are enriched in MDCF-2 treated, upper quartile WLZ-responders.

FIG. 8H shows predicted activities of PUL7 GH26, GH5_4, or GH26-GH5_4 family CAZymes (magenta) on β-1,4 linked mannose residues of galactomannan, yielding products containing 4,6-manose, the most significantly differentially abundant linkage in the upper quartile WLZ-responders (see panel a).

FIG. 9A depicts the experimental design for studying the relationship between P. copri colonization efficiency and pre-colonization with B. longum subsp. Infantis. Mice were weaned at P28 and P25 for experiments 1 and 2, respectively.

FIG. 9B shows the phylogenetic tree of P. copri isolates and MAGs. The phylogenetic distance between each pair of comparisons is shown in the matrix.

FIG. 9C provides the total absolute abundance of P. copri strains in fecal samples collected from pups at P42. Mean values±SD are shown. Each dot represents a separate mouse. P-values (Mann-Whitney U test) are noted.

FIG. 10A Energy contribution from different modules of the ‘weaning diet supplemented with MDCF-2’.

FIG. 10B shows the study design outlining the timing of bacterial colonization of dams and diet switches.

FIG. 10C shows study shows the gavages administered to members of each treatment arm.

FIG. 10D provides the absolute abundance of B. infantis Bg2D9 (Arm 1) and B. infantis Bg463 (Arm 2) in fecal samples obtained from pups.

FIG. 10E provides absolute abundance of P. copri in fecal samples collected from pups in the indicated treatment arms at the indicated postnatal time points. Inset: the absolute abundance of P. copri in fecal samples collected from pups at P21 (Mann-Whitney U test)

FIG. 10F provides body weights of the offspring of dams, normalized to postnatal day 23. [linear mixed effects model (see Methods)]. Mean values±SD are shown. Each dot in panels d-f represent an individual animal. P values were calculated using a Mann-Whitney U test (panel e insert) or a linear mixed effect model.

FIG. 11A shows ultra-high performance liquid chromatography-triple quadrupole mass spectrometric (UHPLC-QqQ-MS) quantitation of levels of arabinose-containing glycosidic linkages in cecal glycans.

FIG. 11B shows ultra-high performance liquid chromatography-triple quadrupole mass spectrometric (UHPLC-QqQ-MS) quantitation of levels of total arabinose in cecal glycans.

FIG. 11C provides GC-MS quantitation of cecal acetate levels. Mean values±SD are shown. P-values were calculated using a Mann-Whitney U test.

FIG. 11D is an illustration of the singular value decomposition and its application to microbial RNA-seq analysis. Matrix M stores the TPM value for each bacterium in each sample. Reads mapped to P. copri, P. stercorea, and the two strains of B. longum subsp. infantis were removed and transcripts with low expression were filtered out using edgeR before generating matrix M.

FIG. 11E shows projection of samples onto a space determined by PC1 and PC2. Centroids are denoted by a white “X”. Shaded ellipses represent the 95% confidence interval of the sample distribution.

FIG. 11F shows projection of the transcriptional responses of reconstructed metabolic pathways for each bacterium listed in M on the same PC space as depicted. Bacteria that can utilize arabinose, based on mcSEED metabolic reconstruction, are highlighted using bold font.

FIG. 11G shows differential expression analysis of genes involved in carbohydrate utilization, amino acid biosynthesis, and fermentation in arabinose-utilizing bacteria. Violin plots show the distribution of log 2 fold-differences for all expressed genes in the indicated strain. Abbreviations: Glu, glutamate; Gln, glutamine; Leu, leucine; Ile, isoleucine; Val, valine.

FIG. 12A provides the number of Recon2 reactions with statistically significant differences in their predicted flux between the w/ P copri and w/o P. copri groups.

FIG. 12B provides the number of Recon2 reactions in each Recon2 subsystem that are predicted to have statistically significant differences in their activities between the two treatment groups. Colors denote values normalized to the sum of all statistically significantly different Recon2 reactions found in all selected cell clusters for a given Recon2 subsystem in each treatment group.

FIG. 12C is a proportional representation of cell clusters identified by snRNA-Seq. Asterisks denote ‘statistically credible differences’ as defined by scCODA.

FIG. 12D shows selected Recon2 reactions in enterocyte clusters distributed along the villus involved in the urea cycle and glutamine metabolism.

FIG. 12E provides targeted mass spectrometric quantifications of citrulline levels along the length of the gut and in plasma. Mean values±SD and P-values from the Mann-Whitney U test are shown.

FIG. 12F shows the effect of colonization with bacterial consortia containing or lacking P. copri on extracellular transporters for monosaccharides, amino acids and dipeptides. Sar: sarcosine. These transporters were selected and the spatial information of their expressed region along the length of the villus was assigned based on published experimental evidence. Arrows in panels b and e indicate the “forward” direction of each Recon2 reaction. The Wilcoxon Rank Sum test was used to evaluate the statistical significance of the net reaction scores (FIG. 12A, FIG. 12B, FIG. 12D and FIG. 12E) between the two treatment groups. P-values were calculated from Wilcoxon Rank Sum tests and adjusted for multiple comparisons (Benjamini-Hochberg method); a q-value <0.05 was used as the cut-off for statistical significance.

FIG. 13A is a dot plot of marker gene expression across epithelial cell types. The average expression level and percentage of nuclei that express a given gene within a cell type are indicated by dot color and size, respectively.

FIG. 13B provides an integrated UMAP plot for all jejunal nuclei isolated from 8 animals representing the two treatment arms (n=4 mice/arm) in the parameter screen experiment.

FIG. 13C provides the number and directionality of statistically significant differentially expressed genes in each cell cluster.

FIG. 14 illustrates NicheNet-based analysis of the effects of P. copri colonization on cell-cell signaling activities. Each row represents different sender cell clusters. Each column represents ligands expressed by these sender cells. Cells are colored based on the log 2-fold difference in expression of ligands in the sender cell clusters between w/ P. copri and w/o P. copri mice. Ligands (columns) are grouped based on receiver cell clusters and the indicated functions of downstream signaling pathways in these receiver cells.

FIG. 15A provides the study design for validating the effects of P. copri colonization in gnotobiotic mother-pup dyads.

FIG. 15B provides body weights of the offspring of dams, normalized to postnatal day 23 linear mixed effects model.

FIG. 15C provides a targeted mass spectrometric analysis of jejunal citrulline. Each dot represents a single animal. Mean values±SD are shown. P-values were calculated from the linear mixed effect model (panel b) or Mann-Whitney U test. N.S., P-value >0.05.

FIG. 15D provides a targeted mass spectrometric analysis of acylcarnitine levels. Each dot represents a single animal. Mean values±SD are shown. P-values were calculated from the linear mixed effect model (panel b) or Mann-Whitney U test. N.S., P-value >0.05.

FIG. 15E provides a targeted mass spectrometric analysis of colonic acylcarnitine levels.

FIG. 15F provides plasma levels of non-esterified fatty acids. Each dot represents a single animal. Mean values±SD are shown. P-values were calculated from the linear mixed effect model (panel b) or Mann-Whitney U test. N.S., P-value >0.05. Each dot represents a single animal. Mean values±SD are shown. P-values were calculated from the linear mixed effect model (panel b) or Mann-Whitney U test. N.S., P-value >0.05.

FIG. 16 shows normalized number of Recon2 reactions in Recon2 subsystems predicted to have statistically significant differences in their activities between the w/ P. copri and w/o P. copri treatment groups.

FIG. 17A shows the study design for testing the effects of pre-weaning colonization with two P. copri strains closely related to MAGs Bg0018 and Bg0019 on host weight gain, and MDCF-2 glycan degradation.

FIG. 17B provides absolute abundance of P. copri strains and total bacterial load in cecal contents collected at P53.

FIG. 17C provides body weights of the offspring of dams, normalized to postnatal day 23 [linear mixed effects model (see Methods)].

FIG. 17D shows the comparison of polysaccharide utilization loci (PULs) highly conserved in the two P. copri MAGs (Bg0018 and Bg0019) identified in the RCT as being significantly positively associated with WLZ and MDCF-2 glycan metabolism, with their representation in the three cultured P. copri strains.

FIG. 17E provides UHPLC-QqQ-MS analysis of total arabinose and galactose in glycans present in cecal contents collected at euthanasia (P53).

FIG. 17F provides UHPLC-QqQ-MS of glycosidic linkages containing arabinose in cecal contents. Mean values±SD are shown. P-values were calculated using a Mann-Whitney U test.

FIG. 17G provides UHPLC-QqQ-MS of glycosidic linkages containing galactose in cecal contents. Mean values±SD are shown. P-values were calculated using a Mann-Whitney U test.

FIG. 18A provides comparison of weight-for-length z-score (WLZ) between the MDCF-2 and RUSF groups at different time points up to 2 years after cessation of the 3-month intervention in 12-18 month children with primary MAM.

FIG. 18B provides comparison of length-for-age z-score (LAZ) between MDCF-2 and RUSF groups at different time points up to 2 years after cessation of the 3-month intervention in 12-18 month children with primary MAM.

FIG. 18C provides comparison of weight-for-age z-score (WAZ) between MDCF and RUSF group at different time points up to 2 years after cessation of the 3-month intervention in 12-18 month children with primary MAM.

FIG. 19 shows LC-MS of ileal and colonic acylcarnitines in gnotobiotic mice colonized with P. copri D5.2 and F5.2. Mean values±SD are shown. P-values were calculated using a Mann-Whitney U test.

The drawing figures do not limit the present inventive concept to the specific embodiments disclosed and described herein. The drawings are not necessarily to scale, emphasis instead being placed on clearly illustrating principles of certain embodiments of the present inventive concept.

### DETAILED DESCRIPTION

The present disclosure encompasses compositions and methods of treatment for subjects in need thereof, where the methods of treatment comprise administering a disclosed composition. In some embodiments, the methods of treatment address malnutrition, including undernutrition, in part by modifying the gut microbiota of the subject. The global burden of childhood undernutrition is great, causing 3.1 million deaths annually and accounting for 21% of life years lost among children younger than 5 years. More than 18 million children in this age range are affected by severe acute malnutrition (SAM), the most extreme form of undernutrition. SAM is responsible for nearly half of all undernutrition-related mortality. Various aspects of this invention demonstrate that there is a correlation between childhood malnutrition and deficiencies in components of the gut microbiota whose restoration is associated with improved outcomes for acutely malnourished children. In one aspect, the present disclosure is a result of extensive experimental studies that correlate the evolution of the gut microbiome with the various therapeutic and dietary interventions that help improve the health of SAM patients. The presence of one particular bacterial strain Prevotella copri (P. copri) in these synbiotic studies was correlated with much better outcomes for the patients. In another aspect, the present disclosure also stems from extensive screening and in-depth characterization of the gut microbiome for identification of bacterial strains for enhanced survival (fitness) in children who consume diets with limited breastmilk content. While exclusive breastfeeding of infants is recommended by the WHO for the first 6 months, in many low-income settings, gruels, animal milk and complementary foods are often introduced into the diet at an early age for economic and/or cultural reasons. Surprisingly, Prevotella copri obtained from these extensive screening efforts exhibits superior fitness over multiple other strains, in population with complementary plant-based diets. Metagenomic characterization of the strains helped define DNA sequences involved in the uptake, or utilization or both of xylooligosaccharides, fructooligosaccharides, oligogalacturonate, galactooligosaccharides, galactose, glucuronate, galacturonate and arabinooligosaccharides, or combinations thereof by the isolated strain compared to comparable strains without these DNA sequences.

The current disclosure describes isolated and engineered strains of Prevotella copri comprising one or more of these DNA sequences, and therapeutic or synbiotic formulations comprising these strains, that when administered into a subject in need thereof, enhance the capacity for uptake or utilization of certain plant-based polysaccharides. Such treatments improve outcomes for malnourished children. In some aspects, the disclosed strain compositions can be administered alone. In some aspects, the disclosed strain compositions can be administered in combination with food formulations. In some aspects, the disclosed strain compositions can be administered with additional probiotic compositions. In some aspects, the strain compositions can be administered with additional food and probiotic formulations. Some aspects of this invention further provide methods for modifying gut microbiota, thus providing advantageous outcomes including but not limited to reducing symptoms of, or treating, acute malnutrition, enteric inflammation, necrotizing enterocolitis, and allergies, promoting recolonization of the gut after diarrhea or antibiotic consumption, and improving vaccine performance by administering therapeutically effective quantities of these formulations.

### Definitions

Unless defined otherwise, all technical and scientific terms used herein have the meaning commonly understood by a person skilled in the art to which this invention belongs. The following references provide one of skill with a general definition of many of the terms used in this invention: Singleton et al., Dictionary of Microbiology and Molecular Biology (2nd ed. 1994); The Cambridge Dictionary of Science and Technology (Walker ed., 1988); The Glossary of Genetics, 5th Ed., R. Rieger et al. (eds.), Springer Verlag (1991); and Hale & Marham, The Harper Collins Dictionary of Biology (1991). As used herein, the following terms have the meanings ascribed to them unless specified otherwise.

The phraseology and terminology employed herein are for the purpose of description and should not be regarded as limiting. For example, the use of a singular term, such as, “a” is not intended as limiting of the number of items. Also, the use of relational terms such as, but not limited to, “top,” “bottom,” “left,” “right,” “upper,” “lower,” “down,” “up,” and “side,” are used in the description for clarity in specific reference to the figures and are not intended to limit the scope of the present inventive concept or the appended claims.

Further, as the present inventive concept is susceptible to embodiments of many different forms, it is intended that the present disclosure be considered as an example of the principles of the present inventive concept and not intended to limit the present inventive concept to the specific embodiments shown and described. Any one of the features of the present inventive concept may be used separately or in combination with any other feature. References to the terms “embodiment,” “embodiments,” and/or the like in the description mean that the feature and/or features being referred to are included in, at least, one aspect of the description. Separate references to the terms “embodiment,” “embodiments,” and/or the like in the description do not necessarily refer to the same embodiment and are also not mutually exclusive unless so stated and/or except as will be readily apparent to those skilled in the art from the description. For example, a feature, structure, process, step, action, or the like described in one embodiment may also be included in other embodiments but is not necessarily included. Thus, the present inventive concept may include a variety of combinations and/or integrations of the embodiments described herein. Additionally, all aspects of the present disclosure, as described herein, are not essential for its practice. Likewise, other systems, methods, features, and advantages of the present inventive concept will be, or become, apparent to one with skill in the art upon examination of the figures and the description. It is intended that all such additional systems, methods, features, and advantages be included within this description, be within the scope of the present inventive concept, and be encompassed by the claims.

Any term of degree such as, but not limited to, “substantially” as used in the description and the appended claims, should be understood to include an exact, or a similar, but not exact configuration. For example, “a substantially planar surface” means having an exact planar surface or a similar, but not exact planar surface. Similarly, the terms “about” or “approximately,” as used in the description and the appended claims, should be understood to include the recited values or a value that is three times greater or one third of the recited values. For example, about 3 mm includes all values from 1 mm to 9 mm, and approximately 50 degrees includes all values from 16.6 degrees to 150 degrees. For example, they can refer to less than or equal to ±5%, such as less than or equal to ±2%, such as less than or equal to ±1%, such as less than or equal to ±0.5%, such as less than or equal to ±0.2%, such as less than or equal to ±0.1%, such as less than or equal to ±0.05%. As used herein, “about” refers to numeric values, including whole numbers, fractions, percentages, etc., whether or not explicitly indicated. The term “about” generally refers to a range of numerical values, for instance, ±0.5-1%, ±1-5% or ±5-10% of the recited value, that one would consider equivalent to the recited value, for example, having the same function or result.

Lastly, the terms “or” and “and/or,” as used herein, are to be interpreted as inclusive or meaning any one or any combination. Therefore, “A, B or C” or “A, B and/or C” mean any of the following: “A,” “B” or “C”; “A and B”; “A and C”; “B and C”; “A, B and C.” An exception to this definition will occur only when a combination of elements, functions, steps or acts are in some way inherently mutually exclusive.

When introducing elements of the present disclosure or the preferred aspects(s) thereof, the articles “a”, “an”, “the” and “said” are intended to mean that there are one or more of the elements. The terms “comprising”, “including” and “having” are intended to be inclusive and mean that there may be additional elements other than the listed elements.

The term “comprising” means “including, but not necessarily limited to”; it specifically indicates open-ended inclusion or membership in a so-described combination, group, series and the like. The terms “comprising” and “including” as used herein are do not exclude additional, unrecited elements or method processes. The term “consisting essentially of” is more limiting than “comprising” but not as restrictive as “consisting of.” Specifically, the term “consisting essentially of” limits membership to the specified materials or steps and those that do not materially affect the essential characteristics of the claimed invention.

The terms “nucleic acid”, “nucleic acid molecule”, and “polynucleotide” are used interchangeably herein. The terms “nucleic acid encoding . . . ”, or “nucleic acid molecule encoding . . . ” should be understood as referring to the sequence of nucleotides which encodes a polypeptide.

As used herein, the term “polynucleotide”, which may be used interchangeably with the term “nucleic acid” generally refers to a biomolecule that comprises two or more nucleotides. In some aspects, a polynucleotide comprises at least two, at least five at least ten, at least twenty, at least 30, at least 40, at least 50, at least 100, at least 200, at least 250, at least 500, or any number of nucleotides. For example, the polynucleotides may include at least 500 nucleotides, at least about 600 nucleotides, at least about 700 nucleotides, at least about 800 nucleotides, at least about 900 nucleotides, at least about 1000 nucleotides, at least about 2000 nucleotides, at least about 3000 nucleotides, at least about 4000 nucleotides, at least about 4500 nucleotides, or at least about 5000 nucleotides. A polynucleotide may be single-stranded or double-stranded. In some aspects, a polynucleotide is a site or region of genomic DNA. In some aspects, a polynucleotide is an endogenous gene that is comprised within the genome of an unmodified cell or universal donor cell. In some aspects, a polynucleotide is an exogenous polynucleotide that is not integrated into genomic DNA. In some aspects, a polynucleotide is an exogenous polynucleotide that is integrated into genomic DNA. In some aspects, a polynucleotide is a plasmid. In some aspects, a polynucleotide is a circular or linear molecule.

The term “DNA sequence” refers to a heritable sequence of DNA, i.e., a genomic sequence, with functional significance. The term “gene” can be used to refer to, e.g., a cDNA and/or an mRNA encoded by a genomic sequence, as well as to that genomic sequence.

Nucleic acid is “operably linked” when it is placed into a functional relationship with another nucleic acid sequence. For example, a promoter or enhancer is operably linked to a coding sequence if it affects the transcription of the sequence; or a ribosome binding site is operably linked to a coding sequence if it is positioned so as to facilitate translation. Generally, “operably linked” means that the DNA sequences being linked are contiguous, and, in the case of a secretory leader, contiguous and in reading phase. However, enhancers do not have to be contiguous.

The isolated strains of “Prevotella copri” for use in compositions as disclosed herein refers to P. copri strains available at Professor Jeffery I. Gordon's laboratory at Washington University, School of Medicine at St. Louis and corresponds to NRRL deposit nos. xxxx, yyyy or zzzz at the ARS Culture Collection (NRRL). A genome sequence of the three strains has also been deposited at the European Nucleotide Archive under project number PRJEB45356 and correspond to accession numbers ERZ17359655a corresponding to Prevotella copri Bg131, ERZ17359674 corresponding to Prevotella copri BgF5_2 and ERZ17359677 corresponding to Prevotella copri BgD5_2 respectively.

The term “carbohydrate”, as used herein, refers to an organic compound with the formula Cm(H2O)n, where m and n may be the same or different number, provided the number is greater than 3.

The term “glycan” refers to a linear or branched homo- or heteropolymer of two or more monosaccharides linked glycosidically. As such, the term “glycan” includes disaccharides, oligosaccharides and polysaccharides. The term also encompasses a polymer that has been modified, whether naturally or otherwise; non-limiting examples of such modifications include acetylation, alkylation, esterification, etherification, oxidation, phosphorylation, selenization, sulfonation, or any other manipulation.

The term “N-glycan,” as used herein, refers to a polymer of sugars that has been released from a glycoconjugate but was formerly linked to the glycoconjugate via a nitrogen linkage (see definition of N-linked glycan below). “N-linked glycans” are glycans that are linked to a glycoconjugate via a nitrogen linkage. A diverse assortment of N-linked glycans exist.

As used herein “Polysaccharide Utilization Loci” or “PUL” is used interchangeably and corresponds to PUL predictions as provided in the PUL database (Terrapon et al. 2018). The “fiber degrading capacity” of a subject's gut microbiota may be defined by its compositional state and/or its functional state. For instance, the compositional stage of a subject's gut microbiota may be defined by the absence, presence and abundance of primary and secondary consumers of dietary fiber, while the functional state may be defined by the representation of relevant genomic loci (polysaccharide utilization loci (PULs), carbohydrate-active enzymes (CAZymes), etc.), expression from these loci, and/or activity of proteins encoded by these loci. An increase in the fiber degrading capacity of a subject may be effected by increasing the abundance of microorganisms with genomic loci for import and metabolism of glycans, as exemplified by PULs and/or loci encoding CAZymes; and/or increasing the abundance or expression of one or more proteins encoded by a PUL and/or one or more CAZyme (with or without concomitant changes in microorganism abundance). Thus, for example PUL17 on the genome of P. copri refers to the genome loci encoding pectin degrading enzymes.

As used herein, the term “malnutrition” refers to one or more forms of undernutrition—for example, wasting (low weight-for-length), stunting (low length-for-age), underweight (low weight-for age), deficiencies in vitamins and minerals, etc. A subject in need of treatment for malnutrition may also be referred to herein as a malnourished subject.

A length-for-age Z Score (LAZ) refers to the number of standard deviations of the actual length of a child from the median length of the children of his/her age as determined from the standard sample. This is prefixed by a positive sign (+) or a negative sign (−) depending on whether the child's actual length is more than the median length or less than the median length. The terms length and height are used interchangeably herein. Therefore, length-for-age Z Score (LAZ) and height-for-age Z Score (HAZ) refer to the same measurement.

A weight-for-age Z score (WAZ) refers to the number of standard deviations of the actual weight of a child from the median weight of the children of his/her age as determined from the standard sample. This is prefixed by a positive sign (+) or a negative sign (−) depending on whether the child's actual weight is more than the median weight or less than the median weight.

A weight-for-length Z score (WLZ) refers to the number of standard deviations of the actual weight of a child from the median weight of the children of his/her length as determined form the standard sample. This is prefixed by a positive sign (+) or a negative sign (−) depending on whether the child's actual weight is more than the median weight or less than the median weight for the same length. The terms length and height are used interchangeably herein. Therefore, weight-for-height Z score (WHZ) and weight-for-length Z score (WLZ) refer to the same measurement.

A mid-upper-arm-circumference score (MUAC) is an independent anthropometric measurement used to identify malnutrition.

Moderate acute malnutrition (MAM) is defined by a WHZ less than or equal to −2 and greater than or equal to −3.

Severe acute malnutrition (SAM) is defined by a WHZ less than −3 and/or bipedal edema, and/or a mid-upper arm circumference (MUAC) less than 11.5 cm.

As used herein, a “healthy child” has a LAZ and WLZ consistently no more than 1.5 standard deviations below the median calculated from a World Health Organization (WHO) reference healthy growth cohort as described in WHO Multicentre Reference Study (MGRS), 2006 (www.who.int/childgrowth/mgrs/en).

As used herein, “stunting” or linear growth faltering is defined by a LAZ of less than or equal to −2. In some aspects, shunting can occur in the absence of wasting (MAM, SAM), but is often a co-morbidity in children with MAM or SAM.

As used herein, “statistically significant” is a p-value <0.05, <0.01, <0.001, <0.0001, or <0.00001.

The terms “treat,” “treating,” or “treatment” as used herein, refer to both therapeutic treatment and prophylactic or preventative measures, wherein the object is to prevent or slow down (lessen) an undesired physiological change or disease/disorder. Beneficial or desired clinical results include, but are not limited to, alleviation of symptoms, diminishment of extent of disease, stabilization (i.e., not worsening) of disease, a delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the disease, condition, or disorder as well as those prone to have the disease, condition or disorder or those in which the disease, condition or disorder is to be prevented.

As used herein, the term “effective amount” means an amount of a substance (e.g. a composition including formulations and combinations of the present disclosure) that leads to measurable and beneficial effects for the subject administered the substance, i.e., significant efficacy. As used herein the term “therapeutically effective amount” refers to an amount of the formulation or therapeutic combination that alleviates, in whole or in part, symptoms associated with the disorder or condition, or halts or slows further progression or worsening of those symptoms or prevents or provides prophylaxis for the disorder or condition. A therapeutically effective amount is also one in which any toxic or detrimental effects of compositions of the invention are outweighed by the therapeutically beneficial effects.

As used herein, the term “raw banana” refers to an unripe, green banana in the genus Musa. “Raw bananas” are also referred to as “green bananas” in the art, and the terms are used interchangeably herein. As is understood in the art, raw bananas are processed (e.g., baked, boiled, steamed, etc.) after which the pulp may or may not be dried prior to use.

The term “modifying” as used in the phrase “modifying the gut microbiota” is to be construed in its broadest interpretation to mean a change in the representation of microbes in the gastrointestinal tract of a subject. The change may be a decrease or an increase in the presence of a particular microbial strain, species, genus, family, order, or class. In some aspects, “modifying the gut microbiota” can “repair the gut microbiota” or “improve gut microbiota health”. To “repair the gut microbiota of a subject,” which is synonymous with “improve gut microbiota health,” means to change the microbiota of a subject, in particular the relative abundances of age- and health-discriminatory taxa, in a statistically significant manner towards chronologically-age matched reference healthy subjects. The term encompasses complete repair and levels of repair that are less than complete. The term also encompasses preventing or lessening a change in the relative abundances of age- and health-discriminatory taxa, wherein the change would have been significantly greater absent intervention.

As used herein the term “enhanced uptake” is intended to mean that the presence of the DNA sequence enhances the active transport of glycans, polysaccharides, or both into the bacterial cell compared to the same cell, or a cell of a similar background without the DNA sequence. In some aspects, the DNA sequence is known (based on assays known to a person of ordinary skill in the art including but not limited to binding assays, assays using glycan-recognizing probes comprising one or more of antibodies, lectins, carbohydrate molecules coupled with enzyme assays, immunohistochemistry, confocal microscopy, electron microscopy and flow cytometry) or predicted (based on sequence homology studies or curation using mcSEED analysis) to increase binding and intracellular transport of glycans, or plant derived oligosaccharides, or both by the microbe.

As used herein the term “enhanced utilization” is intended to mean that the presence of the DNA sequence enhances one or more of transport of glycans, transport of plant-derived polysaccharides, or both into the bacterial cell, and their subsequent metabolic processing [or metabolism]. In some aspects the DNA sequence is known (based on assays known to a person of ordinary skill in the art including but not limited to carbohydrate fermentation assays or glycan-recognizing probes comprising one or more of antibodies, lectins, carbohydrate molecules or enzyme assays) or predicted to (based on sequences homology studies or curation using mcSEED analysis) to increase microbial breakdown of N-glycans or plant derived oligosaccharides, or both.

As used herein, the term “subject” refers to a mammal. In some aspects, a subject is non-human primate or rodent. In some aspects, a subject is a human. In some aspects, a subject has, is suspected of having, or is at risk for, a disease or disorder. In some aspects, a subject has one or more symptoms of a disease or disorder. In particular aspects, a subject is malnourished. In some aspects, the subject is a child of 0-5 years of age. In some aspects, the subject is a child of 0-5 years of age, suspected of developing or having symptoms of malnutrition.

### I. Compositions

In one aspect, the present disclosure encompasses a composition comprising a probiotic strain and at least a carrier, wherein the probiotic bacterial strain is operable to enhance utilization of xylooligosaccharides, fructooligosaccharides, oligogalacturonate, galactooligosaccharides, galactose, glucuronate, galacturonate and arabinooligosaccharides, or combinations thereof, when administered to a subject in need thereof compared to a subject lacking the probiotic strain. In some aspects, the probiotic strain is an isolated strain of Prevotella copri isolated from the gut of Bangladeshi children, which were found to have enhanced capability to absorb and utilize various food substrates including arabinoxylan, pectin, b-mannan, b-glucan, xylan, arabinoxylan, glucomannan, xyloglucan, b-1,3-glucan, pectin galactan, starch or arabinogalactan. The genome of the strains of Prevotella copri of NRRL deposit no. xxxxx or yyyy or zzzz have been deposited in the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2 respectively. These strains were found to be highly beneficial to the children in protecting against undernutrition SAM, MAM, or stunting, either alone or in conjunction with other food supplements and probiotics. As such, in some aspects, the current disclosure encompasses a composition comprising a carrier and an isolated bacterial strain comprising a genome sequence at least 95%, or at least 96%, or at least 97%, or at least 98%, or at least 99% or 100% identical to the genome sequence as deposited in the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2 respectively. These isolated strains correspond to the P. copri strain NRRL deposit no. xxxxx or yyyyy or zzzzz. Thus, in some aspects, the current disclosure also encompasses a composition comprising a carrier and an isolated bacterial strain comprising a genome sequence 100% identical to the genome sequence of any one of P. copri strain NRRL deposit no. xxxxx or yyyyy or zzzzz. In some aspects, the current disclosure also encompasses an isolated bacterial strain comprising a genome sequence at least 95%, or at least 96%, or at least 97%, or at least 98%, or at least 99% identical to the genome sequence of any one of P. copri strain NRRL deposit no. xxxxx or yyyyy or zzzzz. Further characterization of these strain was conducted, and specific genetic loci could be identified that imparted on the strains disclosed the beneficial properties of glycan utilization as provided herein. Table A-D provides the corresponding location of the Polysaccharide Utilization Loci (PUL) in the genome as identified by their short locus tags, that enhance utilization of the one or more of arabinoxylan, pectin, b-mannan, b-glucan, xylan, arabinoxylan, glucomannan, xyloglucan, b-1,3-glucan, pectin galactan, starch or arabinogalactan, in each of the 3 strains

TABLE A

| Consensus | Predicted | | | |
|---|---|---|---|---|
| PUL | Substrate | BgD5_2 | BgF5_2 | Bg131 |
| PUL3 | arabinoxylan | PUL9 | PUL8 | PUL15 (divergent |
| | | | | SusCD + |
| | | | | GH10\|CBM4\|CBM |
| | | | | 4 fragmented to |
| | | | | GH10[Fnc]) |
| PUL4 | pectin | PUL17 (divergent | PUL16 (divergent | PUL6 + PUL7 |
| | | SusD 50%, | SusD 50%, | (divergent |
| | | divergent SusC | divergent SusC | SusCD, extra |
| | | 66%, no GH28, | 66%, no GH28, | SusCD, divergent |
| | | divergent CE8 | divergent CE8 | CE8 39%, no |
| | | 80%) | 80%) | PL1) |
| PUL7 | b-mannan, b- | PUL19 | PUL18 | PUL5 (GH26 |
| | glucan, xylan, | | | divergent 42% |
| | arabinoxylan, | | | and with no |
| | glucomannan, | | | GH5_4 and |
| | xyloglucan | | | misplaced, |
| | | | | missing one |
| | | | | GH26, divergent |
| | | | | SusCD and |
| | | | | directed the |
| | | | | opposite way) |
| PUL8 | b-glucan, b- | PUL18 | PUL17 | isolated GH5_7 |
| | mannan, xylan, | | | |
| | glucomannan | | | |
| PUL9 | b-galactoside, a- | PUL15 | PUL14 | — |
| | glucoside, a- | | | |
| | mannoside | | | |
| PUL10 | pectin | PUL22 (divergent | PUL20 (divergent | PUL3 (divergent |
| | | SusCD) | SusCD, divergent | SusCD) |
| | | | GH97_3) | |
| PUL11 | b-1,3-glucan | isolated GH3 and | | PUL30 |
| | | SusR | | |
| PUL13 | b-glucan, xylan | PUL16 (divergent | PUL15 (divergent | PUL8 |
| | | SusD 86%) | SusD 86%) | |
| PUL16 | pectic galactan | PUL10 (missing | PUL9 (missing | PUL14 (PL1 |
| | | one SusCD, | one SusCD, | without CBM77, |
| | | divergent GH53 | divergent GH53 | missing one |
| | | 89%) | 89%) | SusCD) |
| PUL17a | starch | PUL3a (divergent | PUL2a (divergent | PUL27a |
| | | SusCD) | SusCD) | (divergent |
| | | | | SusCD) |
| PUL17b | arabinogalactan | PUL3b (divergent | PUL2b (divergent | PUL27b (extra |
| | | SusD 78%, | SusD 78%, | SusCD and |
| | | divergent GH43_4 | divergent GH43_4 | divergent, |
| | | 84%, divergent | 84%, divergent | divergent GH43_4 |
| | | GH43_5 80%) | GH43_5 80%) | 71%, no GH51 - |
| | | | | isolated) |

TABLE B

| Prevotella copri BgD5_2 PUL | Locus Tags |
|---|---|
| PUL9 | PFCKPOMF_00840 |
| | PFCKPOMF_00841 |
| | PFCKPOMF_00842 |
| | PFCKPOMF_00843 |
| | PFCKPOMF_00844 |
| | PFCKPOMF_00845 |
| | PFCKPOMF_00846 |
| | PFCKPOMF_00847 |
| | PFCKPOMF_00848 |
| | PFCKPOMF_00849 |
| | PFCKPOMF_00850 |
| PUL17 (divergent SusD 50%, divergent SusC 66%, no | PFCKPOMF_01484 |
| GH28, divergent CE8 80%) | |
| | PFCKPOMF_01485 |
| | PFCKPOMF_01486 |
| | PFCKPOMF_01487 |
| | PFCKPOMF_01488 |
| | PFCKPOMF_01489 |
| | PFCKPOMF_01490 |
| | PFCKPOMF_01491 |
| | PFCKPOMF_01492 |
| | PFCKPOMF_01493 |
| | PFCKPOMF_01494 |
| | PFCKPOMF_01495 |
| | PFCKPOMF_01496 |
| PUL19 | PFCKPOMF_01573 |
| | PFCKPOMF_01574 |
| | PFCKPOMF_01575 |
| | PFCKPOMF_01576 |
| | PFCKPOMF_01577 |
| | PFCKPOMF_01578 |
| | PFCKPOMF_01579 |
| | PFCKPOMF_01580 |
| | PFCKPOMF_01581 |
| | PFCKPOMF_01582 |
| | PFCKPOMF_01583 |
| | PFCKPOMF_01584 |
| PUL18 | PFCKPOMF_01565 |
| | PFCKPOMF_01566 |
| | PFCKPOMF_01567 |
| | PFCKPOMF_01568 |
| | PFCKPOMF_01569 |
| | PFCKPOMF_01570 |
| | PFCKPOMF_01571 |
| PUL15 | PFCKPOMF_01243 |
| | PFCKPOMF_01244 |
| | PFCKPOMF_01245 |
| | PFCKPOMF_01246 |
| | PFCKPOMF_01247 |
| | PFCKPOMF_01248 |
| | PFCKPOMF_01249 |
| | PFCKPOMF_01250 |
| PUL22 (divergent SusCD) | PFCKPOMF_02332 |
| | PFCKPOMF_02333 |
| | PFCKPOMF_02334 |
| | PFCKPOMF_02335 |
| | PFCKPOMF_02336 |
| | PFCKPOMF_02337 |
| | PFCKPOMF_02338 |
| | PFCKPOMF_02339 |
| | PFCKPOMF_02340 |
| PUL16 (divergent SusD 86%) | PFCKPOMF_01326 |
| | PFCKPOMF_01327 |
| | PFCKPOMF_01328 |
| | PFCKPOMF_01329 |
| | PFCKPOMF_01330 |
| | PFCKPOMF_01331 |
| PUL10 (missing one SusCD, divergent GH53 89%) | PFCKPOMF_00908 |
| | PFCKPOMF_00909 |
| | PFCKPOMF_00910 |
| | PFCKPOMF_00911 |
| | PFCKPOMF_00912 |
| | PFCKPOMF_00913 |
| | PFCKPOMF_00914 |
| | PFCKPOMF_00915 |
| | PFCKPOMF_00916 |
| | PFCKPOMF_00917 |
| | PFCKPOMF_00918 |
| | PFCKPOMF_00919 |
| | PFCKPOMF_00920 |
| | PFCKPOMF_00921 |
| | PFCKPOMF_00922 |
| PUL3a (divergent SusCD) | PFCKPOMF_00392 |
| PUL3b (divergent SusD 78%, divergent GH43_4 84%, | PFCKPOMF_00393 |
| divergent GH43_5 80%) | |
| | PFCKPOMF_00394 |
| | PFCKPOMF_00395 |
| | PFCKPOMF_00396 |
| | PFCKPOMF_00397 |
| | PFCKPOMF_00398 |
| | PFCKPOMF_00399 |
| | PFCKPOMF_00400 |
| | PFCKPOMF_00401 |
| | PFCKPOMF_00402 |
| | PFCKPOMF_00403 |
| | PFCKPOMF_00404 |
| | PFCKPOMF_00405 |
| | PFCKPOMF_00406 |
| | PFCKPOMF_00407 |
| | PFCKPOMF_00408 |
| | PFCKPOMF_00409 |
| | PFCKPOMF_00410 |
| | PFCKPOMF_00411 |
| | PFCKPOMF_00412 |
| | PFCKPOMF_00413 |

TABLE C

| Prevotella copri BgF5_2 PUL | Locus Tags |
|---|---|
| PUL8 | BBPDHENA_01083 |
| | BBPDHENA_01084 |
| | BBPDHENA_01085 |
| | BBPDHENA_01086 |
| | BBPDHENA_01087 |
| | BBPDHENA_01088 |
| | BBPDHENA_01089 |
| | BBPDHENA_01090 |
| | BBPDHENA_01091 |
| | BBPDHENA_01092 |
| | BBPDHENA_01093 |
| PUL16 (divergent SusD 50%, divergent SusC 66%, no | BBPDHENA_01728 |
| GH28, divergent CE8 80%) | |
| | BBPDHENA_01729 |
| | BBPDHENA_01730 |
| | BBPDHENA_01731 |
| | BBPDHENA_01732 |
| | BBPDHENA_01733 |
| | BBPDHENA_01734 |
| | BBPDHENA_01735 |
| | BBPDHENA_01736 |
| | BBPDHENA_01737 |
| | BBPDHENA_01738 |
| | BBPDHENA_01739 |
| | BBPDHENA_01740 |
| PUL18 | BBPDHENA_01817 |
| | BBPDHENA_01818 |
| | BBPDHENA_01819 |
| | BBPDHENA_01820 |
| | BBPDHENA_01821 |
| | BBPDHENA_01822 |
| | BBPDHENA_01823 |
| | BBPDHENA_01824 |
| | BBPDHENA_01825 |
| | BBPDHENA_01826 |
| | BBPDHENA_01827 |
| | BBPDHENA_01828 |
| PUL17 | BBPDHENA_01809 |
| | BBPDHENA_01810 |
| | BBPDHENA_01811 |
| | BBPDHENA_01812 |
| | BBPDHENA_01813 |
| | BBPDHENA_01814 |
| | BBPDHENA_01815 |
| PUL14 | BBPDHENA_01487 |
| | BBPDHENA_01488 |
| | BBPDHENA_01489 |
| | BBPDHENA_01490 |
| | BBPDHENA_01491 |
| | BBPDHENA_01492 |
| | BBPDHENA_01493 |
| | BBPDHENA_01494 |
| PUL20 (divergent SusCD, divergent GH97_3) | BBPDHENA_02103 |
| | BBPDHENA_02104 |
| | BBPDHENA_02105 |
| | BBPDHENA_02106 |
| | BBPDHENA_02107 |
| | BBPDHENA_02108 |
| | BBPDHENA_02109 |
| | BBPDHENA_02110 |
| | BBPDHENA_02111 |
| PUL15 (divergent SusD 86%) | BBPDHENA_01570 |
| | BBPDHENA_01571 |
| | BBPDHENA_01572 |
| | BBPDHENA_01573 |
| | BBPDHENA_01574 |
| | BBPDHENA_01575 |
| PUL9 (missing one SusCD, divergent GH53 89%) | BBPDHENA_01151 |
| | BBPDHENA_01152 |
| | BBPDHENA_01153 |
| | BBPDHENA_01154 |
| | BBPDHENA_01155 |
| | BBPDHENA_01156 |
| | BBPDHENA_01157 |
| | BBPDHENA_01158 |
| | BBPDHENA_01159 |
| | BBPDHENA_01160 |
| | BBPDHENA_01161 |
| | BBPDHENA_01162 |
| | BBPDHENA_01163 |
| | BBPDHENA_01164 |
| | BBPDHENA_01165 |
| PUL2a (divergent SusCD) | BBPDHENA_00634 |
| PUL2b (divergent SusD 78%, divergent GH43_4 84%, | BBPDHENA_00635 |
| divergent GH43_5 80%) | |
| | BBPDHENA_00636 |
| | BBPDHENA_00637 |
| | BBPDHENA_00638 |
| | BBPDHENA_00639 |
| | BBPDHENA_00640 |
| | BBPDHENA_00641 |
| | BBPDHENA_00642 |
| | BBPDHENA_00643 |
| | BBPDHENA_00644 |
| | BBPDHENA_00645 |
| | BBPDHENA_00646 |
| | BBPDHENA_00647 |
| | BBPDHENA_00648 |
| | BBPDHENA_00649 |
| | BBPDHENA_00650 |
| | BBPDHENA_00651 |
| | BBPDHENA_00652 |
| | BBPDHENA_00653 |
| | BBPDHENA_00654 |
| | BBPDHENA_00655 |

TABLE D

| Prevotella copri Bg131 PUL | Locus Tag |
|---|---|
| PUL15 (divergent SusCD + GH10\|CBM4\|CBM4 | NJCFFJJN_02552 |
| fragmented to GH10[Fnc]) | |
| | NJCFFJJN_02553 |
| | NJCFFJJN_02554 |
| | NJCFFJJN_02555 |
| | NJCFFJJN_02556 |
| | NJCFFJJN_02557 |
| | NJCFFJJN_02558 |
| | NJCFFJJN_02559 |
| | NJCFFJJN_02560 |
| | NJCFFJJN_02561 |
| PUL6 + PUL7 (divergent SusCD, extra SusCD, divergent | NJCFFJJN_01898 |
| CE8 39%, no PL1) | |
| | NJCFFJJN_01899 |
| | NJCFFJJN_01900 |
| | NJCFFJJN_01901 |
| | NJCFFJJN_01902 |
| | NJCFFJJN_01903 |
| | NJCFFJJN_01904 |
| | NJCFFJJN_01905 |
| | NJCFFJJN_01907 |
| | NJCFFJJN_01908 |
| | NJCFFJJN_01909 |
| | NJCFFJJN_01910 |
| | NJCFFJJN_01911 |
| | NJCFFJJN_01912 |
| | NJCFFJJN_01913 |
| PUL5 (GH26 divergent 42% and with no GH5_4 and | NJCFFJJN_01811 |
| misplaced, missing one GH26, divergent SusCD and | |
| directed the opposite way) | |
| | NJCFFJJN_01812 |
| | NJCFFJJN_01813 |
| | NJCFFJJN_01814 |
| | NJCFFJJN_01815 |
| | NJCFFJJN_01816 |
| | NJCFFJJN_01817 |
| PUL3 (divergent SusCD) | NJCFFJJN_00575 |
| | NJCFFJJN_00576 |
| | NJCFFJJN_00577 |
| | NJCFFJJN_00578 |
| | NJCFFJJN_00579 |
| | NJCFFJJN_00580 |
| | NJCFFJJN_00581 |
| PUL30 | NJCFFJJN_03307 |
| | NJCFFJJN_03308 |
| | NJCFFJJN_03309 |
| | NJCFFJJN_03310 |
| | NJCFFJJN_03311 |
| | NJCFFJJN_03312 |
| PUL8 | NJCFFJJN_02065 |
| | NJCFFJJN_02066 |
| | NJCFFJJN_02067 |
| | NJCFFJJN_02068 |
| | NJCFFJJN_02069 |
| | NJCFFJJN_02070 |
| PUL14 (PL1 without CBM77, missing one SusCD) | NJCFFJJN_02485 |
| | NJCFFJJN_02486 |
| | NJCFFJJN_02487 |
| | NJCFFJJN_02488 |
| | NJCFFJJN_02489 |
| | NJCFFJJN_02490 |
| | NJCFFJJN_02491 |
| | NJCFFJJN_02492 |
| | NJCFFJJN_02493 |
| | NJCFFJJN_02494 |
| | NJCFFJJN_02495 |
| | NJCFFJJN_02496 |
| | NJCFFJJN_02497 |
| | NJCFFJJN_02498 |
| PUL27a (divergent SusCD) | NJCFFJJN_03225 |
| PUL27b (extra SusCD and divergent, divergent GH43_4 | NJCFFJJN_03226 |
| 71%, no GH51 - isolated) | |
| | NJCFFJJN_03227 |
| | NJCFFJJN_03228 |
| | NJCFFJJN_03229 |
| | NJCFFJJN_03230 |
| | NJCFFJJN_03231 |
| | NJCFFJJN_03232 |
| | NJCFFJJN_03233 |
| | NJCFFJJN_03234 |
| | NJCFFJJN_03235 |
| | NJCFFJJN_03236 |
| | NJCFFJJN_03237 |
| | NJCFFJJN_03238 |
| | NJCFFJJN_03239 |
| | NJCFFJJN_03240 |
| | NJCFFJJN_03241 |
| | NJCFFJJN_03242 |
| | NJCFFJJN_03243 |
| | NJCFFJJN_03244 |
| | NJCFFJJN_03245 |

In some aspects, the isolated strain of P. copri as disclosed herein comprises at least one polynucleotide sequence from P. copri of NRRL deposit no. xxxxx or yyyy or zzzz that enhances utilization of arabinoxylan, pectin, b-mannan, b-glucan, xylan, arabinoxylan, glucomannan, xyloglucan, b-1,3-glucan, pectin galactan, starch or arabinogalactan as provided in Table A. In some aspects, the current disclosure encompasses a composition comprising a carrier and an isolated strain of P. copri comprising at least two, at least three, at least 4, at least 5, at least 6, at least 7, at least 8, at least 9, at least 10 or at least 20, at least 30 or more of a polynucleotide sequence encoding a protein from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of a genome sequence deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2. In some aspects, the isolated strain comprises at least two, at least three, at least 4, at least 5, at least 6, at least 7, at least 8, at least 9, at least 10 or at least 20, at least 30 or more of polynucleotide sequences from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of P. copri strain NRRL deposit no. xxxxx or yyyyy or zzzzz.

In some aspects, the current disclosure also encompasses composition comprising a carrier and a probiotic strain comprising at least one polynucleotide sequence from P. copri of NRRL deposit no. xxxxx or yyyy or zzzz that enhances utilization of arabinoxylan, pectin, b-mannan, b-glucan, xylan, arabinoxylan, glucomannan, xyloglucan, b-1,3-glucan, pectin galactan, starch or arabinogalactan as provided in Table A. In some aspects, the current disclosure encompasses a composition comprising a carrier and a probiotic strain comprising at least two, at least three, at least 4, at least 5, at least 6, at least 7, at least 8, at least 9, at least 10 or at least 20, at least 30 or more of a polynucleotide sequence encoding a protein from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of a genome sequence deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2. In some aspects, the probiotic strain comprises at least two, at least three, at least 4, at least 5, at least 6, at least 7, at least 8, at least 9, at least 10 or at least 20, at least 30 or more of polynucleotide sequences from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of P. copri strain NRRL deposit no. xxxxx or yyyyy or zzzzz. In some aspects the probiotic bacterial strain comprises a genome sequence at least about 90% identical to any one of the sequences deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2. In some aspects, the probiotic bacterial strain has a genome at least about 90%, or at least about 91%, or at least about 92%, or at least about 93%, or at least about 94%, or at least about 95%, or at least about 96%, or at least about 97%, or at least about 98%, or at least about 99% or more to the genome of any one of P. copri strain NRRL deposit no. xxxxx or yyyyy or zzzzz or the genome as deposition at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2.

In some aspects, the current disclosure also encompasses composition comprising a carrier and an engineered probiotic strain comprising at least one polynucleotide sequence from P. copri of NRRL deposit no. xxxxx or yyyy or zzzz that enhances utilization of arabinoxylan, pectin, b-mannan, b-glucan, xylan, arabinoxylan, glucomannan, xyloglucan, b-1,3-glucan, pectin galactan, starch or arabinogalactan as provided in Table A. In some aspects, the current disclosure encompasses a composition comprising a carrier and an engineered probiotic strain comprising at least two, at least three, at least 4, at least 5, at least 6, at least 7, at least 8, at least 9, at least 10 or at least 20, at least 30 or more of a polynucleotide sequence encoding a protein from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of a genome sequence deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2. In some aspects, the engineered probiotic strain comprises at least two, at least three, at least 4, at least 5, at least 6, at least 7, at least 8, at least 9, at least 10 or at least 20, at least 30 or more of polynucleotide sequences from one or more of the polysaccharide utilization loci PUL3a, PUL3b, PUL9, PUL10, PUL15, PUL16, PUL17, PUL18, PUL 19, PUL20, PUL22, or PUL30 or any combination thereof, of P. copri strain NRRL deposit no. xxxxx or yyyyy or zzzzz. In some aspects the engineered probiotic strain comprises a genome sequence at least about 90% identical to any one of the sequences deposited at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2. In some aspects, the engineered probiotic bacterial strain has a genome at least about 90%, or at least about 91%, or at least about 92%, or at least about 93%, or at least about 94%, or at least about 95%, or at least about 96%, or at least about 97%, or at least about 98%, or at least about 99% or more to the genome of any one of P. copri strain NRRL deposit no. xxxxx or yyyyy or zzzzz or the genome as deposition at the European Nucleotide Archive with accession numbers ERZ17359655a corresponding to Prevotella copri Bg131, ERZ17359674 corresponding to Prevotella copri BgF5_2 and ERZ17359677 corresponding to Prevotella copri BgD5_2.

In some aspects, the current disclosure encompasses compositions comprising more than about 10², or more than about 10³, or more than about 10⁵, or more than about 10⁷, or more than about 10⁹, or more than about 10¹¹, or more than about 10¹³ cfu per gram of P. copri (NRRL deposit #XXXX or YYYY or both). In some aspects, the composition may comprise more than about 10², or more than about 10³, or more than about 10⁵, or more than about 10⁷, or more than about 10⁹, or more than about 10¹¹, or more than about 10¹³ cfu of per gram of one or more isolated P. copri strains as disclosed herein. In some aspects, the composition may comprise more than about 10², or more than about 10³, or more than about 10⁵, or more than about 10⁷, or more than about 10⁹, or more than about 10¹¹, or more than about 10¹³ cfu of per gram of an engineered probiotic strain as disclosed herein. In some aspects, the composition may comprise more than about 10², or more than about 10³, or more than about 10⁵, or more than about 10⁷, or more than about 10⁹, or more than about 10¹¹, or more than about 10¹³ cfu per gram of a combination of strains comprising at least one of the DNA sequences as disclosed herein. In some aspects, the compositions disclosed herein comprise at least one suitable carrier.

In some aspects, the composition may comprise viable P. copri, engineered probiotic cells, or combination thereof. In some aspects, the composition may comprise a mixture of viable and non-viable cells. In some aspects, the compositions disclosed herein comprise at least one suitable carrier.

In some aspects the composition may further comprise additional bacterial strains thus forming a mixture of probiotic strains. As used herein, the term “probiotic” refers to any live microorganism which when administered to a subject in adequate amounts confers a health benefit. In some aspect, the compositions of the current disclosure may comprise an isolated P. copri or engineered probiotic strain as disclosed herein and an additional probiotic strain. In some aspects the additional probiotic strains may include one of more of naturally occurring or engineered strains, particular but non-limiting examples of which include Arthrobacter agilis, Arthrobacter citreus, Arthrobacter globiformis, Arthrobacter leuteus. Arthrobacter simplex, Azotobacter chroococcum, Azotobacter paspali, Azospirillum brasiliencise, Azospriliium lipoferum, Bacillus brevis, Bacillus macerans, Bacillus pumilus, Bacillus polymyxa, Bacillus subtilis, Bacteroides lipolyticum, Bacteroides succinogenes, Brevibacterium lipolyticum, Brevibacterium stationis, Bacillus laterosporus, Bacillus bifidum, Bacillus laterosporus, Bifidophilus infantis, Streptococcus thermophilous, Bifidophilus longum, Bifidobacterium infantis, Bifidobacteria animalis, Bifidobacteria bifidus, Bifidobacteria breve, Bifidobacteria longum, Kurtha zopfil, Lactobacillus paracasein, Lactobacillus acidophilus, Lactobacillus planetarium, Lactobacillus salivarius, Lactobacillus rueteri, Lactobacillus bulgaricus, Lactobacillus helveticus, Lactobacillus casei, Lactobacillus rhamnosus. Lactobacillus sporogenes, Lactococcus lactis, Myrothecium verrucaris, Prevotella spp., Pseudomonas calcis, Pseudomonas dentrificans, Pseudomonas flourescens, Pseudomonas glathei, Phanerochaete chrysosporium, Saccharomyces boulardii, Streptmyces fradiae, Streptomyces cellulosae, Stretpomyces griseoflavus and combinations thereof.

In some aspects the formulation may comprise a viable mixture of probiotic cells. In some aspects the formulation may comprise non-viable mixture of probiotic cells. In some aspects the formulation may comprise a mixture of viable and non-viable mixture of pro-biotic cells.

In some aspects, the compositions as disclosed herein further comprise a suitable carrier. “Carrier” is understood as any substance that facilitates the growth, transportation and/or administration of the strains of the present invention. Depending on the purpose and/or use to which said strains are intended for, the “carriers” could be of different nature. The present invention relates to pharmaceutically acceptable “carriers” such as those commonly associated to capsules, tablets or powder, as well as a “carriers” formed by ingredients or food products. In some aspects, the carrier is an ingestible carrier. Non-limiting examples of ingestible carriers include milk components, baby formula, baby food including but not limited to F-75 or F-100 formulas used for the management of malnutrition, human milk oligosaccharides, breast milk, sugar, flavor enhancers.

In some aspects the formulation may further comprise a prebiotic material, an excipient, an adjuvant, stabilizers, a biological compound, dietary supplements, proteins, a vitamin, a drug, a vaccine or a combination thereof. “Prebiotic” means one or more non-digestible food substance that promotes the growth of health beneficial micro-organisms, or probiotics in the intestines. They are not broken down in the stomach, or upper intestine or absorbed in the GI tract of the person ingesting them, but they are fermented by the gastrointestinal microbiota or by probiotics. In some aspects, the current disclosure also encompasses synbiotic formulations comprising the at least a probiotic strain as disclosed herein. Synbiotics refer to nutritional supplements combining probiotics and prebiotics in a form of synergism. Non-limiting examples of prebiotics include acacia gum, alpha glucan, arabinogalactans, beta glucan, dextrans, fructooligosaccharides, fucosyllactose, galactooligosaccharides, galactomannans, gentiooligosaccharides, glucooligosaccharides, guar gum, inulin, isomaltooligosaccharides, lactoneotetraose, lactosucrose, lactulose, levan, maltodextrins, milk oligosaccharides, partially hydrolyzed guar gum, pecticoligosaccharides, resistant starches, retrograded starch, sialooligosaccharides, sialyllactose, soyoligosaccharides, sugar alcohols, xylooligosaccharides, or their hydrolysates, or combinations thereof. Non-limiting examples of proteins include dairy based proteins, plant-based proteins, animal-based proteins and artificial proteins. Dairy based proteins include, for example, casein, caseinates (e.g., all forms including sodium, calcium, potassium caseinates), casein hydrolysates, whey (e.g., all forms including concentrate, isolate, demineralized), whey hydrolysates, milk protein concentrate, and milk protein isolate. Plant based proteins include, for example, soy protein (e.g., all forms including concentrate and isolate), pea protein (e.g., all forms including concentrate and isolate), canola protein (e.g., all forms including concentrate and isolate), other plant proteins that commercially are wheat and fractionated wheat proteins, corn and it fractions including zein, rice, oat, potato, peanut, green pea powder, green bean powder, and any proteins derived from beans, lentils, and pulses. As used herein the term “vitamin” is understood to include any of various fat-soluble or water-soluble organic substances (non-limiting examples include vitamin A, Vitamin B1 (thiamine), Vitamin B2 (riboflavin), Vitamin B3 (niacin or niacinamide), Vitamin B5 (pantothenic acid), Vitamin B6 (pyridoxine, pyridoxal, or pyridoxamine, or pyridoxine hydrochloride), Vitamin B7 (biotin), Vitamin B9 (folic acid), and Vitamin B12 (various cobalamins; commonly cyanocobalamin in vitamin supplements), vitamin C, vitamin D, vitamin E, vitamin K, folic acid and biotin) essential in minute amounts for normal growth and activity of the body and obtained naturally from plant and animal foods or synthetically made, pro-vitamins, derivatives, analogs. Non-limiting examples of excipients include binders, emulsifiers, diluents, fillers, disintegrants, effervescent disintegration agents, preservatives, antioxidants, flavor-modifying agents, lubricants and glidants, dispersants, coloring agents, pH modifiers, chelating agents, and release-controlling polymers. Non-limiting list of adjuvants include potassium alum, aluminum hydroxide, aluminum phosphate, calcium phosphate hydroxide, paraffin oil, adjuvant 65, killed bacteria of the species Bordetella pertussis, Mycobacterium bovis, toxoids, plant saponins from quillaja and soybean, cytokines: IL-1, IL-2, IL-1, Freund's complete adjuvant, Freund's incomplete adjuvant and squalene.

In some aspects, the current disclosure also encompasses synbiotic formulations comprising the compositions as disclosed herein and further comprising a food formulation. In some aspects, any suitable food formulation can be combined with the disclosed compositions.

In some aspects, the food formulation as disclosed herein is an edible composition that impacts the subject's gut microbiota in a manner to modulate expression of nucleic acids encoding proteins in particular enzyme families, such that physiological parameters of the subject are improved, e.g., ponderal growth or rate of ponderal growth. Components of the food formulation and some exemplary formulations are provided below in sections a-f. In some aspects, the food formulations as disclosed herein can be used with the probiotic compositions disclosed herein. However, the current disclosure also encompasses the use of these food formulation without the use of additional compositions comprising a probiotic bacterial strain, but to promote the beneficial functions of the target P. copri strains already present in a subject's microbiota.

### (a) Food Formulation Comprising Chickpea Flour, Peanut Flour, Soy Flour, Raw Banana

In one aspect, a food formulation of the present disclosure comprises chickpea flour, peanut flour, soy flour, and raw banana, wherein the chickpea flour, the peanut flour, the soy flour, and the raw banana provide at least 8.5 g of protein per 100 g of the food formulation. In preferred aspects, the food formulation contains no cow's milk or powdered cow's milk, or no milk or powdered milk of any kind, or no milk, powdered milk, or milk product of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (a) no seed, nuts, and nut butter, and/or (b) no cocoa nibs, cocoa powder or chocolate, and/or (c) no rice flour and lentil flour, and/or (d) no dried fruit. In another example, food formulations of the present disclosure comprising chickpea flour, peanut flour, soy flour, and raw banana may contain no milk or powdered milk of any kind and (a) no seed, nuts, and nut butter, and/or (b) no cocoa nibs, cocoa powder or chocolate, and/or (c) no rice flour and lentil flour, and/or (d) no dried fruit.

In some aspects, the chickpea flour, the peanut flour, the soy flour, and the raw banana, in total, provide 8.5 g to about 40 g of protein per 100 g of the food formulation. In some aspects, the chickpea flour, the peanut flour, the soy flour, and the raw banana, in total, provide about 9 g to about 40 g of protein per 100 g of the food formulation. In some aspects, the chickpea flour, the peanut flour, the soy flour, and the raw banana, in total, provide about 10 g to about 40 g of protein per 100 g of the food formulation. In some aspects, the chickpea flour, the peanut flour, the soy flour, and the raw banana, in total, provide about 11 g to about 40 g of protein per 100 g of the food formulation. In some aspects, the chickpea flour, the peanut flour, the soy flour, and the raw banana, in total, provide about 9 g to about 30 g of protein per 100 g of the food formulation. In some aspects, the chickpea flour, the peanut flour, the soy flour, and the raw banana, in total, provide about 10 g to about 28 g of protein per 100 g of the food formulation. In some aspects, the chickpea flour, the peanut flour, the soy flour, and the raw banana, in total, provide about 11 g to about 26 g of protein per 100 g of the food formulation. In some aspects, the chickpea flour, the peanut flour, the soy flour, and the raw banana, in total, provide about 12 g to about 24 g of protein per 100 g of the food formulation. In some aspects, the chickpea flour, the peanut flour, the soy flour, and the raw banana, in total, provide about 12 g to about 14 g of protein per 100 g of the food formulation. In some aspects, the chickpea flour, the peanut flour, the soy flour, and the raw banana, in total, provide about 13 g to about 15 g of protein per 100 g of the food formulation. In other aspects, the chickpea flour, the peanut flour, the soy flour, and the raw banana, in total, provide 8.5 g, about 9 g, about 9.5 g, about 10 g, about 10.5 g, about 11 g, about 11.5 g, about 12 g, about 12.5 g, about 13 g, about 13.5 g, about 14 g, about 14.5 g, or about 15 g, about 15.5 g, about 16 g, about 16.5 g, about 17 g, about 17.5 g, about 18 g, about 18.5 g, about 19 g, about 19.5, about 20 g, about 20.5, about 21 g about 21.5, about 22 g about 22.5, about 23 g, about 23.5, about 24 g, about 24.5, about 25 g, about 25.5, about 26 g, about 26.5, about 27 g, about 27.5, about 28 g, about 28.5, about 29 g, about 29.5, about 30 g, about 30.5, about 31 g, about 31.5, about 32 g, about 32.5 g, about 33, about 33.5 g, about 34 g, about 34.5 g, about 35 g, about 35.5 g, about 36, about 36.5 g, about 37 g, about 37.5 g, about 38 g, about 38.5 g, about 39 g, about 39.5 g, about 40 g of protein per 100 g of the food formulation.

In each of the above aspects, the weight ratio of the chickpea flour to the peanut flour to the soy flour to the raw banana may vary. Typically, chickpea flour has about 20%-40% protein by weight, peanut flour has about 20%-50% protein by weight, soy flour has about 20%-50% protein by weight, and raw banana has about 1-30% protein by weight. The weight percentages of protein in each ingredient may vary however, depending upon the varietal of plant and, in the case of the flours, the method used to manufacture the flour. In some aspects, the weight ratio is about 1:about 1:about 0.8:about 1.9, respectively (chickpea flour:peanut flour:soy flour:raw banana), or a weight ratio adjusted as needed to reflect differences in the ingredients.

In an exemplary aspect, a food formulation of the present disclosure comprises about 9-11 g of chickpea flour, about 9-11 g of peanut flour, about 7-9 g of soy flour, and about 17-21 g of raw banana. In preferred aspects, the food formulation contains no cow's milk or powdered cow's milk, or no milk or powdered milk of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (i) no seed, nuts, and nut butter, and/or (ii) no cocoa nibs, cocoa powder or chocolate, and/or (iii) no rice flour and lentil flour, and/or (iv) no dried fruit. In another example, food formulations of the present disclosure comprising chickpea flour, peanut flour, soy flour, and raw banana may contain no milk or powdered milk of any kind and (i) no seed, nuts, and nut butter, and/or (ii) no cocoa nibs, cocoa powder or chocolate, and/or (iii) no rice flour and lentil flour, and/or (iv) no dried fruit.

In another exemplary aspect, a food formulation of the present disclosure comprises about 10 g of chickpea flour, about 10 g of peanut flour, about 8 g of soy flour, and about 19 g of raw banana. In preferred aspects, the food formulation contains no cow's milk or powdered cow's milk, or no milk or powdered milk of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (i) no seed, nuts, and nut butter, and/or (ii) no cocoa nibs, cocoa powder or chocolate, and/or (iii) no rice flour and lentil flour, and/or (iv) no dried fruit. In another example, food formulations of the present disclosure comprising chickpea flour, peanut flour, soy flour, and raw banana may contain no milk or powdered milk of any kind and (i) no seed, nuts, and nut butter, and/or (ii) no cocoa nibs, cocoa powder or chocolate, and/or (iii) no rice flour and lentil flour, and/or (iv) no dried fruit.

In another exemplary aspect, a food formulation of the present disclosure comprises about 11.9 g of chickpea flour, about 10 g of peanut flour, about 13 g of soy flour, and about 15 g of raw banana. In preferred aspects, the food formulation contains no cow's milk or powdered cow's milk, or no milk or powdered milk of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (i) no seed, nuts, and nut butter, and/or (ii) no cocoa nibs, cocoa powder or chocolate, and/or (iii) no rice flour and lentil flour, and/or (iv) no dried fruit. In another example, food formulations of the present disclosure comprising chickpea flour, peanut flour, soy flour, and raw banana may contain no milk or powdered milk of any kind and (i) no seed, nuts, and nut butter, and/or (ii) no cocoa nibs, cocoa powder or chocolate, and/or (iii) no rice flour and lentil flour, and/or (iv) no dried fruit.

In another exemplary aspect, a food formulation of the present disclosure comprises about 13 g of chickpea flour, about 13 g of peanut flour, about 11 g of soy flour, and about 14.90 g of raw banana. In preferred aspects, the food formulation contains no cow's milk or powdered cow's milk, or no milk or powdered milk of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (i) no seed, nuts, and nut butter, and/or (ii) no cocoa nibs, cocoa powder or chocolate, and/or (iii) no rice flour and lentil flour, and/or (iv) no dried fruit. In another example, food formulations of the present disclosure comprising chickpea flour, peanut flour, soy flour, and raw banana may contain no milk or powdered milk of any kind and (i) no seed, nuts, and nut butter, and/or (ii) no cocoa nibs, cocoa powder or chocolate, and/or (iii) no rice flour and lentil flour, and/or (iv) no dried fruit.

In another exemplary aspect, a food formulation of the present disclosure comprises about 8.68 g of chickpea flour, about 13.87 g of peanut flour, about 16.30 g of soy flour, and about 8.75 g of raw banana. In preferred aspects, the food formulation contains no cow's milk or powdered cow's milk, or no milk or powdered milk of any kind. In still further aspects, the food formulation also contains no seeds, nuts, nut butters, dried fruit, cocoa nibs, cocoa powder, chocolate, rice flour, lentil flour, or any combination thereof. For example, food formulations of the present disclosure comprising chickpea flour, peanut flour, soy flour, and raw banana may contain no cow's milk or powdered cow's milk and (i) no seed, nuts, and nut butter, and/or (ii) no cocoa nibs, cocoa powder or chocolate, and/or (iii) no rice flour and lentil flour, and/or (iv) no dried fruit. In another example, food formulations of the present disclosure comprising chickpea flour, peanut flour, soy flour, and raw banana may contain no milk or powdered milk of any kind and (i) no seed, nuts, and nut butter, and/or (ii) no cocoa nibs, cocoa powder or chocolate, and/or (iii) no rice flour and lentil flour, and/or (iv) no dried fruit.

### (b) Food Formulation Comprising Glycan Equivalents of Chickpea Flour, Peanut Flour, Soy Flour, Raw Banana

In another aspect, a food formulation of the present disclosure is a food formulation of (a), wherein some or all the chickpea flour, the peanut flour, the soy flour, and/or the raw banana is replaced with a glycan equivalent thereof. As used herein, a “glycan equivalent” refers to a food formulation with a similar glycan content. The term “similar” generally refers to a range of numerical values, for instance, ±0.5-1%, ±1-5% or ±5-10% of the recited value, that one would consider equivalent to the recited value, for example, having the same function or result. Because a glycan equivalent has a similar glycan content to the ingredient it is replacing, it may be substituted about 1:1. For instance, if 3 g of chickpea flour is to be replaced with a glycan equivalent thereof, one of skill in the art would use about 3 g of the chickpea glycan equivalent. A glycan equivalent may be defined in terms of its monosaccharide content and optionally by an analysis of the glycosidic linkages. Methods for measuring monosaccharide content and analyzing glycosidic linkages are known in the art.

In some aspects, some or all the chickpea flour is replaced with a glycan equivalent of chickpea flour. For instance, a food formulation of (a) may comprise a glycan equivalent of about 0.5 g or more of chickpea flour. In another example, a food formulation of (a) may comprise a glycan equivalent of about 1 g, about 2 g, about 3 g, about 4 g, about 5 g, about 6 g, about 7 g, about 8 g, about 9 g, or about 10 g, or about 11 g, or about 12 g, or about 13 g, or about 14 g, or about 15 g of chickpea flour. In another example, a food formulation of (a) may comprise a glycan equivalent of about 0.1 g to about 15 g of chickpea flour, or about 0.5 to about 5 g of chickpea flour. In another example, a food formulation of (a) may comprise a glycan equivalent of about 1 g to about 15 g of chickpea flour, or about 1 g to about 5 g of chickpea flour, or about 2.5 g to about 7.5 g of chickpea flour, to about 5 g to about 15 g of chickpea flour. In further aspects, some or all the peanut flour is also replaced with a glycan equivalent of peanut flour, some or all the soy flour is also replaced with a glycan equivalent of soy flour, and/or some or all the raw banana is also replaced with a glycan equivalent of raw banana.

In some aspects, some or all the peanut flour is replaced with a glycan equivalent of peanut flour. For instance, a food formulation of (a) may comprise a glycan equivalent of about 0.5 g or more of peanut flour. In another example, a food formulation of (a) may comprise a glycan equivalent of about 1 g, about 2 g, about 3 g, about 4 g, about 5 g, about 6 g, about 7 g, about 8 g, about 9 g, or about 10 g, or about 11 g, or about 12 g, or about 13 g, or about 14 g, or about 15 g of peanut flour. In another example, a food formulation of Section I (a) may comprise a glycan 15 g of peanut flour. In another example, a food formulation of Section I (a) may comprise a glycan equivalent of about 0.1 g to about 15 g of peanut flour, or about 0.5 to about 5 g of peanut flour. In another example, a food formulation of (a) may comprise a glycan equivalent of about 1 g to about 10 g of peanut flour, or about 1 g to about 15 g of peanut flour, or about 2.5 g to about 12.5 g of peanut flour, to about 5 g to about 10 g of peanut flour. In further aspects, some or all the chickpea flour is also replaced with a glycan equivalent of chickpea flour, some or all the soy flour is also replaced with a glycan equivalent of soy flour, and/or some or all the raw banana is also replaced with a glycan equivalent of raw banana.

In some aspects, some or all the soy flour is replaced with a glycan equivalent of soy flour. For instance, a food formulation of (a) may comprise a glycan equivalent of about 0.5 g or more of soy flour. In another example, a food formulation of (a) may comprise a glycan equivalent of about 1 g, about 2 g, about 3 g, about 4 g, about 5 g, about 6 g, about 7 g, or about 8 g, or about 9 g, or about 10 g, or about 11 g, or about 12 g, or about 13 g, or about 14 g, or about 15 g of soy flour. In another example, a food formulation of (a) may comprise a glycan equivalent of about 0.1 g to about 15 g of soy flour, or about 0.5 to about 10 g of soy flour. In another example, a food formulation of (a) may comprise a glycan equivalent of about 1 g to about 15 g of soy flour, or about 1 g to about 5 g of soy flour, or about 2 g to about 7.5 g of soy flour, to about 10 g to about 15 g of soy flour. In further aspects, some or all the chickpea flour is also replaced with a glycan equivalent of chickpea flour, some or all the peanut flour is also replaced with a glycan equivalent of peanut flour, and/or some or all the raw banana is also replaced with a glycan equivalent of raw banana.

In some aspects, some or all the raw banana is replaced with a glycan equivalent of raw banana. For instance, a food formulation of (a) may comprise a glycan equivalent of about 0.5 g or more of raw banana. In another example, a food formulation of (a) may comprise a glycan equivalent of about 1 g, about 2 g, about 3 g, about 4 g, about 5 g, about 6 g, about 7 g, about 8 g of raw banana, about 9 g of raw banana, about 10 g of raw banana, about 11 g of raw banana, about 12 g of raw banana, about 13 g of raw banana, about 14 g of raw banana, about 15 g of raw banana, about 16 g of raw banana, about 17 g of raw banana, about 18 g of raw banana, or about 19 g of raw banana. In another example, a food formulation of (a) may comprise a glycan equivalent of about 0.1 g to about 8 g of raw banana, or about 0.5 to about 5 g of raw banana. In another example, a food formulation of (a) may comprise a glycan equivalent of about 1 g to about 8 g of raw banana, or about 1 g to about 4 g of raw banana, or about 2 g to about 6 g of raw banana, to about 4 g to about 8 g of raw banana. In further aspects, some or all the chickpea flour is also replaced with a glycan equivalent of chickpea flour, some or all the peanut flour is also replaced with a glycan equivalent of peanut flour, and/or some or all the soy flour is also replaced with a glycan equivalent of soy flour.

### (c) Micronutrient Premix

A micronutrient premix in a food formulation of the present disclosure is present in an amount that provides at least 60% of the recommended daily allowance (RDA), for a given age group, of minimally vitamin A, vitamin C, vitamin D, vitamin E, vitamin B, calcium, copper, iron, magnesium, manganese, phosphorus, potassium, and zinc. The RDA of vitamin A, vitamin C, vitamin D, vitamin E, vitamin B, calcium, copper, iron, magnesium, manganese, phosphorus, potassium, and zinc, for various age groups, is known in the art. Given that different age groups may have different RDA's, it will be appreciated by a person of skill in the art that certain food formulations may not be suitable for subjects of all ages. For example, a food formulation with 60% of the Vitamin C RDA for a subject 7-12 months in age (e.g., 40 mg) will not contain at least 60% of the Vitamin C RDA for a subject 21 years of age (e.g., 75-90 mg). The term “vitamin “B,” as used herein, is inclusive of all B vitamins, unless otherwise specified. Although food formulations of the present disclosure are described as comprising a micronutrient premix, the addition of each vitamin and mineral separately, or the use of multiple premixes, is also contemplated and encompassed by the aspects described herein. Similarly, in alternative aspects, the micronutrient premix can be formulated separately and administered as a distinct food formulation in conjunction with a food formulation comprising chickpea flour or a glycan equivalent thereof, peanut flour or a glycan equivalent thereof, soy flour or a glycan equivalent thereof, raw banana or a glycan equivalent thereof.

In various aspects, a micronutrient premix provides at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% of the recommended daily allowance (RDA), for a given age group, of minimally vitamin A, vitamin B, vitamin C, vitamin D, vitamin E, calcium, copper, iron, magnesium, manganese, phosphorous, potassium and zinc. In certain aspects, a micronutrient premix provides more than 100% of the RDA, for a given age group, of minimally vitamin A, vitamin B, vitamin C, vitamin D, vitamin E, calcium, copper, iron, magnesium, manganese, phosphorous, potassium and zinc. In a specific aspect, the micronutrient premix provides at least 75% of the recommended daily allowance (RDA), for a given age group, of minimally vitamins A, C, D and E, all B vitamins, calcium, copper, iron, magnesium, manganese, phosphorous, potassium and zinc. The RDA of vitamins and minerals for different age groups is well known in the art.

In a specific aspect, a micronutrient premix provides at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 77%, at least 78%, at least 79%, or at least 80% of the recommended daily allowance (RDA) for children aged 12-24 months of vitamin A, vitamin B, vitamin C, vitamin D, vitamin E, calcium, copper, iron, magnesium, manganese, phosphorous, potassium and zinc.

In another specific aspect, the micronutrient premix provides at least 70% of the recommended daily allowance (RDA) for children aged 12-24 months of minimally vitamin A, vitamin B, vitamin C, vitamin D, vitamin E, calcium, copper, iron, magnesium, manganese, phosphorous, potassium and zinc.

In another specific aspect, the micronutrient premix provides at least 75% of the recommended daily allowance (RDA) for children aged 12-24 months of minimally vitamin A, vitamin B, vitamin C, vitamin D, vitamin E, calcium, copper, iron, magnesium, manganese, phosphorous, potassium and zinc.

A micronutrient premix may further comprise vitamins and minerals in addition to the vitamin A, vitamin B, vitamin C, vitamin D, vitamin E, calcium, copper, iron, magnesium, manganese, phosphorous, potassium and zinc.

In an exemplary aspect, a food formulation of the present disclosure contains vitamin A, vitamin C, vitamin D, vitamin E, vitamin B, calcium, copper, iron, magnesium, phosphorus, potassium, and zinc in the amounts listed in Table E and Table F. In a preferred aspect, a food formulation of the present disclosure contains the nutrients of Table E in the amounts listed in Table E. In another preferred aspect, a food formulation of the present disclosure contains the nutrients of Table F in the amounts listed in Table F. In yet another preferred aspect, a food formulation of the present disclosure contains the nutrients of both Table A and Table B, in the amounts listed in Table E and Table F respectively.

TABLE E
Vitamin Premix

| | | | Units of |
|---|---|---|---|
| | | | Measurement per |
| | Minimum | Maximum | gram of the |
| Nutrients | Amount | Amount | Vitamin Premix |
| Vitamin A | 12655.013 | 16170.294 | IU |
| Thiamine | 6.765 | 8.644 | mg |
| Mononitrate | | | |
| Vitamin B12 | 11.700 | 17.550 | mcg |
| Vitamin B2 - | 5.485 | 7.008 | mg |
| Riboflavin | | | |
| Pyridoxine | 6.153 | 7.863 | mg |
| Hydrochloride | | | |
| Vitamin C | 236.250 | 301.875 | mg |
| Sodium | 29.213 | 37.327 | mg |
| Calcium | 20.798 | 26.574 | mg |
| D-Pantothenate | | | |
| Vitamin D3 | 7593.960 | 9703.599 | IU |
| Vitamin E | 120.690 | 154.215 | IU |
| (as E Acetate) | | | |
| Folic acid | 2531.007 | 3234.065 | mcg |
| Vitamin K1 | 405.009 | 584.991 | mcg |
| Niacinamide | 60.750 | 77.625 | mg |

For a 100 g food formulation, 160 mg of the Vitamin Premix is used. Accordingly, to calculate the amount of a given mineral in a 100 g food formulation, the amounts listed above are multiplied by 160.

In an exemplary aspect, a food formulation of the present disclosure contains the micronutrients in Table F, in the amounts in Table F.

TABLE F
Mineral Premix

| | Minimum | Maximum | Units of Measurement per |
|---|---|---|---|
| Nutrients | Amount | Amount | gram of the mineral premix |
| Calcium | 170.000 | 216.000 | Mg |
| Phosphorus | 93.000 | 118.000 | Mg |
| Calcium | 0.000 | 0.000 | Q.S. |
| Copper | 0.181 | 0.231 | Mg |
| Iodine | 52.945 | 67.652 | Mcg |
| Iron | 3.169 | 4.049 | Mg |
| Magnesium | 27.163 | 34.708 | mg |
| Manganese | 0.543 | 0.694 | mg |
| Potassium (K) | 89.342 | 114.159 | Mg |
| Selenium | 11.770 | 15.040 | Mcg |
| Zinc | 2.415 | 3.085 | Mg |

For a 100 g food formulation, 2.982 g of the Mineral Premix is used. Accordingly, to calculate the amount of a given mineral in a 100 g food formulation, the amounts listed above are multiplied by 2.982.

For a 100 g food formulation, 2.982 g of the Mineral Premix is used. Accordingly, to calculate the amount of a given mineral in a 100 g food formulation, the amounts listed above are multiplied by 2.982.

### (d) Macronutrient Content

A micronutrient premix in a composition of the present disclosure is present in an amount that provides at least 60% of the recommended daily allowance (RDA), for a given age group, of minimally vitamin A, vitamin C, vitamin D, vitamin E, vitamin B, calcium, copper, iron, magnesium, manganese, phosphorus, potassium, and zinc. The RDA of vitamin A, vitamin C, vitamin D, vitamin E, vitamin B, calcium, copper, iron, magnesium, manganese, phosphorus, potassium, and zinc, for various age groups, is known in the art. Given that different age groups may have different RDA's, it will be appreciated by a person of skill in the art that certain compositions may not be suitable for subjects of all ages. For example, a composition with 60% of the Vitamin C RDA for a subject 7-12 months in age (e.g., 40 mg) will not contain at least 60% of the Vitamin C RDA for a subject 21 years of age (e.g., 75-90 mg). The term “vitamin “B,” as used herein, is inclusive of all B vitamins, unless otherwise specified. Although compositions of the present disclosure are described as comprising a micronutrient premix, the addition of each vitamin and mineral separately, or the use of multiple premixes, is also contemplated and encompassed by the embodiments described herein. Similarly, in alternative embodiments, the micronutrient premix can be formulated separately and administered as a distinct composition in conjunction with a composition comprising chickpea flour or a glycan equivalent thereof, peanut flour or a glycan equivalent thereof, soy flour or a glycan equivalent thereof, raw banana or a glycan equivalent thereof. [0083] In various embodiments, a micronutrient premix provides at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 77%, at least 78%, at least 79%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99%, or at least 100% of the recommended daily allowance (RDA), for a given age group, of minimally vitamin A, vitamin B, vitamin C, vitamin D, vitamin E, calcium, copper, iron, magnesium, manganese, phosphorous, potassium and zinc. In certain embodiments, a micronutrient premix provides more than 100% of the RDA, for a given age group, of minimally vitamin A, vitamin B, vitamin C, vitamin D, vitamin E, calcium, copper, iron, magnesium, manganese, phosphorous, potassium and zinc. In a specific embodiment, the micronutrient premix provides at least 75% of the recommended daily allowance (RDA), for a given age group, of minimally vitamins A, C, D and E, all B vitamins, calcium, copper, iron, magnesium, manganese, phosphorous, potassium and zinc. The RDA of vitamins and minerals for different age groups is well known in the art.

In a specific embodiment, a micronutrient premix provides at least 60%, at least 61%, at least 62%, at least 63%, at least 64%, at least 65%, at least 66%, at least 67%, at least 68%, at least 69%, at least 70%, at least 71%, at least 72%, at least 73%, at least 74%, at least 75%, at least 76%, at least 77%, at least 77%, at least 78%, at least 79%, or at least 80% of the recommended daily allowance (RDA) for children aged 12-18 months of vitamin A, vitamin B, vitamin C, vitamin D, vitamin E, calcium, copper, iron, magnesium, manganese, phosphorous, potassium and zinc.

In another specific embodiment, the micronutrient premix provides at least 70% of the recommended daily allowance (RDA) for children aged 12-18 months of minimally vitamin A, vitamin B, vitamin C, vitamin D, vitamin E, calcium, copper, iron, magnesium, manganese, phosphorous, potassium and zinc. In another specific embodiment, the micronutrient premix provides at least 75% of the recommended daily allowance (RDA) for children aged 12-18 months of minimally vitamin A, vitamin B, vitamin C, vitamin D, vitamin E, calcium, copper, iron, magnesium, manganese, phosphorous, potassium and zinc.

A micronutrient premix may further comprise vitamins and minerals in addition to the vitamin A, vitamin B, vitamin C, vitamin D, vitamin E, calcium, copper, iron, magnesium, manganese, phosphorous, potassium and zinc.

### (e) Additional Ingredients

Food formulations of the present disclosure may further comprise one or more additional ingredient listed in Table G.

TABLE G

| | | Names Found |
|---|---|---|
| Ingredients | What They Do | on Product Labels |
| Preservatives | Prevent food spoilage from | Ascorbic acid, citric acid, sodium |
| | bacteria, molds, fungi, or yeast | benzoate, calcium propionate, sodium |
| | (antimicrobials); slow or prevent | erythorbate, sodium nitrite, calcium |
| | changes in color, flavor, or | sorbate, potassium sorbate, BHA, BHT, |
| | texture and delay rancidity | EDTA, tocopherols (Vitamin E) |
| | (antioxidants); maintain | |
| | freshness | |
| Sweeteners | Add sweetness with or without | Sucrose (sugar), glucose, fructose, |
| | the extra calories | sorbitol, mannitol, corn syrup, high |
| | | fructose corn syrup, saccharin, |
| | | aspartame, sucralose, acesulfame |
| | | potassium (acesulfame-K), neotame |
| Color Additives | Offset color loss due to | FD&C Blue Nos. 1 and 2, FD&C Green |
| | exposure to light, air, | No. 3, FD&C Red Nos. 3 and 40, FD&C |
| | temperature extremes, moisture | Yellow Nos. 5 and 6, Orange B, Citrus |
| | and storage conditions; correct | Red No. 2, annatto extract, beta-carotene, |
| | natural variations in color; | grape skin extract, cochineal extract or |
| | enhance colors that occur | carmine, paprika oleoresin, caramel color, |
| | naturally; provide color to | fruit and vegetable juices, saffron (Note: |
| | colorless and “fun” foods | Exempt color additives are not required to |
| | | be declared by name on labels but may be |
| | | declared simply as colorings or color |
| | | added) |
| Flavors and | Add specific flavors (natural and | Natural flavoring, artificial flavor, and |
| Spices | synthetic) | spices |
| Flavor Enhancers | Enhance flavors already present | Monosodium glutamate (MSG), |
| | in foods (without providing their | hydrolyzed soy protein, autolyzed yeast |
| | own separate flavor) | extract, disodium guanylate or inosinate |
| Fat Replacers | Provide expected texture and a | Olestra, cellulose gel, carrageenan, |
| (and components | creamy “mouth-feel” in reduced- | polydextrose, modified food starch, |
| of formulations | fat foods | microparticulated egg white protein, guar |
| used to replace | | gum, xanthan gum, whey protein |
| fats) | | concentrate |
| Nutrients | Replace vitamins and minerals | Thiamine hydrochloride, riboflavin |
| | lost in processing (enrichment), | (Vitamin B₂), niacin, niacinamide, folate or |
| | add nutrients that may be | folic acid, beta carotene, potassium |
| | lacking in the diet (fortification) | iodide, iron or ferrous sulfate, alpha |
| | | tocopherols, ascorbic acid, Vitamin D, |
| | | amino acids (L-tryptophan, L-lysine, L- |
| | | leucine, L-methionine, L-cysteine, L- |
| | | threonine) |
| Emulsifiers | Allow smooth mixing of | Soy lecithin, mono- and diglycerides, egg |
| | ingredients, prevent separation | yolks, polysorbates, sorbitan |
| | Keep emulsified products | monostearate |
| | stable, reduce stickiness, | |
| | control crystallization, keep | |
| | ingredients dispersed, and to | |
| | help products dissolve more | |
| | easily | |
| Stabilizers and | Produce uniform texture, | Gelatin, pectin, guar gum, carrageenan, |
| Thickeners, | improve “mouth-feel” | xanthan gum, whey |
| Binders, | | |
| Texturizers | | |
| pH Control | Control acidity and alkalinity, | Lactic acid, citric acid, ammonium |
| Agents and | prevent spoilage | hydroxide, sodium carbonate |
| acidulants | | |
| Leavening Agents | Promote rising of baked goods | Baking soda, monocalcium phosphate, |
| | | calcium carbonate |
| Anti-caking | Keep powdered foods free- | Calcium silicate, iron ammonium citrate, |
| agents | flowing, prevent moisture | silicon dioxide |
| | absorption | |
| Humectants | Retain moisture | Glycerin, sorbitol |
| Firming Agents | Maintain crispness and firmness | Calcium chloride, calcium lactate |
| Enzyme | Modify proteins, | Enzymes, lactase, papain, rennet, |
| Preparations | polysaccharides and fats | chymosin |
| Gases | Serve as propellant, aerate, or | Carbon dioxide, nitrous oxide |
| | create carbonation | |

In some aspects, a food formulation further comprises at least one sweetener. In one aspect, a food formulation further comprises sugar (i.e. sucrose), and optionally one or more additional sweetener. The amount of sugar may vary. In one example, a food formulation comprises up to about 30 g of sugar per 100 g of the food formulation. In another example, a food formulation comprises about 0.1 g to about 30 g of sugar, or about 1 g to about 30 g of sugar, per 100 g of the food formulation. In another example, a food formulation comprises about 10 g to about 30 g of sugar per 100 g of the food formulation. In another example, a food formulation comprises about 20 g to about 30 g of sugar per 100 g of the food formulation. In another example, a food formulation comprises about 25 g to about 30 g of sugar per 100 g of the food formulation. In another example, a food formulation comprises about 27 g to about 30 g of sugar, or about 28 g to about 30 g of sugar, per 100 g of the food formulation. In another example, a food formulation comprises about 27 g, 27.1 g, 27.2 g, 27.3 g, 27.4 g, 27.5 g, 27.6 g, 27.7 g, 27.8 g, 27.9 g or 28 g of sugar per 100 g of the food formulation. In another example, a food formulation of the disclosure comprises about 28 g, 28.1 g, 28.2 g, 28.3 g, 28.4 g, 28.5 g, 28.6 g, 28.7 g, 28.8 g, 28.9 g or 29 g of sugar per 100 g of the food formulation. In another example, a food formulation of the disclosure comprises about 29 g, 29.1 g, 29.2 g, 29.3 g, 29.4 g, 29.5 g, 29.6 g, 29.7 g, 29.8 g, 29.9 g or 30 g of sugar per 100 g of the food formulation.

In some aspects, a food formulation further comprises at least one fat. A fat may be an animal fat, or more preferably a vegetable oil. In some aspects, a fat is chosen from avocado oil, canola oil, coconut oil, corn oil, cottonseed oil, flaxseed oil, grape seed oil, hemp seed oil, olive oil, palm oil, peanut oil, rice bran oil, safflower oil, soybean oil, or sunflower oil. In further aspects, one fat provides at least 50% by weight (wt %) of the total fat in the food formulation. For instance, one fat may provide about 50%, about 55%, about 60%, about 65%, about 70%, about 75%, about 80%, about 85%, about 90%, or about 95% by weight of the total fat in the food formulation. In one example the fat is soybean oil. In one example the fat is canola oil. In still further aspects, two or more fats provide at least 50% by weight of the fat in the food formulation. For instance, two or more fats may provide about 50%, about 55%, about 60%, about 65%, about 70%, about 75%, about 80%, about 85%, about 90%, or about 95% by weight of the total fat in the food formulation. In one example, at least one fat is soybean oil or canola oil. In one example, the fat is soybean oil and canola oil.

In other aspects, a food formulation further comprises soybean oil, and the soybean oil provides at least 50% by weight of the total fat in the food formulation. In further aspects, the soybean oil provides at least 75% by weight of the total fat in the food formulation. In still further aspects, the soybean oil provides at least 90% by weight of the total weight of fat in the food formulation. In still further aspects, the soybean oil provides at least 95% by weight of the total fat in the food formulation. In each of the above aspects, the food formulation may further comprise a fat chosen from animal fat or vegetable oil.

In still other aspects, a food formulation further comprises about 20 g of soybean oil. In one aspect, a food formulation comprises about 15 g, about 16 g, about 17 g, about 18 g, about 19 g, about 20 g, or about 21 g of soybean oil per 100 g of the food formulation. In another aspect, a food formulation further comprises about 15 g to about 21 g, about 16 g to about 21 g, about 17 g to about 21 g, about 18 g to about 21 g, about 19 g to about 21 g, about 20 g to about 21 g, about 15 g to about 20 g, about 16 g to about 20 g, about 17 g to about 20 g, about 18 g to about 20 g, or about 19 g to about 20 g of soybean oil per 100 g of the food formulation. In still another aspect, a food formulation of the disclosure comprises about 17 g, 17.1 g, 17.2 g, 17.3 g, 17.4 g, 17.5 g, 17.6 g, 17.7 g, 17.8 g, 17.9 g or 18 g of soybean oil per 100 g of the food formulation. In still yet another aspect, a food formulation of the disclosure comprises about 18 g, 18.1 g, 18.2 g, 18.3 g, 18.4 g, 18.5 g, 18.6 g, 18.7 g, 18.8 g, 18.9 g or 19 g of soybean oil per 100 g of the food formulation. In still yet another different aspect, a food formulation further comprises about 19 g, 19.1 g, 19.2 g, 19.3 g, 19.4 g, 19.5 g, 19.6 g, 19.7 g, 19.8 g, 19.9 g or 20 g of soybean oil. In a different aspect, a food formulation of the disclosure comprises about 20 g, 20.1 g, 20.2 g, 20.3 g, 20.4 g, 20.5 g, 20.6, 20.7 g, 20.8 g, 20.9 g or 21 g of soybean oil per 100 g of the food formulation.

### (f) Exemplary Food Formulations

In one aspect, a food formulation of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix. In another aspect, a food formulation of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix. In preferred aspects, the micronutrient premix referenced in this paragraph contains the nutrients listed in Table A and Table B in the amount specified in Table E and Table F, respectively.

In some aspects, a food formulation of the present disclosure as described in this section (f), has total protein of about 11.6 g, total fat of about 20.8 g, total carbohydrate of about 46.2 g, and total fiber of about 4.5 g. For example, a food formulation of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g. In another example, a food formulation of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g. In preferred aspects, the micronutrient premix referenced in this paragraph contains the nutrients listed in Table E and Table F in the amount specified in Table E and Table F, respectively.

In exemplary aspects, a food formulation of the present disclosure as described in this section (f), has a protein energy ratio (PER) of about 11.4, a fat energy ratio (FER) of about 46.0, and total calories of about 400 to about 560 kcal per 100 g of the food formulation. For example, a food formulation of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, wherein the food formulation has a protein energy ratio (PER) of about 11.4, a fat energy ratio (FER) of about 46.0, and total calories of about 400 to about 560 kcal per 100 g of the food formulation. In another example, a food formulation of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, wherein the food formulation has a protein energy ratio (PER) of about 11.4, a fat energy ratio (FER) of about 46.0, and total calories of about 400 to about 560 kcal per 100 g of the food formulation. In yet another example, a food formulation of the present disclosure may contain (per 100 g) about 10 g chickpea flour or a glycan equivalent thereof, about 10 g peanut flour or a glycan equivalent thereof, about 8 g soy flour or a glycan equivalent thereof, about 19 g raw banana or a glycan equivalent thereof, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g, wherein the food formulation has a protein energy ratio (PER) of about 11.4, a fat energy ratio (FER) of about 46.0, and total calories of about 400 to about 560 kcal per 100 g of the food formulation. In still another example, a food formulation of the present disclosure may contain (per 100 g) about 10 g chickpea flour, about 10 g peanut flour, about 8 g soy flour, about 19 g raw banana, about 29.9 g sugar, about 20 g soybean oil, and about 3.1 g micronutrient premix, and have total protein of about 11.6 g, total fat of about 20.8 g, total carbohydrate of about 46.2 g, and total fiber of about 4.5 g, wherein the food formulation has a protein energy ratio (PER) of about 11.4, a fat energy ratio (FER) of about 46.0, and total calories of about 400 to about 560 kcal per 100 g of the food formulation. In preferred aspects, the micronutrient premix referenced in this paragraph contains the nutrients listed in Table A and Table B in the amount specified in Table A and Table B, respectively.

Food formulations of the present disclosure may be formulated into a beverage, a food or a supplement. Non-limiting examples include a bar, a paste, a gel, a cookie, a cracker, a powder, a pellet, a powdered drink to be reconstituted, a blended beverage, a carbonated beverage, and the like. When food formulations of the present disclosure are intended to be administered and consumed by humans, the ingredients in the food formulations are typically Food Chemicals Codex (FCC) purity or U.S. Pharmacopeia (USP)—National Formulary quality, as appropriate, and free from foreign materials. In some aspects, a food formulation may be a therapeutic food. In some aspects, a food formulation may be a ready-to-use food. The term “ready-to-use food” refers to a food that comes ready to use as provided. Specifically, a ready-to-use food doesn't require reconstitution or refrigeration, and stays fresh for at least 6 months, preferably one year, or more preferably two years. In some aspects, a food formulation may be a ready-to-use therapeutic food, as defined in U.S. Department of Agriculture, “Commercial Item Description: Ready-to-Use Therapeutic Food (RUTF)” A-A-20363B (2012), which is designed to meet the guidelines established at the FAO-WHO 45th session of the Codex Alimentarius Commission (Nov. 21, 2022).

Table H provides a list of exemplary food formulations that may be used with the compositions disclosed herein.

TABLE H

| Components (g/100 g) | RUSF | MDCF-1 | MDCF-2 | MDCF-3 |
|---|---|---|---|---|
| Chickpea flour | 0 | 8 | 10 | 30 |
| Peanut flour | 0 | 7 | 10 | 0 |
| Soy flour | 0 | 5 | 8 | 14 |
| Raw Banana | 0 | 19 | 19 | 0 |
| Rice | 18.9 | 0 | 0 | 0 |
| Lentil | 21.5 | 0 | 0 | 0 |
| Powdered Skimmed Milk | 10.5 | 11.5 | 0 | 0 |
| Sugar | 17 | 24.3 | 29.9 | 30.9 |
| Soybean oil | 29 | 22 | 20 | 22 |
| Micronutrient Premix | 3.14 | 3.14 | 3.1 | 3.1 |
| Protein | 10.2 | 12.4 | 11.6 | 13.9 |
| Fat | 29.5 | 22.8 | 20.8 | 24.1 |
| Total Carbohydrates | 48.8 | 42.9 | 46.2 | 52.9 |
| Fiber | 4.7 | 3.3 | 4.5 | 5.6 |
| Protein Energy Ratio (PER) | 8.2 | 11.8 | 11.4 | 11.7 |
| Fat Energy Ratio (FER) | 53.6 | 49.0 | 46.0 | 45.6 |
| Total Calories per 100 g | 494.6 | 418.1 | 406.8 | 475.8 |

Tables I(a), J(a), K(a) and L(a) further provides food formulations modified from the formulations listed in Table H. The corresponding metrics for the formulation including PER, FER and SERs are provided in Tables I(b), J(b), K(b) and L(b). The 4 exemplary formulations include MDCF-2, MDCF-2SS, MDSF, and MD_RUTF. The formulations provided here are exemplary only, and ingredients can be changed based on factors like availability, target age, function, regulatory requirements etc.

TABLE Ia
Formulation for MDCF-2

| | Amount | Energy | | Protein | | Carbohydrate | | Fat | |
|---|---|---|---|---|---|---|---|---|---|
| Ingredients | (g) | (kcal) | | (g) | | (g) | | (g) | |
| CHICKPEA, FLOUR | 10.00 | 3.87 | 38.70 | 0.22 | 2.24 | 0.58 | 5.78 | 0.07 | 0.67 |
| PEANUT, PASTE | 10.00 | 5.87 | 58.70 | 0.24 | 2.44 | 0.21 | 2.13 | 0.50 | 4.97 |
| SOY, FLOUR | 8.00 | 4.34 | 34.72 | 0.38 | 3.02 | 0.32 | 2.55 | 0.21 | 1.65 |
| SUGAR, CRUSHED | 29.86 | 3.89 | 116.16 | 0.00 | 0.00 | 1.00 | 29.79 | 0.00 | 0.00 |
| SOYBEAN OIL | 20.00 | 8.84 | 176.80 | 0.00 | 0.00 | 0.00 | 0.00 | 1.00 | 20.00 |
| Green Banana Pulp | 19.00 | 0.89 | 16.91 | 0.01 | 0.19 | 0.23 | 4.37 | 0.00 | 0.06 |
| Canola Oil | 0.00 | 8.84 | 0.00 | 0.00 | 0.00 | 0.00 | 0.00 | 1.00 | 0.00 |
| Mineral POWDER | 2.98 | 0.00 | 0.00 | 0.00 | 0.00 | 0.00 | 0.00 | 0.00 | 0.00 |
| Vitamin POWDER | 0.16 | 0.00 | 0.00 | 0.00 | 0.00 | 0.00 | 0.00 | 0.00 | 0.00 |
| Amino acid POWDER | 0.00 | 0.00 | 0.00 | 0.00 | 0.00 | 0.00 | 0.00 | 0.00 | 0.00 |

| | | | | | |
|---|---|---|---|---|---|
| Total | 100.00 | 441.99 | 7.89 | 44.62 | 27.35 |

TABLE Ib
Metrics for MDCF-2

| | | | | n-3 (ω-3) | | % of | n-6 (ω-6) | | % of | (ω-6/ |
|---|---|---|---|---|---|---|---|---|---|---|
| | | | | fatty acids | | Energy | fatty acids | | Energy | ω-3) |
| Ingredients | PER | FER | SER | (g) | | from n-3 | (g) | | from n-6 | ratio |
| CHICKPEA, FLOUR | 7.14 | 55.68 | 26.28 | 0.00 | 0.00 | 2.54 | 0.00 | 0.00 | 23.25 | 9.15 |
| PEANUT, PASTE | | | | 0.00 | 0.00 | | 0.10 | 0.97 | | |
| SOY, FLOUR | | | | 0.00 | 0.00 | | 0.00 | 0.00 | | |
| SUGAR, CRUSHED | | | | 0.00 | 0.00 | | 0.00 | 0.00 | | |
| SOYBEAN OIL | | | | 0.06 | 1.20 | | 0.50 | 10.00 | | |
| Green Banana POWDER | | | | 0.00 | 0.04 | | 0.02 | 0.45 | | |
| Canola Oil | | | | 0.09 | 0.00 | | 0.19 | 0.00 | | |
| Mineral POWDER | | | | — | — | | — | — | | |
| Vitamin POWDER | | | | — | — | | — | — | | |
| Amino acid POWDER | | | | — | — | | — | — | | |

| | | | | | | | | |
|---|---|---|---|---|---|---|---|---|
| Total | 7.14 | 55.68 | 26.28 | 1.25 | 2.54 | 11.42 | 23.25 | 9.15 |

TABLE J(a)
Formulation for MDCF-2SS

| | Amount | Energy | | Protein | | Carbohydrate | | Fat | |
|---|---|---|---|---|---|---|---|---|---|
| Ingredients | (g) | (kcal) | | (g) | | (g) | | (g) | |
| CHICKPEA, FLOUR | 11.90 | 3.87 | 46.05 | 0.22 | 2.67 | 0.58 | 6.88 | 0.07 | 0.80 |
| PEANUT, PASTE | 10.00 | 5.87 | 58.70 | 0.24 | 2.44 | 0.21 | 2.13 | 0.50 | 4.97 |
| SOY, FLOUR | 13.00 | 4.34 | 56.42 | 0.38 | 4.92 | 0.32 | 4.15 | 0.21 | 2.68 |
| SUGAR, CRUSHED | 25.00 | 3.89 | 97.25 | 0.00 | 0.00 | 1.00 | 24.94 | 0.00 | 0.00 |
| SOYBEAN OIL | 22.00 | 8.84 | 194.48 | 0.00 | 0.00 | 0.00 | 0.00 | 1.00 | 22.00 |
| Green Banana | 15.00 | 3.88 | 58.20 | 0.05 | 0.69 | 0.84 | 12.53 | 0.00 | 0.00 |
| POWDER | | | | | | | | | |
| Canola Oil | 0.00 | 8.84 | 0.00 | 0.00 | 0.00 | 0.00 | 0.00 | 1.00 | 0.00 |
| Mineral | 2.94 | 0.00 | 0.00 | 0.00 | 0.00 | 0.00 | 0.00 | 0.00 | 0.00 |
| POWDER | | | | | | | | | |
| Vitamin | 0.16 | 0.00 | 0.00 | 0.00 | 0.00 | 0.00 | 0.00 | 0.00 | 0.00 |
| POWDER | | | | | | | | | |
| Amino acid | 0.00 | 0.00 | 0.00 | 0.00 | 0.00 | 0.00 | 0.00 | 0.00 | 0.00 |
| POWDER | | | | | | | | | |

| | | | | | |
|---|---|---|---|---|---|
| Total | 100.00 | 511.10 | 10.71 | 50.62 | 30.45 |

TABLE J(b)
Metrics for MDCF-2SS

| | | | | | | | | |
|---|---|---|---|---|---|---|---|---|
| | | | | n-3 (ω-3) | % of | n-6 (ω-6) | % of | (ω- |
| | | | | fatty acids | Energy | fatty acids | Energy | 6/ω-3) |
| Ingredients | PER | FER | SER | (g) | from n-3 | (g) | from n-6 | ratio |

| | | | | | | | | | |
|---|---|---|---|---|---|---|---|---|---|
| CHICKPEA, FLOUR | 53.62 | 19.03 | 0.00 | 0.00 | 2.39 | 0.00 | 0.00 | 21.70 | 9.07 |
| PEANUT, PASTE | | | 0.00 | 0.00 | | 0.10 | 0.97 | | |
| SOY, FLOUR | | | 0.00 | 0.00 | | 0.00 | 0.00 | | |
| SUGAR, CRUSHED | | | 0.00 | 0.00 | | 0.00 | 0.00 | | |
| SOYBEAN OIL | | | 0.06 | 1.32 | | 0.50 | 11.00 | | |
| Green Banana | | | 0.00 | 0.03 | | 0.02 | 0.35 | | |
| POWDER | | | | | | | | | |
| Canola Oil | | | 0.09 | 0.00 | | 0.19 | 0.00 | | |
| Mineral | | | — | — | | — | — | | |
| POWDER | | | | | | | | | |
| Vitamin | | | — | — | | — | — | | |
| POWDER | | | | | | | | | |
| Amino acid | | | — | — | | — | — | | |
| POWDER | | | | | | | | | |

| | | | | | | | | |
|---|---|---|---|---|---|---|---|---|
| Total | 8.38 | 53.62 | 19.03 | 1.36 | 2.39 | 12.32 | 21.70 | 9.07 |

TABLE K(a)
Formulation for MDSF

| | Amount | Energy | | Protein | | Carbohydrate | | Fat | |
|---|---|---|---|---|---|---|---|---|---|
| Ingredients | (g) | (kcal) | | (g) | | (g) | | (g) | |
| CHICKPEA, FLOUR | 13.00 | 3.87 | 50.31 | 0.22 | 2.91 | 0.58 | 7.51 | 0.07 | 0.87 |
| PEANUT, PASTE | 13.00 | 5.87 | 76.31 | 0.24 | 3.17 | 0.21 | 2.76 | 0.50 | 6.46 |
| SOY, FLOUR | 11.00 | 4.34 | 47.74 | 0.38 | 4.16 | 0.32 | 3.51 | 0.21 | 2.27 |
| SUGAR, CRUSHED | 22.00 | 3.89 | 85.58 | 0.00 | 0.00 | 1.00 | 21.95 | 0.00 | 0.00 |
| SOYBEAN OIL | 23.00 | 8.84 | 203.32 | 0.00 | 0.00 | 0.00 | 0.00 | 1.00 | 23.00 |
| Green Banana | 14.90 | 3.88 | 57.81 | 0.05 | 0.69 | 0.84 | 12.44 | 0.00 | 0.00 |
| POWDER | | | | | | | | | |
| Canola Oil | 0.00 | 8.84 | 0.00 | 0.00 | 0.00 | 0.00 | 0.00 | 1.00 | 0.00 |
| Mineral | 2.94 | 0.00 | 0.00 | 0.00 | 0.00 | 0.00 | 0.00 | 0.00 | 0.00 |
| POWDER | | | | | | | | | |
| Vitamin | 0.16 | 0.00 | 0.00 | 0.00 | 0.00 | 0.00 | 0.00 | 0.00 | 0.00 |
| POWDER | | | | | | | | | |
| Amino acid | 0.00 | 0.00 | 0.00 | 0.00 | 0.00 | 0.00 | 0.00 | 0.00 | 0.00 |
| POWDER | | | | | | | | | |

| | | | | | |
|---|---|---|---|---|---|
| Total | 100.00 | 521.07 | 10.92 | 48.18 | 32.60 |

TABLE K(b)
Metrics for MDSF

| | | | | n-3 (ω-3) | | % of | n-6 (ω-6) | | % of | (ω- |
|---|---|---|---|---|---|---|---|---|---|---|
| | | | | fatty acids | | Energy | fatty acids | | Energy | 6/ω-3) |
| Ingredients | PER | FER | SER | (g) | | from n-3 | (g) | | from n-6 | ratio |
| CHICKPEA, FLOUR | 8.38 | 56.31 | 16.42 | 0.00 | 0.00 | 2.45 | 0.00 | 0.00 | 22.65 | 9.24 |
| PEANUT, PASTE | | | | 0.00 | 0.00 | | 0.10 | 1.26 | | |
| SOY, FLOUR | | | | 0.00 | 0.00 | | 0.00 | 0.00 | | |
| SUGAR, CRUSHED | | | | 0.00 | 0.00 | | 0.00 | 0.00 | | |
| SOYBEAN OIL | | | | 0.06 | 1.38 | | 0.50 | 11.50 | | |
| Green Banana | | | | 0.00 | 0.03 | | 0.02 | 0.35 | | |
| POWDER | | | | | | | | | | |
| Canola Oil | | | | 0.09 | 0.00 | | 0.19 | 0.00 | | |
| Mineral | | | | — | — | | — | — | | |
| POWDER | | | | | | | | | | |
| Vitamin | | | | — | — | | — | — | | |
| POWDER | | | | | | | | | | |
| Amino acid | | | | — | — | | — | — | | |
| POWDER | | | | | | | | | | |

| | | | | | | | | |
|---|---|---|---|---|---|---|---|---|
| Total | 8.38 | 56.31 | 16.42 | 1.42 | 2.45 | 13.11 | 22.65 | 9.24 |

TABLE L(a)
Formulation for MD-RUTF

| | Amount | Energy | | Protein | | Carbohydrate | | Fat | |
|---|---|---|---|---|---|---|---|---|---|
| Ingredients | (g) | (kcal) | | (g) | | (g) | | (g) | |
| Chickpea, flour | 8.68 | 3.87 | 33.59 | 0.22 | 1.94 | 0.58 | 5.02 | 0.07 | 0.58 |
| Peanut, high oleic | 13.87 | 5.87 | 81.42 | 0.24 | 3.38 | 0.21 | 2.91 | 0.50 | 6.89 |
| Soy flour, defatted | 16.30 | 3.27 | 53.30 | 0.51 | 8.39 | 0.34 | 5.53 | 0.01 | 0.20 |
| Sugar, crushed | 23.00 | 3.89 | 89.47 | 0.00 | 0.00 | 1.00 | 22.95 | 0.00 | 0.00 |
| Green Banana | 8.75 | 3.88 | 33.95 | 0.05 | 0.40 | 0.84 | 7.31 | 0.00 | 0.00 |
| powder | | | | | | | | | |
| Canola oil | 8.10 | 8.84 | 71.60 | 0.00 | 0.00 | 0.00 | 0.00 | 1.00 | 8.10 |
| Palm oil | 18.00 | 8.84 | 159.12 | 0.00 | 0.00 | 0.00 | 0.00 | 1.00 | 18.00 |
| Mineral powder | 2.30 | 0.00 | 0.00 | 0.00 | 0.00 | 0.00 | 0.00 | 0.00 | 0.00 |
| Vitamin powder | 0.15 | 0.00 | 0.00 | 0.00 | 0.00 | 0.00 | 0.00 | 0.00 | 0.00 |
| Amino acid | 0.45 | 0.00 | 0.00 | 0.00 | 0.00 | 0.00 | 0.00 | 0.00 | 0.00 |
| powder | | | | | | | | | |
| Stabilizers | 0.40 | 0.00 | 0.00 | 0.00 | 0.00 | 0.00 | 0.00 | 0.00 | 0.00 |

| | | | | | |
|---|---|---|---|---|---|
| Total | 100.00 | 522.45 | 14.12 | 43.71 | 33.78 |

TABLE L(b)
Metrics for MD-RUTF

| | | | | n-3 (ω-3) | | % of | n-6 (ω-6) | | % of | (ω- |
|---|---|---|---|---|---|---|---|---|---|---|
| | | | | fatty acids | | Energy | fatty acids | | Energy | 6/ω-3) |
| Ingredients | PER | FER | SER | (g) | | from n-3 | (g) | | from n-6 | ratio |
| Chickpea, flour | 10.81 | 58.18 | 17.12 | 0.00 | 0.01 | 1.22 | 0.03 | 0.25 | 7.39 | 6.05 |
| Peanut, high oleic | | | | 0.00 | 0.00 | | 0.05 | 0.67 | | |
| Soy flour, defatted | | | | 0.00 | 0.04 | | 0.02 | 0.27 | | |
| Sugar, crushed | | | | 0.00 | 0.00 | | 0.00 | 0.00 | | |
| Green Banana | | | | 0.00 | 0.02 | | 0.00 | 0.03 | | |
| powder | | | | | | | | | | |
| Canola oil | | | | 0.07 | 0.60 | | 0.18 | 1.44 | | |
| Palm oil | | | | 0.00 | 0.04 | | 0.09 | 1.64 | | |
| Mineral | | | | — | — | | — | — | | |
| powder | | | | | | | | | | |
| Vitamin | | | | — | — | | — | — | | |
| powder | | | | | | | | | | |
| Amino acid | | | | — | — | | — | — | | |
| powder | | | | | | | | | | |
| Stabilizers | | | | — | — | | — | — | | |

| | | | | | | | | |
|---|---|---|---|---|---|---|---|---|
| Total | 10.81 | 58.18 | 17.12 | 0.71 | 1.22 | 4.29 | 7.39 | 6.05 |

In some aspects, the current disclosure also encompasses a food formulation as disclosed herein, for example MDCF-1, MDCF-2, MDCF-3, MDCF-2SS, MDSF, or MD-RUTF or variants thereof, for treatment of MAM, SAM or stunting. In some aspects, the food formulation may be administered to augment the benefits of P. copri in the gut microbiome. In some aspects, the P. copri is administered as a composition as disclosed herein. In some aspects, the P. copri is not externally administered but exists in the subject's gut microbiome.

In some aspects, the compositions of the current disclosure may be formulated for any route of administration, for example oral, gastric, orogastric, nasogastric, implanted, buccal, and rectal.

In some aspects, the compositions of the current disclosure may be formulated in unit dosage form as a solid, semi-solid, liquid, capsule, powder, emulsions, suspensions, tablets and suitably packaged. In some aspects, the strains of the disclosure, or combination of strains and food formulations disclosed herein may be encapsulated. These formulations are a further aspect of the invention. In some aspect the formulations may be mixed with liquids for suitable for orogastric or nasogastric delivery. Usually, the amount of a strain of the invention, or a combination of strains of the invention, is between 0.1-95% by weight of the formulation, or between 0.1-1% or 1%-10% or 10%-20%, or 20%-30%, or 30%-40%, or 40%-50%, or 50%-60%, or 60%-70%, or 70%-80% or 80%-90% or 90%-99% by weight of the formulation. Methods of formulating compositions are discussed in, for example, Hoover, John E., Remington's Pharmaceutical Sciences, Mack Publishing Co., Easton, Pa. (1975), and Liberman, H. A. and Lachman, L., Eds., Pharmaceutical Dosage Forms, Marcel Decker, New York, N.Y. (1980).

In some aspects, administration of the compositions comprising at least one probiotic strain as disclosed herein, can be combined with simultaneous, or staggered administration of other probiotic strains, for example Bifidobacterium longum subspecies infantis (B. infantis) ID number Bg40721_2D9_SN_2018, food formulations, for example MDF (revisions 1 and 2), or both. Dosage and forms of such formulations can be empirically determined by a person of skill in the art.

### II. Methods

In some aspects, the current disclosure encompasses a method of treatment, the method comprising administering to a subject in need thereof, a therapeutically effective quantity of a composition as disclosed in Section I. In some aspects, the methods disclosed herein may be used in the prevention or treatment of malnutrition, Moderate Acute Malnutrition (MAM), Severe Acute Malnutrition (SAM), stunting, necrotizing enterocolitis, nosocomial infections, enteric inflammation, inflammatory disorders, immunodeficiency, inflammatory bowel disease, irritable bowel syndrome, cancer (particularly of the gastrointestinal and immune systems), diarrheal disease, antibiotic associated diarrhea, pediatric diarrhea, appendicitis, allergies, autoimmune disorders, multiple sclerosis, Alzheimer's disease, rheumatoid arthritis, coeliac disease, diabetes mellitus, organ transplantation, bacterial infections, viral infections, fungal infections, periodontal disease, urogenital disease, sexually transmitted disease, HIV infection, HIV replication, HIV associated diarrhea, surgical associated trauma, surgical-induced metastatic disease, sepsis, weight loss, anorexia, fever control, cachexia, wound healing, ulcers, gut barrier function, allergy, asthma, respiratory disorders, circulatory disorders, coronary heart disease, anemia, disorders of the blood coagulation system, renal disease, disorders of the central nervous system, hepatic disease, ischemia, nutritional disorders, osteoporosis, endocrine disorders, epidermal disorders, psoriasis, acne vulgaris, panic disorder, behavioral disorder and/or post-traumatic stress disorders. In some aspects, the current disclosure also encompasses a method for modifying, repairing, or improving the gut microbiota of a subject in need thereof by administration of a therapeutically effective quantity of a composition as provided in Section I, to a subject in need thereof. In some aspects, the current disclosure also encompasses administration of a therapeutically effective quantity of the disclosed compositions to a subject in need thereof, to enhance the uptake, or utilization, or both of milk N-glycans, or plant-derived polysaccharides, or both.

As used herein the term “therapeutically effective quantity” refers to an amount of the formulation that alleviates, in whole or in part, symptoms associated with the disorder or condition, or halts or slows further progression or worsening of those symptoms or prevents or provides prophylaxis for the disorder or condition. An “effective amount” refers to an amount effective, at dosages and for periods of time necessary, to achieve the desired therapeutic result. A therapeutically effective amount is also one in which any toxic or detrimental effects of compounds of the invention are outweighed by the therapeutically beneficial effects. In some aspects the therapeutically effective quantity may be a quantity that results in reduction in biomarkers of enteric inflammation in the subject. In some aspects the therapeutically effective quantity may be an amount that results in increases in the levels of beneficial plasma protein biomarkers. In some aspects the therapeutically effective quantity may be a quantity that results in significant improvement in ponderal growth as evidenced from weight-for-age z score (WAZ) or mid-upper arm circumference (MUAC) or any other objective measure known in the art. In some aspects the therapeutically effective quantity may be an amount that is sufficient to bring about improvement in musculoskeletal and brain development as demonstrated by objective measures known in the art. In some aspects the therapeutically effective quantity may be amounts that result in enhanced colonization of the beneficial probiotic populations in the gut as demonstrated by various objective means used in the art including but not limited to fecal cultures, genomic analysis of fecal or intestinal swabs. In some aspects, the therapeutically effective quantity may be an amount of the formulation that when administered in conjunction with a vaccine, improves the immunogenicity and efficacy of the vaccine for the subject. In some aspects, the therapeutically effective quantity may be an amount of the formulation that improves the overall health of the subject, as measured by objective measures known in the art.

In some aspects, the amount of a composition administered to a subject and the frequency of administration may vary depending upon the subject or host treated and the particular mode of administration. It will be appreciated by those skilled in the art that the unit content of agent contained in an individual dose of each dosage form need not in itself constitute a therapeutically effective amount, as the necessary therapeutically effective amount could be reached by administration of a number of individual doses.

Additionally, compositions as disclosed herein may be combined with food formulations as described herein or additional probiotic strains or both. The formulations may be administered together, or the administration may be staggered. Amounts of food formulations or probiotic formulations or both can vary and may be determined by a person of skill in the art. A detailed description of suitable amounts of food formulation for administration is provided in US 2022/0312817, the entire contents of which are hereby incorporated by reference.

As discussed above, administration can be oral, gastric, orogastric, nasogastric, implanted, buccal, and rectal. In some aspects the compositions in section I may be administered orally as any one of but not limited to a solid, semi-solid, liquid, capsule, powder, emulsions, suspensions and tablet or combinations thereof. In some aspects the compositions in section I may be administered, mixed with any one of but not limited to water, juice, gruel, milk, breast milk, baby food, baby formula including F-75 and F-100 or any other commercially available formula, beverage, food products, fruits and vegetables, raw foods and cooked foods. In some aspects the compositions may be administered once daily. In some aspects the compositions may be administered more than once daily. In some aspects the compositions in section I may be administered orogastrically. In some aspect the compositions may be administered nasogastrically.

Compositions described herein can be administered in a variety of methods well known in the arts. Administration can include, for example, methods involving oral ingestion, direct injection, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

In some aspects, the methods disclosed herein comprise administration of therapeutically effective quantities of the compositions in a subject exhibiting symptoms of or diagnosed with malnutrition. A subject in need of treatment for malnutrition may have a LAZ≤1, a MUAC≤1, a WAZ≤1, a WLZ≤1, deficiencies in vitamins and minerals, or any combination thereof. In some embodiments, a subject in need of treatment for malnutrition has a LAZ≤1, ≤2, or ≤3. In some embodiments, a subject in need of treatment for malnutrition has a MUAC≤1, ≤2, or ≤3. In some embodiments, a subject in need of treatment for malnutrition has a WAZ≤1, ≤2, or ≤3. In some embodiments, a subject in need of treatment for malnutrition has a WLZ≤1, ≤2, or ≤3. In some embodiments, a subject in need of treatment for malnutrition has a LAZ≤2, a MUAC ≤2, a WAZ≤2, a WLZ≤2, or any combination thereof. In some embodiments, a subject in need of treatment for malnutrition has a WAZ≤1.5 and a WLZ≤1.5. In some embodiments, a subject in need of treatment for malnutrition has a WAZ≤2 and a WLZ≤2. In some embodiments, the subject has moderate acute malnutrition. In some embodiments, the subject has severe acute malnutrition (SAM). In some aspects the subject is a child or an infant who consume diets with limited breastmilk content. As used herein the term “limited breastmilk diet” is where breastmilk comprises less than 50% of an infant's total caloric intake. In some aspects breastmilk may comprise 0% of the infant's total caloric intake. In some aspects breastmilk may comprise less than 10% of the infant's total caloric intake. In some aspects breastmilk may comprise less than 20% of the total caloric intake. In some aspects breastmilk may comprise less than 30% of the total caloric intake. In some aspects breastmilk may comprise less than 40% of the total caloric intake. In some aspects breastmilk may comprise less than 50% of the total caloric intake. In some aspects the child is exhibiting one or more of the symptoms including but not limited to a very low weight-for-height (WHZ, less than 3 z-scores below the median WHO growth standards) or a mid-upper arm circumference (MUAC) of less than 11.5 cm, visible severe wasting, or nutritional oedema. In some aspects the child is an infant of age 0-24 months. In some aspect the child is of 0-5 years of age. In some aspects the child is from a underdeveloped or developing country. In some aspects the child is from a developed country. In some aspects the child is from an household below the poverty line for a particular country or earning an income below the objective measure of poverty defined for the country of residence. In some aspect the child is exhibiting symptoms of or has been clinically diagnosed with malnutrition.

In some aspects the present disclosure also encompasses methods for modifying, repairing or improving the health of the gut microbiota of a subject in need thereof. As used herein the term “modifying the gut microbiota” means any intervention that results in change in the gut microbiome as measured by one of many methods available in the art. The change may be a decrease or an increase in the presence of a particular microbial strain, species, genus, family, order, or class. These methods to monitor gut microbiota are well known in the art and may include but are not restricted to fecal cultures, genomic analysis of the feces, or analysis of fecal or intestinal swabs. In some aspects, the present disclosure encompasses methods for repairing or improving the health of the gut microbiota of a subject in need thereof. The “health” of a subject's gut microbiota may be defined by relative abundances of microbial community members, expression of microbial genes, biomarkers, mediators of gut barrier function. To “repair the gut microbiota of a subject,” which is synonymous with “improve gut microbiota health,” means to change the microbiota of a subject, in particular the relative abundances of age- and health-discriminatory taxa, in a statistically significant manner towards chronologically-age matched reference healthy subjects. The term encompasses complete repair (i.e., the measure of gut microbiota health does not deviate by 1.5 standard deviation or more) and levels of repair that are less than complete. The term also encompasses preventing or lessening a change in the relative abundances of age- and health-discriminatory taxa, wherein the change would have been significantly greater absent intervention. A subject with a gut microbiota in need of repair (e.g., a microbiota in “disrepair”, an “immature” gut microbiota, etc.) has a measure of gut microbiota health that deviates by 1.5 standard deviation or more (e.g., 2 std. deviation, 2.5 std. deviation, 3 std. deviation, etc.) from that of chronologically-age matched subjects, wherein the term “chronological age” means the amount of time a subject has lived from birth. Subjects five years or younger are grouped (or binned) by month. Subjects older than 5 years may be grouped by longer intervals of time (e.g., months or years). In some embodiments, a subject with a gut microbiota in need of repair is a subject with malnutrition, SAM, a subject at risk of malnutrition, a subject with a diarrheal disease, a subject recently treated for diarrheal disease (e.g., within 1 week, 2 weeks, 3 weeks, 4 weeks, 5 weeks, 6 weeks, 7 weeks, or 8 weeks), a subject recently treated with antibiotics (e.g., within 1 week, 2 weeks, 3 weeks, 4 weeks, 5 weeks, 6 weeks, 7 weeks, or 8 weeks), a subject undergoing treatment with an antibiotic, a subject who will be undergoing treatment with an antibiotic with about 1-4 weeks or about 1-2 weeks.

In some aspects the subject may be an individual clinically diagnosed with a disease or disorder or syndrome or exhibiting symptoms of disease or disorder or syndrome. In some aspects the subject may be a healthy individual.

The aforementioned methods are not limited to subjects of a particular age. In one aspect, a subject may be less than six months of age. In one aspect, a subject may be at least six months of age. In one example, a subject may be at least six months of age. In another example, a subject may be eighteen years or younger. In still other examples, a subject may be ≤15 years, ≤14 years, ≤13 years, ≤12 years, ≤11 years, ≤10 years, ≤9 years, ≤8 years, ≤7 years, ≤6 years, ≤5 years, ≤4 years, ≤3 years, ≤2 years. In still other examples, a subject may be a newborn to six months of age, six months to five years of age, six months to 2 years of age, or six months to 18 months of age. In some aspects the subject is a pre-term baby. In some aspects the subject may be an animal. In some aspect the animal may be a mouse model.

An additional aspect of this invention is a method of improving immunogenicity and efficacy of a vaccine in children who consume diets with limited breast milk, the method comprising administration of effective amounts of the compositions detailed in section I of detailed description.

Microbiome can transfer from mother to infant. In some aspects of the invention, the compositions detailed in section I, may be administered to women during pregnancy to facilitate colonization of the probiotic in the infant gut.

In some aspects, effective amounts of the compositions detailed in section I may be administered prophylactically to reduce the occurrence of malnutrition in children growing up in an household below the poverty line of a particular country or earning an income below the objective measure of poverty defined for the country of residence. In some aspects, the compositions disclosed herein may be administered to “improve a subject's health”. To “improve a subject's health” means to change one or more aspects of a subject's health in a statistically significant manner towards chronologically-age matched reference healthy subjects, as well as to prevent or lessen a change in one or more aspects of the subject's health wherein the change would have been significantly greater absent intervention. The improved aspect of the subject's health may be growth or rate of growth, for example as measured by a score on an anthropometric index; signs or symptoms of disease; relative abundances of health discriminatory plasma proteins, including but not limited to biomarkers, mediators of gut barrier function, bone growth, neurodevelopment, acute and inflammation, and the like. Those in need of treatment to improve their health include those already with a disease, condition, or disorder as well as those prone to have the disease, condition or disorder or those in which the disease, condition or disorder is to be prevented.

### EXAMPLES

The following examples are included to demonstrate preferred embodiments of the disclosure. It should be appreciated by those of skill in the art that the techniques disclosed in the examples that follow represent techniques discovered by the inventor to function well in the practice of the present disclosure, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the present disclosure.

### Example 1: Methods for Examples 2-6

The following examples 2-6 describes characterization of the bacterial targets and structure-function relationships of a microbiome-directed complementary food prototype, MDCF-2. Evidence is accumulating that perturbed postnatal development of the gut microbiome contributes to childhood malnutrition. Designing effective microbiome-directed therapeutic foods to repair these perturbations requires knowledge about how food components interact with the microbiome to alter its expressed functions. Herein is described the use of biospecimens from a randomized, controlled study of a microbiome-directed complementary food prototype (MDCF-2) that produced superior rates of weight gain compared to a commonly used ready-to-use supplementary food (RUSF) in 12-18-month-old Bangladeshi children with moderate acute malnutrition (MAM).

Collection and Handling of Biospecimens Obtained from Participants in the Randomized Controlled Clinical Study of the Efficacy of MDCF-2

The human study entitled ‘Community-based Clinical Trial With Microbiota-Directed Complementary Foods (MDCFs) Made of Locally Available Food Ingredients for the Management of Children With Primary Moderate Acute Malnutrition (MAM)’, was approved by the Ethical Review Committee at the icddr,b (Protocol PR-18073; ClinicalTrials.gov identifier: NCT04015999). Informed consent was obtained for all participants. The objective of the study was to determine whether twice daily, controlled administration of a locally produced, microbiota-directed complementary food (MDCF-2) for 3 months to children with MAM provided superior improvements in weight gain, microbiota repair, and improvements in the levels of key plasma biomarkers/mediators of healthy growth, compared to a commonly used rice- and lentil-based ready-to-use supplementary food (RUSF) composition. A total of 124 male and female children with MAM (WLZ-2 to -3) between 12- and 18-months-old who satisfied the inclusion criteria were enrolled, with 62 children randomly assigned to each treatment group using the permuted block randomization method. Children in each treatment group were fed the corresponding dietary intervention (MDCF-2 or RUSF) twice daily at a study center for the first month, once daily at a study center and once daily at home for the second month, and twice daily at home for the third month, after which children returned to their normal feeding routine with continued intensive monitoring for one additional month. Fifty-nine participants in each treatment group completed the 3-month intervention and 1-month post-treatment follow-up. To ensure sample integrity for DNA and RNA analyses, fecal biospecimens were collected within 20 minutes of their production and immediately transferred to liquid nitrogen-charged vapor shippers for transport to a −80° C. freezer at the study center. Coded biospecimens were shipped to Washington University on dry ice where they were stored at −80° C., along with associated metadata, in a dedicated repository with approval from the Washington University Human Research Protection Office.

### Defining the Relationship Between MAG Abundances and WLZ

Linear mixed-effects models were used to relate the abundances of MAGs identified in each trial participant to WLZ using the formula:

$$WLZ ∼ {β}_{1} ( MAG ) + {β}_{2} ( study week ) + ( 1 \vert PID )$$

The data normalization strategies prior to linear modeling did not include a consideration of MAG assembly length. Therefore, the TPM was analyzed (reads per kilobase per million) output of Kallisto (v0.43.0) by applying a filter requiring each MAG's abundance >5 TPM in >40% of the 707 fecal samples collected at time points where anthropometry was also measured. This filtering approach yielded 837 MAGs. The unfiltered count output from Kallisto was then used to perform a variance stabilizing transformation [DESeq2] to control for heteroskedasticity, and the dataset was filtered to the same 837 MAGs. Subsequently the linear mixed effects models were fitted to the transformed abundances of each MAG across all 707 fecal samples (Ime430, v1.1-27.1; ImerTest31, v3.1-3). ANOVA was used to determine the statistical significance of the fixed effects in the model—specifically, the relationship between MAG abundance and WLZ. ‘WLZ-associated MAGs’ were defined as those having P-values adjusted for false discovery rate (q-values)<0.05.

### Determining the Effects of MDCF-2 Supplementation on the Abundances of WLZ-Associated MAGs and MAGs Belonging to a Given Species

Dream32 (variancePartition R package, v1.24.0) an empirical Bayesian linear mixed-effects modeling framework, was employed to model MAG abundance as a function of treatment group, study week, and their interaction, controlling for the repeated measurements taken from each study participant with random effect term for participant. The equation used to quantify the effects of treatment on MAG abundance took the form:

$${MAG}_{i} ∼ {β}_{1} ( treatment group ) + {β}_{2} ( study week ) + {β}_{3} ( treatment group \times study week ) + ( 1 \vert PID )$$

The ‘treatment group’ coefficient β1 indicates whether MDCF-2 produced changes in the mean abundance of a given MAG relative to RUSF over the 3-month intervention, while the ‘treatment group×study week—interaction’ coefficient β3 indicates whether MDCF-2 affected the rate of change of a given MAG more so than RUSF (i.e., was a MAG increasing or decreasing more rapidly in the microbiomes of participants in the MDCF-2 versus the RUSF treatment group). Each coefficient for each MAG abundance analysis is described by an associated t-statistic—a standardized measure, based on standard error, of a given coefficient's deviation from 0 which can be used to calculate a P-value and infer the significance of the effect of a given coefficient on the dependent variable. The t-statistics produced by this method can also be used as a ranking factor for input to GSEA. For this analysis, gene sets were defined as groups of MAGs that were either significantly positively (n=75) or significantly negatively (n=147) associated with WLZ. This analysis was conducted for both the ‘treatment group; β1’ coefficient and the ‘interaction; β3’ coefficient. Statistical significance is reported as q-values after adjustment for false-discovery rate (Benjamini-Hochberg method).

### Microbial RNA-Seq Analysis of MAG Gene Expression

For RNA extraction, approximately 50 mg of a fecal sample, collected from each participant at the baseline, 1-month, or 3-month timepoints, was pulverized under liquid nitrogen with a mortar and pestle and transferred to 2 mL cryotubes. A 3.97 mm steel ball and the equivalent of 250 μL of 0.1 mm zirconia/silica beads were subsequently added to each sample tube, together with 500 μL of a mixture of phenol: chloroform: isoamyl alcohol (25:24:1, pH 7.8-8.2), 210 μL of 20% SDS, and 500 μL of 2× Qiagen buffer A (200 mM NaCl, 200 mM Trizma base, 20 mM EDTA). After a 1-minute treatment in a bead beater (Biospec Minibeadbeater-96), samples were centrifuged at 3,220×g for 4 minutes at 4° C. One hundred microliters of the resulting aqueous phase was transferred by a liquid handling robot (Tecan) to a deep 96-well plate along with 70 μL of isopropanol and 10 μL of 3M NaOAc, pH 5.5. The solution was mixed by pipetting 10 times. The crude DNA/RNA mixture was incubated at −20° C. for 1 hour and then centrifugated at 3,220×g at 4° C. for 15 minutes before removing 210 μL of the supernatant to yield nucleic acid-rich pellets. A Biomek FX robot was used to add 300 μL Qiagen Buffer RLT to the pellets and to resuspend the RNA/DNA by pipetting up and down 50 times. A 400 μL aliquot was transferred from each well to an Qiagen AllPrep 96 DNA plate, which was centrifuged at 3,220×g for 1 minute at room temperature. The RNA flow-through was purified as described in the AllPrep 96 protocol. cDNA libraries were prepared from extracted RNA using an Illumina Total RNA Prep with Ribo-Zero Plus and dual unique indexes. Libraries were balanced, pooled, and sequenced in two runs of an Illumina NovaSeq using S4 flow cells.

As an initial pre-processing step, raw reads were aggregated across the two NovaSeq runs, resulting in a total of 5.0×10⁷±4.7×10⁶ paired-end 150 nt reads per sample (mean±SD). Adapter sequences and low-quality bases were removed from raw reads (Trim Galore33, v0.6.4), and pairs of trimmed reads were filtered out if either one of the paired reads was less than 100 nt long. Pre- and post-trimmed sequence quality and adapter contamination were assessed using FastQC34 (v0.11.7). Filtered reads were pseudoaligned to the set of 1,000 annotated, dereplicated high quality MAGs to quantify transcripts with Kallisto35. Reads that pseudoaligned to rRNA genes were excluded, leaving an average of 7.1×10⁶±3.9×10⁶ bacterial mRNA reads (mean±SD) per sample. Counts tables were further filtered to retain only transcripts that pseudoaligned to the 837 MAGs that passed the abundance and prevalence thresholds described above. To minimize inconsistently quantified counts related to low-abundance MAGs, a transcript count of zero was assigned, on a per-sample basis, to any MAG with a DNA abundance <0.5 TPM in that sample.

Differential expression analysis (edgeR36, v3.32.1) was conducted using the following steps: (i) transcript filtering for presence/absence and prevalence; (ii) library size normalization using TMM (trimmed mean of M-values); (iii) estimating per-gene count dispersions; and (iv) testing for differentially expressed genes. Transcripts were first filtered using edgeR default parameters, followed by a parameter sweep of transcript abundance and prevalence threshold combinations. Based on this analysis, transcripts with >5 counts per million mapped reads (CPM) in >35% of samples were retained for differential expression analysis. The transcripts that passed this filtering were normalized using a TMM-based scaling factor. Negative binomial dispersions was estimated and fit trended per-gene dispersions (using the power method) to negative binomial generalized linear models, which were used to characterize (i) the effect of treatment group and study week among all participants and (ii) the effect of WLZ-quartile and study week among MDCF-2 participants in the upper and lower quartile of WLZ-response using the following model formulae:

$${Transcripts}_{i} ∼ {β}_{1} ( treatment group ) + {β}_{2} ( study week ) + {β}_{3} ( treatment group \times study week )$$

$${Transcripts}_{i} ∼ {β}_{1} ( WLZ response quartile ) + {β}_{2} ( study week ) + {β}_{3} ( WLZ response quartile \times study week )$$

From these models, genes that exhibited significant differential expression were identified using the quasi-likelihood F-test (edgeR, function glmQLFTest) which accounts for the uncertainty in estimating the dispersion for each gene.

For subsequent functional metabolic pathway enrichment analyses, the following was undertaken (i) ordered transcripts assigned to WLZ-associated MAGs based on a ranking metric calculated as the direction of the fold change×−log₁₀ (P-value) for a given differential expression analysis, (ii) defined gene sets as groups of these transcripts assigned to the same metabolic pathway, and (iii) performed GSEA (fgsea37, v3.14). This set of analyses allowed the identification of differentially expressed metabolic pathways comprised of >10 genes over time (i) between treatment groups, (ii) between WLZ response quartiles or (iii) as a function of interacting terms in the linear mixed effect models (treatment group×study week; WLZ-response quartile×study week). Enrichment results were considered statistically significant if they exhibited q-values <0.1 after controlling for false-discovery rate (Benjamini-Hochberg method).

For targeted transcriptional analyses of the CAZymes encoded by P. copri MAGs Bg0018 and Bg0019, Dream32 was employed in R with no additional filtering, and the formula above relating transcripts to WLZ response quartile, study week, and the interaction of both terms, with the addition of a random effect for participant.

### Principal Components Analysis

Principal Components Analysis (PCA) was performed on VST-transformed DNA or transcript counts for the 837 MAGs passing the filter described in the section entitled ‘Defining the relationship between MAG abundances and WLZ’ above. The PCA related to transcripts encompassed 27,518 genes expressed by these MAGs at thresholds for levels and prevalence that are described in the section entitled ‘Microbial RNA-Seq analysis of MAG gene expression’ above. PCA was performed in R using the ‘prcomp’ function, with each data type centered but not scaled, since the dataset was already VST-normalized. The functions ‘get_eigenvalues,’ ‘get_pca_ind,’ and ‘get_pca_var’ from the factoextra (v1.0.7) package were utilized to extract (i) the variance explained by each principal component, (ii) the coordinates for each sample along principal components, and (iii) the contributions of each variable to principal components 1-3. ‘Adonis2’ function within the vegan library (v2.5-7) was used to test for the statistical significance of baseline differences in the microbiome (MAGs) or meta-transcriptome between the two treatment groups.

LC-MS Analyses of Carbohydrates Present in MDCF-2, RUSF, their Component Food Ingredients, and Fecal Biospecimens

Sample preparation for glycan structure analysis—Samples of MDCF-2, RUSF, their respective ingredients, and fecal biospecimens were ground with a mortar and pestle while submerged in liquid nitrogen. A 50 mg aliquot of each homogenized sample was lyophilized to dryness. Lyophilized samples were shipped to the Department of Chemistry at the University of California, Davis. On receipt, samples were pulverized to a fine powder using 2 mm stainless steel beads (for foods) or 2 mm glass beads (for feces). A 10 mg/mL stock solution of each sample was prepared in Nanopure water. All stock solutions were again bead homogenized, incubated at 100° C. for 1 h, bead homogenized again, and stored at −20° C. until further analysis.

Monosaccharide composition analysis—Briefly, 10 μL aliquots were withdrawn from homogenized stock solutions and transferred to a 96-well plate containing 2 mL wells. Each sample aliquot was acid hydrolyzed (4 M trifluoroacetic acid for 1 h at 121° C.) and quenched by adding 855 μL of ice-cold Nanopure water. Hydrolyzed samples, plus an external calibration standard comprised of 14 monosaccharides with known concentrations (0.001-100 g/mL each) were derivatized with 0.2 M 1-phenyl-3-methyl-5-pyrazolone (PMP) in methanol plus 28% NH4OH for 30 min at 70° C. The derivatized glycosides were fully dried by vacuum centrifugation, reconstituted in Nanopure water (Thermo Fischer Scientific), and excess PMP was extracted with chloroform. A 1 μL aliquot of the aqueous layer was injected into an Agilent 1290 Infinity II ultrahigh-performance liquid chromatography (UHPLC) system, separated using a 2-minute isocratic elution on a C18 column (Poroshell HPH, 2.1×50 mm, 1.9 μm particle size, Agilent Technologies), and analyzed using an Agilent 6495A triple quadrupole mass spectrometer (QqQ-MS) operated in dynamic multiple reaction monitoring (dMRM) mode. Monosaccharides in the food and fecal samples were identified and quantified by comparison to the external calibration curve.

Glycosidic linkage analysis—Under an argon atmosphere, a 5 μL aliquot from each homogenized stock solution was permethylated in a 200 μL reaction that contained 5 μL saturated NaOH and 40 μL iodomethane in 150 μL of DMSO. Permethylated glycosides were extracted with dichloromethane, and the extract was dried by vacuum centrifugation. The extracted glycosides were subjected to acid hydrolysis (4 M trifluoroacetic acid for 2 h at 100° C.) followed by vacuum centrifugation to dryness. Samples were then derivatized with PMP as described above for monosaccharide analysis, followed by another vacuum centrifugation to complete dryness. Methylated monosaccharides were then reconstituted with 100 μL of 70% methanol in water. A 1 μL aliquot of the aqueous layer was injected into an Agilent 1290 Infinity II UHPLC system, separated using a 16-minute gradient elution on a C18 column (ZORBAX RRHD Eclipse Plus, 2.1×150 mm, 1.8 μm particle size, Agilent Technologies), and analyzed using an Agilent 6495A QqQ-MS operated in multiple reaction monitoring (MRM) mode. A standard pool of oligosaccharides and reference MRM library were used to identify and quantify glycosidic linkages in all samples.

Fenton's initiation toward defined oligosaccharide groups (FITDOG) polysaccharide analysis—To separate endogenous oligosaccharides from the background food matrix, polysaccharides were precipitated with 80% aqueous ethanol. Dried precipitates were reconstituted, homogenized, and 10 mg/mL stock solutions were prepared. The FITDOG reaction was carried out using a 100 μL aliquot of the 10 mg/mL resuspended food pellet and 900 μL of reaction buffer (44 mM sodium acetate, 1.5% H₂O₂, 73 μM Fe₂(SO₄)₃(H₂O)₅). The reaction mixture was incubated at 100° C. for 45 minutes, quenched with 500 μL 2 M NaOH, and then neutralized with 61 μL of glacial acetic acid. The resulting oligosaccharides were then reduced to their corresponding alditols with sodium borohydride (NaBH₄) to prevent anomerization during chromatographic separation. For the reduction of oligosaccharides, a 400 μL aliquot of the reaction mixture was incubated with 400 μL 1 M NaBH₄ at 65° C. for 60 minutes. Oligosaccharide products were then enriched using C18 and porous graphitized carbon (PGC) 96-well solid-phase extraction plates. For the C18 enrichment, cartridges were primed with 2×250 μL acetonitrile (ACN) and then 5×250 μL water washes prior to loading the reduced sample. Cartridge effluent was collected and subjected to subsequent PGC clean-up. PGC cartridges were primed with 400 μL water, 400 μL 80% ACN/0.1% (v/v) trifluoroacetic acid (TFA), and then 400 μL water prior to loading the C18 effluent. Washing was performed with 8×400 μL water, and the oligosaccharides were eluted with 40% ACN/0.05% (v/v) TFA and then dried using a vacuum centrifugal dryer. Oligosaccharides were reconstituted with 100 μL Nanopure water and a 10 μL aliquot was injected into the HPLC-Q-TOF instrument. Separation was carried out using an Agilent 1260 Infinity II HPLC with a PGC column (Hypercarb, 1×150 mm, 5 μm particle size, Thermo Scientific) coupled to an Agilent 6530 Accurate-Mass Q-TOF mass spectrometer. Oligosaccharide identification was based on MS/MS fragmentation and retention time (RT) compared to reacted polysaccharide standards (amylose, cellulose, mannan, galactan, linear arabinan, and xylan). Food polysaccharides were quantified using an external calibration curve that included the three most abundant oligosaccharides from each parent polysaccharide as the quantifier species.

Statistical analysis of carbohydrate composition—The abundance trends of glycosidic linkages over time and between WLZ-response quartiles using linear mixed-effects models (Ime4, ImerTest packages in R) of the following form:

$${Linkage}_{i} ∼ {β}_{1} ( WLZ response quartile ) + {β}_{2} ( study week ) + {β}_{3} ( WLZ response quartile \times study week ) + ( 1 \vert PID )$$

Linkages displaying a significant interaction (q<0.05) between WLZ response quartile and study week (β3 coefficient) were identified.

### Metagenome Assembled Genomes (MAGs)

Short-read shotgun sequencing—DNA was isolated from 942 fecal samples and shotgun sequencing libraries were prepared using a reduced-volume Nextera XT (Illumina) protocol. Libraries were quantified, balanced, pooled, and sequenced [Illumina NovaSeq 6000, S4 flow cell; 2.3±1.4×10⁷ 150 nt paired-end reads/sample (mean±SD)]. Reads were demultiplexed (bcl2fastq, Illumina), trimmed to remove low quality bases, and processed to remove read-through adapter sequences (Trim Galore33, v0.6.4). Read pairs where the length of either read was <50 nt after quality and adapter trimming were discarded. The remaining reads were mapped to the human genome (UCSC hg19) using bowtie2 (v2.3.4.1) and were filtered to remove H. sapiens sequences.

Preprocessed, short-read shotgun data were aggregated from each participant's fecal sample set (n=7-8 samples/participant; 118 participants) prior to MAG assembly. This strategy was adopted to enable the contig abundance calculations required by the MAG assembly algorithms employed below, while at the same time mitigating the risk of chimeric assemblies inherent to co-assembly across individuals. Assemblies were generated for all 118 datasets using MegaHit (v1.1.4), and the resulting contigs were quantified in each assembly by mapping preprocessed reads to the assembled contigs with Kallisto. Contigs were assembled into MAGs using MaxBin2 (v2.2.5) and MetaBAT2 (v2.12.1). The parallel results of both binning strategies were merged and dereplicated using DAS Tool (v1.1.2) on a per-participant basis.

Long-read shotgun sequencing—Long-read sequencing was applied to fecal samples obtained at the 0- and 3-month time points from each of the 15 upper quartile WLZ responders in the MDCF-2 treatment group. Aliquots containing 400-1000 ng of DNA from each biospecimen were transferred to a 96-well, 0.8 mL, deep-well plate (Nunc, ThermoScientific) and prepared for long-read sequencing using the SMRTbell Express Template Prep Kit 2.0 (PacBio). All subsequent DNA handling and transfer steps were performed with wide-bore, genomic DNA pipette tips (ART, ThermoScientific). Barcoded adapters were ligated to A-tailed DNA fragments by overnight incubation at 20° C. Adapter-ligated fragments were then treated with the SMRTbell Enzyme Cleanup Kit to remove damaged or partial SMRTbell templates. A high molecular weight DNA fraction was purified using AMPure beads (ratio of 0.45× well-mixed AMPure bead volume to sample) and eluted in 12 mL of PacBio elution buffer. DNA libraries were sequenced on a Sequel System (Pacific Biosciences) using a Sequel Binding Kit 3.0 and Sequencing Primer v4 with 24 hours of data collection. A total of 3.0×10⁹±9.8×10⁸ bp/sample were collected, with an average subread length of 5,654±871 bp (mean±SD).

Hybrid assembly of short- and long-read data was performed using OPERA-MS (v0.9.0). OPERA-MS uses assembly graph and coverage-based methods to cluster contigs into MAGs based on optimizing per-cluster Bayesian information criterion (BIC). Prior to hybrid assembly, continuous long reads (CLR) were combined across the two available timepoints for each participant and reads that mapped to the human genome were removed. Illumina short reads and PacBio long reads (CLR) were provided to OPERA-MS and assembled using the built-in OPERA-MS genome database and default settings (the latter includes polishing of output MAGs with Pilon).

MAG dereplication, curation and abundance calculations—After assembling MAGs by both short-read only and short-plus long-read strategies, all MAGs from all assembly strategies were assessed for completeness and contamination (‘lineage_wf’ command in CheckM, v1.1.3) and refined (‘tetra’, ‘outliers’, and ‘modify’ commands) to remove contaminating contigs. Additional refinement based on the distribution of phylogenetic markers present in each MAG was performed [‘phylo-markers’, ‘clade-markers’, and ‘clean-bin’ commands in MAGpurify (v2.1.2)]. A final MAG quality assessment was performed using CheckM, followed by a stringent (≥90% complete, ≤5% contaminated, ANI≥99%) bulk dereplication (options ‘-I 50000’, ‘--completeness 90’, ‘--contamination 5’, ‘-pa 0.9’, ‘-sa 0.99’ in dRep (v2.6.2). The final dataset contained 681±99.4 (mean±SD) MAGs/participant. All MAGs satisfied the threshold criteria of having an abundance ≥5 TPM when present at any time point in an individual. MAG assembly summary statistics were collected from CheckM and quast analyses (v4.5) and aggregated. Initial MAG annotations were performed using prokka (v1.14.6). To quantify the abundance of each MAG in each sample MAGs were processed to create a single Kallisto quantification index. Reads from each fecal DNA sample were then mapped to this index.

MAG taxonomy—Taxonomic assignments were initially made by employing the Genome Taxonomy Database Toolkit (GTDB-Tk) and corresponding database (release 95). MAG assignments were complimented by using Kraken2 (v2.0.8) and Bracken (v2.5) and a Kraken2-compatible version of the GTDB reference.

P. copri has been partitioned into four distinct clades (‘A-D’) based on marker gene phylogeny. To classify Prevotella MAGs in this study, an unrooted, marker gene-based phylogeny was constructed using Phylophlan (v3.0.60). This tree encompassed 17 reference isolate genomes and 1006 MAGs from a previous study plus any MAGs from the set classified by GTDB-Tk as belonging to the genera Prevotella (n=51) or Prevotella massilia (n=13). Putative Prevotella MAGs from the present study that clustered within the four previously identified P. copri clades were assigned to the corresponding clade based on visualization with Graphlan (v1.1.4).

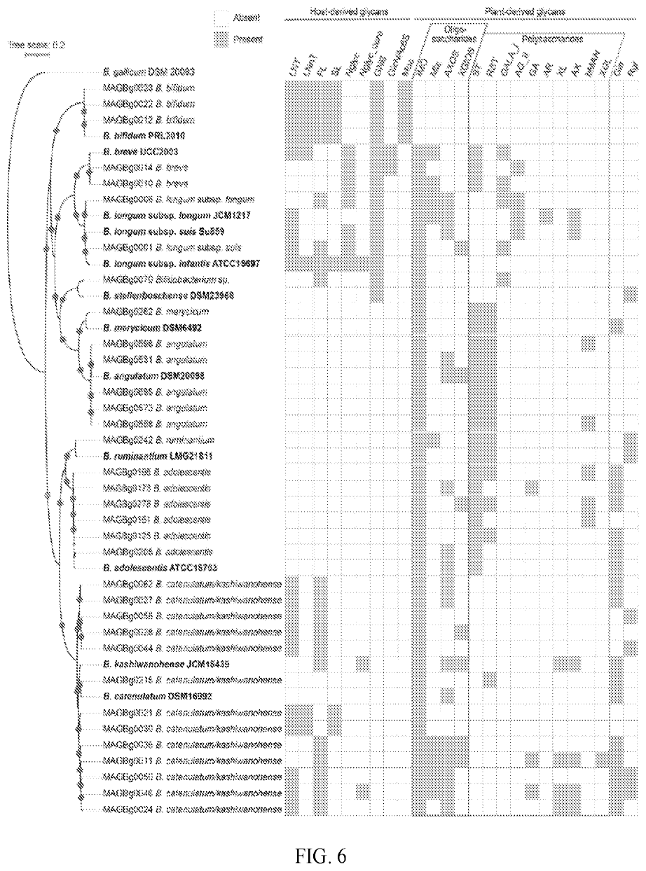

Certain Bifidobacterium species consist of multiple closely related subspecies (e.g., B. longum). Therefore, a pan-genome for 34 Bifidobacterium MAGs was calculated in the dataset, plus 14 reference isolate genomes (FIG. 6), using Roary (v3.12.0) and a 60% minimum sequence identity threshold for blastp. The reference isolate genomes included 10 Bifidobacterium species and three subspecies of Bifidobacterium longum (subsp. longum, infantis, and suis). Concatenated nucleotide sequences of 142 identified core genes were aligned using MAFFT (v7.313). The resulting alignment was trimmed [microseq R package (v2.1.4)] and was then used to construct a maximum likelihood phylogenetic tree [IQ-TREE (v1.6.12)]. The Bifidobacterium gallicum DSM 20093 genome was selected as an outgroup. Putative Bifidobacterium MAGs from this study that clustered together with reference genome clades were assigned to the corresponding clade. Using this method, the initial GTDB-Tk-based classifications of all Bifidobacterium MAGs were confirmed or updated or resolved nearly all closely related subspecies (FIG. 6).

### Subsystems-Based Annotation and Prediction of Functional Capabilities (‘Inferred Metabolic Phenotypes’) of MAGs

MAG genes were assigned functions, and metabolic pathways were reconstructed using a combination of (i) public domain tools for sequence alignment and clustering, (ii) custom scripts to process the results of sequence alignments (e.g., for domain annotation in multifunctional proteins), and (iii) a reference collection of 2,856 human gut bacterial genomes for which reconstructed and manually-curated metabolic pathways were available related to 98 distinct metabolites and 106 metabolic phenotypes. These annotations are captured in the mcSEED database, a microbial community-centered adaptation of the SEED genomic platform, featuring subsystems-based annotation and pathway reconstruction applied to representative human gut bacterial genomes that were initially automatically annotated by RAST or downloaded from the PATRIC database. Each mcSEED subsystem includes a set of functional roles (e.g., enzymes, transporters, transcriptional regulators) that contribute to the prediction of functional metabolic pathways and pathway variants involved in utilization and catabolism carbohydrates and amino acids, biosynthesis of vitamins/cofactors and amino acids, and generation of fermentation end-products such as short-chain fatty acids.

Briefly, a reference database was constructed containing 995,591 functionally annotated proteins comprising the entire set of curated metabolic subsystems from the 2,856 reference genomes, plus an additional 2,988,751 proteins (‘outgroup’ not included in these metabolic subsystems), clustered at 90% amino acid identity (‘cluster’ command, MMSeqs; v1-c7a). The predicted protein sequences were aligned from the set of 1,000 high-quality MAGs against this reference protein database (DIAMOND, v2.0.0). To account for any influence of MAG fragmentation on metabolic reconstruction, gene fragments were identified using prodigal (v2.6.3) and were annotated in parallel. The following method were implemented to account for instances of multidomain structure that require multiple annotations. For each MAG query protein, top 50 hits were used based on the bitscore, and the start and end position coordinates of the corresponding alignments were clustered using DBSCAN (Scikit-learn), center of each clustered start and end position was used as potential domain boundary coordinates, and split query proteins into domains with database hits attributed to the corresponding domains. Next, for each domain ≥35 amino acids gaussian kernel density modeling was used (Kernel Density function, neighbors module, Scikit-learn, v0.22.1) of the sequence identity distribution of each set of hits to that domain. A highest local minimum (argrelextrema function, signal module, Scikit-learn) was employed as a threshold to remove low confidence hits. Finally, functional annotations were applied from the reference database to each query protein or domain by majority rule within each set of high-scoring, domain-specific reference hits. High-identity hits to proteins from the outgroup of the reference database were used as criteria to vote “against” applying annotation to each query. This procedure yielded a set of 199,334 annotated MAG proteins, representing 1,308 unique protein products across a set of 80 mcSEED subsystems.

Phenotype prediction strategies—The results of gene-level functional annotation were integrated into in silico predictions of the presence or absence (denoted as binary: “1” for presence or “0” for absence) of 106 functional metabolic pathways using a semi-automated process based on a combination of the following three approaches:

Pathway Rules (PR)-based phenotype predictions—This approach uses explicit logic-based “pathway rules” to assign binary phenotypes. These rules combine (i) expert curators' knowledge regarding the gene composition of various metabolic pathway variants contained in the mcSEED database with (ii) a decision tree method to identify patterns of gene representation in reference genomes corresponding to an intact functional pathway variant (and a respective binary phenotype value denoted as “1”). A total of 106 functional pathway-specific decision trees were generated (Rpart, v4.1.15), where the presence or absence of a particular phenotype was the response variable, and the presence or absence of functional roles (encoded by genes) in each reference pathway were predictor variables. The resulting pathway rules were formally encoded into a custom R script that allowed us to process MAG gene data and assign values (1 or 0) for each of the 106 functional metabolic pathways.

Machine Learning (ML)-based phenotype predictions—>30 ML methods (Caret, v6.0.86) were compared, using a ‘leave one out’ cross-validation approach in which a single reference genome was removed from the set of 2,856 reference genomes, trained ML models on the remaining genomes, then applied the models to the “test” genome to predict phenotypes. This procedure was then repeated for each genome and each metabolic phenotype. The results of this analysis identified Random Forest as the best-performing method (i.e., it produced the greatest number of correctly predicted phenotypes in the reference training dataset). Random Forest models were built for each phenotype based on the reference dataset, optimized model parameters using a grid search, and used these models to predict binary (I/O) values for the same set of 106 phenotypes for all MAGs.

Neighbor Group (NG)-based phenotype predictions—This approach identifies reference bacteria that are closely related to the MAGs in this study and uses these high-quality reference genomes for phenotype predications that are robust to variation in MAG quality. Examination of groups of closely related reference organisms suggested that close phylogenetic neighbor genomes tend to either possess or lack an entire pathway variant, whereas more distant neighbors (e.g., other neighbor groups) often carry more divergent pathway variants that specify the same phenotype. This observation was used to develop heuristics that minimize false negative phenotype assignments emerging from the other two prediction strategies. A set of NGs was compiled that comprised of MAGs and closely related reference genomes (Mash/MinHash distance ≤0.1, corresponding to ANI≥90%). At this similarity threshold, 640 of the 1,000 MAGs from this study were assigned to NGs containing from as few as four to more than 100 members. Within each NG and for each metabolic pathway, a binary phenotype value was tentatively assigned for a given MAG based on the NG genome with the closest matching gene annotation pattern (based on Hamming distance), even if some of the genes were absent in the query MAG. Limited comparisons of genes was required for the function of each respective pathway.

Consensus phenotype predictions—A procedure was developed to reconcile inconsistent phenotype predictions between the three strategies described above, based on observing discordant gene patterns and/or discordant predicted phenotypes within a given group of neighbor genomes. In the rare case of irreconcilable disagreement between the prediction methods, assignment of a consensus phenotype defaulted to that produced by the ML method. Consensus confidence scores were assigned to each prediction based on the degree of concordance between the three techniques. The complete phenotype prediction process was validated using the 2,856 reference genomes in the mcSEED database, their functionally annotated genes and the accompanying patterns of presence/absence of functional metabolic pathways (curator-inferred binary phenotypes). The consensus phenotype predictions were combined into a binary phenotype matrix (BPM) containing 1,000 MAGs and 106 phenotypes.

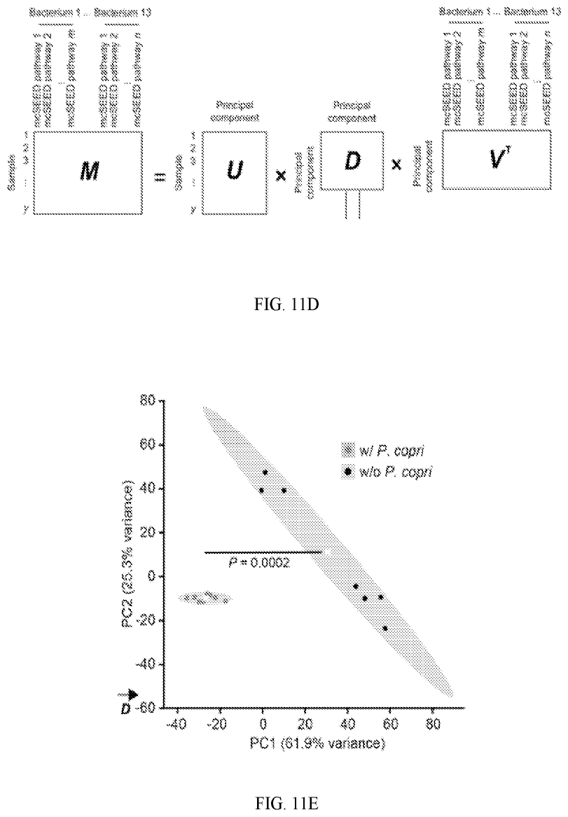

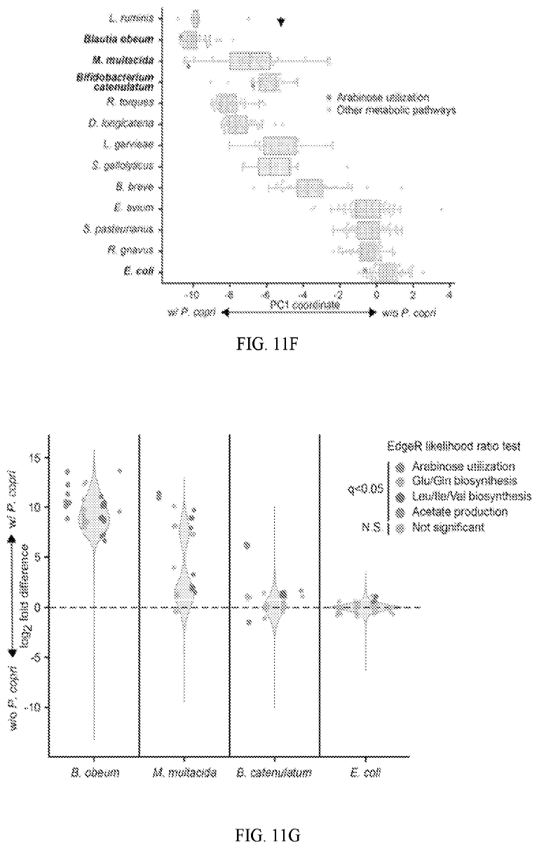

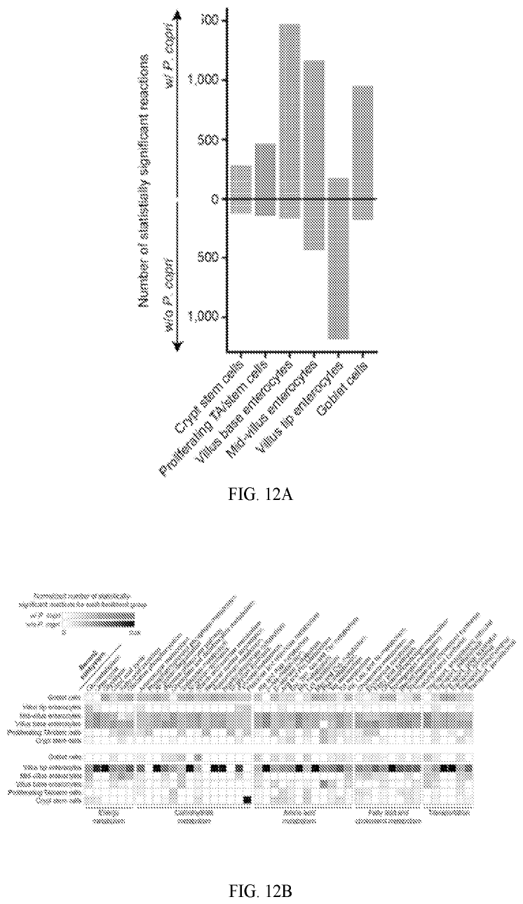

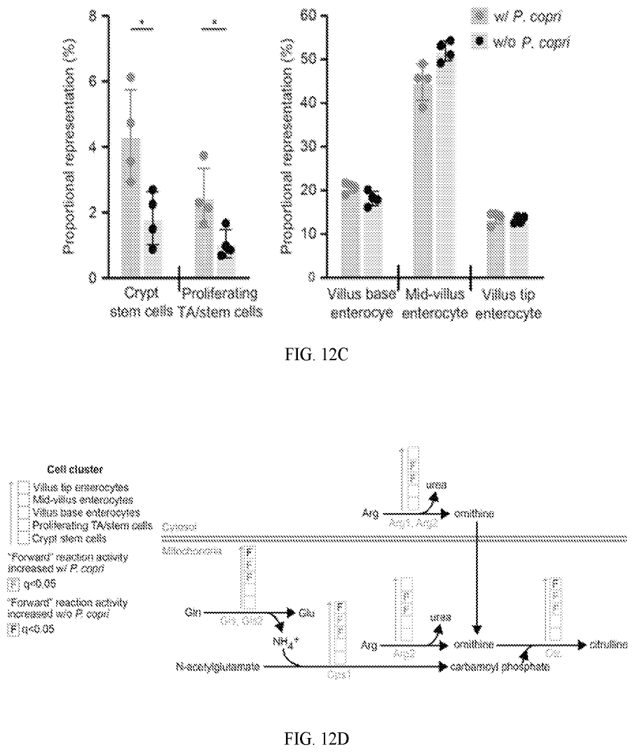

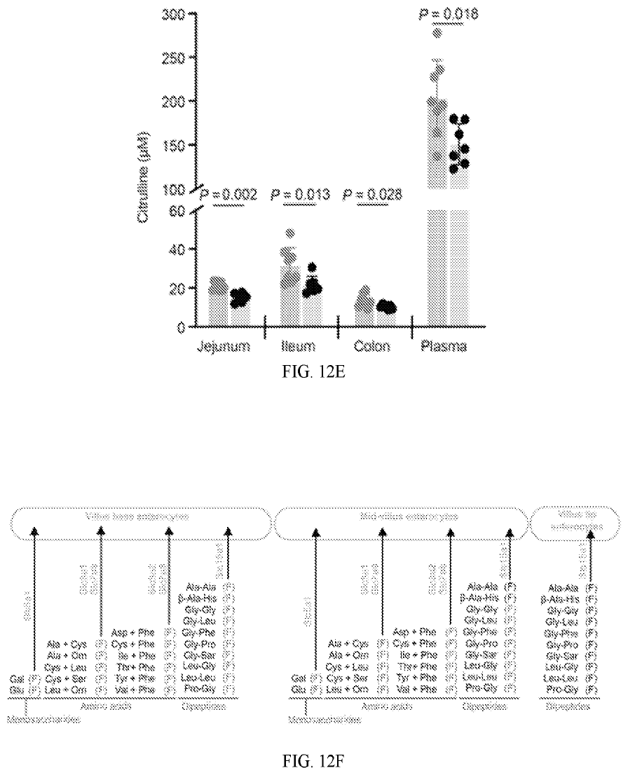

Gene annotation and phenotype prediction for Bifidobacteria-specific carbohydrate utilization pathways—MAG annotation pipeline described above was adapted (also see FIGS. 12D, 12E and 12F) to obtain functional annotations of genes comprising 25 additional carbohydrate utilization pathways for a set of 34 Bifidobacterium MAGs followed by inference of respective binary phenotypes. As input data for this set of Bifidobacterium-specific phenotypes, a set of 14 metabolic subsystems were curated in 387 reference human gut-derived Bifidobacterium genomes using the mcSEED framework. The reconstructed metabolic pathways and a corresponding BPM for reference Bifidobacterium genomes were used to predict carbohydrate utilization phenotypes in the 34 Bifidobacterium MAGs. Finally, the automatically generated BPM was further manually curated to account for the variability of certain pathways in this taxonomically restricted set of predictions.

Applying enrichment analyses to predicted MAG phenotypes—Not all successfully annotated MAG genes were components of an intact functional pathway. To enable inferred phenotype-based analysis, gene annotations were filtered to those that were part of a complete functional pathway (with a respective binary phenotype value denoted as “1”). This filter resulted in a list of 208,246 genes used for microbiome and meta transcriptome phenotype enrichment analyses.

### Example 2: Reconstructing Bacterial Genomes Associated with Ponderal Growth Responses

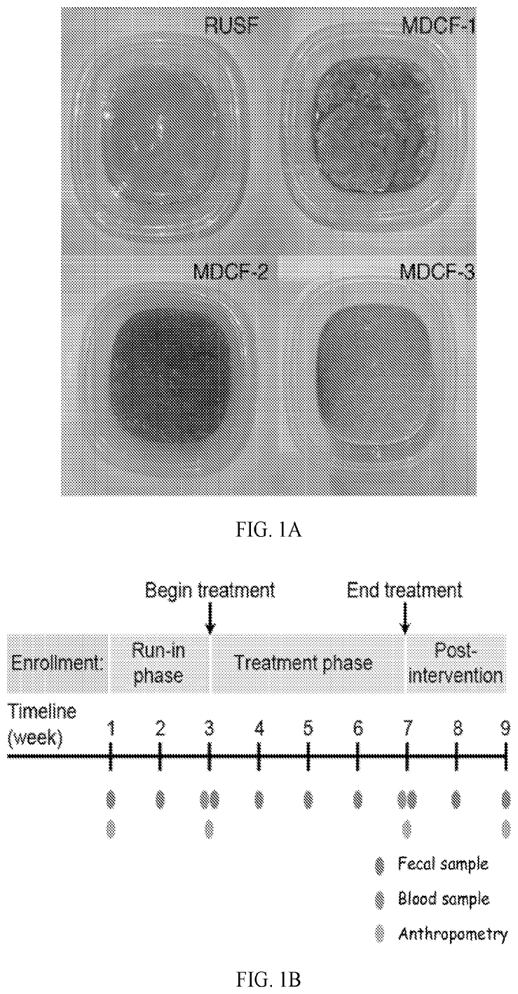

Children aged 12-18 months, with MAM (WLZ-2 to -3) were fed two 25 g servings/d corresponding to 100-125 kcal/serving, with fresh daily produced RUSF, MDCF-1, MDCF-2, MDCF-3 as shown in FIGS. 1A and 1B. Levels of >1300 plasma proteins were monitored that are key regulators of many aspects of growth and health. The effects on gut microbiota were also monitored. Further experiments were conducted with MDCF-2.

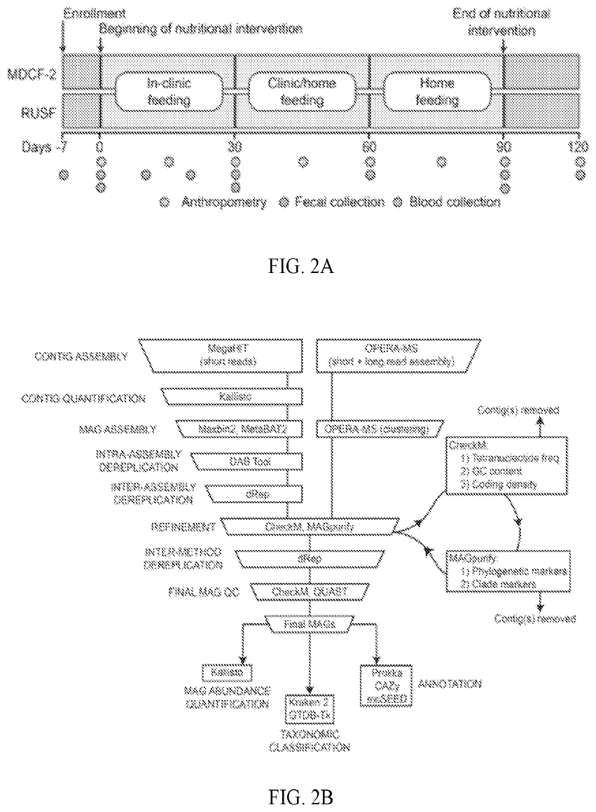

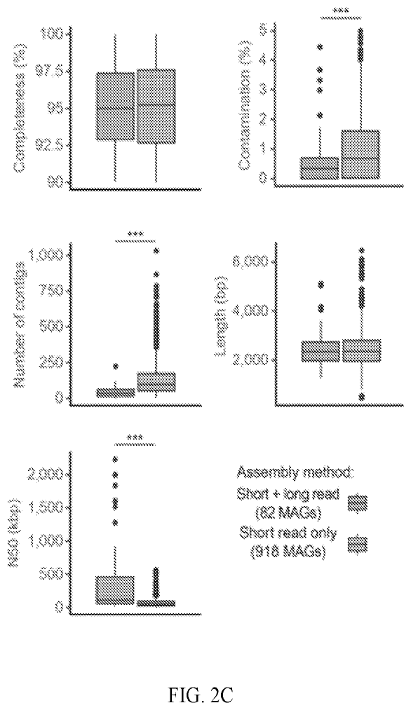

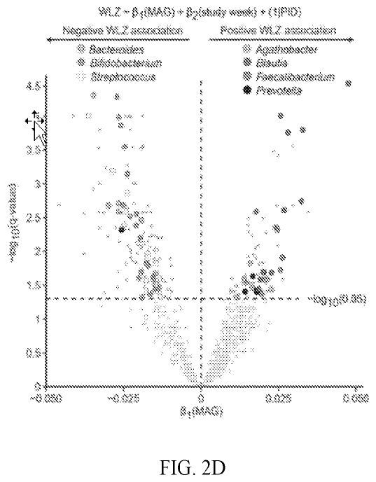

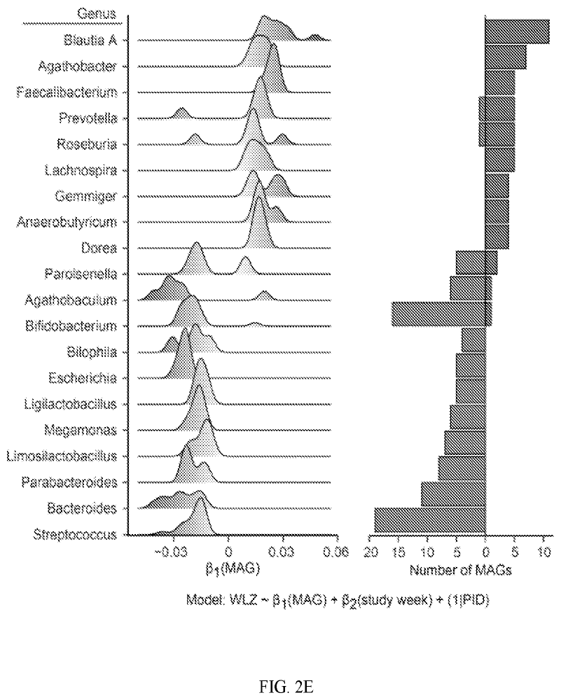

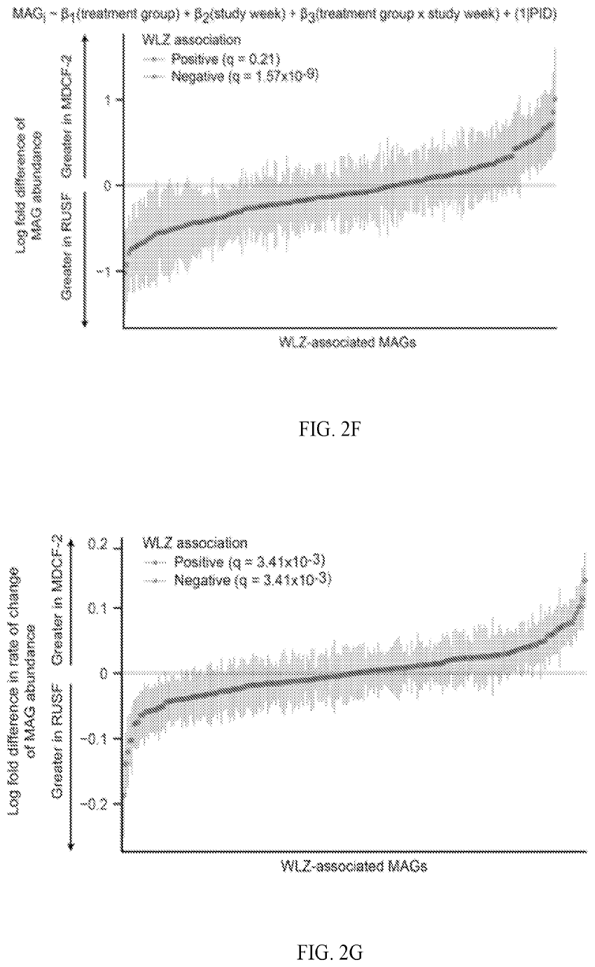

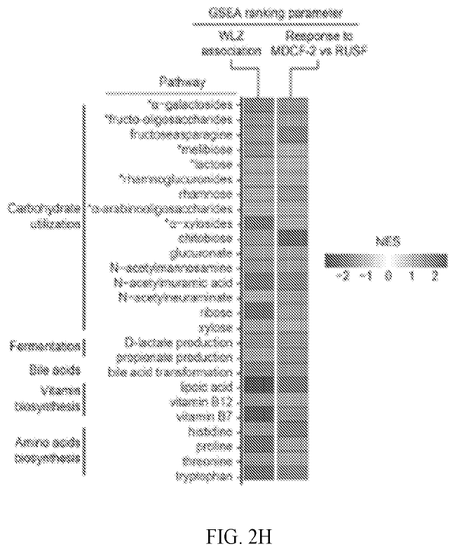

FIG. 2A summarizes the design of the completed randomized, controlled feeding study of children with MAM, aged 15.4±2.0 months (mean±SD) at enrollment. These children lived in an impoverished urban area (Mirpur) located in Dhaka, Bangladesh. The 3-month intervention involved twice-daily dietary supplementation with either MDCF-2 or RUSF. A total of 59 children in each treatment group completed the intervention and a 1-month follow-up 4. There were no statistically significant differences in the amount of nutritional supplement consumed between children receiving MDCF-2 versus RUSF, no differences in the proportion of children who satisfied current World Health Organization requirements for minimum meal frequency or minimum acceptable diet, and no differences in the amount of breast milk consumed between the two treatment groups. Fecal samples were collected every 10 days during the first month and every 4 weeks thereafter.

To reconstruct the genomes of bacterial taxa present in the gut microbiomes of study participants, DNA was isolated from all fecal samples (n=942; 7-8 samples/participant) and performed short-read shotgun sequencing. DNA recovered from fecal biospecimens collected at t=0 and 3 months from the subset of participants comprising the upper-quartile of the ponderal growth response to MDCF-2 (n=15) were subjected to additional long-read sequencing. Pooled shotgun sequencing data was assembled from each participant's fecal samples (short-read only, or short-plus long-reads when available) and aggregated contigs into metagenome-assembled genomes (MAGs) (FIGS. 2B and 2C). The resulting set of 1,000 high-quality MAGs (defined as ≥90% complete and ≤5% contaminated based on marker gene analysis) represented 65.6±8.0% and 66.2±7.9% of all quality controlled, paired-end shotgun reads generated from all 942 fecal DNA samples analyzed in the MDCF-2 and RUSF treatment groups, respectively [2.3±1.4×10⁷ 150 nt paired-end reads/sample (mean±SD)]. Taxonomy was assigned to MAGs using a consensus approach that included marker gene and kmer-based classification together with the Genome Taxonomy Database. Abundances were calculated for each MAG in the 707 fecal samples that spanned the beginning of treatment through the 1-month post-intervention timepoint and for which matching anthropometric measurements from children had been collected. A total of 837 MAGs satisfied the abundance and prevalence thresholds. A linear mixed-effects models was used to identify 222 MAGs whose abundances were significantly associated with WLZ [31 (MAG), q<0.05, FIG. 2D] over the 90-day course of the intervention and 30-day follow-up. MAGs that were significantly positively associated with WLZ were predominantly members of the genera Agathobacter, Blautia, Faecalibacterium and Prevotella while members of Bacteroides, Bifidobacterium and Streptococcus were prevalent among MAGs negatively associated with WLZ (FIGS. 2D and 2E).

Changes in MAG abundances were subsequently modeled as a function of treatment group, study week, and the interaction between treatment group and study week, controlling for repeated measurements taken from the same individual (see equation in FIG. 2F and Methods). The ‘treatment group’ coefficient describes the mean difference in abundance of a given MAG between the MDCF-2 and RUSF groups over the course of the intervention (FIG. 2F), while the interaction coefficient in the equation describes the difference in the rate of change in abundance of a given MAG (FIG. 2G). Restricting this analysis to the time of initiation of treatment did not reveal any statistically significant differences in MAG abundances between the two groups (q>0.05, one linear model per MAG). Expanding the analysis to include all time points disclosed that MAGs whose abundances increased faster in the MDCF-2 group compared to in the RUSF group were significantly enriched for those positively associated with WLZ [q=3.41×10⁻³, gene set enrichment analysis (GSEA); FIG. 2F]. In contrast, MAGs with a higher mean abundance as well as those that increased more rapidly in RUSF-treated children were significantly enriched for those negatively associated with WLZ (q=1.57×10⁻⁹ and q=3.41×10⁻³, respectively; GSEA) (FIGS. 2E and 2F).

A ‘subsystems’ approach was adapted from the SEED genome annotation platform to identify genes that comprise metabolic pathways represented in WLZ-associated MAGs. To do so, genes were aligned to a reference collection of 2,856 human gut bacterial genomes that had been subjected to in silico reconstructions of metabolic pathways in mcSEED, a microbial community-centered implementation of SEED. Putative functions were assigned to a subset of 199,334 proteins in all 1,000 MAGs; these proteins, which represented 1,308 nonredundant functions, formed the basis for predicting which of 106 metabolic pathways, curated across a reference collection of 2,856 representative human gut bacterial genomes and reflecting major nutrient utilization capabilities, were present or absent in each MAG. This effort generated a set of inferred metabolic phenotypes for each MAG. GSEA disclosed multiple metabolic pathways involved in utilization of carbohydrates that were significantly (q<0.05) enriched in WLZ-associated MAGs, and in MAGs ranked by abundance response to MDCF-2 compared to RUSF treatment. While other non-carbohydrate pathways were also identified using this approach (e.g., those involved in aspects of amino acid and bile acid metabolism), pathways involved in carbohydrate utilization predominated (P=0.006, Fisher's test; FIG. 2H; Tables 1, 2 and 3).

TABLE 1
GSEA for the presence or absence of a functional pathway in MAGs ranked by WLZ association

| | | | Number | | | |
|---|---|---|---|---|---|---|
| | | | of | | | |
| | | Scoring | MAGs | | | |
| | | for | with | | | |
| | | functional | functional | | | |
| | | pathway | pathway | Normalized | | |
| | Functional | presence | present | enrichment | | P-value |
| | pathway | or | or | score | | (FDR- |
| Functional pathway | abbreviation | absence | absent | (NES) | P-value | adjusted) |
| lacto-N-biose | Lnb | present | 109 | 2.2 | 1.8E−07 | 1.2E−06 |
| utilization | | | | | | |
| bile acid | BA_t | present | 257 | 2.1 | 1.9E−10 | 2.4E−09 |
| transformation | | | | | | |
| alpha-galactosides | Aga | present | 143 | 2.1 | 2.1E−07 | 1.4E−06 |
| utilization | | | | | | |
| melibiose utilization | Mel | present | 248 | 1.8 | 6.7E−07 | 3.6E−06 |
| cobalamin cofactors, | B12 | present | 367 | 1.8 | 5.9E−08 | 5.0E−07 |
| de novo synthesis | | | | | | |
| fructooligosaccharides | FOS | present | 283 | 1.8 | 1.0E−06 | 5.3E−06 |
| utilization | | | | | | |
| butyrate production | Butyrate | present | 233 | 1.8 | 6.2E−06 | 2.4E−05 |
| production | | | | | | |
| fructoseasparagine | FruAsn | present | 10 | 1.7 | 1.8E−02 | 3.1E−02 |
| utilization | | | | | | |
| propanediol utilization | PD_ut | present | 97 | 1.6 | 2.8E−03 | 6.3E−03 |
| glucuronides utilization | GlcAs | present | 70 | 1.6 | 1.1E−02 | 1.8E−02 |
| oligogalacturonate | GalAs | present | 142 | 1.5 | 5.6E−03 | 1.1E−02 |
| utilization | | | | | | |
| rhamnogalacturonides | Rhi | present | 144 | 1.4 | 4.7E−03 | 9.4E−03 |
| utilization | | | | | | |
| beta-glucosides | Bgl | present | 355 | 1.4 | 1.0E−03 | 2.9E−03 |
| utilization | | | | | | |
| folate cofactors, de | B9 | absent | 296 | 1.4 | 7.2E−04 | 2.0E−03 |
| novo synthesis | | | | | | |
| PLP/PMP cofactors, de | B6 | absent | 367 | 1.3 | 2.6E−03 | 6.0E−03 |
| novo synthesis | | | | | | |
| L-lactate production | L-Lactate | absent | 260 | −1.4 | 2.1E−02 | 3.3E−02 |
| production | | | | | | |
| N-acetylneuraminate | NANA | present | 187 | −1.4 | 2.3E−02 | 3.6E−02 |
| utilization | | | | | | |
| N-acetylglucosamine | GlcNAc | present | 379 | −1.4 | 7.9E−03 | 1.4E−02 |
| utilization | | | | | | |
| xylooligosaccharides | XOS | present | 178 | −1.4 | 3.2E−02 | 4.8E−02 |
| utilization | | | | | | |
| alpha- | aAOS | present | 128 | −1.4 | 2.7E−02 | 4.1E−02 |
| arabinooligosaccharides | | | | | | |
| utilization | | | | | | |
| coenzyme A, de novo | B5 | present | 308 | −1.4 | 4.5E−03 | 9.2E−03 |
| synthesis | | | | | | |
| cholic acid | CA_d | absent | 347 | −1.4 | 3.3E−03 | 7.2E−03 |
| deconjugation | | | | | | |
| lactose utilization | Lac | present | 368 | −1.5 | 2.0E−03 | 4.9E−03 |
| ethanolamine | EA_ut | present | 43 | −1.5 | 2.3E−02 | 3.6E−02 |
| utilization | | | | | | |
| chorismate | Chor | absent | 38 | −1.5 | 3.8E−02 | 5.8E−02 |
| biosynthesis | | | | | | |
| rhamnose utilization | Rha | present | 102 | −1.5 | 1.1E−02 | 1.9E−02 |
| propionate production | Propionate | present | 193 | −1.6 | 2.3E−03 | 5.5E−03 |
| production | | | | | | |
| mannitol utilization | Mtl | present | 132 | −1.6 | 4.4E−03 | 9.1E−03 |
| xylose utilization | Xyl | present | 144 | −1.6 | 4.3E−03 | 9.1E−03 |
| glucuronate | GlcA | present | 189 | −1.6 | 1.4E−03 | 3.8E−03 |
| utilization | | | | | | |
| Beta- | bMnOS | present | 173 | −1.6 | 1.5E−03 | 3.8E−03 |
| mannooligosaccharides | | | | | | |
| utilization | | | | | | |
| queuosine, de novo | Q | present | 344 | −1.6 | 9.7E−05 | 3.1E−04 |
| synthesis | | | | | | |
| tagatose utilization | Tag | present | 22 | −1.7 | 2.0E−02 | 3.2E−02 |
| maltose utilization | Mal | absent | 243 | −1.7 | 4.2E−05 | 1.4E−04 |
| N-acetylmannosamine | ManNAc | present | 34 | −1.7 | 6.9E−03 | 1.2E−02 |
| utilization | | | | | | |
| galactitol utilization | Gtl | present | 31 | −1.7 | 6.3E−03 | 1.2E−02 |
| vitamin K, de novo | MQ | present | 153 | −1.7 | 2.1E−04 | 6.5E−04 |
| synthesis | | | | | | |
| galactose utilization | Gal | absent | 368 | −1.7 | 2.3E−06 | 1.0E−05 |
| histidine degradation | His_d | present | 117 | −1.7 | 6.2E−04 | 1.8E−03 |
| glucoselysine | GlcLys | present | 32 | −1.7 | 6.6E−03 | 1.2E−02 |
| utilization | | | | | | |
| chitobiose utilization | Chb | present | 132 | −1.8 | 3.4E−04 | 1.0E−03 |
| unsaturated | ddGlcA | present | 28 | −1.8 | 5.7E−03 | 1.1E−02 |
| glucuronate utilization | | | | | | |
| allose utilization | All | present | 22 | −1.8 | 8.5E−03 | 1.5E−02 |
| maltooligosaccharides | MOS | absent | 326 | −1.8 | 1.4E−06 | 6.3E−06 |
| utilization | | | | | | |
| threonine degradation | Thr_d | present | 160 | −1.8 | 5.6E−05 | 1.9E−04 |
| trehalose utilization | Tre | present | 74 | −1.8 | 1.4E−03 | 3.8E−03 |
| D-lactate production | D-Lactate | present | 292 | −1.8 | 1.1E−06 | 5.5E−06 |
| production | | | | | | |
| psicoselysine utilization | PsiLys | present | 13 | −1.9 | 3.3E−03 | 7.3E−03 |
| fucose utilization | Fuc | present | 139 | −1.9 | 1.6E−05 | 5.5E−05 |
| lysine degradation | Lys_d | present | 17 | −1.9 | 1.5E−03 | 3.8E−03 |
| urea degradation | Urea_d | present | 100 | −2.0 | 8.8E−06 | 3.2E−05 |
| gluconate utilization | Gnt | present | 102 | −2.1 | 2.4E−06 | 1.0E−05 |
| arabinose utilization | Ara | present | 119 | −2.2 | 2.3E−07 | 1.4E−06 |
| fucosyllactose | FL | present | 13 | −2.2 | 7.3E−06 | 2.8E−05 |
| mannose utilization | Man | present | 270 | −2.2 | 5.4E−13 | 1.4E−11 |
| raffinose utilization | Raf | present | 31 | −2.3 | 4.6E−06 | 1.9E−05 |
| N-acetylmuramic acid | MurNac | present | 106 | −2.3 | 3.3E−09 | 3.7E−08 |
| utilization | | | | | | |
| galactosamine | GalN | present | 66 | −2.4 | 3.9E−08 | 3.6E−07 |
| utilization | | | | | | |
| alpha-xylosides | aXyl | present | 62 | −2.4 | 1.5E−08 | 1.5E−07 |
| utilization | | | | | | |
| xylitol utilization | Xlt | present | 22 | −2.4 | 6.5E−07 | 3.6E−06 |
| ribose utilization | Rbs | present | 210 | −2.4 | 9.2E−14 | 3.1E−12 |
| N-acetylgalactosamine | GalNAc | present | 38 | −2.4 | 1.0E−07 | 7.5E−07 |
| utilization | | | | | | |
| proline degradation | Pro_d | present | 44 | −2.4 | 6.9E−08 | 5.4E−07 |
| tryptophan degradation | Trp_d | present | 108 | −2.5 | 1.3E−11 | 1.9E−10 |
| Mevalonate synthesis 2 | IDX | absent | 75 | −2.7 | 1.0E−11 | 1.8E−10 |
| Mevalonate synthesis 1 | IMV | present | 69 | −2.7 | 1.1E−12 | 2.2E−11 |
| biotin cofactor, de | B7 | present | 141 | −2.8 | 1.2E−18 | 6.3E−17 |
| novo synthesis | | | | | | |
| lipoate cofactor, de | LA | present | 84 | −3.0 | 6.0E−19 | 6.2E−17 |
| novo synthesis | | | | | | |

TABLE 2
GSEA of pathway enrichment in MAGs ranked by change in abundance in response
to MDCF-2 compared to RUSF treatment (‘treatment group’ coefficient)

| | | | Number | | | |
|---|---|---|---|---|---|---|
| | | | of | | | |
| | | Scoring | MAGs | | | |
| | | for | with | | | |
| | | functional | functional | | | |
| | | pathway | pathway | Normalized | | |
| | Functional | presence | present | enrichment | | P-value |
| | pathway | or | or | score | | (FDR- |
| Functional pathway | abbreviation | absence | absent | (NES) | P-value | adjusted) |
| fructoseasparagine | FruAsn | present | 10 | 2.1 | 4.1E−05 | 3.4E−04 |
| utilization | | | | | | |
| sorbitol utilization | Srl | present | 141 | 1.9 | 1.1E−05 | 1.5E−04 |
| bile acid | BA_t | present | 257 | 1.8 | 1.4E−06 | 3.6E−05 |
| transformation | | | | | | |
| alpha-galactosides | Aga | present | 143 | 1.7 | 9.9E−05 | 7.4E−04 |
| utilization | | | | | | |
| proline biosynthesis | Pro | absent | 118 | 1.7 | 6.0E−04 | 3.6E−03 |
| fructooligosaccharides | FOS | present | 283 | 1.6 | 4.2E−05 | 3.4E−04 |
| utilization | | | | | | |
| folate cofactors, de | B9 | absent | 296 | 1.6 | 3.0E−05 | 2.9E−04 |
| novo synthesis | | | | | | |
| alpha- | aAOS | present | 128 | 1.5 | 5.4E−03 | 2.2E−02 |
| arabinooligosaccharides | | | | | | |
| utilization | | | | | | |
| cobalamin cofactors, | B12 | present | 367 | 1.5 | 6.0E−04 | 3.6E−03 |
| de novo synthesis | | | | | | |
| melibiose utilization | Mel | present | 248 | 1.4 | 4.1E−03 | 1.7E−02 |
| lactose utilization | Lac | present | 368 | 1.4 | 3.7E−03 | 1.6E−02 |
| glucose utilization | Glc | absent | 303 | 1.4 | 5.8E−03 | 2.2E−02 |
| xylose utilization | Xyl | present | 144 | −1.4 | 1.7E−02 | 4.9E−02 |
| NAD(P) cofactors, de | B3 | absent | 312 | −1.4 | 6.2E−03 | 2.3E−02 |
| novo synthesis | | | | | | |
| formate production | Formate | absent | 149 | −1.4 | 9.2E−03 | 3.0E−02 |
| production | | | | | | |
| rhamnogalacturonides | Rhi | present | 144 | −1.4 | 6.8E−03 | 2.5E−02 |
| utilization | | | | | | |
| threonine degradation | Thr_d | present | 160 | −1.4 | 1.0E−02 | 3.3E−02 |
| fructose utilization | Fru | absent | 226 | −1.5 | 2.8E−03 | 1.3E−02 |
| phenylalanine | Phe | absent | 83 | −1.5 | 1.3E−02 | 4.1E−02 |
| biosynthesis | | | | | | |
| alpha-xylosides | aXyl | present | 62 | −1.5 | 1.6E−02 | 4.6E−02 |
| utilization | | | | | | |
| ethanol production | Ethanol | absent | 333 | −1.5 | 6.2E−04 | 3.6E−03 |
| production | | | | | | |
| L-lactate production | L-Lactate | absent | 260 | −1.5 | 8.4E−04 | 4.2E−03 |
| production | | | | | | |
| proline degradation | Pro_d | present | 44 | −1.6 | 1.5E−02 | 4.5E−02 |
| ribose utilization | Rbs | present | 210 | −1.6 | 6.7E−04 | 3.7E−03 |
| glucuronate utilization | GlcA | present | 189 | −1.6 | 7.8E−04 | 4.1E−03 |
| N-acetylneuraminate | NANA | present | 187 | −1.6 | 1.2E−03 | 5.7E−03 |
| utilization | | | | | | |
| N-acetylmannosamine | ManNAc | present | 34 | −1.6 | 8.3E−03 | 2.8E−02 |
| utilization | | | | | | |
| methionine | Met_d | present | 42 | −1.7 | 7.1E−03 | 2.5E−02 |
| degradation | | | | | | |
| D-lactate production | D-Lactate | present | 292 | −1.7 | 1.2E−05 | 1.5E−04 |
| production | | | | | | |
| rhamnose utilization | Rha | present | 102 | −1.8 | 2.5E−04 | 1.7E−03 |
| propionate production | Propionate | present | 193 | −1.8 | 2.1E−05 | 2.2E−04 |
| production | | | | | | |
| biotin cofactor, de novo | B7 | present | 141 | −1.8 | 2.1E−05 | 2.2E−04 |
| synthesis | | | | | | |
| N-acetylmuramic acid | MurNac | present | 106 | −2.0 | 5.0E−06 | 8.8E−05 |
| utilization | | | | | | |
| tryptophan degradation | Trp_d | present | 108 | −2.0 | 4.4E−06 | 8.8E−05 |
| histidine degradation | His_d | present | 117 | −2.1 | 1.8E−07 | 6.3E−06 |
| lipoate cofactor, de | LA | present | 84 | −2.3 | 3.6E−08 | 1.9E−06 |
| novo synthesis | | | | | | |
| chitobiose utilization | Chb | present | 132 | −2.6 | 6.5E−14 | 6.8E−12 |

TABLE 3
GSEA of metabolic pathways in MAGs ranked by change in abundance
in response to MDCF-2 compared to RUSF treatment (interaction
between ‘treatment group’ and ‘study week’ coefficients)

| | | | Number | | | |
|---|---|---|---|---|---|---|
| | | | of | | | |
| | | Scoring | MAGS | | | |
| | | for | with | | | |
| | | functional | functional | | | |
| | | pathway | pathway | Normalized | | |
| | Functional | presence | present | enrichment | | P-value |
| | pathway | or | or | score | | (FDR- |
| Functional pathway | abbreviation | absence | absent | (NES) | P-value | adjusted) |
| lacto-N-biose | Lnb | present | 109 | 2.3 | 2.5E−09 | 2.6E−07 |
| utilization | | | | | | |
| inositol utilization | Ino | present | 103 | 1.9 | 1.2E−05 | 2.6E−04 |
| fructoselysine | FruLys | present | 96 | 1.9 | 4.5E−05 | 7.8E−04 |
| utilization | | | | | | |
| folate cofactors, de | B9 | absent | 296 | 1.6 | 1.1E−04 | 1.1E−03 |
| novo synthesis | | | | | | |
| PLP/PMP cofactors, de | B6 | absent | 367 | 1.6 | 1.2E−04 | 1.1E−03 |
| novo synthesis | | | | | | |
| beta-glucosides | Bgl | present | 355 | 1.6 | 1.0E−04 | 1.1E−03 |
| utilization | | | | | | |
| bile acid | BA_t | present | 257 | 1.5 | 1.4E−03 | 9.4E−03 |
| transformation | | | | | | |
| tryptophan | Trp | absent | 340 | 1.5 | 9.7E−04 | 7.3E−03 |
| biosynthesis | | | | | | |
| glucose utilization | Glc | absent | 303 | 1.5 | 2.3E−03 | 1.3E−02 |
| maltooligosaccharides | MOS | absent | 326 | 1.5 | 1.6E−03 | 9.4E−03 |
| utilization | | | | | | |
| rhamnose utilization | Rha | present | 102 | 1.5 | 1.1E−02 | 4.4E−02 |
| D-lactate production | D-Lactate | present | 292 | 1.5 | 1.6E−03 | 9.4E−03 |
| production | | | | | | |
| galactooligosaccharides | GOS | present | 176 | −1.4 | 4.2E−03 | 2.0E−02 |
| utilization | | | | | | |
| vitamin K, de novo | MQ | present | 153 | −1.5 | 2.7E−03 | 1.4E−02 |
| synthesis | | | | | | |
| mannitol utilization | Mtl | present | 132 | −1.5 | 5.0E−03 | 2.2E−02 |
| cysteine biosynthesis | Cys | absent | 130 | −1.6 | 1.5E−03 | 9.4E−03 |
| glutamine biosynthesis | Gln | absent | 47 | −1.6 | 8.1E−03 | 3.4E−02 |
| xylooligosaccharides | XOS | present | 178 | −1.6 | 1.4E−04 | 1.2E−03 |
| utilization | | | | | | |
| fructooligosaccharides | FOS | present | 283 | −1.7 | 4.3E−06 | 1.1E−04 |
| utilization | | | | | | |
| glutamate biosynthesis | Glu | absent | 45 | −1.7 | 4.3E−03 | 2.0E−02 |
| xylose utilization | Xyl | present | 144 | −1.7 | 1.2E−04 | 1.1E−03 |
| alpha-xylosides | aXyl | present | 62 | −1.9 | 6.1E−04 | 4.9E−03 |
| utilization | | | | | | |
| xylitol utilization | Xlt | present | 22 | −1.9 | 2.4E−03 | 1.3E−02 |
| fucosyllactose | FL | present | 13 | −2.2 | 8.0E−05 | 1.1E−03 |
| alpha- | aAOS | present | 128 | −2.3 | 6.9E−09 | 3.6E−07 |
| arabinooligosaccharides | | | | | | |
| utilization | | | | | | |
| raffinose utilization | Raf | present | 31 | −2.4 | 3.8E−06 | 1.1E−04 |

### Example 3: Carbohydrate Composition of MDCF-2 and RUSF

Prior to analyzing the transcriptional responses of MAGs to each nutritional intervention, the carbohydrates present in MDCF-2 and RUSF were characterized, as well as their constituent Bangladeshi-sourced food ingredients [chickpea flour, soybean flour, peanut paste and mashed green banana pulp in the case of MDCF-2; rice, lentil and milk powder in the case of RUSF (Table 4 and Table 30A-C).

TABLE 4
Composition of MDCF-2 and RUSF diets.

| | |
|---|---|
| MDCF2 | RUSF |

| | |
|---|---|
| Component | g/100 g diet |

| | | |
|---|---|---|
| Raw banana | 19 | 0 |
| Chickpea flour | 10 | 0 |
| Peanut flour | 10 | 0 |
| Soy flour | 8 | 0 |
| Lentil | 0 | 21.5 |
| Rice | 0 | 18.9 |
| Powdered skimmed milk | 0 | 10.5 |
| Sugar | 29.9 | 17 |
| Soybean oil | 20 | 29 |
| Micronutrient premix | 3.14 | 3.14 |
| Energy content (kcal/g dry wt.)^{a} | 4.66 | 5.34 |

Ultrahigh-performance liquid chromatography-triple quadrupole mass spectrometry (UHPLC-QqQ-MS) was used to quantify 14 monosaccharides and 49 unique glycosidic linkages. Polysaccharide content was defined using a procedure in which polysaccharides were chemically cleaved into oligosaccharides, after which the structures of these liberated oligosaccharides were used to characterize and quantify their ‘parent’ polysaccharide.

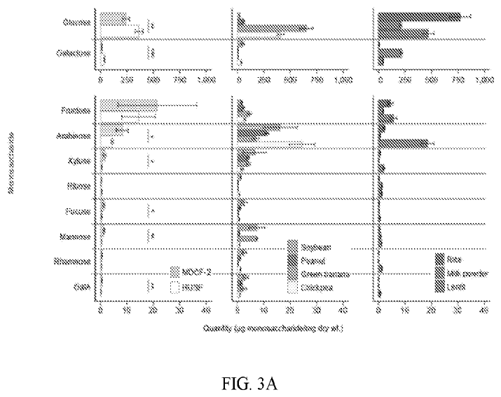

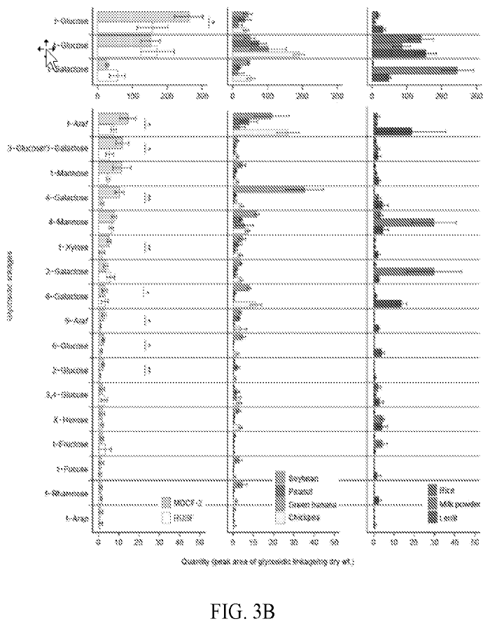

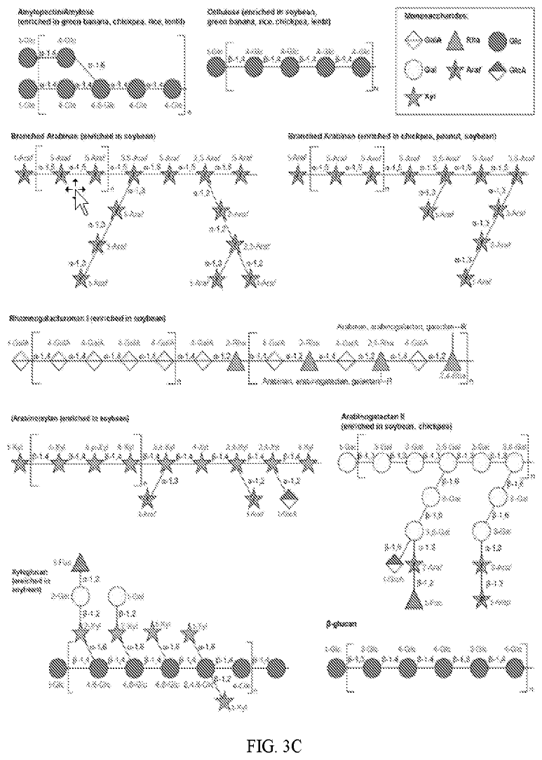

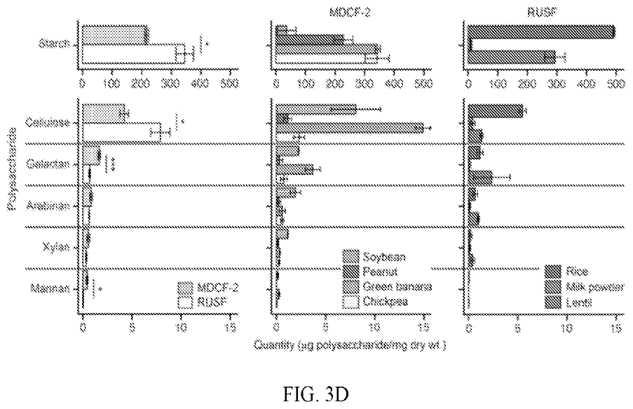

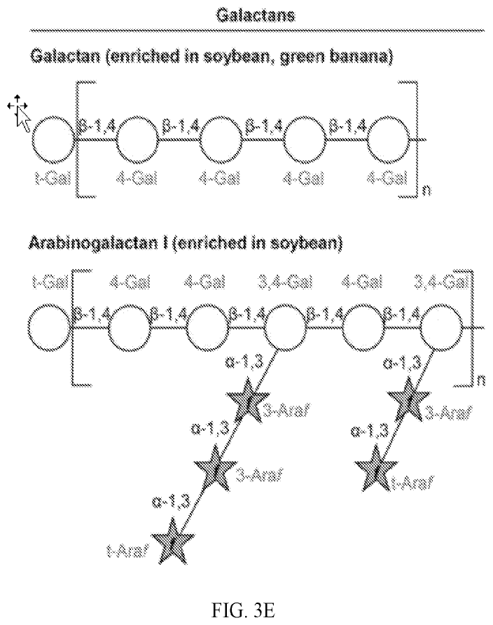

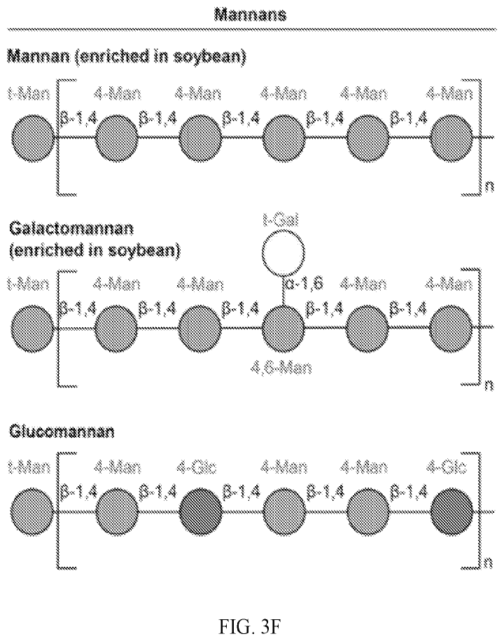

The results revealed that L-arabinose, D-xylose, L-fucose, D-mannose, and D-galacturonic acid (GalA) are significantly more abundant in MDCF-2 (q<0.05; t-test) as are eight linkages, three of which contain these monosaccharides (FIGS. 3A and 3B; Table 5 and 6).

TABLE 5
Difference in monosaccharide composition between MDCF-2
and RUSF (μg of monosaccharide/mg of dried diet.

| | | | | | | | Comparison of MDCF-2 and | | |
|---|---|---|---|---|---|---|---|---|---|
| | MDCF-2 | | | RUSF | | | RUSF (t-test) | P-value | |
| Monosaccharide | mean ± SD | | | mean ± SD | | | log2 (MDCF-2/RUSF) | q-value | |
| Galactose | 14.29 | ± | 2.40 | 36.85 | ± | 4.04 | −1.37 | 0.000 | 0.003 |
| GalA | 0.64 | ± | 0.12 | 0.23 | ± | 0.07 | 1.49 | 0.002 | 0.011 |
| Glucose | 243.80 | ± | 31.51 | 364.21 | ± | 36.65 | −0.58 | 0.003 | 0.011 |
| Mannose | 1.39 | ± | 0.24 | 0.41 | ± | 0.04 | 1.76 | 0.003 | 0.011 |
| Fucose | 1.24 | ± | 0.37 | 0.46 | ± | 0.25 | 1.42 | 0.016 | 0.046 |
| Xylose | 1.58 | ± | 0.53 | 0.56 | ± | 0.08 | 1.50 | 0.029 | 0.068 |
| Arabinose | 8.22 | ± | 2.25 | 4.45 | ± | 0.48 | 0.88 | 0.041 | 0.082 |
| Ribose | 0.38 | ± | 0.07 | 0.52 | ± | 0.09 | −0.46 | 0.050 | 0.088 |
| Rhamnose | 0.58 | ± | 0.19 | 0.36 | ± | 0.06 | 0.69 | 0.094 | 0.147 |
| GlcNAc | 0.02 | ± | 0.02 | 0.08 | ± | 0.09 | −1.69 | 0.288 | 0.403 |
| Fructose | 21.44 | ± | 14.90 | 14.46 | ± | 6.38 | 0.57 | 0.437 | 0.516 |
| GlcA | 0.14 | ± | 0.05 | 0.12 | ± | 0.05 | 0.32 | 0.451 | 0.516 |
| Allose | 0.01 | ± | 0.01 | 0.00 | ± | 0.00 | 1.08 | 0.479 | 0.516 |
| GalNAc | 0.02 | ± | 0.02 | 0.03 | ± | 0.04 | −0.77 | 0.563 | 0.563 |

TABLE 6
Difference in glycosidic linkage content between MDCF-2
and RUSF (peak area, arbitrary units/ng dried diet)

| | | | | | | | Comparison of MDCF-2 | | |
|---|---|---|---|---|---|---|---|---|---|
| | MDCF-2 | | | RUSF | | | and RUSF (t-test) | | |
| Glycosidic linkage | mean ± SD | | | mean ± SD | | | log2 (MDCF-2/RUSF) | P-value | q-value |
| 2-Xylose | 0.77 | ± | 0.16 | 1.8E−01 | ± | 1.5E−01 | 2.11 | 0.002 | 0.055 |
| 4-Galactose | 10.30 | ± | 2.21 | 1.7E+00 | ± | 8.3E−01 | 2.60 | 0.002 | 0.055 |
| T-P-Xylose | 5.23 | ± | 0.92 | 1.6E+00 | ± | 1.3E+00 | 1.71 | 0.004 | 0.072 |
| 4,6-Mannose | 0.13 | ± | 0.03 | 4.1E−02 | ± | 9.4E−03 | 1.67 | 0.007 | 0.080 |
| 2-Glucose | 2.12 | ± | 0.49 | 8.3E−01 | ± | 1.9E−01 | 1.35 | 0.008 | 0.080 |
| T-Glucose | 262.65 | ± | 40.36 | 1.6E+02 | ± | 4.4E+01 | 0.74 | 0.013 | 0.106 |
| T-GlcA | 0.07 | ± | 0.02 | 2.9E−02 | ± | 6.3E−03 | 1.28 | 0.018 | 0.122 |
| 3-Glucose_3- | 11.68 | ± | 3.12 | 5.5E+00 | ± | 1.9E+00 | 1.09 | 0.020 | 0.122 |
| Galactose | | | | | | | | | |
| 2,4-P-Xylose | 0.12 | ± | 0.04 | 4.9E−02 | ± | 2.8E−02 | 1.28 | 0.027 | 0.148 |
| 6-Glucose | 2.26 | ± | 0.51 | 1.2E+00 | ± | 5.4E−01 | 0.88 | 0.032 | 0.153 |
| T-F-Arabinose | 14.36 | ± | 3.97 | 7.4E+00 | ± | 1.2E+00 | 0.95 | 0.034 | 0.153 |
| T-GalA | 0.33 | ± | 0.10 | 1.6E−01 | ± | 2.2E−02 | 1.07 | 0.042 | 0.167 |
| 3-Arabinose | 0.42 | ± | 0.12 | 2.3E−01 | ± | 5.7E−02 | 0.87 | 0.044 | 0.167 |
| 5-F-Arabinose | 2.73 | ± | 1.14 | 9.5E−01 | ± | 2.4E−01 | 1.53 | 0.049 | 0.172 |
| T-Mannose | 11.48 | ± | 4.53 | 4.7E+00 | ± | 8.9E−01 | 1.29 | 0.055 | 0.179 |
| T-Galactose | 27.06 | ± | 4.57 | 5.6E+01 | ± | 2.2E+01 | −1.06 | 0.070 | 0.210 |
| 2-F-Arabinose | 0.59 | ± | 0.19 | 3.5E−01 | ± | 1.2E−01 | 0.78 | 0.073 | 0.210 |
| 2-Galactose | 3.53 | ± | 1.16 | 5.9E+00 | ± | 2.0E+00 | −0.73 | 0.100 | 0.246 |
| 2,5-F-Arabinose | 0.05 | ± | 0.01 | 3.5E−02 | ± | 1.7E−02 | 0.66 | 0.101 | 0.246 |
| 2,X,X-Hexose(II) | 0.25 | ± | 0.09 | 3.9E−01 | ± | 1.1E−01 | −0.63 | 0.106 | 0.246 |
| 3,4-P-Xylose_3,5- | 0.58 | ± | 0.27 | 2.8E−01 | ± | 9.5E−02 | 1.05 | 0.107 | 0.246 |
| Arabinose | | | | | | | | | |
| 4-Mannose | 7.71 | ± | 1.20 | 6.3E+00 | ± | 9.5E−01 | 0.30 | 0.110 | 0.246 |
| 3-Mannose | 0.21 | ± | 0.09 | 1.2E−01 | ± | 6.7E−02 | 0.76 | 0.169 | 0.360 |
| 2-Mannose | 0.16 | ± | 0.10 | 7.3E−02 | ± | 1.7E−02 | 1.10 | 0.198 | 0.404 |
| 3,4,6-Galactose | 0.06 | ± | 0.01 | 4.5E−02 | ± | 2.2E−02 | 0.44 | 0.255 | 0.465 |
| 3,4,6-Glucose | 0.42 | ± | 0.13 | 7.8E−01 | ± | 5.1E−01 | −0.89 | 0.255 | 0.465 |
| 2,3-F-Arabinose | 0.30 | ± | 0.13 | 2.0E−01 | ± | 7.9E−02 | 0.59 | 0.258 | 0.465 |
| 4,6-Galactose | 0.47 | ± | 0.20 | 3.3E−01 | ± | 1.2E−01 | 0.53 | 0.267 | 0.465 |
| 2,4-Glucose | 0.26 | ± | 0.08 | 3.8E−01 | ± | 1.9E−01 | −0.59 | 0.275 | 0.465 |
| 3,4-Glucose | 1.71 | ± | 1.07 | 2.8E+00 | ± | 1.6E+00 | −0.71 | 0.303 | 0.492 |
| 2-Rhamnose | 0.29 | ± | 0.17 | 1.5E−01 | ± | 1.8E−01 | 0.93 | 0.311 | 0.492 |
| T-Fucose | 1.34 | ± | 1.13 | 6.3E−01 | ± | 7.4E−01 | 1.10 | 0.337 | 0.516 |
| 3,4,6-Mannose | 0.04 | ± | 0.01 | 2.9E−02 | ± | 2.4E−02 | 0.52 | 0.373 | 0.549 |
| 3,4-Galactose | 0.16 | ± | 0.11 | 1.0E−01 | ± | 6.5E−02 | 0.70 | 0.381 | 0.549 |
| T-Fructose | 1.58 | ± | 0.70 | 3.2E+00 | ± | 3.1E+00 | −1.00 | 0.393 | 0.550 |
| 4,6-Glucose | 0.67 | ± | 0.40 | 1.1E+00 | ± | 9.8E−01 | −0.73 | 0.449 | 0.611 |
| 4-Rhamnose | 0.11 | ± | 0.06 | 6.9E−02 | ± | 9.2E−02 | 0.70 | 0.462 | 0.611 |
| 4-Glucose | 152.46 | ± | 27.73 | 1.7E+02 | ± | 4.8E+01 | −0.17 | 0.515 | 0.658 |
| 2,3,6-Glucose | 0.07 | ± | 0.02 | 8.3E−02 | ± | 4.0E−02 | −0.29 | 0.524 | 0.658 |
| 6-Mannose | 0.01 | ± | 0.00 | 4.1E−03 | ± | 3.6E−03 | 0.44 | 0.540 | 0.658 |
| 2,4,6-Galactose | 0.03 | ± | 0.01 | 4.7E−02 | ± | 4.7E−02 | −0.57 | 0.566 | 0.658 |
| 2,X,X-Hexose(I) | 0.04 | ± | 0.02 | 3.0E−02 | ± | 2.8E−02 | 0.46 | 0.569 | 0.658 |
| T-Rhamnose | 1.15 | ± | 0.52 | 1.3E+00 | ± | 2.2E−01 | −0.20 | 0.578 | 0.658 |
| T-P-Arabinose | 0.97 | ± | 1.04 | 7.0E−01 | ± | 1.2E+00 | 0.47 | 0.747 | 0.832 |
| 4-P-Xylose | 0.52 | ± | 0.83 | 4.0E−01 | ± | 5.5E−01 | 0.37 | 0.822 | 0.890 |
| 3,6-Galactose | 0.22 | ± | 0.08 | 2.0E−01 | ± | 1.7E−01 | 0.14 | 0.835 | 0.890 |
| X-Hexose | 1.70 | ± | 1.09 | 1.6E+00 | ± | 5.7E−01 | 0.07 | 0.903 | 0.929 |
| 2,4,6-Glucose | 0.01 | ± | 0.00 | 1.4E−02 | ± | 9.0E−03 | −0.06 | 0.917 | 0.929 |
| 6-Galactose | 2.95 | ± | 1.27 | 3.0E+00 | ± | 1.8E+00 | −0.05 | 0.929 | 0.929 |

Integrating the quantitative polysaccharide and glycoside linkage data allowed to conclude that MDCF-2 contains significantly more galactans and mannans than RUSF (q<0.05; t-test), while RUSF contains significantly more starch and cellulose (q<0.05; t-test) (FIG. 3D; Tablo 7).

TABLE 7
Difference in polysaccharide content between MDCF-2
and RUSF (μg polysaccharide/mg of dried diet)

| | | | | | | | Comparison of MDCF-2 and | | |
|---|---|---|---|---|---|---|---|---|---|
| | MDCF-2 | | | RUSF | | | RUSF (t-test) | | |
| Polysaccharide | mean ± SD | | | mean ± SD | | | log2 (MDCF-2/RUSF) | P-value | q-value |
| Galactan | 1.67 | ± | 0.12 | 0.68 | ± | 0.08 | 1.30 | 0.001 | 0.003 |
| Starch | 216.00 | ± | 5.80 | 345.00 | ± | 30.00 | −0.68 | 0.015 | 0.022 |
| Cellulose | 4.21 | ± | 0.43 | 7.88 | ± | 0.98 | −0.90 | 0.013 | 0.022 |
| Mannan | 0.43 | ± | 0.08 | 0.07 | ± | 0.01 | 2.73 | 0.015 | 0.022 |
| Arabinan | 0.84 | ± | 0.13 | 0.64 | ± | 0.04 | 0.40 | 0.112 | 0.112 |
| Xylan | 0.55 | ± | 0.14 | 0.35 | ± | 0.06 | 0.66 | 0.112 | 0.112 |

Galactans are represented in MDCF-2 as unbranched 1-1,4-linked galactan as well as arabinogalactan I (FIG. 3E). Mannans are present as unbranched 1-1,4-linked mannan (1-mannan), galactomannan and glucomannan (FIGS. 3C, and 3F). Arabinan is abundant in both compositions, although the representation of arabinose and glycosidic linkages containing arabinose are significantly greater in MDCF-2 than in RUSF (see FIGS. 3A and 3B for results of statistical tests). Arabinan in MDCF-2 is largely derived from its soybean, banana, and chickpea components, while in RUSF, this polysaccharide originates from rice and lentil. Arabinans in both compositions share a predominant 1,5-linked-L-arabinofuranose (Araf) backbone. Soybean arabinans are characterized by diverse side chains composed of 1,2- and 1,3-linked-L-Araf connected by 1,2,3-, 1,2,5-, and 1,3,5-L-Araf branch points, while chickpea, lentil, and banana arabinans primarily contain 1,3-linked side chains from 1,3,5-L-Araf branch points (FIG. 3C).

### Example 4: MDCF-2 Effects on WLZ-Associated MAG Gene Expression

Microbial RNA-Seq was performed using RNA isolated from fecal samples collected from all study participants just prior to initiation of treatment, and at the 1-, and 3-month time points (n=350 samples). Transcripts were then quantified by mapping reads from each sample to all 1,000 MAGs. The resulting counts tables were filtered based on the abundance and prevalence of MAGs in the full set of all fecal samples. These filtering steps were designed to exclude MAGs with minimal contributions to the meta-transcriptome from subsequent differential expression analysis (exclusion criteria were benchmarked against a simulated meta-transcriptomic dataset using the approach described in the Methods).

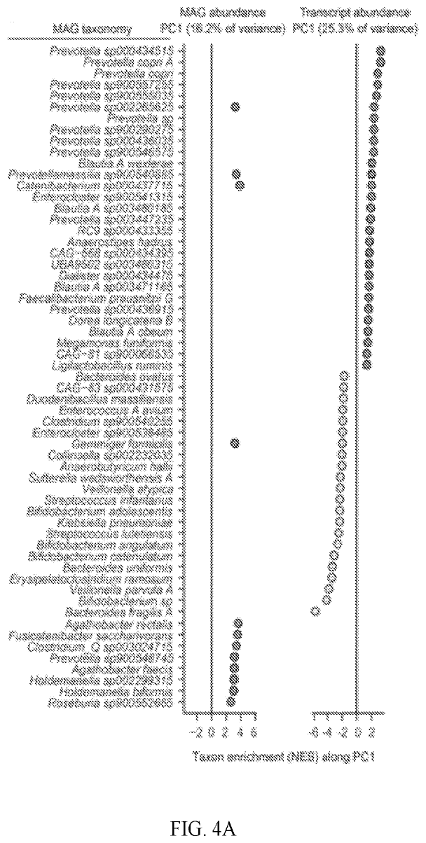

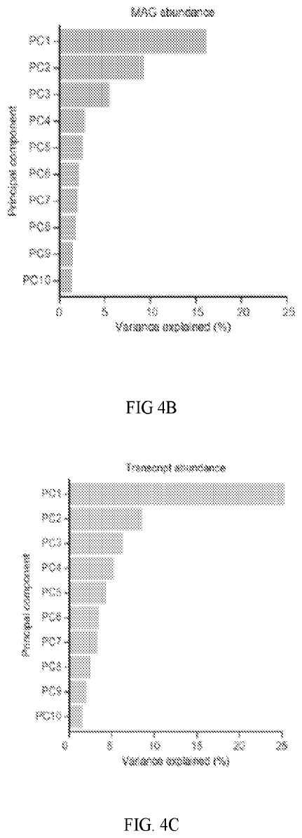

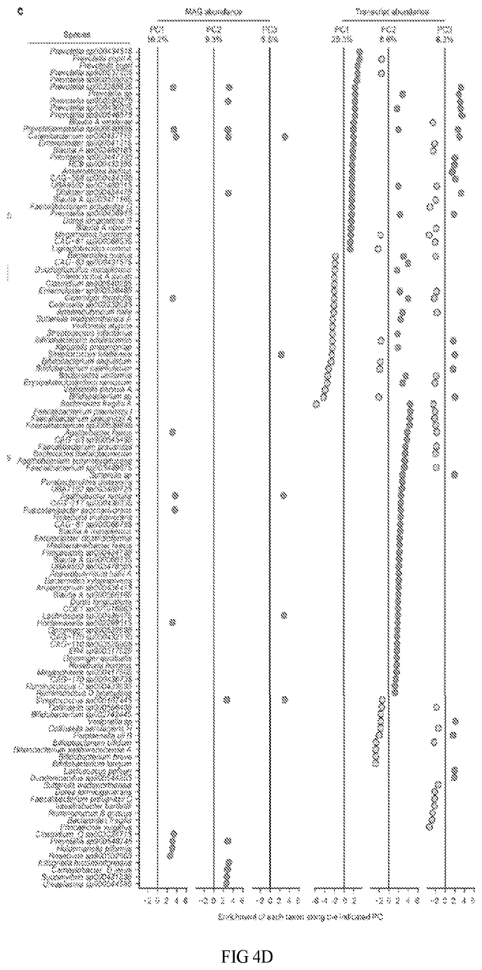

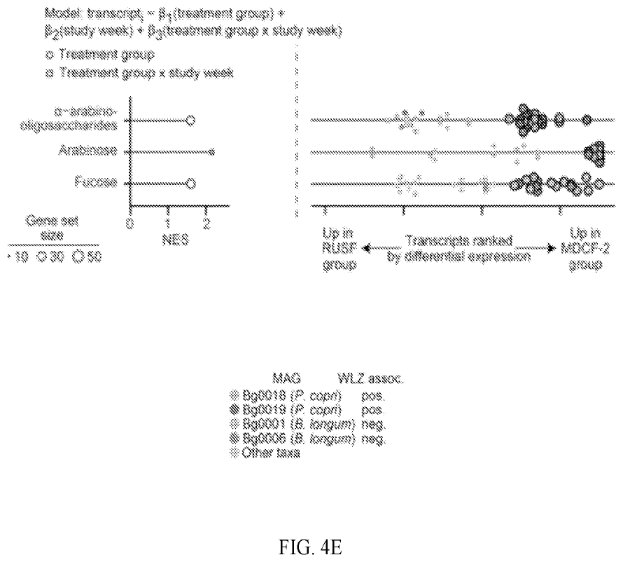

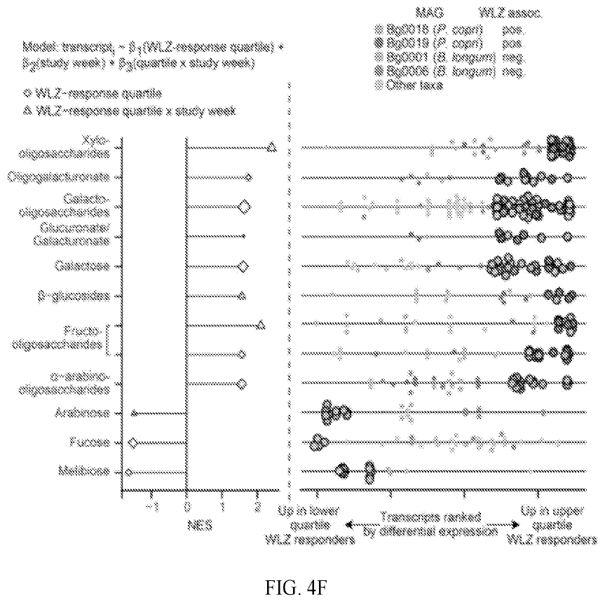

Principal components analysis (PCA) was used to determine baseline differences in overall microbiome or meta-transcriptome configurations between the treatment groups, and to subsequently identify microbes that were principal drivers in shifts during treatment. FIG. 4A-4D plot (i) the percent variance explained by the top 10 principal components (PCs) in analyses of 837 MAGs in fecal samples collected across all timepoints from all study participants (FIG. 4B-4D) and (ii) the taxa enriched (q<0.05; GSEA) along the first three principal components of the MAG abundance and meta-transcriptome datasets (FIG. 4A). There were no statistically significant differences in microbiome or meta-transcriptome configuration between groups prior to treatment (P>0.1; PERMANOVA). Analysis of MAG contributions to each PCA analysis highlights the remarkable enrichment of Prevotella spp., and to a lesser extent, Bifidobacterium spp., along the principal axis of variation (PC1) of the transcript PCA, and the absence of enrichment of these organisms along PC1 of the DNA-based PCA.

Next the transcripts expressed by the 222 MAGs whose abundances were significantly associated with WLZ were studied. Transcripts were ranked by their response to MDCF-2 versus RUSF treatment or by their response over time (negative binomial generalized linear model; see equation in FIG. 4E). GSEA was then performed to identify metabolic pathways enriched in these ranked transcripts. The analysis revealed a MDCF-2-associated pattern of gene expression characterized by significant enrichment (q<0.1; GSEA) of three metabolic pathways related to carbohydrate utilization [a-arabinooligosaccharide (aAOS), arabinose and fucose; FIG. 4E], three pathways related to de novo amino acid synthesis (arginine, glutamine, and lysine biosynthesis), and one pathway for de novo vitamin synthesis (folate). In contrast, none of the 106 metabolic pathways exhibited statistically significant enrichment in their expression in children who received RUSF.

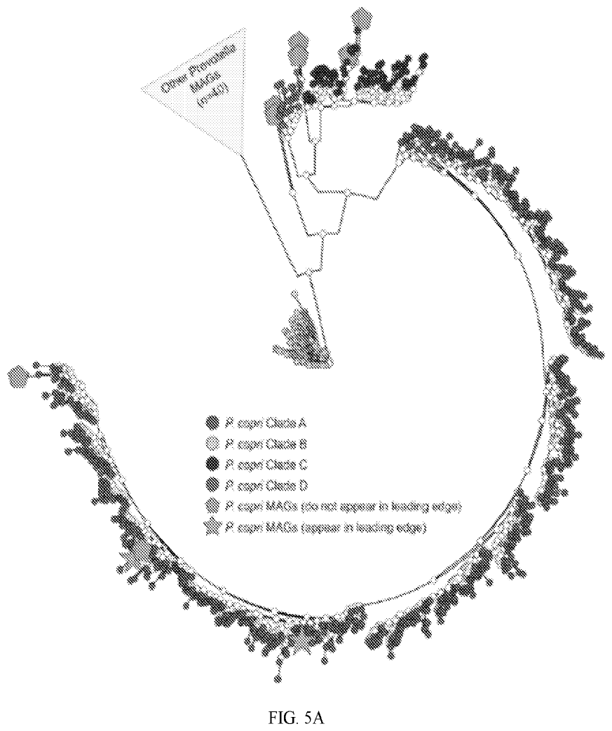

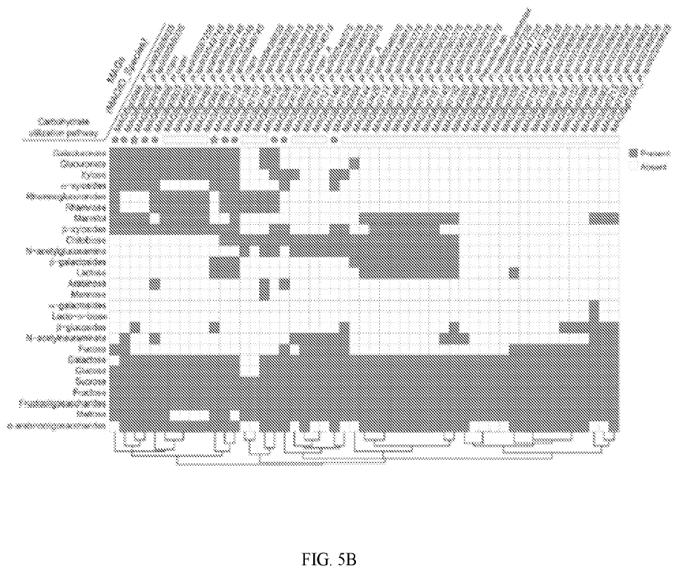

MAGs which were responsible for the observed enrichment of expressed pathways were investigated. To do so, ‘leading edge’ transcripts were turned; a term defined by GSEA as those transcripts responsible for enrichment of a given pathway (Methods). Among positively WLZ-associated MAGs, two belonging to P. copri (MAG Bg0018 and MAG Bg0019) were the source of 11 of the 14 leading-edge transcripts related to aAOS utilization-a pathway whose expression was significantly elevated in children treated with MDCF-2 compared to RUSF (FIG. 4E). Of the 11 P. copri MAGs in the dataset, these two were the only MAGs assigned to this species that were significantly positively correlated with WLZ. Both MAGs are members of a P. copri clade (Clade ‘A’) that is broadly distributed geographically (FIG. 5A); furthermore, P. copri exhibits substantial strain-level genomic and functional diversity (FIG. 5B) for the predicted carbohydrate utilization pathways represented in all 51 MAGs assigned to the genus Prevotella that were identified in the 1,000 MAG dataset).

Although P. copri MAGs were the greatest source of leading-edge transcripts related to aAOS utilization, other MAGs in the microbiome display expression responses consistent with their participation in metabolizing MDCF-2 glycans (or their breakdown products); these include MAGs that are negatively correlated with WLZ. For example, MAGs expressing leading-edge transcripts assigned to aAOS, arabinose and fucose utilization arose from Bifidobacterium longum subsp. longum (Bg0006), Bifidobacterium longum subsp. suis (Bg0001), Bifidobacterium breve (Bg0010; Bg0014), Bifidobacterium sp. (Bg0070), and Ruminococcus gnavus (Bg0067).

Features of the metabolism of these glycans in Bifidobacterium and Ruminococcus MAGs are distinct from those expressed by the P. copri MAGs. For example, B. longum subsp. longum MAG Bg0006 encodes extracellular exo-α-1,3-arabinofuranosidases that belong to glycoside hydrolase (GH) family (e.g., BIArafA); these enzymes cleave terminal 1,3-linked-L-Araf residues present at the ends of branched arabinans and arabinogalactans, two abundant glycans found in MDCF-2 (FIGS. 3C and 3E). In contrast, P. copri possesses an endo-α-1,5-L-arabinanase that cleaves interior α-1,5-L-Araf linkages, generating aAOS. Integrating these predictions suggests a complex set of interactions between primary arabinan degraders like P. copri and members of B. longum, such as Bg0001 and Bg0006, that are capable of metabolizing products of arabinan degradation (see FIG. 6 for reconstructions of carbohydrate utilization pathways in Bifidobacterium MAGs). It could not be discerned whether the arabinose available to Bifidobacterium is derived from free arabinose or the breakdown products of arabinan polysaccharides. It is important to consider that in these 12- to 18-month-old children with MAM, responses to MDCF-2 are occurring in the context of the underlying co-development of their microbial community and host biology, during the period of transition from exclusive milk feeding to a fully weaned state. A MAG defined as positively associated with WLZ by linear modeling is an organism whose fitness (abundance) increases. The studies in healthy 1- to 24-month-old children living in Mirpur have documented how B. longum and other members of Bifidobacterium decrease in absolute abundance during the period of complementary feeding. For the negatively WLZ-associated Bifidobacterium MAGs described above, the levels of consumption of MDCF-2 metabolic products during the period of complementary feeding, and the nature of the changes in metabolism that occurs in these organisms as a result, may not be sufficient to overcome a more dominant effect exerted on their abundance/fitness and impact on ponderal growth by background diet and/or the state of community-host co-development.

Based on these observations, further evidence that the two P. copri MAGs are related to the magnitude of ponderal growth responses, and to levels of fecal glycan structures generated from MDCF-2 metabolism, was sought.

### Example 5: Carbohydrate Utilization Pathways and Clinical Responses

As noted above, the primary outcome measure of the clinical trial was the rate of change of WLZ over the 3-month intervention. Participants receiving MDCF-2 were stratified into WLZ-response quartiles and analysed on (i) children in the upper- and lower-WLZ-quartiles (n=15/group) and (ii) transcripts expressed by the 222 MAGs whose abundances were significantly associated with WLZ. Enrichment of carbohydrate utilization pathways were tested in transcripts rank-ordered by the strength and direction of their relationship with WLZ-quartile or, in a separate analysis, the interaction between WLZ-quartile and study week; GSEA to identify enriched pathways were performed.

Eight carbohydrate utilization pathways were significantly enriched in transcripts differentially expressed in upper compared to lower WLZ quartile responders. One of these pathways (fructooligosaccharides utilization), plus three other pathways that are involved in arabinose, b-glucoside, and xylooligosaccharide utilization, were enriched in transcripts with a positive ‘WLZ quartile×study week’ interaction coefficient, suggesting that the extent of the difference in expression of these pathways increases over the course of treatment (FIG. 4E).

Remarkably, over half of the leading-edge transcripts (67/99; 68%) from the eight, upper WLZ-quartile enriched carbohydrate utilization pathways were expressed by P. copri MAGs Bg0018 and Bg0019. Moreover, these two MAGs contributed no leading-edge transcripts to lower WLZ-response quartile enriched pathways.

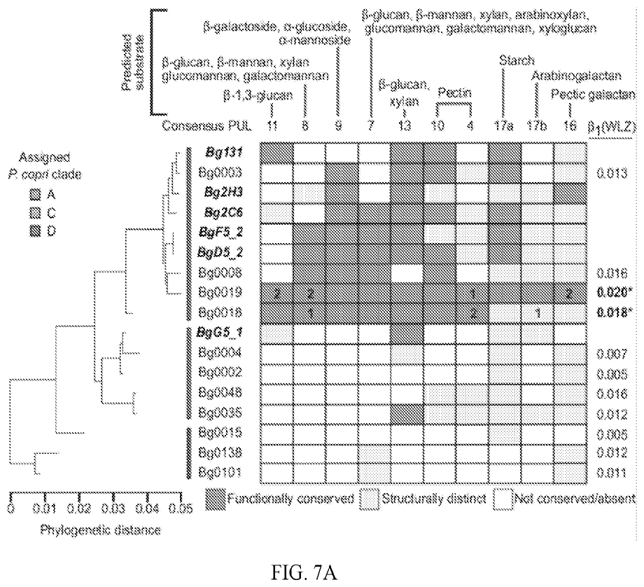

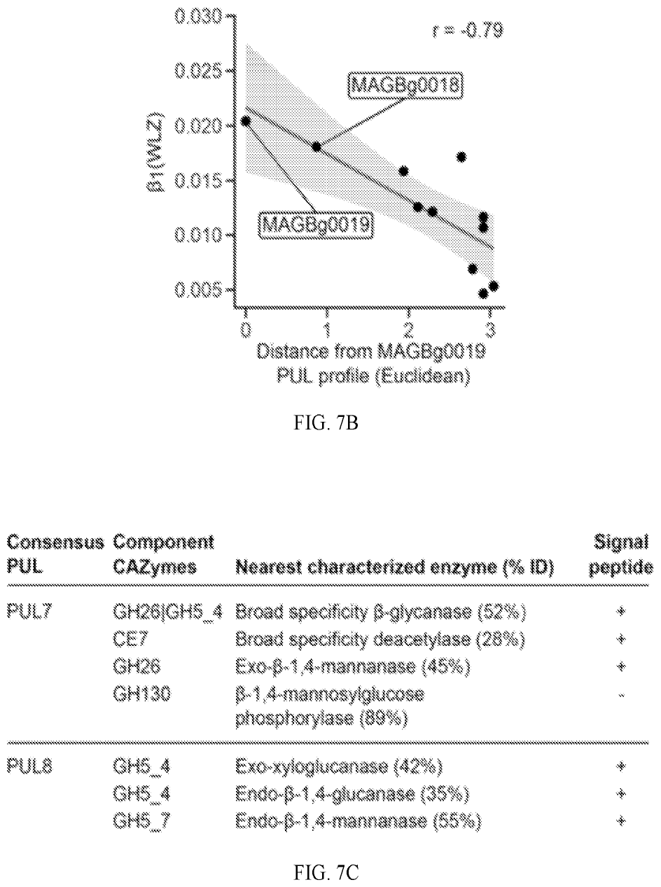

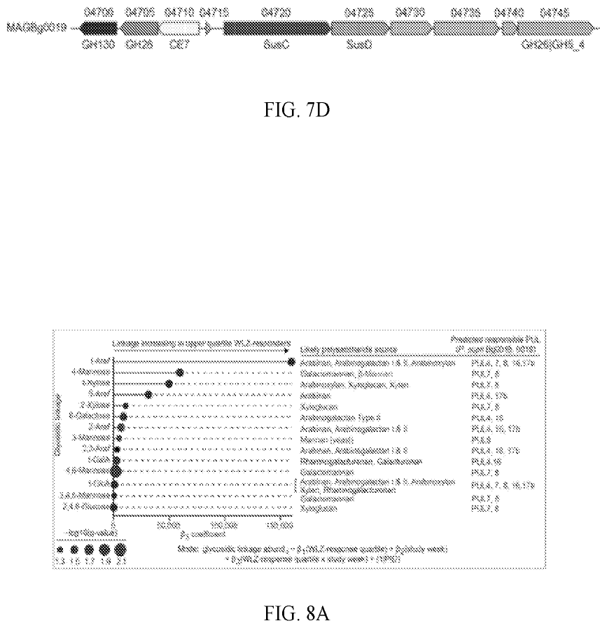

P. copri is a member of the phylum Bacteroidota. Members of this phylum contain syntenic sets of genes known as polysaccharide utilization loci (PULs) that mediate detection, import and metabolism of a specific glycan or set of glycans25. To further define how expressed genomic features distinguish the capacity of Bg0019 and Bg0018 to respond to MDCF-2, PULs were identified and compared to those present in the nine other P. copri MAGs in this study. These two WLZ-associated P. copri MAGs share (i) seven PULs designated as highly conserved (i.e., a given pair of shared PULs that encode protein products with ≥90% amino acid identity and have identical genomic organization) plus (ii) three PULs designated as present but ‘structurally distinct’ (i.e., displaying divergence expected to impact function). The representation of these 10 PULs varied among the other nine P. copri MAGs which span three of the four principal clades of this organism (FIG. 7A). Strikingly, the representation of these PULs is significantly associated with the relationship between each of the 11 P. copri MAGs in the 1,000 MAG dataset and WLZ across both treatment groups [Pearson r between Euclidean distance from Bg0019 PUL profile and 131 (MAG)=−0.79 (P=0.0035); FIG. 7B]. Five of the seven highly conserved PULs are related to utilization of mannan and galactan—glycans that are significantly more abundant in MDCF-2 than RUSF. Expression of three of these seven PULs, as well as two of the conserved but structurally distinct PULs, are also related to the enrichment of transcripts in carbohydrate utilization pathways that distinguish upper from lower WLZ-quartile responders (‘WLZ-response quartile’ or ‘WLZ quartile×study week’ terms in FIG. 7F). PULs that generate these leading-edge transcripts are predicted to metabolize 13-glucan, glucomannan, 13-mannan, xylan, pectin/pectic galactan and arabinogalactan (see FIG. 7A for which of these 10 PULs contribute differentially-expressed transcripts).

A comparative analysis of MAGs Bg0018 and Bg0019 and 22 reference P. copri genomes in PULDB26 indicated that one of the highly conserved PULs (PUL7) contains a bimodular GH26|GH5_4 13-glycanase with 52% amino acid sequence identity to an enzyme known to cleave 13-glucan, 13-mannan, xylan, arabinoxylan, glucomannan, and xyloglucan (FIGS. 7C and 7D). The gene encoding this multifunctional enzyme did not satisfy the criteria for statistically significant differential expression between MDCF-2 and RUSF treatment, nor between upper versus lower quartile WLZ-responders. However, it was consistently expressed across these conditions/comparisons and its enzymatic product is expected to contribute to the utilization of a broad range of plant glycans, including those represented in MDCF-2. Together, these results highlight both the versatility in carbohydrate metabolic capabilities of these two WLZ-associated P. copri MAGs, as well as the specificity of their treatment-inducible metabolic pathways for carbohydrates prominently represented in MDCF-2.

To contextualize our observations regarding conserved polysaccharide-degradation features of our P. copri MAGs, we selected a set of six P. copri isolates, obtained from Bangladeshi children who participated in our clinical trials, and representing a diverse PUL conservation repertoire and phylogenetic distance from the WLZ-associated Bg0018 and Bg0019 (FIG. 7A) for further analysis. These isolates include BgD5_2 and BgF5_2, strains which are highly phylogenetically related to Bg0018 and Bg0019 and possess 9/10 conserved PULs when compared to these MAGs (see Tables 28 and 29 for details of functional conservation between the genomes of these and other P. copri strains and MAGs).

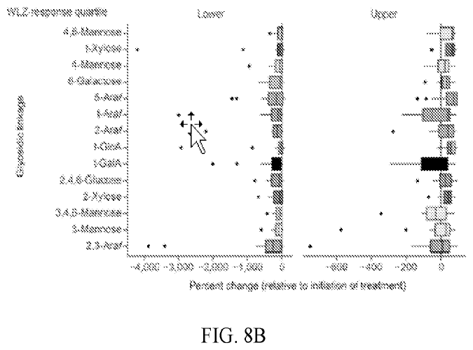

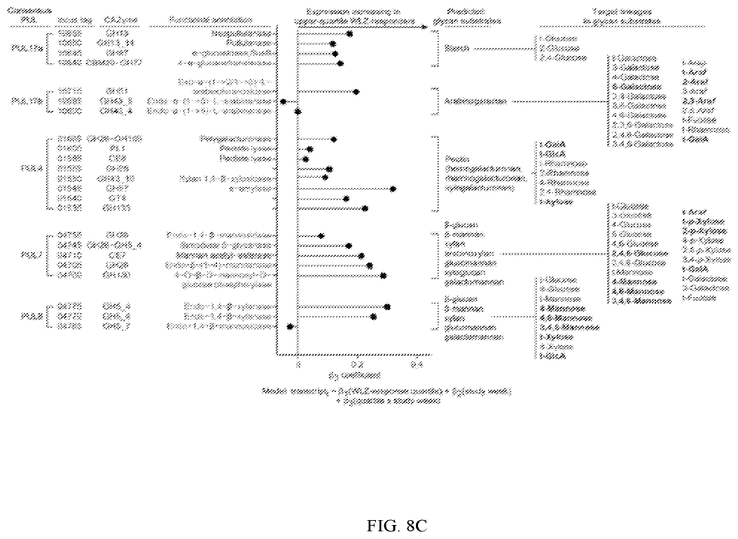

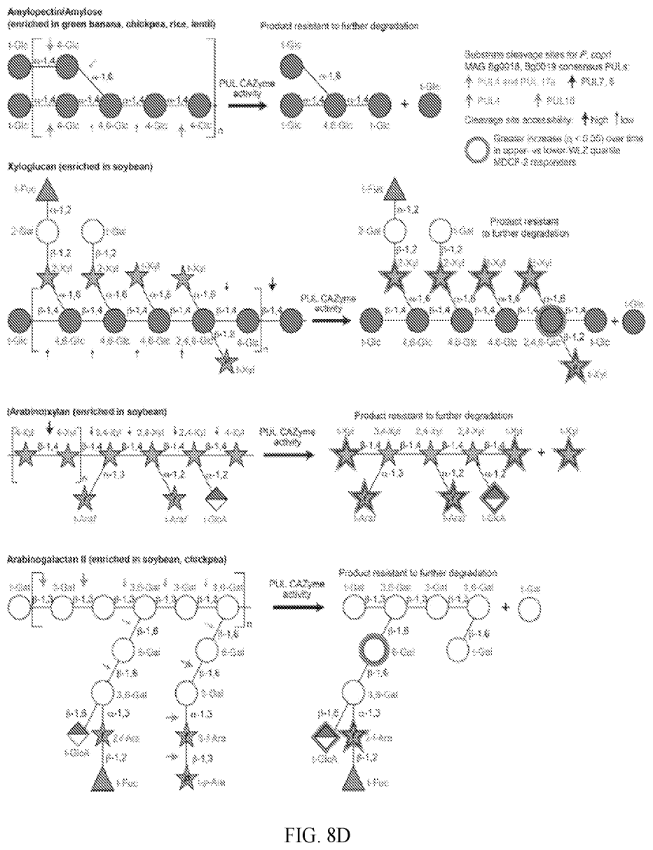

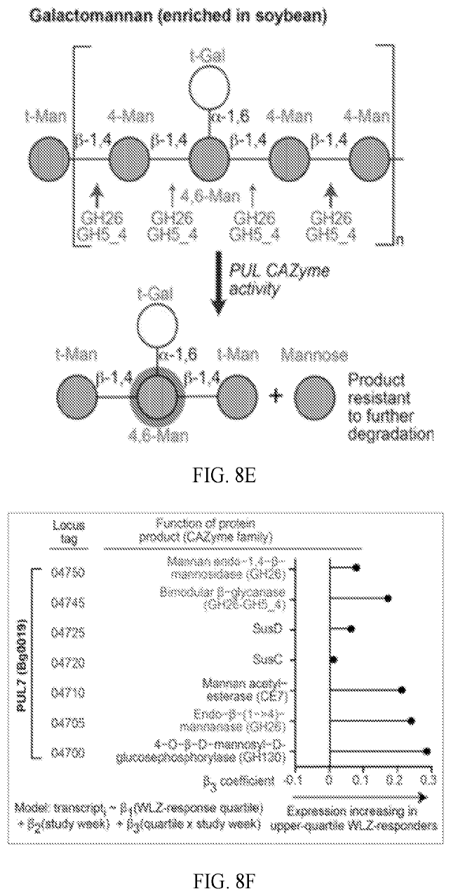

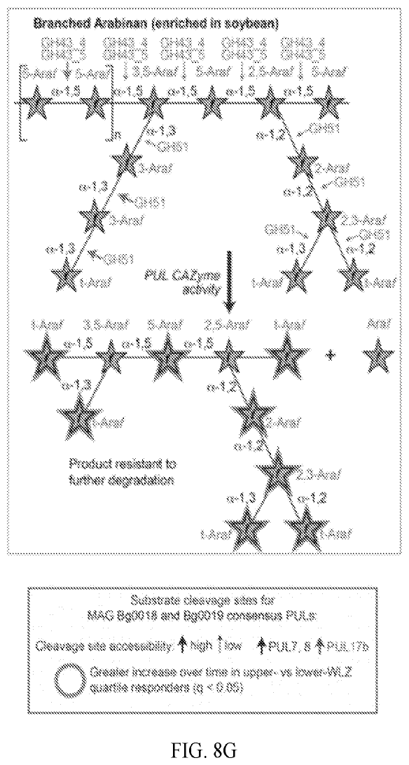

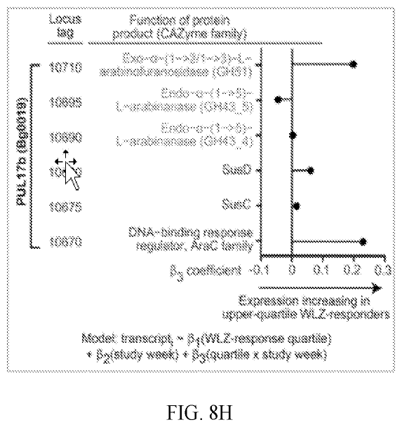

The same fecal samples collected at the 0- and 3-month time points from participants in the upper and lower WLZ quartiles in the MDCF-2 treatment group that had been used for the DNA- and RNA-level analyses were subjected to UHPLC-QqQ-MS-based quantitation of 49 glycosidic linkages. These linkages were measured after their liberation by in vitro hydrolysis of fecal glycans. Linear mixed-effects modeling demonstrated that, with treatment, fecal levels of 14 of these linkages increased significantly more (q<0.05) in participants in the upper compared to the lower WLZ response quartile (FIGS. 8A and 8B, Table 8). These 14 differentially abundant glycosidic linkages are all represented in MDCF-2.

TABLE 8
Changes in fecal glycosidic linkage levels over time
in upper- compared to lower- WLZ quartile responders

| | Model | | | P- | q- |
|---|---|---|---|---|---|
| Glycosidic linkage | term | Coefficient | SEM | value | value |
| 4,6-Mannose | WLZ quartile*study | 1669.3 | 413.7 | 0.000 | 0.008 |
| | week | | | | |
| T-F-Arabinose | WLZ quartile*study | 159957.1 | 53526.3 | 0.004 | 0.029 |
| | week | | | | |
| T-GalA | WLZ quartile*study | 2491.0 | 869.1 | 0.006 | 0.029 |
| | week | | | | |
| T-GlcA | WLZ quartile*study | 937.9 | 321.8 | 0.005 | 0.029 |
| | week | | | | |
| T-P-Xylose | WLZ quartile*study | 49871.5 | 15620.3 | 0.002 | 0.029 |
| | week | | | | |
| 2,4,6-Glucose | WLZ quartile*study | 215.0 | 75.2 | 0.006 | 0.029 |
| | week | | | | |
| 2-F-Arabinose | WLZ quartile*study | 6589.4 | 2235.6 | 0.005 | 0.029 |
| | week | | | | |
| 4-Mannose | WLZ quartile*study | 58684.9 | 19354.6 | 0.004 | 0.029 |
| | week | | | | |
| 5-F-Arabinose | WLZ quartile*study | 31291.5 | 10470.0 | 0.004 | 0.029 |
| | week | | | | |
| 6-Galactose | WLZ quartile*study | 8603.8 | 2858.0 | 0.004 | 0.029 |
| | week | | | | |
| 2,3-F-Arabinose | WLZ quartile*study | 3299.1 | 1304.4 | 0.014 | 0.050 |
| | week | | | | |
| 2-P-Xylose | WLZ quartile*study | 10446.1 | 4072.0 | 0.013 | 0.050 |
| | week | | | | |
| 3,4,6-Mannose | WLZ quartile*study | 258.3 | 101.2 | 0.013 | 0.050 |
| | week | | | | |
| 3-Mannose | WLZ quartile*study | 4572.7 | 1805.1 | 0.014 | 0.050 |
| | week | | | | |
| 4-P-Xylose | WLZ quartile*study | 36558.2 | 15499.0 | 0.022 | 0.071 |
| | week | | | | |
| T-Mannose | WLZ quartile*study | 20361.5 | 9430.0 | 0.035 | 0.103 |
| | week | | | | |
| 3-Arabinose | WLZ quartile*study | 6494.8 | 3020.9 | 0.036 | 0.103 |
| | week | | | | |
| 2,4-Glucose | WLZ quartile*study | 1647.5 | 784.9 | 0.040 | 0.109 |
| | week | | | | |
| 4-Galactose | WLZ quartile*study | 32452.4 | 16439.2 | 0.053 | 0.137 |
| | week | | | | |
| T-Fucose | WLZ quartile*study | 48377.3 | 24842.7 | 0.056 | 0.138 |
| | week | | | | |
| 2-Galactose | WLZ quartile*study | 18409.3 | 9915.8 | 0.068 | 0.160 |
| | week | | | | |
| 3,4-P-Xylose/3,5- | WLZ quartile*study | 5466.2 | 3179.3 | 0.091 | 0.202 |
| Arabinose | week | | | | |
| 4-Glucose | WLZ quartile*study | 197550.5 | 117723.2 | 0.099 | 0.210 |
| | week | | | | |
| 2,X,X-Hexose_I | WLZ quartile*study | 554.3 | 335.4 | 0.104 | 0.212 |
| | week | | | | |
| T-Galactose | WLZ quartile*study | 65092.9 | 39983.9 | 0.109 | 0.214 |
| | week | | | | |
| 2,4-P-Xylose | WLZ quartile*study | 1817.0 | 1246.8 | 0.150 | 0.283 |
| | week | | | | |
| 3,4-Galactose | WLZ quartile*study | 3017.2 | 2211.3 | 0.178 | 0.322 |
| | week | | | | |
| 2,5-F-Arabinose | WLZ quartile*study | 428.2 | 326.2 | 0.194 | 0.340 |
| | week | | | | |
| T-Glucose | WLZ quartile*study | 39135.5 | 30733.5 | 0.208 | 0.351 |
| | week | | | | |
| T-Rhamnose | WLZ quartile*study | 5282.9 | 4353.6 | 0.230 | 0.375 |
| | week | | | | |
| 2,X,X-Hexose_II | WLZ quartile*study | 355.3 | 305.6 | 0.250 | 0.395 |
| | week | | | | |
| T-P-Arabinose | WLZ quartile*study | −9661.7 | 9387.6 | 0.308 | 0.471 |
| | week | | | | |
| 4-Rhamnose | WLZ quartile*study | −4812.0 | 5054.6 | 0.345 | 0.512 |
| | week | | | | |
| 3,4-Glucose | WLZ quartile*study | 2565.5 | 2763.6 | 0.357 | 0.515 |
| | week | | | | |
| 3,4,6-Galactose | WLZ quartile*study | 395.3 | 465.0 | 0.399 | 0.558 |
| | week | | | | |
| 2,3,6-Glucose | WLZ quartile*study | 73.7 | 100.0 | 0.464 | 0.632 |
| | week | | | | |
| X-Hexose | WLZ quartile*study | 19948.1 | 29591.2 | 0.503 | 0.666 |
| | week | | | | |
| 6-Mannose | WLZ quartile*study | −62.5 | 101.9 | 0.542 | 0.699 |
| | week | | | | |
| 4,6-Galactose | WLZ quartile*study | 3794.2 | 7822.2 | 0.629 | 0.791 |
| | week | | | | |
| 2-Rhamnose | WLZ quartile*study | 2280.7 | 5181.3 | 0.661 | 0.796 |
| | week | | | | |
| 3-Glucose/3-Galactose | WLZ quartile*study | 4629.0 | 10670.1 | 0.666 | 0.796 |
| | week | | | | |
| 3,4,6-Glucose | WLZ quartile*study | 153.3 | 461.4 | 0.741 | 0.864 |
| | week | | | | |
| 2-Glucose | WLZ quartile*study | 1149.5 | 4471.3 | 0.798 | 0.909 |
| | week | | | | |
| 2,4,6-Galactose | WLZ quartile*study | −26.0 | 151.0 | 0.864 | 0.928 |
| | week | | | | |
| 2-Mannose | WLZ quartile*study | 361.3 | 2373.9 | 0.880 | 0.928 |
| | week | | | | |
| 3,6-Galactose | WLZ quartile*study | 223.4 | 1376.9 | 0.872 | 0.928 |
| | week | | | | |
| 6-Glucose | WLZ quartile*study | −199.6 | 1434.5 | 0.890 | 0.928 |
| | week | | | | |
| T-Fructose | WLZ quartile*study | −740.6 | 8812.9 | 0.933 | 0.953 |
| | week | | | | |
| 4,6-Glucose | WLZ quartile*study | 64.7 | 2280.9 | 0.977 | 0.977 |
| | week | | | | |

Differences in levels of these 14 glycosidic linkages can be explained in part by the specificity of the expressed CAZymes encoded by PULs conserved between P. copri MAGs Bg0018 and Bg0019. Among the 14 significantly differentially abundant linkages, t-Araf, 4-Mannose, t-Xylopyranose, 5-Araf and 2-Xylopyranose exhibit the greatest difference in fecal levels between upper and lower quartile responders over time; notably, all are elevated in upper quartile responders. FIGS. 8A, 8C and 8D describe their likely polysaccharide sources in MDCF-2, show the P. copri PULs predicted to generate glycan fragments containing these linkages, and highlight that these fragments are likely resistant to further degradation and thus can accumulate in the feces (FIG. 8A). For example, t-Araf is a component of arabinan, arabinoxylan and arabinogalactan type I/II in soybean, chickpea, peanut and banana (FIGS. 3A and 3B), and would be expected to accumulate in the intestine as CAZymes encoded by P. copri Bg0019 PULs 4, 7, 8, 16 and 17b cleave accessible linkages, exposing additional t-Araf (FIG. 8B-E). Exo-α-1,2/1,3-L-arabinofuranosidase and endo-α-1,5-L-arabinanase encoded by PUL17b (FIG. 8E-H) are predicted to remove successive residues from the 1,2 and 1,3-linked-L-Araf chains of branched arabinan and hydrolyze the 1,5-linked-L-Araf backbone from this polysaccharide. In P. copri Bg0019, this activity is complemented by two PUL4-encoded pectate lyases that assist in cleaving branched arabinan sidechains. In another example, CAZyme activities encoded by these two WLZ-associated P. copri MAGs also explain the greater increase in fecal levels of 4,6-mannose over time in upper-compared to lower-WLZ quartile responders (FIG. 8A). This linkage is a characteristic component of soybean galactomannan and is expected to accumulate in the feces upon partial degradation of this glycan by endo-1,4-β-mannosidases encoded by PUL7 and PUL8 (FIG. 8F).

CAZyme transcripts assigned to PULs 4, 7, 8, 16 and 17b were detectable in all but one of the 30 participants assigned to the two WLZ responder quartiles, with levels of expression of the majority of these CAZymes being modestly elevated in upper compared to lower WLZ-quartile responders over the course of treatment [these include the GH51 CAZyme encoded by PUL17b plus the GH26, GH26-GH5_4, GH130 and carbohydrate esterase family 7 (CE7) transcripts from PUL7; see FIGS. 8B and 8C]. However, their differential expression did not satisfy the criteria for statistical significance. This latter finding raised the question of what other factors might contribute to the observed differences in fecal linkage content between upper and lower quartile responders. Intake of MDCF-2 was not significantly different between the upper- and lower-WLZ quartile participants [P>0.05; linear mixed-effects model; daily MDCF-2 consumption˜days-on-treatment+WLZ-response quartile+WLZ-response quartile: days-on-treatment+(1|PID)]. Data from a food frequency questionnaire (FFQ) administered at each fecal sampling disclosed that the mean correlation between the abundances of the 14 glycosidic linkages elevated in upper WLZ-quartile responders and FFQ queries was strongest for the question related to consumption of legumes and nuts and the levels of t-Araf, 5-Araf, 2,3-Araf, t-GalA, and 2,4,6-Glucose. Consumption of this food group was also the most discriminatory response between upper compared to lower WLZ quartile responders (Table 9).

TABLE 9
Effect of food groups on upper compared
to lower WLZ quartile responders

| | | WLZ response quartile | | |
|---|---|---|---|---|
| | | (mean frequency ± SD) | | |
| Query | Description | Lower | Upper | Ratio |
| FFQ110a | Tea, coffee, or any other | 0.12 ± 0.36 | 0.26 ± 0.48 | 2.08 |
| | warm/hot drinks? | | | |
| FFQ118a | Foods made with beans, | 0.96 ± 1.22 | 1.66 ± 2.73 | 1.72 |
| | lentils, peas, corn, | | | |
| | ground nuts or any other | | | |
| | legumes? | | | |
| FFQ121a | Liver, kidney, or other | 0.08 ± 0.36 | 0.1 ± 0.41 | 1.38 |
| | organ meats? | | | |
| FFQ124a | Fresh or dried fish or | 0.41 ± 0.83 | 0.56 ± 0.87 | 1.37 |
| | shellfish? | | | |
| FFQ105 | Last night, how many | 0.54 ± 0.88 | 0.7 ± 1.46 | 1.28 |
| | times did you feed your | | | |
| | child animal milks from | | | |
| | sunset to sunrise? | | | |
| FFQ114a | Rice, bread, noodles, or | 3.49 ± 2.09 | 4.25 ± 2.9 | 1.22 |
| | other foods made from | | | |
| | grains? | | | |
| FFQ107a | Is your child eating any | 3.17 ± 2.07 | 3.78 ± 2.92 | 1.19 |
| | semi-solid, mashed, or | | | |
| | solid foods (homemade, | | | |
| | not snacks)? | | | |
| FFQ125a | Cheese, yogurt, or other | 0.15 ± 0.43 | 0.18 ± 0.48 | 1.19 |
| | dairy products? | | | |
| FFQ123a | Eggs? | 0.42 ± 0.69 | 0.48 ± 0.68 | 1.14 |
| FFQ131a | Yesterday during food | 0.27 ± 0.71 | 0.3 ± 0.77 | 1.11 |
| | preparation, did oil was | | | |
| | mixed with it? | | | |
| FFQ117a | Any dark green or other | 0.24 ± 0.6 | 0.24 ± 0.61 | 1 |
| | leafy vegetables such | | | |
| | as spinach? | | | |
| FFQ129 | Yesterday, counting | 6.96 ± 2.85 | 6.99 ± 3.2 | 1 |
| | meals and snacks, how | | | |
| | many times did the | | | |
| | participant ate? | | | |
| FFQ132 | How would the | 2.64 ± 1.18 | 2.61 ± 1.12 | 0.99 |
| | responder describe | | | |
| | participant's appetite? | | | |
| FFQ119a | Ripe mangoes, | 0.24 ± 0.71 | 0.23 ± 0.52 | 0.96 |
| | papayas, or other sweet | | | |
| | yellow/orange or red | | | |
| | fruit? | | | |
| FFQ109a | Plain water? | 9.3 ± 2.49 | 8.83 ± 2.64 | 0.95 |
| FFQ115a | White potatoes or other | 0.92 ± 1.1 | 0.87 ± 1.04 | 0.94 |
| | foods made from roots? | | | |
| FFQ126a | Any sugary foods such | 0.67 ± 0.91 | 0.61 ± 0.8 | 0.91 |
| | as pastries, cakes, or | | | |
| | biscuits? | | | |
| FFQ106 | Yesterday, during the | 0.88 ± 1.31 | 0.77 ± 1.38 | 0.88 |
| | day, how many times | | | |
| | did you feed your child | | | |
| | animal milk? | | | |
| FFQ128a | Any locally | 0.52 ± 0.84 | 0.45 ± 0.69 | 0.85 |
| | produced/vendor foods | | | |
| | (such as rice cakes, | | | |
| | chanachur, | | | |
| | icecreametc.)? | | | |
| FFQ104 | Do you give your child | 0.57 ± 0.5 | 0.44 ± 0.5 | 0.77 |
| | any other milk, such as | | | |
| | tinned, packed, | | | |
| | powdered or fresh | | | |
| | animal milk? | | | |
| FFQ120a | Any other fruits or | 1.43 ± 1.67 | 1.1 ± 1.26 | 0.77 |
| | vegetables such as | | | |
| | banana, apple, oranges, | | | |
| | tomatoes, squash etc.? | | | |
| FFQ102 | Last night, how many | 4.44 ± 2.38 | 3.21 ± 2.53 | 0.72 |
| | times did you breastfeed | | | |
| | your child from sunset to | | | |
| | sunrise? | | | |
| FFQ122a | Any meat, such as | 0.51 ± 0.98 | 0.37 ± 0.7 | 0.72 |
| | chicken, beef, lamb, | | | |
| | goat, ducks (others)? | | | |
| FFQ103 | Yesterday, during the | 5.1 ± 3.08 | 3.28 ± 2.74 | 0.64 |
| | day, how many times | | | |
| | did you breastfeed your | | | |
| | child? | | | |
| FFQ127a | Any commercially | 0.59 ± 0.78 | 0.37 ± 0.67 | 0.63 |
| | available foods? | | | |
| FFQ111a | Fruit or vegetable juices | 0.05 ± 0.21 | 0.03 ± 0.17 | 0.6 |
| | (prepared at home)? | | | |
| FFQ112a | Any other liquids, such | 0.38 ± 0.7 | 0.16 ± 0.42 | 0.43 |
| | as sugar water, thin | | | |
| | soup or broth, | | | |
| | carbonated drinks, | | | |
| | commercially packed | | | |
| | juices. | | | |
| FFQ116a | Carrots or sweet | 0.3 ± 0.87 | 0.1 ± 0.46 | 0.35 |
| | potatoes that are yellow | | | |
| | or orange inside? | | | |
| FFQ130a | Yesterday during the | 0.05 ± 0.21 | 0 ± 0 | 0 |
| | day and at night, did the | | | |
| | participant eat anything | | | |
| | else other than the | | | |
| | foods that were | | | |
| | mentioned right now? | | | |
| fg5 | eggs | 0.33 ± 0.47 | 0.38 ± 0.49 | 1.14 |
| fg4 | flesh foods (meat, fish, | 0.54 ± 0.5 | 0.58 ± 0.5 | 1.07 |
| | poultry and liver/organ | | | |
| | meats) | | | |
| fg2 | legumes and nuts | 0.53 ± 0.5 | 0.55 ± 0.5 | 1.04 |
| fg1 | grains, roots and tubers | 1 ± 0 | 0.99 ± 0.1 | 0.99 |
| fg7 | other fruits and | 0.61 ± 0.49 | 0.59 ± 0.49 | 0.97 |
| | vegetables | | | |
| fg6 | vitamin-A rich fruits and | 0.38 ± 0.49 | 0.35 ± 0.48 | 0.93 |
| | vegetables | | | |
| fg3 | dairy products (milk, | 0.67 ± 0.47 | 0.53 ± 0.5 | 0.8 |
| | yogurt, cheese) | | | |
| mdd | Minimum dietary | 0.67 ± 0.47 | 0.67 ± 0.47 | 1 |
| | diversity | | | |
| mmf | Minimum meal | 0.97 ± 0.17 | 0.95 ± 0.21 | 0.98 |
| | frequency | | | |
| mad | Minimum acceptable | 0.61 ± 0.49 | 0.53 ± 0.5 | 0.88 |
| | diet | | | |

Together, these observations suggest that children consuming more of the classes of complementary food ingredients present in MDCF-2 may also exhibit enhanced growth responses; they also provided a rationale for performing a direct test in gnotobiotic mice, described in the accompanying paper, that a P. copri isolate, which shares features of the carbohydrate metabolic apparatus present in Bg0018 and Bg0019, is a key mediator of the degradation of MDCF-2 glycans, promotes ponderal growth, and has marked effects on multiple aspects of metabolism in intestinal epithelial cell lineages.

### Example 6: Discussion

The current study illustrates an approach for characterizing the bacterial targets and structure-function relationships of a microbiome-directed complementary food prototype, MDCF-2. This MDCF produced significantly greater weight gain during a 3-month-long, randomized controlled study of 12- to 18-month-old Bangladeshi children with moderate acute malnutrition compared to a calorically more dense, commonly employed, ready-to-use supplementary food (RUSF). Metagenome-assembled genomes (MAGs) were studied, specifically (i) treatment-induced changes in expression of carbohydrate metabolic pathways in MAGs whose abundances were significantly associated with WLZ, and (ii) mass spectrometric analysis of the metabolism of glycans present in the two therapeutic food compositions. Quantifying monosaccharides, glycosidic linkages and polysaccharides present in MDCF-2, RUSF and their component foods disclosed that MDCF-2 contains a greater content of galactans and mannans (e.g., galactan, arabinogalactan I, galactomannan, 13-mannan, glucomannan). Two types of comparisons were performed of the transcriptional responses of MAGs that were found to be significantly associated with WLZ: one involved participants who consumed MDCF-2 versus RUSF and the other focused on MDCF-2 treated children in the upper versus in lower quartiles of WLZ responses. The results revealed that two P. copri MAGs, both positively associated with WLZ, were the principal contributors to MDCF-2-induced expression of metabolic pathways involved in the utilization of its component glycans (13-glucan, glucomannan, 13-mannan, xylan, arabinoxylan, pectin/pectic galactan and starch).

UHPLC-QqQ-MS was able to identify statistically significant changes in glycan composition in a complex matrix like feces in children consuming a therapeutic food, even in the face of varied (non-uniform) background diets. Moreover, the approach of identifying MAGs, characterizing their gene expression as a function of treatment type and host response, and correlating gene expression with fecal glycosidic linkage content revealed just two P. copri strains among 75 WLZ-positively correlated MAGs. The findings that (i) these two MAGs possess PULs that are uniquely conserved compared to other P. copri MAGs in the study population, and (ii) PUL content correlates with WLZ association and levels of a number of glycosidic linkages from therapeutic food ingredients, highlight how this approach can be used to identify the strain-level specificity and genomic features of bacterial targets of MDCF-2, as well as the chemical structures present in the food components of MDCF-2 that these strains utilize.

Intriguingly, although intake of MDCF-2 did not differ in children in the upper quartile of WLZ improvement, children in the upper quartile trended toward diets containing more legumes and nuts than their lower WLZ quartile counterparts. The “legumes and nuts” food group includes major components of MDCF-2. It is postulated herein that MDCF-2 ‘kick-starts’ a microbiome response that includes changes in the fitness and expressed metabolic functions of key growth-associated bacterial strains, such as P. copri. Background diet can further modify this response, as evidenced by the higher levels of microbial metabolic products of legume/nut-associated glycans in the feces of children with upper quartile WLZ responses. More detailed, quantitative assessments of food consumption during future clinical studies of MDCF-2 could serve to not only facilitate design of improved compositions, but also to inform future recommendations regarding complementary feeding practices-recommendations that recognize the important role of the gut microbiome in the healthy growth of children.

Linking dietary glycans and microbial metabolism in this fashion provides a starting point for culture-based initiatives designed to retrieve isolates of these ‘effector’ taxa for use as potential probiotic agents, or if combined with key nutrients that they covet, synbiotic compositions for repairing the microbiomes of children who already manifest undernutrition or who are judged to be at risk for growth faltering. This repair could take the form of rebalancing the representation and/or expressed functions of beneficial organisms so that the microbiome assumes an age-appropriate configuration for healthy microbiome-host co-development.

Much remains unknown about whether or how the direct breakdown products of MDCF-2 glycan metabolism, or other secondary P. copri metabolites, are related to weight gain. Furthermore, interactions between P. copri, MDCF-2 glycans, and WLZ response does not exclude the contribution of other macro- or micronutrients. Direct tests of the role played by organisms such as P. copri in mediating microbial community and host responses to components of microbiome-targeted therapeutic foods can come from ‘reverse translation’ experiments of the type illustrated in the study that accompanies this report. To study this gnotobiotic mouse model colonized with defined collections of cultured were used, WLZ-associated gut bacterial taxa with or without P. copri, (ii) single nucleus RNA-Seq and microbial RNA-Seq and (iii) UHPLC-QqQ-MS to characterize the contributions of P. copri to regulating gene expression in gut epithelial cell lineages, processing of MDCF-2 glycans, and metabolism in intestinal and extra-intestinal tissues.

Some additional observations from the current study are provided below.

### Short Sequencing Read Only Versus Hybrid (Long and Short Read) MAG Assembly

The impact of the addition of long read sequencing data on various quality characteristics of MAGs assembled from data collected from the 0- and 3-month time points from all upper WLZ quartile responders (n=15) in the MDCF-2 treatment group was explored. The final set of high-quality, dereplicated MAGs, 918 MAGs represented contigs assembled from short read only data, while 82 were derived from hybrid short and long read assemblies. Although the mean quality characteristics of MAGs from each assembly type did not differ in completeness (determined by marker gene analysis) or total length, MAGs derived from hybrid assemblies displayed a significantly lower rate of contamination, fewer contigs, and greater N.

### Comparing MAG Assembly Accuracy and Quantitation Using a Pseudo-Alignment and Expectation Maximization Approach

MAG assembly algorithms that synthesize both contig sequence characteristics and contig abundance to assemble MAGs (e.g., MaxBin2, MetaBAT2) require accurate contig quantitation. Alignment-free quantitation approaches (e.g., Kallisto) have demonstrated superior speed and accuracy compared to read mapping-based quantitation in the context of metagenomic analyses where read-mapping ambiguity is common.

The utility of Kallisto-based quantitation was studied for (i) contigs, prior to MAG assembly, and (ii) MAGs themselves after assembly and curation. For this analysis, we employed a ‘mouse gut metagenome toy dataset’ from CAMI II that included 64 ‘mock fecal samples’; these mock samples were produced using sequencing data from 791 publicly available bacterial genomes (representing 549 species) and genomic abundances that mirrored bacterial 16S rRNA gene profiles of 64 actual mouse fecal biospecimens. Three components of this reference dataset were utilized for the analyses: (i) simulated sequencing data (1.67×10⁷ Illumina paired-end 150 nt reads) from each of the 64 mock fecal samples, (ii) anonymized reference contigs from the 791 reference genomes, and (iii) reference abundances of contigs/genomes in each fecal sample.

The effect of Kallisto quantification of contigs on the fidelity of MAG assembly was first investigated. The reference contigs using either Kallisto or bowtie2 and the short-read simulated Illumina data. Next, MAGs were assembled using MaxBin248, MetaBAT249 and CONCOCT82 with data from either Kallisto or bowtie2 contig quantitation as input. The output of each MAG assembly method for each sample was combined using DAS Tool. Finally, each MAG set was compared against 791 intact reference genomes using AMBER83. MAGs generated using Kallisto contig quantification and DAS Tool dereplication were more complete (P=6.4×10-14; Wilcoxon test) and less contaminated (P<2.2×10-16; Wilcoxon test) than those generated using bowtie2. Additionally, a significantly greater number of MAGs (P<0.05; Fisher's exact test) were detected using Kallisto contig quantitation.

Next, the same simulated dataset was employed to test the accuracy of Kallisto-based MAG quantitation. The short-read data was mapped for each of the 64 fecal samples to the set of 791 reference genomes using Kallisto and bowtie2. The abundance profiles generated by each quantitation method were then correlated to the ‘true’ abundance profile for each sample. The correlations between true genome abundances and Kallisto genome abundances were stronger than those calculated using bowtie (mean Pearson's r2=0.99 for Kallisto versus r2=0.97 for bowtie; P<2.2×10-16, Wilcoxon test comparing each distribution of correlation coefficients).

The false positive and false negative rate of MAG detection across all samples were determined. Notably, Kallisto quantitation resulted in more false positive abundances across the 64 mock fecal samples [300.2±50.1 versus 69.3±28.4 for bowtie2, respectively (mean±SD); P<2.2×10-16, Wilcoxon test] while bowtie2 quantitation resulted in more false negative abundances [0.09±0.42 versus 17.2±26.1 (mean±SD), respectively; P<2.2×10-16, Wilcoxon test]. Importantly, analysis of the average values of false positive abundance generated using Kallisto suggested that a low abundance filter would significantly reduce the false positive rate. For example, applying a filter to this dataset that required >5 TPM for a MAG to be designated as ‘detected’ resulted in a false positive rate significantly lower than that of bowtie2 (P=0.02, Wilcoxon test).

As a greater number of high quality (less contaminated and more complete) MAGs assembled could be assembled using Kallisto quantitation, plus the increased accuracy of MAG quantitation using this method, Kallisto was used for all quantitation tasks in the MAG analysis workflow described in the current study.

### Analysis of Consistency in MAG Functional Metabolic Pathway Annotation

A global comparison of binary phenotype assignments derived using the Pathway Rules (PR), Machine Learning (ML), and Neighbor Group (NG) approaches described in Methods revealed a remarkably low frequency of inconsistencies: in a subset of 640 MAGs where all three methods could be applied, only 4.5% of NG-based phenotype assignments were inconsistent between one or more other methods. These inconsistencies reflect different biases associated with each approach. The NG-based approach exhibits limited performance for small (<5-member) NGs with underrepresented local diversity of gene patterns. Alternatively, PR/ML-based methods appear to be less robust with respect to genome incompleteness in MAGs, resulting in omission (absence) of genes essential for the function of a pathway and, more generally, for pathways with less than three essential genes. Our consensus approach (Methods) resolved 70% of observed inconsistencies toward PR/ML-based assignments. In the remaining cases, a consensus phenotype was assigned in favor of the NG-based method. The overall level of inconsistencies between PR- and ML-based phenotype assignments (across the entire set of 199,334 assignments in 1,000 MAGs) was much lower (<0.7%). A detailed investigation of selected cases showed that, in general, the ML-based method yielded higher accuracy phenotype assignments. Therefore, in rare cases of irreconcilable disagreement between these two methods in the set of 360 MAGs without NGs, the semi-automated assignment of the consensus phenotype was made in favor of the ML-based approach. These assignments were considered low confidence.

### Non-Carbohydrate Related Differentially Expressed Transcripts in Upper Versus Lower WLZ Quartile Responders

Transcripts expressed at greater levels in upper WLZ response quartile participants (β₁ WLZ quartile term) were also enriched for pathways involved in biosynthesis of vitamin B3 and B9 and the essential amino acids tryptophan, lysine, histidine and leucine. Leading-edge analysis revealed that P. copri Bg0018 and Bg0019 were major contributors to increased expression of transcripts involved in the biosynthesis of vitamins B3 and B9 plus four essential amino acids (tryptophan, histidine, leucine, lysine) among the upper quartile participants but contributed minimally (2 transcripts assigned to the arginine biosynthetic pathway) to enrichment of functional pathways among the lower WLZ quartile responders.

### Example 7: Methods for Examples 8-12

### Bacterial Genome Sequencing and Annotation

Monocultures of each isolate were grown overnight at 37° C. in Wilkins-Chalgren Anaerobe Broth (Oxoid Ltd.; catalog number: CM0643) in a Coy Chamber under anaerobic conditions (atmosphere; 75% N2, 20% CO2 and 5% H2) without shaking. Cells were recovered by centrifugation (5000×g for 10 minutes at 4° C.) and high molecular weight genomic DNA was purified (MagAttract® HMW DNA kit, Qiagen) following the manufacturer's protocol and the amount quantified (Qubit fluorometer). The sample was passed up and down through a 29-gauge needle 6-8 times and the fragment size distribution was determined (˜30 kbp; TapeStation, Agilent) (Tables 10, 11 and 12).

TABLE 10
Bacterial strains used in the defined community gnotobiotic mouse experiments

| Taxonomic | | Contig length | Completeness | Contamination |
|---|---|---|---|---|
| assignment | Strain name | (bp) | (%; Check M) | (%; Check M) |
| Bifidobacterium | Bgsng463.m5.93 | 2365689* | 100 | 0.12 |
| breve | | | | |
| Bifidobacterium | Bgsng468.m22.84 | 2200049* | 99.77 | 0 |
| catenulatum | | | | |
| Bifidobacterium | Bg2D9 | 2505499, 5155 | 100 | 0.12 |
| longum subsp. | | | | |
| Infantis | | | | |
| Bifidobacterium | Bg463 | 32 contigs | 100 | 0.46 |
| longum subsp. | | | | |
| Infantis | | | | |
| Blautia luti | Bg7063 | 4077864*, 10061* | 99.37 | 0.32 |
| Blautia obeum | Bg7063_SSTS2015 | 3814509* | 99.37 | 0 |
| Dorea | Bg7063 | 3592951* | 99.42 | 0.58 |
| formicigenerans | | | | |
| Dorea | Bg7063 | 3598408* | 99.42 | 0 |
| longicatena | | | | |
| Enterococcus | Bang_SAM2.39.S1 | 3098697*, 1322944* | 98.94 | 4.62 |
| avium | | | | |
| Escherichia coli | PS131.S11 | 5063301*, 130375*, 83058*, 12672* | 99.97 | 0.28 |
| Faecalibacterium | Bg7063 | 2952590* | 99.32 | 0 |
| prausnitzii | | | | |
| Ligilactobacillus | ATCC | 2197604* | 99.37 | 1.05 |
| ruminis | 25644 | | | |
| Lactococcus | Bang155.08_4B6_JG2017 | 2007537*, 17753*, 12330* | 100 | 0 |
| garvieae | | | | |
| Mitsuokella | DSM 20544 | 2489477*, 98237* | 100 | 0.31 |
| multacida | | | | |
| Prevotella copri | PS131.S11 | 3321430, 494952, 138774*, 64774*, | 98.65 | 1 |
| | | 55930, 18865*, 3286, 3284, 3213, | | |
| | | 3213, 2484* | | |
| Prevotella | DSM 18206 | 1670248, 1126277, 477506, 49178 | 98.89 | 0.37 |
| stercorea | | | | |
| Ruminococcus | M8243_3A11_TMS_2014 | 4132922* | 99.42 | 0.24 |
| gnavus | | | | |
| Ruminococcus | Bg7063 | 3251172*, 13996*, 12811* | 99.12 | 0 |
| torques | | | | |
| Streptococcus | PS.064.S07 | 1949038*, 5031* | 100 | 0 |
| gallolyticus | | | | |
| Streptococcus | Bang_SAM2.39.S1 | 2287651*, 5260* | 100 | 0.75 |
| pasteurianus | | | | |

*circular contig verified with Flye algorithm

TABLE 11
P. copri PS131.S11 PULs

| Prevotella | | Genomic | | | |
|---|---|---|---|---|---|
| copri | Predicted PUL | location | PUL homologues | | |
| PS131.S11 | target(s) | (nt) | Bg0018 | Bg0019 | P. stercorea |
| PUL 1 | O-glycans/mucins | 53,824- | | | |
| | | 67,570 | | | |
| PUL 2 | a-L-fucoside + b- | 176,961- | | | |
| | galactoside | 188,692 | | | |
| PUL 3 | pectin | 416,027- | PUL 2¹ | PUL 10¹ | |
| | | 432,131 | | | |
| PUL 4 | no CAZyme | 977,609- | | PUL 18¹ | |
| | | 985,788 | | | |
| PUL 5 | b-mannan | 1,789,183- | | | |
| | | 1,801,010- | | | |
| PUL 6 | homogalacturonan | 1,898,685- | | | |
| | | 1,916,067 | | | |
| PUL 7 | pectin | 1,917,980- | | | |
| | | 1,931,862 | | | |
| PUL 8 | b-glucan, xylan | 2,100,128- | PUL 12¹ | PUL 13¹ | |
| | | 2,114,131 | | | |
| PUL 9 | b-1,6-glucan | 2,115,602- | | | |
| | | 2,125,069 | | | |
| PUL 10 | no CAZyme | 2,308,873- | | PUL 14¹ | |
| | | 2,314,214 | | | |
| PUL 11 | sucrose, inulin, levan | 2,329,974- | PUL 11¹ | CAZyme | PUL 4² |
| | | 2,336,605 | | cluster² | |
| PUL 12 | xylan | 2,385,086- | | | |
| | | 2,404,967 | | | |
| PUL 13 | arabinoxylan | 2,414,929- | PUL 8² | | |
| | | 2,436,160 | | | |
| PUL 14 | pectic galactan | 2,600,825- | PUL 6² | PUL 16² | |
| | | 2,626,492 | | | |
| PUL 15 | arabinoxylan | 2,691,562- | PUL 5² | PUL 3² | |
| | | 2,710,398 | | | |
| PUL 16 | no CAZyme | 2,713,851- | | PUL 2¹ | |
| | | 2,719,492 | | | |
| PUL 17 | unknown b- | 2,948,082- | | | |
| | galactoside | 2,961,522 | | | |
| PUL 18 | no CAZyme | 3,126,507- | | | |
| | | 3,131,588 | | | |
| PUL 19 | b-1,2-glucan | 3,143,047- | | | |
| | | 3,153,155 | | | |
| PUL 20 | no CAZyme | 3,182,564- | | PUL 12¹ | |
| | | 3,187,111 | | | |
| PUL 21 | Nand O-glycans | 3,188,964- | | | |
| | | 3,206,116 | | | |
| PUL 22 | a-glucoside, a-1,6- | 3,288,752- | | | |
| | glucan (dextran) | 3,307,423 | | | |
| PUL 23 | unknown | 16,235- | | | |
| | | 40,890 | | | |
| PUL 24 | no CAZyme | 61,128- | | | |
| | | 75,818 | | | |
| PUL 25 | type II | 126,212- | | | |
| | rhamnogalacturonan | 176,300 | | | |
| PUL 26 | a-glucan (starch) | 186,195- | | | |
| | | 197,081 | | | |
| PUL 27a | starch | 208,477- | PUL 18a² | PUL 17a¹ | |
| | | 227,992 | | | |
| PUL 27b | arabinogalactan | 228,820- | PUL 18b² | PUL 17b² | |
| | | 247,973 | | | |
| PUL 28 | no CAZyme | 252,400- | | | |
| | | 260,174 | | | |
| PUL 29 | no CAZyme | 296,439- | PUL 15¹ | | |
| | | 303,639 | | | |
| PUL 30 | b-1,3-glucan | 325,753- | PUL 16¹ | PUL 11¹ | |
| | | 337,891 | | | |
| PUL 31 | a-glucan (starch) | 362,093- | PUL 1¹ | CAZyme | |
| | | 372,659 | | cluster² | |

¹Functionally conserved
²Structurally distinct

TABLE 12
Bacterial strains used in the P. copri colonization dependency gnotobiotic mouse experiments.

| Taxonomic | Strain | | Completeness | Contamination |
|---|---|---|---|---|
| assignment | name | Contig length (bp) | (%; CheckM) | (%; CheckM) |
| Prevotella copri | G8 | 3992021*, 138455*, 90197*, | 98.65 | 1.86 |
| | | 26100* | | |
| | 2C6 | 3661021, 125949, 85415*, 52243, | 98.99 | 2.36 |
| | | 45123, 39569 | | |
| | 2D7 | 3872203*, 170001*, 71716* | 99.32 | 2.43 |
| | PS131.S11 | — | — | — |
| | 1A8_2 | 3690832*, 40375*, 22048* | 98.65 | 2.03 |

*circular contig verified with Flye algorithm

Fragmented genomic DNA (400-1000 ng) was prepared for long-read sequencing using a SMRTbell Express Template Prep Kit 2.0 (Pacific Biosciences) adapted to a deep 96-well plate (Fisher Scientific) format. All DNA handling and transfer steps were performed with wide-bore, genomic DNA pipette tips (ART). Barcoded adapters were ligated to A-tailed fragments (overnight incubation at 20° C.) and damaged or partial SMRTbell templates were subsequently removed (SMRTbell Enzyme Cleanup Kit). High molecular weight templates were purified (volume of added undiluted AMPure beads=0.45 times the volume of the DNA solution). Libraries prepared from different strains were pooled (3-6 libraries/pool). A second round of size selection was then performed; AMPure beads were diluted to a final concentration of 40% (v/v) with SMRTbell elution buffer with the resulting mixture added at 2.2 times the volume of the pooled libraries. DNA was eluted from the AMPure beads with 12 μL of SMRTbell elution buffer. Pooled libraries were quantified (Qubit), their size distribution was assessed (TapeStation) and sequenced [Sequel System, Sequel Binding Kit 3.0 and Sequencing Primer v4 (Pacific Biosystems)]. The resulting reads were demultiplexed and Q20 circular consensus sequencing (CCS) reads were generated (Cromwell workflow configured in SMRT Link software). Genomes were assembled using Flye (v2.8.1) with hifi-error set to 0.003, min-overlap set at 2000, and other options set to default. Genome quality was evaluated using CheckM (v1.1.3).

Prokka (v1.14) was applied to identify potential open reading frames (ORF) in each assembled genome. Additional functional annotation of these ORFs using a ‘subsystems’ approach adapted from the SEED genome annotation platform was performed. Functions were assigned to 9,820 ORFs in 20 isolate genomes using a collection of mcSEED metabolic subsystems that capture the core metabolism of 98 nutrients/metabolites in four major categories (amino acids, vitamins, carbohydrates, and fermentation products) projected over 2,856 annotated human gut bacterial genomes. In silico reconstructions of selected mcSEED metabolic pathways were based on functional gene annotation and prediction using homology-based methods and genome context analysis. Reconstructions were represented as a binary phenotype matrix (BPM) where for amino acids and B vitamins, “1” denotes a predicted prototroph and “0” an auxotroph, for carbohydrates, “1” and “0” refer to a strain's predicted ability or inability, respectively, to utilize the indicated mono-, di- or oligosaccharide, and for fermentation end products, a “1” and “0” indicate a strain's predicted ability/inability to produce the indicated compound, respectively.

To calculate phylogenetic relationships between five P. copri isolates and MAGs Bg0018 and Bg0019, CheckM (v1.1.3) was first used to extract and align the amino acid sequences of 43 single copy marker genes in each isolate or each of the two MAGs, plus an isolate genome sequence of Bacteroides thetaiotaomicron VPI-5482 (accession number: 226186.12). Concatenated marker gene sequences were analyzed using fasttree (v2.1.10) to construct a phylogenetic tree using the Jones-Taylor-Thornton model and ‘CAT’ evolution rate approximation, followed by tree rescaling using the ‘Gamma20’ optimization. The tree was subsequently processed in R using ‘ape’ (v5.6-2) to root the tree with the B. thetaiotaomicron genome and extract phylogenetic distances between genomes, followed by ‘ggtree’ (v3.2.1) for tree plotting.

The similarity between the genomes of these strains and MAGs was quantified by calculating the ANI score with pyani (ANIm implementation of ANI, v0.2.10). Firstly, ANIm scores were calculated for all possible combinations between MAGs and the genomes of cultured bacterial strains, and subsequently removed any MAG-strain genome combination with <10% alignment coverage. For the remaining MAGs, a “highly similar” genome in the collection of cultured bacterial strains was defined as having >94% ANIm score. The degree of binary phenotype concordance was then defined between each genome in the collection of cultured bacterial strains and its “highly similar” MAG. A binary phenotype concordance score was calculated by dividing the number of binary phenotypes shared between a cultured strain's genome and a MAG by the total number of binary phenotypes annotated in the strain and MAG. A ‘Representative MAG’ for each genome was defined as having a binary phenotype concordance score >90%.

PULs were predicted for P. copri PS131.S1 based on methods described in Terrapon et al. (Terrapon et al. Automatic prediction of polysaccharide utilization loci in Bacteroidetes species. Bioinformatics. 2015. 31, 647-655) and displayed with the PULDB interface. PULs were placed into three categories: (i) ‘functionally conserved’ (PULs containing shared ORFs encoding the same CAZymes and SusC/SusD proteins in the same organization in their respective genomes with ≥90% amino acid identity between proteins); (ii) ‘structurally distinct’ (PULs present in respective genomes but where one or more CAZymes or one or both SusC/SusD proteins are missing or fragmented in a way likely to impact function, or where extra PUL elements are present), and (iii) ‘not conserved’ (PULs present in respective genomes but with mutations likely to completely compromise function, or no PUL identified).

### Colonization and Husbandry

Gnotobiotic mouse experiments were performed using protocols approved by the Washington University Animal Studies Committee. Germ-free C57BL/6J mice were maintained in plastic flexible film isolators (Class Biologically Clean Ltd) at 23° C. under a strict 12-hour light cycle (lights on a 0600h). Autoclaved paper ‘shepherd shacks’ were kept in each cage to facilitate natural nesting behaviors and provide environmental enrichment.

A weaning diet containing MDCF-2 was formulated. Ingredients represented in the different diet modules were combined and the mixture was dried, pelleted, and sterilized by gamma irradiation (30-50 KGy). Sterility was confirmed by culturing the pellets in LYBHI medium and Wilkins-Chalgren Anaerobe Broth under aerobic and anaerobic conditions for 7 days at 37° C. followed by plating on LYBHI- and blood-agar plates. Nutritional analysis of each irradiated diet was performed by Nestlé Purina Analytical Laboratories (St. Louis, MO) (Table 13).

TABLE 13
Nutritional analysis of the diets

| | | | Weaning diet |
|---|---|---|---|
| | | MDCF-2 | supplemented |
| | Unit | module | with MDCF-2 |
| Calories | Kcal/g | 5.05 | 4.72 |
| Protein | % | 12.6 | 10.7 |
| Total fat | g/100 g | 20.1 | 18.4 |
| Total dietary fiber | % | 3.89 | 2.81 |
| Moisture | % | 8.63 | 12.9 |
| Sodium | ppm | 251.7 | 2419 |
| Potassium | ppm | 5676 | 5217 |
| Calcium | ppm | 1156 | 2957 |
| Phosphorus | ppm | 1936 | 2603 |
| Magnesium | ppm | 798.3 | 539.5 |
| Iron | ppm | 24.27 | 75.69 |
| Manganese | ppm | 10.39 | 5.6 |
| Zinc | ppm | 17.91 | 31.61 |
| Copper | ppm | 3.967 | 3.811 |
| Alanine | g/100 g | 0.51 | 0.43 |
| Arginine | g/100 g | 1.1 | 0.63 |
| Aspartic acid | g/100 g | 1.41 | 0.99 |
| Glutamic acid | g/100 g | 2.35 | 2.11 |
| Glycine | g/100 g | 0.56 | 0.34 |
| Histidine | g/100 g | 0.32 | 0.28 |
| Isoleucine | g/100 g | 0.49 | 0.47 |
| Leucine | g/100 g | 0.85 | 0.87 |
| Lysine | g/100 g | 0.63 | 0.6 |
| Methionine | g/100 g | 0.11 | 0.16 |
| Phenylalanine | g/100 g | 0.65 | 0.54 |
| Proline | g/100 g | 0.59 | 0.8 |
| Serine | g/100 g | 0.57 | 0.51 |
| Threonine | g/100 g | 0.43 | 0.41 |
| Tyrosine | g/100 g | 0.41 | 0.47 |
| Valine | g/100 g | 0.52 | 0.57 |
| Cholesterol | % | 0.00321 | 0.00329 |

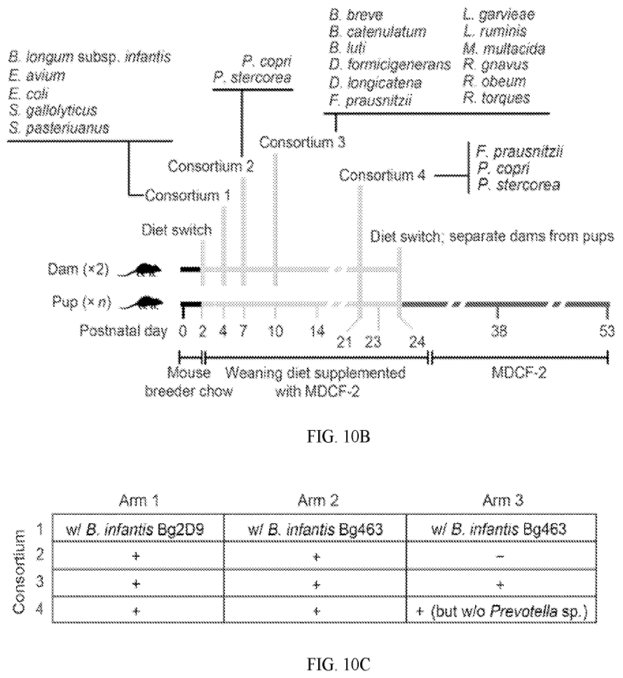

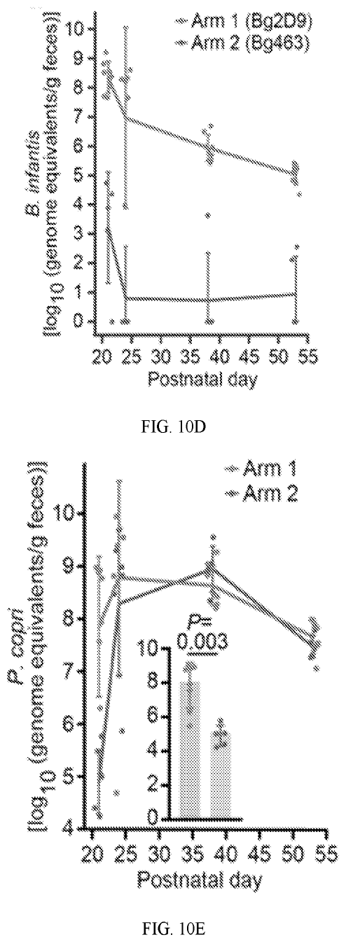

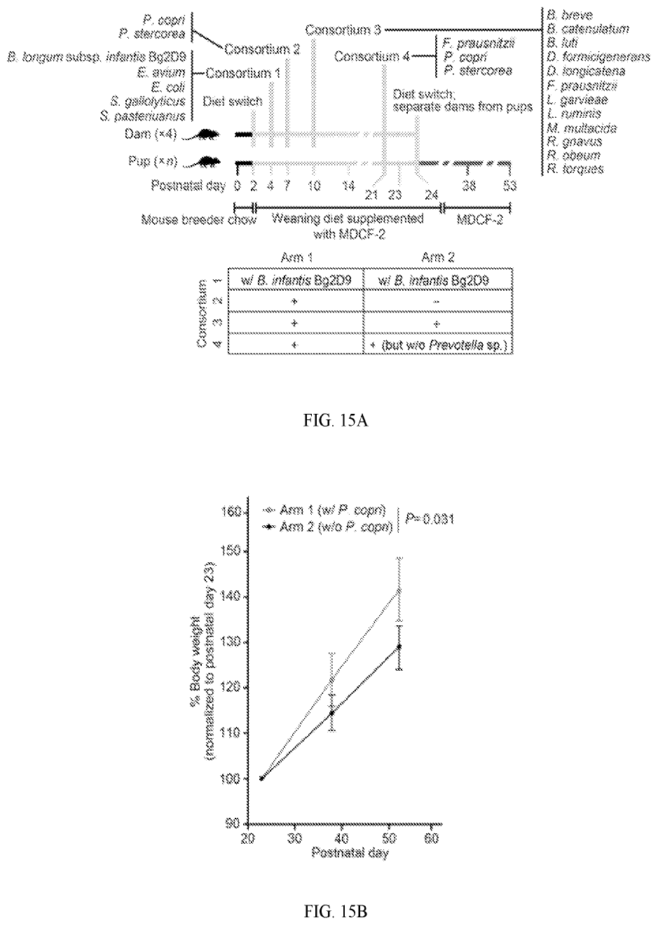

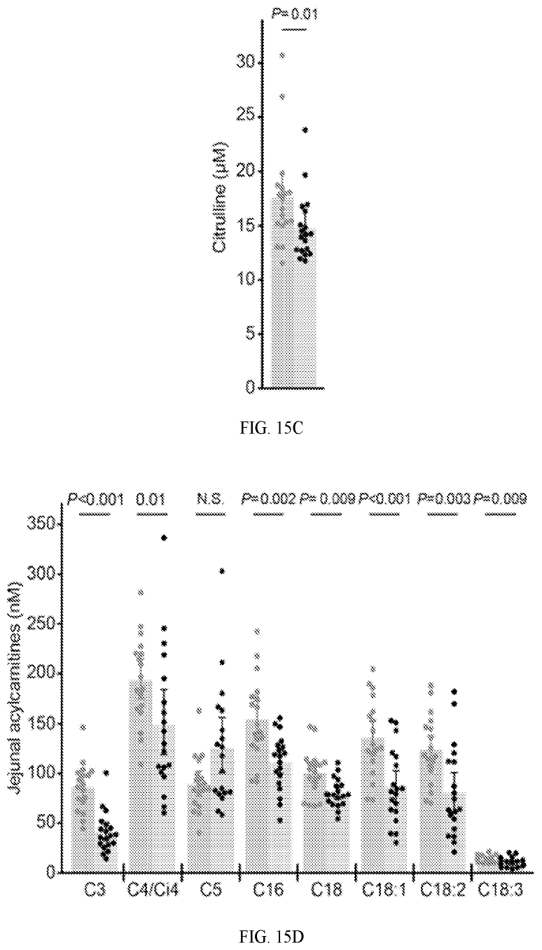

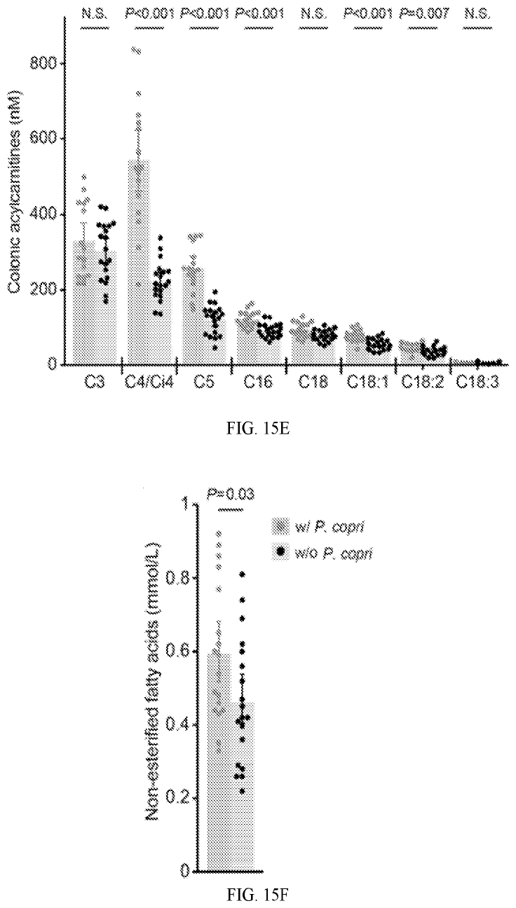

Pregnant C57Bl/6J mice originating from trio matings were given ad libitum access to an autoclaved breeder chow (Purina Mills; Lab Diet 5021) throughout their pregnancy and to postpartum day 2. Key points about the experimental design doe the gnotobiotic mouse experiments described in FIG. 10B and FIG. 15A are: (i) all bacterial strains were cultured in Wilkins-Chalgren Anaerobe Broth (except for F. prausnitzii which was cultured in LYBHI medium) and were harvested after overnight growth at 37° C. (Table 13); (ii) all gavage mixtures contained equivalent amounts (by OD600) of their constituent bacterial strains except for F. prausnitzii which was concentrated 100-fold before preparing the gavage mixture; (iii) each bacterial consortium was administered to the postpartum dams in a volume of 200 μL using an oral gavage needle (Cadence Science; catalog number: 7901); (iv) the number of dams and pups per treatment group [two dams and 7-8 pups/treatment group (FIG. 10B); four dams and 18-19 pups/treatment group (FIG. 15A)]; (v) half of the bedding was replaced with fresh bedding in each cage each day from postpartum day 1 to 14, after which time bedding was changed every 7 days; (vi) diets were provided to mothers as well as to their weaning and post-weaning pups ad libitum, (vii) fecal samples were collected from mice when they were euthanized (without prior fasting) and snap frozen in liquid nitrogen and stored at −80° C. before use.

Pups were weighed on P23, P35, and P53, and normalized to the weight on P23. A linear mixed-effects model was used to evaluate the effect of different microbial communities on normalized mouse weight gain:

$$normalized weight ∼ {β}_{1} ( Arm ) + {β}_{2} ( postnatal day ) + ( 1 \vert mouse ) \qquad ( 1 )$$

### Defining the Absolute Abundances of Bacterial Strains in Ileal, Cecal and Fecal Communities

The absolute abundances of bacterial strains were determined in the fecal microbiota. In brief, 3.3×10⁶ cells of Alicyclobacillus acidiphilus DSM 14558 and 1.49×10⁷ cells of Agrobacterium radiobacter DSM 30147 (ref. 49) were added to each weighed frozen sample prior to DNA isolation and preparation of barcoded libraries for shotgun sequencing. Sequencing was performed for 136 samples (Illumina NextSeq instrument; unidirectional 75 nt reads) at an average depth of 2.0×10⁶±4.0×10⁵ reads/sample (mean±SD). Bacterial abundances were determined by assigning reads to each bacterial genome, followed by a normalization for genome uniqueness in the context of a given community. The resulting count table was imported into R (v4.0.4). The absolute abundance of a given strain i in sample j in reference to the spike-in A. acidiphilus (Aa) and A. radiobacter (Ar) genomes was calculated using the following equation:

$${AAAAAA}_{AA} = \frac{{AAAAAA}_{AA} \times {AAAAAAAAAAAA}_{A}}{{AAAAAAAA}_{A} \times {AAAAAAAAAAhA}_{A}} + \frac{{AAAAAA}_{AA} \times {AAAAAAAAAAAA}_{A}}{{AAAAAAAA}_{A} \times {AAAAAAAAAAhA}_{A}} \times 0.5 \qquad ( 2 )$$

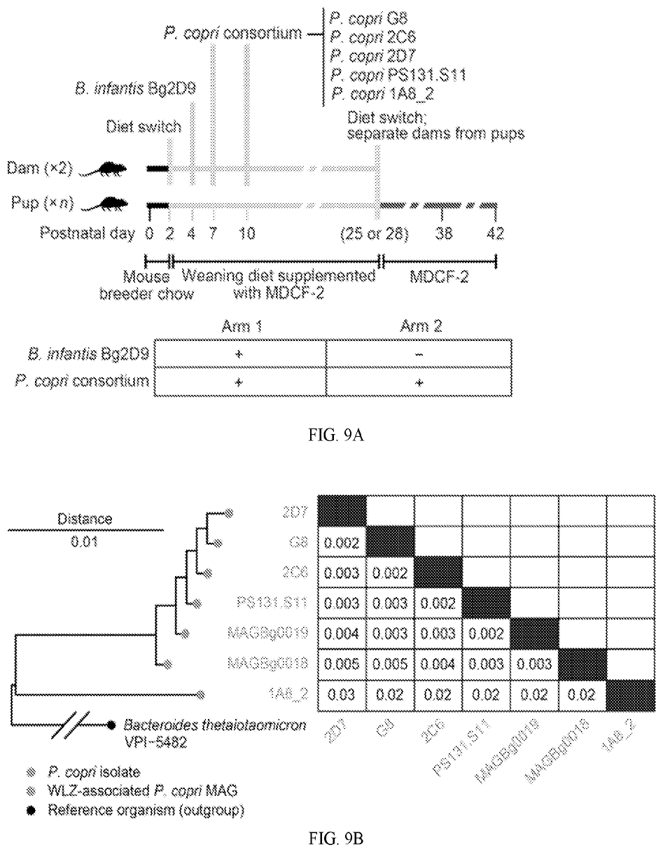

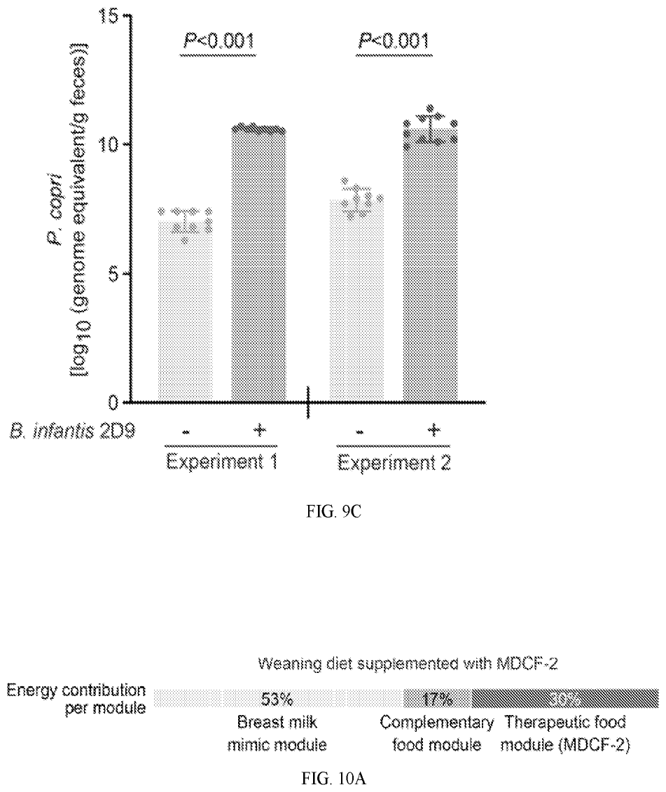

The statistical significance of observed differences in the abundance of a given strain across different treatment groups and time was tested using a linear mixed effects model within the R packages Ime4 (v1.1-27) and ImerTest (v3.1-3). For the experiment described in FIGS. 9A-C, 37 fecal samples were sequenced [5.8×10⁶±1.6×10⁶ unidirectional 75 nt reads/sample (mean±SD)] (Tables 14 and 15)] while for the experiment described in FIG. 15A, 37 cecal samples were sequenced [1.3×10⁶±1.3×10⁵ unidirectional 75 nt reads/sample] and absolute abundances were determined as above. The change in P. copri absolute abundance in fecal samples during the course of the experiment was determined by a linear mixed-effects model:

$$P . copri absolute abundance - {β}_{1} ( Arm ) + {β}_{2} ( postnatal day ) + ( 1 \vert mouse ) \qquad ( 3 )$$

TABLE 14
Sample metadata

| | Bifido- | | | | |
|---|---|---|---|---|---|
| | bacterium | | | | |
| Experi- | longum subsp. | | | Number | |
| ment | infantis_2D9 | Mouse | | of raw | Combined |
| No. | colonization | ID | Sample_ID | reads | Index |
| 1 | w/ | 1 | P. | 4,768,348 | TACCTGAC- |
| | | | copri_colonization_experiment_ | | GACCGCCA |
| | | | 1.with_2D9.mouse_1_CoProSeq | | |
| | | 2 | P | 4,332,109 | AGGACCGC- |
| | | | copri_colonization_experiment_ | | GACCGCCA |
| | | | 1.with_2D9.mouse_2_CoProSeq | | |
| | | 3 | P. | 7,051,041 | GTCCGATT- |
| | | | copri_colonization_experiment_ | | GACCGCCA |
| | | | 1.with_2D9.mouse_3_CoProSeq | | |
| | | 4 | P. | 5,713,730 | CACGAGTT- |
| | | | copri_colonization_experiment_ | | GACCGCCA |
| | | | 1.with_2D9.mouse_4_CoProSeq | | |
| | | 5 | P. | 5,312,529 | CCACGGCC- |
| | | | copri_colonization_experiment_ | | GACCGCCA |
| | | | 1.with_2D9.mouse_5_CoProSeq | | |
| | | 6 | P. | 4,888,067 | ACATGTAA- |
| | | | copri_colonization_experiment_ | | GACCGCCA |
| | | | 1.with_2D9.mouse_6_CoProSeq | | |
| | | 7 | P. | 4,886,218 | TGTTAACT- |
| | | | copri_colonization_experiment_ | | GACCGCCA |
| | | | 1.with_2D9.mouse_7_CoProSeq | | |
| | | 8 | P | 4,391,074 | TTCTTCTA- |
| | | | copri_colonization_experiment_ | | TAAGATGG |
| | | | 1.with_2D9.mouse_8_CoProSeq | | |
| | | 9 | P. | 4,597,974 | TACCTGAC- |
| | | | copri_colonization_experiment_ | | TAAGATGG |
| | | | 1.with_2D9.mouse_9_CoProSeq | | |
| | w/o | 1 | P. | 3,933,342 | TTCTTCTA- |
| | | | copri_colonization_experiment_ | | CGCGGTTA |
| | | | 1.without_2D9.mouse_1_CoProSeq | | |
| | | 2 | P. | 4,389,615 | TACCTGAC- |
| | | | copri_colonization_experiment_ | | CGCGGTTA |
| | | | 1.without_2D9.mouse_2_CoProSeq | | |
| | | 3 | P. | 4,170,047 | AGGACCGC- |
| | | | copri_colonization_experiment_ | | CGCGGTTA |
| | | | 1.without_2D9.mouse_3_CoProSeq | | |
| | | 4 | P. | 4,432,105 | GTCCGATT- |
| | | | copri_colonization_experiment_ | | CGCGGTTA |
| | | | 1.without_2D9.mouse_4_CoProSeq | | |
| | | 5 | P. | 5,602,991 | CACGAGTT- |
| | | | copri_colonization_experiment_ | | CGCGGTTA |
| | | | 1.without_2D9.mouse_5_CoProSeq | | |
| | | 6 | P. | 11,554,583 | CCACGGCC- |
| | | | copri_colonization_experiment_ | | CGCGGTTA |
| | | | 1.without_2D9.mouse_6_CoProSeq | | |
| | | 7 | P. | 4,804,758 | ACATGTAA- |
| | | | copri_colonization_experiment_ | | CGCGGTTA |
| | | | 1.without_2D9.mouse_7_CoProSeq | | |
| | | 8 | P. | 4,302,251 | TGTTAACT- |
| | | | copri_colonization_experiment_ | | CGCGGTTA |
| | | | 1.without_2D9.mouse_8_CoProSeq | | |
| | | 9 | P. | 5,774,202 | TTCTTCTA- |
| | | | copri_colonization_experiment_ | | GACCGCCA |
| | | | 1.without_2D9.mouse_9_CoProSeq | | |
| 2 | w/ | 1 | P. | 6,239,039 | AGGACCGC- |
| | | | copri_colonization_experiment_ | | CTGAATTC |
| | | | 2.with_2D9.mouse_1_CoProSeq | | |
| | | 2 | P | 6,049,883 | GTCCGATT- |
| | | | copri_colonization_experiment_ | | CTGAATTC |
| | | | 2.with_2D9.mouse_2_CoProSeq | | |
| | | 3 | P. | 6,982,790 | CACGAGTT- |
| | | | copri_colonization_experiment_ | | CTGAATTC |
| | | | 2.with_2D9.mouse_3_CoProSeq | | |
| | | 4 | P. | 9,528,315 | CCACGGCC- |
| | | | copri_colonization_experiment_ | | CTGAATTC |
| | | | 2.with_2D9.mouse_4_CoProSeq | | |
| | | 5 | P. | 7,667,234 | ACATGTAA- |
| | | | copri_colonization_experiment_ | | CTGAATTC |
| | | | 2.with_2D9.mouse_5_CoProSeq | | |
| | | 6 | P. | 8,350,681 | TGTTAACT- |
| | | | copri_colonization_experiment_ | | CTGAATTC |
| | | | 2.with_2D9.mouse_6_CoProSeq | | |
| | | 7 | P. | 5,597,522 | TTCTTCTA- |
| | | | copri_colonization_experiment_ | | CGTACCGG |
| | | | 2.with_2D9.mouse_7_CoProSeq | | |
| | | 8 | P. | 5,437,446 | TACCTGAC- |
| | | | copri_colonization_experiment_ | | CGTACCGG |
| | | | 2.with_2D9.mouse_8_CoProSeq | | |
| | | 9 | P. | 5,200,291 | AGGACCGC- |
| | | | copri_colonization_experiment_ | | CGTACCGG |
| | | | 2.with_2D9.mouse_9_CoProSeq | | |
| | | 10 | P. | 5,534,353 | GTCCGATT- |
| | | | copri_colonization_experiment_ | | CGTACCGG |
| | | | 2.with_2D9.mouse_10_CoProSeq | | |
| | w/o | 1 | P. | 6,269,303 | CACGAGTT- |
| | | | copri_colonization_experiment_ | | CGTACCGG |
| | | | 2.without_2D9.mouse_1_CoProSeq | | |
| | | 2 | P. | 7,431,787 | CCACGGCC- |
| | | | copri_colonization_experiment_ | | CGTACCGG |
| | | | 2.without_2D9.mouse_2_CoProSeq | | |
| | | 3 | P. | 5,640,452 | ACATGTAA- |
| | | | copri_colonization_experiment_ | | CGTACCGG |
| | | | 2.without_2D9.mouse_3_CoProSeq | | |
| | | 4 | P. | 5,737,257 | TGTTAACT- |
| | | | copri_colonization_experiment_ | | CGTACCGG |
| | | | 2.without_2D9.mouse_4_CoProSeq | | |
| | | 6 | P. | 5,392,195 | TTCTTCTA- |
| | | | copri_colonization_experiment_ | | GATGACGG |
| | | | 2.without_2D9.mouse_6_CoProSeq | | |
| | | 7 | P. | 5,093,265 | TACCTGAC- |
| | | | copri colonization experiment_ | | GATGACGG |
| | | | 2.without_2D9.mouse_7_CoProSeq | | |
| | | 8 | P. | 4,992,059 | AGGACCGC- |
| | | | copri_colonization_experiment_ | | GATGACGG |
| | | | 2.without_2D9.mouse_8_CoProSeq | | |
| | | 9 | P. | 5,096,241 | GTCCGATT- |
| | | | copri_colonization_experiment_ | | GATGACGG |
| | | | 2.without_2D9.mouse_9_CoProSeq | | |
| | | 10 | P. | 5,986,785 | CACGAGTT- |
| | | | copri_colonization_experiment_ | | GATGACGG |
| | | | 2.without_2D9.mouse_10_ | | |
| | | | CoProSeq | | |

TABLE 15
Absolute abundance of strains [log10(genome equivalents per gram sample)] in cecal contents collected from progeny of dams at sacrifice

| | Bifidobacterium | | | | | | | | |
|---|---|---|---|---|---|---|---|---|---|
| | longum | | Bifidobacterium | | | | | | |
| | subsp. | | longum | Total | | | | | |
| | infantis | | subsp. | P. | P. | P | P | P. | P. |
| Experiment | 2D9 | Mouse | Infantis | copri | copri | copri | copri | copri | copri |
| No. | colonization | ID | 2D9 | load | 1A8 | 2C6 | 2D7 | G_8 | PS131.S11 |
| 1 | w/ | 1 | 9.99 | 10.54 | 5.83 | 10.29 | 10.17 | 7.26 | 7.39 |
| | | 2 | 9.99 | 10.47 | 6.01 | 10.33 | 9.91 | 7.97 | 7.48 |
| | | 3 | 10.03 | 10.60 | 5.71 | 10.55 | 9.65 | 7.49 | 6.52 |
| | | 4 | 10.09 | 10.64 | 5.72 | 10.39 | 10.27 | 7.67 | 6.71 |
| | | 5 | 9.94 | 10.48 | 5.52 | 10.48 | 7.49 | 7.54 | 7.21 |
| | | 6 | 10.13 | 10.73 | 5.79 | 10.16 | 10.59 | 8.06 | 7.13 |
| | | 7 | 9.94 | 10.61 | 5.79 | 10.48 | 10.02 | 7.63 | 7.27 |
| | | 8 | 10.10 | 10.55 | 5.67 | 10.31 | 10.19 | 6.98 | 6.64 |
| | | 9 | 9.86 | 10.71 | 5.85 | 10.62 | 9.97 | 7.17 | 6.79 |
| | mean ± SD | | 10.01 ± 0.09 | 10.59 ± 0.09 | 5.77 ± 0.14 | 10.40 ± 0.14 | 9.81 ± 0.91 | 7.53 ± 0.36 | 7.02 ± 0.35 |
| | w/o | 1 | ND | 7.04 | 3.81 | 5.68 | 6.31 | 6.76 | 6.43 |
| | | 2 | ND | 7.37 | 4.35 | 6.32 | 6.56 | 7.09 | 6.74 |
| | | 3 | ND | 6.84 | 4.26 | 5.59 | 5.53 | 6.65 | 6.24 |
| | | 4 | ND | 6.79 | 3.68 | 5.58 | 5.95 | 6.63 | 5.82 |
| | | 5 | ND | 6.30 | 2.90 | 5.09 | 5.48 | 6.13 | 5.27 |
| | | 6 | ND | 7.37 | 0.00 | 6.39 | 6.34 | 7.23 | 6.14 |
| | | 7 | ND | 7.38 | 0.00 | 6.10 | 6.62 | 7.19 | 6.49 |
| | | 8 | ND | 6.72 | 0.00 | 5.65 | 5.82 | 6.38 | 6.26 |
| | | 9 | ND | 7.40 | 0.00 | 7.06 | 6.80 | 6.71 | 6.29 |
| | mean ± SD | | N/A | 7.02 ± 0.39 | 2.11 ± 2.04 | 5.94 ± 0.59 | 6.16 ± 0.48 | 6.75 ± 0.37 | 6.19 ± 0.42 |
| 2 | w/ | 1 | 9.57 | 9.93 | 5.63 | 9.07 | 6.99 | 9.86 | 6.68 |
| | | 2 | 10.26 | 10.83 | 6.30 | 10.38 | 7.65 | 10.64 | 7.32 |
| | | 3 | 10.80 | 11.06 | 6.70 | 10.64 | 7.84 | 10.85 | 7.67 |
| | | 4 | 9.81 | 10.20 | 5.86 | 9.46 | 6.96 | 10.12 | 6.97 |
| | | 5 | 10.48 | 11.05 | 6.65 | 10.29 | 7.77 | 10.97 | 7.73 |
| | | 6 | 9.89 | 10.10 | 5.65 | 9.57 | 7.62 | 9.94 | 7.39 |
| | | 7 | 10.37 | 10.75 | 6.34 | 10.15 | 7.57 | 10.62 | 7.34 |
| | | 8 | 10.98 | 11.40 | 6.89 | 10.94 | 8.65 | 11.22 | 8.29 |
| | | 9 | 9.85 | 10.15 | 5.91 | 9.73 | 7.46 | 9.95 | 7.06 |
| | | 10 | 9.95 | 10.43 | 6.06 | 9.88 | 7.21 | 10.28 | 6.80 |
| | mean ± SD | | 10.20 ± 0.46 | 10.59 ± 0.50 | 6.20 ± 0.45 | 10.01 ± 0.57 | 7.57 ± 0.49 | 10.45 ± 0.48 | 7.33 ± 0.48 |
| | w/o | 1 | ND | 8.27 | 4.73 | 7.56 | 7.27 | 8.06 | 7.24 |
| | | 2 | ND | 7.73 | 0.00 | 7.15 | 6.90 | 7.44 | 6.66 |
| | | 3 | ND | 7.67 | 3.71 | 7.05 | 6.86 | 7.38 | 6.55 |
| | | 4 | ND | 7.93 | 0.00 | 7.31 | 7.13 | 7.65 | 6.80 |
| | | 6 | ND | 7.35 | 3.43 | 6.67 | 6.31 | 7.14 | 6.23 |
| | | 7 | ND | 8.00 | 4.94 | 7.30 | 7.08 | 7.76 | 6.99 |
| | | 8 | ND | 8.58 | 5.35 | 7.89 | 7.58 | 8.37 | 7.46 |
| | | 9 | ND | 7.92 | 4.95 | 7.30 | 6.91 | 7.69 | 6.82 |
| | | 10 | ND | 7.22 | 3.97 | 6.61 | 6.40 | 6.94 | 6.11 |

| | | | | | | | |
|---|---|---|---|---|---|---|---|
| mean ± SD | N/A | 7.85 ± 0.42 | 3.45 ± 2.06 | 7.20 ± 0.40 | 6.94 ± 0.40 | 7.60 ± 0.44 | 6.77 ± 0.44 |

### Microbial RNA-Seq

RNA was isolated from cecal contents collected at the end of the experiment. cDNA libraries were generated from isolated RNA samples using the ‘Total RNA Prep with Ribo-Zero Plus’ kit (Illumina). Barcoded libraries were sequenced [Illumina NovaSeq instrument; bidirectional 150 nt reads; 8.0×10⁷±1.6×10⁷ reads/sample (mean±SD); n=40 samples]. Raw reads were trimmed by using TrimGalore (v0.6.4). Trimmed reads longer than 100 bp were mapped to reference genomes with Kallisto (v0.43.0). Mapping of reads was skipped for strains that were not gavaged in all arms (P. copri, P. stercorea, B. longum subsp. infantis strains Bg2D9 and Bg463) in order to compare transcriptional changes induced by the differential presence of these strains. The resulting Kallisto pseudocount dataset, comprised of 48,390 transcripts, was imported into R (v4.0.4). edgeR (v 3.36.0) was used to filter the pseudocount dataset by expression level, resulting in a dataset of 22,387 expressed genes.

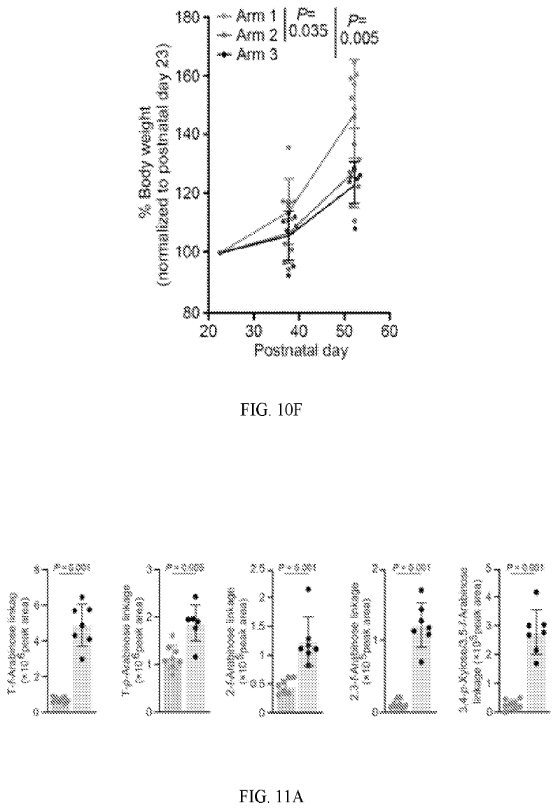

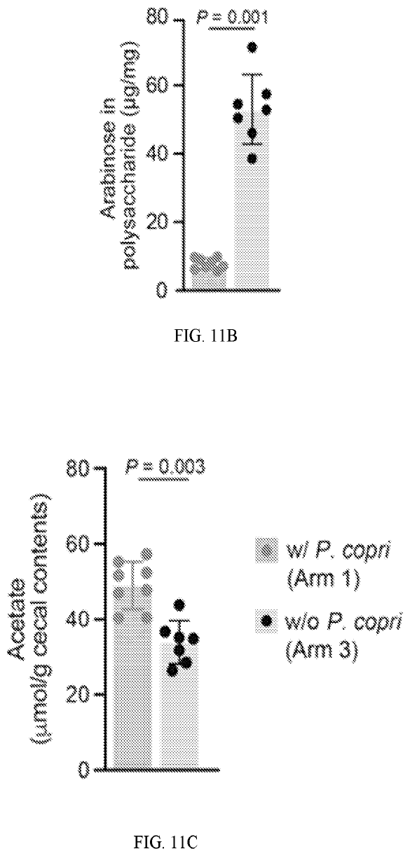

For analysis of metabolic pathway expression, cecal content pseudocounts for each transcript were first normalized by the absolute abundance of the corresponding strain in order to minimize the confounding effects of differences in strain abundance. The r log transformation in the R DESeq2 package (v1.34.0) was then applied to this dataset for variance stabilization. To obtain relative expression of metabolic pathways, only transcripts corresponding to complete mcSEED metabolic pathways (i.e., pathways with binary phenotype scores of 1; see above) were retained and transformed expression values were averaged across genes within a given pathway in each strain. The resulting aggregated pathway expression dataset was then centered prior to singular value decomposition with NumPy (v1.21.5). Principal component analysis of samples was calculated by projecting the centered pathway dataset onto right singular vectors (FIG. 11D). Sample projections along the first two principal components were converted to a Euclidian distance matrix, and PERMANOVA was used to test for the significance of separation of samples by experimental group. Pathway PCA loadings were calculated by projecting the transpose of the centered dataset onto left singular vectors. Metabolic pathways were subsequently ranked by their corresponding loadings, and directionality was resolved such that more negative left singular vector 1 projections corresponded to higher pathway expression in the P. copri colonization context.

For differential expression analysis of microbial transcripts, the filtered version of Kallisto pseudocounts was imported into edgeR. For each community member, ‘within-taxon sum-scaling’ was applied by calculating the trimmed mean of M-value library size corrections based on the total pool of RNA reads from that member. This organism-scaled transcript set was then used for dispersion estimation and fitting of a generalized linear model (GLM). In addition to ‘experiment arm’, the absolute abundance of the organism was included in each cecal sample as a covariate in the GLM to reduce false discoveries due to differences in the abundances of community members. A likelihood ratio test (edgeR) was then used to detect differential expression between samples obtained from members of the w/ P. copri versus w/o P. copri treatment groups. Transcripts with statistically significant differences in their expression were identified [q-value <0.05 (adjusted P value <0.05)] after multiple hypothesis correction was applied to the entire set of transcripts from a given organism via the Benjamini-Hochberg method.

### Histomorphometric Analysis of Villus Height and Crypt Depth

Jejunal and ileal segments were fixed in formalin, embedded vertically in paraffin; 5 gm-thick sections were prepared and stained with hematoxylin and eosin. Slides were scanned (NanoZoomer instrument, Hamamatsu). For each animal, 10 well-oriented crypt-villus units were selected from each intestinal segment for measurement of villus height and crypt depth using QuPath (v0.3.2). Measurements were performed with the investigator blinded with respect to colonization group. A two-tailed Mann-Whitney U test was applied to the resulting datasets.

### Single Nucleus (sn) RNA-Seq

Jejunal segments of 1.5 cm in length were collected from mice and snap frozen in liquid nitrogen [n=4 animals/treatment group (2 males and 2 females); 2 treatment groups in total]. The method for extracting nuclei was adapted from a previously described protocol for the pancreas⁶². Briefly, tissues were thawed and minced in lysis buffer [25 mM citric acid, 0.25M sucrose and 0.1% NP-40, and 1× protease inhibitor (Roche)]. Nuclei were released from cells using a pestle douncer (Wheaton), washed 3 times with buffer [25 mM citric acid, 0.25M sucrose, and 1× protease inhibitor], and filtered successively through 100 tm, 70 tm, 40 tm, 20 tm and finally 5 tm diameter strainers (pluriSelect) to obtain single nuclei in resuspension buffer [25 mM KCl, 3 mM MgCl2, 50 mM Tris, 1 mM DTT, 0.4 U/L RNase inhibitor (Sigma) and 0.4 U/tL Superase inhibitor (ThermoFisher)]. Approximately 10,000 nuclei per sample were subjected to gel bead-in-emulsion (GEM) generation, reverse transcription and construction of libraries for sequencing according to the protocol provided in the 3′ gene expression v3.1 kit manual (10× Genomics). Libraries were balanced, pooled and sequenced [Illumina NovaSeq S4; 3.23×10⁸±1.39×10⁷ paired-end 150 nt reads/nucleus (mean±SD) from jejunal samples, respectively]. Read alignment, feature-barcode matrices and quality controls were processed by using the CellRanger 5.0 pipeline with the flag ‘--include-introns’ to ensure that reads would be allowed to map to intronic regions of the mouse reference genome (GRCm38/mm10). Nuclei with over 2.5% reads from mitochondria-encoded genes reads or ribosomal protein genes were filtered out.

Analysis of snRNA-seq datasets—Sample integration, count normalization, cell clustering and marker gene identification was performed using Seurat 4.0. Briefly, filtered feature-barcode matrices outputted from CellRanger were imported as a Seurat object using CreateSeuratObject (min.cells=5, min.features=200). Each sample was normalized using SCTransform^{63,64} and integrated using SelectIntegrationFeatures, PrepSCTIntegration, FindIntegrationAnchors, and IntegrateData from the Seurat software package. The integrated dataset, incorporating nuclei from all samples, was subject to unsupervised clustering using FindNeighbors (dimensions=1:30) and FindClusters (resolution=1) from the Seurat package, which executes a shared nearest-neighbor graph clustering algorithm to identify putative cell clusters. Cell type assignment was performed manually based on expression of reported markers.

Cross-condition differential gene expression analysis was performed based on a “pseudobulk” strategy; for each cell cluster, gene counts were aggregated to obtain sample-level counts; each pseudo-bulked sample served as an input for edgeR-based differential gene expression analysis.

For NicheNet-based analysis (v1.1.0), all clusters in snRNA-seq dataset were used as senders for crypt stem cells, proliferating TA/stem cells, villus base enterocytes, mid-villus enterocytes and villus tip enterocytes, plus goblet cells). The nichenet_seuratobj_aggregate (assay_oi=“RNA”) function was used with its default settings to incorporate differential gene expression information from Seurat into our NicheNet analysis and to select bona fide ligand-receptor interactions.

Compass-based in silico metabolic flux analysis (v0.9.10.2) was performed using transcripts from each of six epithelial cell clusters (crypt stem cells, proliferating TA cells, villus-base, mid-villus and villus tip enterocytes and goblet cells). The reaction scores calculated by Compass were filtered based on (i) the confidence levels of the Recon2 reactions and (ii) the completeness of information for Recon2 reaction annotations. Only Recon2 reactions that are supported by biochemical evidence (defined by Recon2 as having a confidence level of 4) and that have complete enzymatic information for the reaction were advanced to the follow-on analysis (yield: 2,075 pass filter reactions in 83 Recon2 subsystems).

A “metabolic flux difference” was calculated to determine whether the presence or absence of P. copri affected Compass-based predictions of metabolic activities at the Recon2 reaction level in the six cell clusters. The “net reaction score” was calculated as follows

$$c = {c}_{f} - {c}_{r} \qquad ( 4 )$$

were cf denotes the Compass score for a given reaction in the “forward” direction, and, if the biochemical reaction is reversible, cᵣ denotes the score for the “reverse” reaction.

A Wilcoxon Rank Sum test was used to test significance of the net reaction score between the two treatment groups. P values from the Wilcoxon Rank Sum tests were adjusted for multiple comparisons with the Benjamini-Hochberg method.

Cohen's d can be used to show the effect size of cf or cᵣ for each reaction between two groups (in mice harboring communities with and without P. copri). Briefly, Cohen's d of two groups, j and k, was calculated based on Equations 4 and 5. n, S, and a in Equation 4 represent the number, the variance, and the mean of the observations (in our case, the net reaction scores). Cohen's d was defined as:

$${A}_{AAAA} = \sqrt{\frac{{A}_{A} - 1 {A}_{A}^{2} - ( {A}_{A} - 1 ) {A}_{A}^{2}}{{A}_{A} + {A}_{A} - 2}} \qquad ( 5 )$$

$$A = \frac{{A}_{A} - {A}_{A}}{{A}_{AAAA}} \qquad ( 6 )$$

If both ai and aₖ are non-negative numbers, a positive Cohen's d indicates the mean of group j is greater than that of group k whereas a negative Cohen's d means the mean of group j is smaller in that comparison. The magnitude of Cohen's d represents the effect size and is correlated with the difference between the means of the two groups. Because the mean of the net subsystem scores as well as the net reaction scores could be negative, the following adjustments were made to Cohen's d in order to preserve the concordance of sign and the order of group means. The adjusted Cohen's d represents the metabolic flux difference m, and is defined as:

$$\{ \begin{matrix}{A}_{A} > 0 ; {A}_{A} < 0 ; \vert {A}_{A} \vert < \vert {A}_{A} \vert : A = - A \\ {A}_{A} < 0 ; {A}_{A} > 0 ; \vert {A}_{A} \vert > \vert {A}_{A} \vert : A = - A \\ {A}_{A} < 0 ; {A}_{A} < 0 : A = \frac{\vert {A}_{A} \vert - \vert {A}_{A} \vert}{{A}_{AAAA}} \\ AahAA : A = A\end{matrix} \qquad ( 7 )$$

scCODA (v0.1.8) is a Bayesian probabilistic model for detecting ‘statistically credible differences’ in the proportional representation of cell clusters, identified from snRNA-seq datasets, between different treatment conditions. This method accounts for two main challenges when analyzing snRNA-seq data: (i) low sample number and (ii) the compositionality of the dataset (an increase in the proportional representation of a specific cell cluster will inevitably lead to decreases in the proportional representation of all other cell clusters. Therefore, applying univariate statistical tests, such as a t-test, without accounting for this inherent negative correlation bias will result in reported false positives). scCODA uses a Bayesian generalized linear multivariate regression model to describe the ‘effect’ of treatment groups on the proportional representation of each cell cluster; Hamiltonian Monte Carlo sampling is employed to calculate the posterior inclusion probability of including the effect of treatment in the model. The type I error (false discovery) is derived from the posterior inclusion probability for each effect. The set of “statistically credible effects” is the largest set of effects that can be chosen without exceeding a user-defined false discovery threshold α (α=0.05 by default). Application of scCODA was done using default parameters, including choice of prior probability in the Bayesian model and the setting for Hamiltonian Monte Carlo sampling. The enteroendocrine cell cluster was used as the reference cluster.

### Mass Spectrometry

### UHPLC-QqQ-MS of Cecal Glycosidic Linkages and GC-MS of Short-Chain Fatty Acids

Ultra-high performance liquid chromatography-triple quadrupole mass spectrometric (UHPLC-QqQ-MS) quantification of glycosidic linkages and monosaccharides present in cecal glycans was performed. Levels of short-chain fatty acid levels in cecal contents were measured by GC-MS.

### Lc-MS of Acylcarnitines, Amino Acids, and Biogenic Amines in Host Tissues

Acylcarnitines were measured in jejunum, colon, liver, gastrocnemius, quadriceps, and heart muscle, and plasma, while 20 amino acids plus 19 biogenic amines were quantified in jejunum, liver, and muscle. Plasma levels of non-esterified fatty acids were measured using a UniCel DxC600 clinical analyzer (Beckman Coulter).

### Targeted Mass Spectrometry of Cecal Amino Acids and B-Vitamins

Methods for targeted LC-QqQ-MS of amino acids and B vitamins were adapted from a previous established methods and as described herein. Cecal samples were extracted with ice-cold methanol, and a 200 μL aliquot was dried (vacuum centrifugation; LabConco CentriVap) and reconstituted with 200 μL of a solution containing 80% methanol in water. A 2 μL aliquot of extracted metabolites was then injected into an Agilent 1290 Infinity II UHPLC system coupled with an Agilent 6470 QqQ-MS operated in positive ion dynamic multiple reaction monitor mode (dMRM). The native metabolites were separated on HILIC column (ACQUITY BEH Amide, 2.1×150 mm, 1.7 μm particle size, Waters) using a 20 minute binary gradient with constant flow rate of 0.4 mL/minute. The mobile phases were composed of 10 mM ammonium formate buffer in water with 0.125% formic acid (Phase A) and 10 mM ammonium formate in 95% acetonitrile/H₂O (v/v) with 0.125% formic acid (Phase B). The binary gradient was listed as follows: 0-8 minutes: 91-90% B; 8-14 minutes: 90-70% B; 15-15.1 minutes: 70-91% B; 15.1-20 minutes: 91% B. A pool of 20 amino acids and 7 B vitamins standards with known concentrations (amino acid pool: 0.1 ng/ml-100 ug/mL; B vitamin pool: 0.01 ng/ml-10 μg/mL) was injected along with the samples as an external calibration curve for absolute quantification.

### Example 8: A Manipulatable Model of Maternal-Pup Transmission of Cultured WLZ-Associated Taxa

Selection of bacterial strains—To test the role of P. copri in the context of a defined human gut microbial community that captured features of the developing communities of children who had been enrolled in the clinical study of MDCF-2, 20 bacterial isolates were selected, 16 of which were cultured from the fecal microbiota of 6- to 24-month-old Bangladeshi children living in Mirpur (Table 10). They included strains initially identified by the close correspondence of their 16S rRNA gene sequences to (i) a group of taxa that describe a normal program of development of the microbiota in healthy Bangladeshi children and (ii) taxa whose abundances had statistically significant associations (positive or negative) with the rate of weight gain (b-WLZ) in clinical study participants, and statistically significant correlations with plasma levels of WLZ-associated proteins. The relatedness of these strains to the 1,000 MAGs assembled from fecal samples obtained from all participants in the clinical study was determined by average nucleotide sequence identity (ANI) scores, alignment coverage parameters and their encoded metabolic pathways. A cultured, bacterial strain was deemed as representing a specific MAG if the whole genome alignment coverage was >10%, ANI was >94%, and the binary phenotype concordance score was >90% (see Methods). Based on these criteria, four of the 20 strains were classified as corresponding to MAGs positively associated with WLZ, including P. copri, and eight strains as corresponding to MAGs negatively associated with WLZ.

Liquid chromatography-mass spectrometry (LC-MS) analysis of glycosidic linkages and polysaccharides in MDCF-2 and RUSF disclosed that cellulose, galactan, arabinan, xylan, and mannan represent the principal non-starch polysaccharides in MDCF-2. Gene set enrichment analysis (GSEA) of fecal microbial RNA-Seq datasets generated from children in the MDCF-2 and RUSF arms of the clinical trial disclosed that MDCF-2 produced a meta-transcriptome that was enriched for components of metabolic pathways involved in the utilization of arabinose, a-arabinooligosaccharides (aAOS), and fucose. One-third of the ‘leading-edge’ transcripts associated with these pathways (i.e., transcripts most discriminatory for the pathway response) were derived from the two P. copri MAGs whose abundances were positively correlated with WLZ (MAGs Bg0018 and Bg0019); These leading-edge transcripts include 11 of the 14 related to aAOS utilization. Moreover, a comparison of the fecal meta-transcriptomes of children in the MDCF-2 arm of the clinical study who were classified as being in the upper versus lower quartiles of WLZ responses to treatment revealed that those in the upper quartile exhibited significant enrichment in the expression of metabolic pathways for utilization of xylooligosaccharides, fructooligosaccharides, oligogalacturonate, galactooligosaccharides, galactose, glucuronate, galacturonate and a-arabinooligosaccharides. A majority of the leading-edge transcripts in these pathways were also derived from the two P. copri MAGs. Another feature that distinguished these two MAGs from the other nine P. copri MAGs present in the microbiomes of study participants is that they share 10 functionally conserved PULs, including seven that are completely conserved and three that are partially conserved, albeit structurally distinct (see Methods for the criteria used to classify the degree of PUL conservation). These 10 PULs encode a diverse set of glycoside hydrolases (Table 11) including a multifunctional glycoside hydrolase with broad substrate specificity for glycans present in MDCF-2 (range of substrates: β-glucan, β-mannan, xylan, arabinoxylan, glucomannan, and xyloglucan). Notably, the degree of representation of the seven completely conserved PULs among the 11 P. copri MAGs identified in study participants was highly predictive of each MAG's association with WLZ, suggesting a link between metabolism of carbohydrates by P. copri and growth responses among the malnourished children.

The Bangladeshi P. copri strain PS131.S11 was the only P. copri strain in the 20-member collection. There were several reasons why PS131.S11 was chosen over four other cultured P. copri strains obtained from Bangladeshi children. First, based on phylogenetic distance, P. copri PS131.S11 was most similar to MAGs Bg0018 and Bg0019 (FIGS. 9A-C). Second, it has an overall binary phenotype concordance score of 97% and 96% when compared to Bg0018 and Bg0019, respectively. Among 55 carbohydrate utilization pathways analyzed, 53 are shared across PS131.S11, Bg0018 and Bg0019. Importantly, a total of 93% and 95% of the reconstructed carbohydrate utilization pathways induced in Bg0018 and Bg0019 by MDCF-2 are represented in PS131.S11. Third, P. copri PS131.S11 contains 32 PULs including six of the 11 highly and partially conserved PULs shared by Bg0018 and Bg0019. These six PULs in P. copri PS131.S11 were predicted to be involved in utilizing arabinoxylan (PUL15), 13-glucan (PUL8 and PUL30), pectin (PUL3), pectic galactan (PUL14), starch (PUL27a), and xylan (PUL8) (Table 11). Although the strict criteria for conservation with the Bg0018/Bg0019 PULs was not met, an additional arabinogalactan-targeted PUL (PUL27b) immediately adjacent to the conserved PUL27a was also identified.

P. stercorea was the only other Prevotella species present in the 20-member collection. Although none of the WLZ positively (or negatively) associated MAGs identified in the clinical study belonged to P. stercorea, this isolate was included in the collection to assess the specificity of the responses of P. copri to MDCF-2. The P. stercorea isolate did not possess any of the PULs presents in P. copri PS131.S11 or Bg0018/Bg0019, even after relaxing the criteria for sequence conservation to account for the taxonomic divergence between the two species. The cultured P. stercorea strain has 10 PULs, only five of which encode known carbohydrate utilization enzymes. The glycoside hydrolases in these five PULs were predicted to have very different carbohydrate specificities from those found in the P. copri strain and two P. copri MAGs (the P. stercorea PULs mainly target non-plant glycans) (Table 11).

B. infantis is a prominent early colonizer of the gut. Therefore, it was ensured that it was well represented at the earliest stages of assembly of the defined community so that later colonizers such as P. copri could establish. The collection of cultured isolates also included two strains of Bifidobacterium longum subsp. infantis (B. infantis) recovered from Bangladeshi children—B. infantis Bg463 and B. infantis Bg2D9. The Bg463 strain had been used in earlier preclinical studies that led to development of MDCF-2.

### Example 9: Initial Colonization and Phenotyping

Design—The 20-strain collection was used to perform a 3-arm, fixed diet study that involved ‘successive’ waves of maternal colonization with four different bacterial consortia (FIGS. 10A-C). The sequence of introduction of taxa into dams was designed to emulate temporal features of the normal postnatal development of the human gut community, e.g., consortia 1 and 2 were comprised of strains that are prominent colonizers of healthy infants/children in the first postnatal year while those in consortium 3 are prominent in the second postnatal year. This dam-to-pup colonization strategy also helped overcome the technical challenge of reliable delivery of bacterial consortia to newborn pups via oral gavage.

Dually-housed germ-free dams were switched from a standard breeder chow to a ‘weaning-diet’ supplemented with MDCF-2 on postpartum day 2, two days before initiation of the colonization sequence. This diet was formulated to emulate the diets consumed by children in the clinical trial during MDCF-2 treatment (See Methods; FIG. 10A; Tables 13, 16, 17). It contained (i) powdered human infant formula, (ii) complementary foods consumed by 18-month-old children living in Mirpur, Bangladesh where the study took place, and (iii) MDCF-2. The contributions of the milk, complementary food and MDCF-2 ‘modules’ to total caloric content (53%, 17%, and 30%, respectively) were based on published studies of the diets of cohorts of healthy and undernourished 12- to 23-month-old children from several low- and middle-income countries, including Bangladesh, as well as the amount of MDCF-2 given to the 12-18-month-old children with MAM in the clinical study.

TABLE 16
Ingredients in each diet module

| | | Locally available | |
|---|---|---|---|
| | | complementary | Therapeutic |
| | Breast | foods module | food module |
| | milk mimic | (Mirpur-18 | (MDCF-2 |
| Ingredients (g) | module | module) | module) |
| Cooked potato | | 83.4 | |
| Cooked red lentils | | 197.3 | |
| Cooked rice | | 507.3 | |
| Cooked spinach | | 76.7 | |
| Cooked sweet pumpkin | | 73.3 | |
| Cooked onion | | 45.1 | |
| Iodized Salt | | 5.6 | |
| Turmeric | | 5.6 | |
| Garlic | | 5.6 | |
| Sugar | | | 308.6 |
| Soybean oil | | | 206.4 |
| Chickpea flour | | | 103.2 |
| Peanut flour | | | 103.2 |
| Soybean flour | | | 82.6 |
| Raw banana | | | 196 |
| Similac Sensitive Infant | 1000 | | |
| Powder Formula | | | |
| Total (g) | 1000 | 1000 | 1000 |

TABLE 17
Representation of modules in the weaning
diet supplemented with MDCF-2

| | Weaning diet supplemented |
|---|---|
| Module (g) | with MDCF-2 |
| Breast milk mimic | 295 |
| Locally available complementary foods | 500 |
| Therapeutic food | 205 |
| Total (g) | 1000 |

In Arm 1, dams received the following series of oral gavages: (i) on postpartum day 4, a consortium of five ‘early’ infant gut community colonizers; (ii) on postpartum day 7, P. copri and P. stercorea; (iii) on postpartum days 10 and 12 additional age-discriminatory and WLZ-associated taxa, and (iv) on postpartum day 21, P. copri, P. stercorea, and Faecalibacterium prausnitzii (FIG. 10C). At this last time point, the three strains were given by oral gavage to both the dams and their offspring to help promote successful colonization. In Arm 2, pups were subjected to the same sequence of microbial exposures and the same diet manipulations as in Arm 1, except that B. infantis Bg463 rather than B. infantis Bg2D9 was included in the first gavage mixture. Arm 3 was a replicate of Arm 2 but without the Prevotella gavages. Pups in all three arms were subjected to a diet sequence that began with exclusive milk feeding (from the nursing dam) followed by a weaning period where pups had access to the weaning phase diet supplemented with MDCF-2. Pups were weaned at P24, after which time they received MDCF-2 alone ad libitum until P53 when they were euthanized. The rationale for the timing of the first three gavages was based on the diet sequence [gavage 1 of early colonizers at a time (P4) when mice were exclusively consuming the dam's milk, gavage 2 as the pups were just beginning to consume the human weaning (complementary food) diet, gavage 3 somewhat later during this period of complementary feeding and the fourth gavage to help to ensure a consistent level of P. copri colonization at the end of weaning (and subsequently through the post-weaning period)]. The relative abundances of these strains in fecal samples collected from dams on days postpartum days 21, 24, and 35, as well as the absolute abundances of these strains in fecal samples collected from their offspring on P21, P24, P35, and P53 were quantified by shotgun sequencing of community DNA (n=2 dams and 5-8 pups analyzed/arm).

A relationship between B. infantis and P. copri colonization-B. infantis Bg2D9 successfully colonized pups at P21 in Arm 1 [8.4±0.5 log 10 (genome equivalents/g feces) (mean±SD); relative abundance, 9.0±3.9% (mean±SD)]. In contrast, the abundance of B. infantis Bg463 was 5-8 orders-of-magnitude lower in Arms 2 and 3 [3.22±1.9 and 0.6±1.5 log 10 (genome equivalents/g feces) (mean±SD), respectively]. These differences were sustained through P53 (FIG. 10D). The results also revealed that exposure to B. infantis Bg2D9 in Arm 1 was associated with an absolute abundance of P. copri in the pre-weaning period (P21) that was 3 orders-of-magnitude greater than in Arm 2 mice exposed to B. infantis Bg463; P<0.005, Mann-Whitney U test] (FIG. 10E). Administering the fourth gavage on P21 elevated the absolute abundance of fecal P. copri in Arm 2 to a level comparable to Arm 1; this level was sustained throughout the post-weaning period (P24 to P53) [FIG. 10E; P>0.05; mixed linear effects model (Methods). This effect of the fourth gavage was also evident in the ileal and cecal microbiota.

The effects of B. infantis on P. copri did not generalize to P. stercorea. Unlike P. copri, the absolute abundance of P. stercorea in feces sampled on P21 and P24 was not significantly different in mice belonging to Arms 1 and 2 (P>0.05, Mann-Whitney U test). Prior to weaning at P24, the absolute abundance of P. stercorea was 5-orders of magnitude lower than that of P. copri. Throughout the post-weaning period, the absolute abundance of P. stercorea remained similar in members of both treatment arms (P>0.05, Mann-Whitney U test) but 2-orders of magnitude below that of P. copri.

Based on these results, the colonization dependency of P. copri on B. infantis was directly tested in two independent experiments whose designs are outlined in FIG. 9A. Dually-housed germ-free dams were switched from standard breeder chow to the weaning Bangladeshi diet supplemented with MDCF-2 on postpartum day 2. On postpartum day 4, one group of dams was colonized with B. infantis Bg2D9. On postpartum days 7 and 10, both groups of gnotobiotic mice were gavaged with a consortium containing five P. copri strains. These five P. copri strains (1A8, 2C6, 2D7, G8, and PS131.S11) were all isolated from fecal samples obtained from Bangladeshi children (Table 12). Pups were separated from their dams at the completion of weaning and their diet was transitioned to MDCF-2. The results disclosed that the total absolute abundance of P. copri in feces collected on P42 from mice that had received B. infantis Bg2D9 was three orders of magnitude higher than in animals never exposed to B. infantis (FIG. 9B; Tables 14 and 15)—a finding that confirmed what was observed between Arms 1 and 2 of the initial colonization experiment (see FIG. 10E). There was no statistically significant difference in weight gain from P23 to P42 between the mono- and bi-colonization groups. However, interpretation of this result was confounded by the fact that compared to the bi-colonized animals with significantly higher levels of P. copri, mono-colonized mice with low levels of P. copri had massive, fluid-filled cecums, similar to those commonly seen in germ-free mice. This pronounced cecal enlargement adds substantially to body weight and in a comparison of the two treatment groups obscures the ability to discern whether increased levels of P. copri has ponderal growth-promoting effects.

Effects on weight gain and metabolism of MDCF-2 glycans—Gnotobiotic mice in Arm 1 exhibited a significantly greater increase in weight gain between P23 (the first time point measured, 2 days after the final gavage) and P53 compared to mice in the two other experimental arms [P<0.05 compared to Arm 2; P<0.01 compared to Arm 3; linear mixed-effects model (see Methods)] (FIG. 10F). Unlike the mono- and bi-colonization experiments described above, cecal sizes were comparable across the three treatment groups. Based on these results, we advanced samples collected from mice in Arms 1 and 3 for additional analyses of the metabolism of MDCF-2 glycans.

Integrating results from mass spectrometric and microbial RNA-Seq data generated from cecal contents harvested from mice at the time of euthanasia (P53) provided several lines of evidence for the important role played by P. copri in metabolizing the principal polysaccharide components of MDCF-2. First, unlike P. stercorea, P. copri PS131.S11 contains and expresses PULs involved in processing MDCF-2 glycans: i.e., PUL27a and PUL27b specify and express CAZymes known or predicted to digest starch and arabinogalactan, while PUL2 possesses and expresses a fucosidase that could target the terminal residues found in arabinogalactan II (Table 11). Second, UHPLC-QqQ-MS-based measurements of 49 glycosidic linkages in cecal contents disclosed that animals in Arm 1 harboring P. copri had (i) significantly lower levels of t-p-Ara, t-f-Ara, 2-f-Ara, 2,3-f-Ara, and 3,4-p-Xyl/3,5-f-Ara (P<0.05; Mann-Whitney U Test; FIG. 11A) and (ii) significantly lower amounts of arabinose in cecal glycans (P<0.05; Mann-Whitney U test; FIG. 11B). Third, GC-MS-based measurements of cecal short-chain fatty acids showed significantly higher levels of acetate, indicating increased fermentation by the P. copri-containing microbial community (P<0.01; Mann-Whitney U test) (FIG. 11C; Table 18). Together, these results indicate that mice with the P. copri-containing community exhibit a greater degree of liberation of arabinose from MDCF-2 glycans.

TABLE 9
Targeted mass spectrometric analysis of short-chain fatty acids in the
cecal contents of gnotobiotic mice colonized with defined consortia

| Treatment | Mouse no. | Acetate¹ | Propionate¹ | Butyrate¹ | Lactate¹ | Succinate¹ |
|---|---|---|---|---|---|---|
| w/P. copri | 1 | 55.19 | 0.14 | 0.08 | 0.06 | 36.38 |
| (Arm 1) | 2 | 47.76 | 0.22 | 0.06 | 0.04 | 29.63 |
| | 3 | 51.66 | 0.13 | 0.04 | 0.06 | 24.81 |
| | 4 | 52.32 | 0.18 | 0.01 | 0.05 | 24.10 |
| | 5 | 57.25 | 0.16 | 0.04 | 0.07 | 29.20 |
| | 6 | 46.91 | 0.15 | 0.06 | 0.14 | 26.01 |
| | 7 | 40.40 | 0.09 | 0.05 | 0.07 | 36.60 |
| | 8 | 40.35 | 0.13 | 0.05 | 0.18 | 19.42 |
| | mean ± SD | 48.98 ± 6.32 | 0.15 ± 0.04 | 0.05 ± 0.02 | 0.08 ± 0.05 | 28.27 ± 5.98 |
| w/o P. copri | 1 | 36.67 | 0.09 | 0.06 | 0.63 | 4.27 |
| (Arm 3) | 2 | 26.40 | 0.11 | 0.03 | 0.29 | 2.28 |
| | 3 | 31.78 | 0.09 | 0.05 | 0.40 | 1.70 |
| | 4 | 28.46 | 0.09 | 0.05 | 0.17 | 1.71 |
| | 5 | 43.90 | 0.12 | 0.07 | 0.58 | 3.22 |
| | 6 | 35.10 | 0.10 | 0.05 | 0.44 | 1.24 |
| | 7 | 34.82 | 0.17 | 0.06 | 0.36 | 1.45 |
| | mean ± SD | 33.88 ± 5.78 | 0.11 ± 0.03 | 0.05 ± 0.01 | 0.41 ± 0.16 | 2.27 ± 1.10 |

¹Unit: μmol per g of cecal contents

Increased levels of enzyme-resistant arabinose linkages, such as 5-f-Ara, 2-f-Ara, and 2,3-f-Ara, has been previously reported in the feces of MDCF-2 treated children in the upper-compared to lower quartile of WLZ response. The lower levels of these resistant arabinose-containing linkages documented in gnotobiotic mice harboring P. copri versus those lacking the organism indicate more complete degradation of branched arabinans in their cecums-a portion of the gastrointestinal tract that is specialized for microbial fermentation. Because (i) P. copri was the only Prevotella sp. in the defined community that encodes and expresses CAZymes capable of degrading linkages in MDCF-2 glycans (Table 11), (ii) P. copri has higher absolute abundance than P. stercorea, and (iii) previous analyses linked the abundance of P. copri but not P. stercorea MAGs to host growth, Arm 1 of this experiment is referred as ‘w/ P. copri’ and Arm 3 as ‘w/o P. copri’, as described herein.

Effects on expressed metabolic functions in other community members—To investigate the transcripts driving the observed differences in microbial glycan processing in the ‘w/ P. copri’ versus ‘w/o P. copri’ arms, we performed microbial RNA-Seq on cecal contents collected at the time of euthanasia (Tables 19 and 20). Transcript abundance tables were filtered and counts were aggregated based on mcSEED reconstructions of metabolic pathways to give an average expression value across the genes in a given metabolic pathway in a given organism (see Methods). Principal component analysis (PCA) was performed on these aggregated tables and compared the contribution of each expressed metabolic pathway to each principal component and the clustering of samples from each experimental Arm in a space determined by PC1 and PC2 (FIG. 11D). The results revealed significant separation of meta-transcriptomes aggregated by metabolic pathway between cecal samples from the with and without P. copri Arms (P<0.001; PERMANOVA) (FIG. 11E).

TABLE 19
Sample metadataP

| | | | Number | |
|---|---|---|---|---|
| Treat- | Mouse | | of raw | |
| ment | no. | Sample ID | reads | Index |
| w/ P. | 1 | defined_community.preweaning_P_copri_ | 71,226,054 | TATGATGG |
| copri | | colonization.postnatal_day_53.pup.m1. | | CCGATTGT |
| (Arm 1) | | cecal_contents.microbial_RNAseq | | CATA |
| | 2 | defined_community.preweaning_P_copri_ | 90,313,136 | CGCAGCAA |
| | | colonization.postnatal_day_53.pup.m2. | | TTATTCCG |
| | | cecal_contents.microbial_RNAseq | | CTAT |
| | 3 | defined_community.preweaning_P_copri_ | 75,913,993 | ACGTTCCT |
| | | colonization.postnatal_day_53.pup.m3. | | TAGACCGC |
| | | cecal_contents.microbial_RNAseq | | TGTG |
| | 4 | defined_community.preweaning_P_copri_ | 78,791,984 | CCGCGTAT |
| | | colonization.postnatal_day_53.pup.m4. | | AGTAGGAA |
| | | cecal_contents.microbial_RNAseq | | CCGG |
| | 5 | defined_community.preweaning_P_copri_ | 86,856,561 | GATTCTGA |
| | | colonization.postnatal_day_53.pup.m5. | | ATAGCGGT |
| | | cecal_contents.microbial_RNAseq | | GGAC |
| | 6 | defined_community.preweaning_P_copri_ | 77,222,488 | TAGAGAAT |
| | | colonization.postnatal_day_53.pup.m6. | | ACTATAGA |
| | | cecal_contents.microbial_RNAseq | | TTCG |
| | 7 | defined_community.preweaning_P_copri_ | 73,231,552 | TTGTATCA |
| | | colonization.postnatal_day_53.pup.m7. | | GGACAGAG |
| | | cecal_contents.microbial_RNAseq | | GCCA |
| | 8 | defined_community.preweaning_P_copri_ | 81,265,744 | CACAGCGG |
| | | colonization.postnatal_day_53.pup.m8. | | TCATTCCT |
| | | cecal_contents.microbial_RNAseq | | ATTG |
| w/o P. | 1 | defined_community.no_P_copri_coloni- | 86,127,619 | GTGACGGA |
| copri | | zation.postnatal_day_53.pup.m1.cecal_ | | GCTGGCGG |
| (Arm 3) | | contents.microbial_RNAseq | | TCCA |
| | 2 | defined_community.no_P_copri_coloni- | 63,004,611 | AATTCCAT |
| | | zation.postnatal_day_53.pup.m2.cecal_ | | CTCTTCAG |
| | | contents.microbial_RNAseq | | TTAC |
| | 3 | defined_community.no_P_copri_coloni- | 77,209,161 | TTAACGGT |
| | | zation.postnatal_day_53.pup.m3.cecal_ | | GTTCCTGA |
| | | contents.microbial_RNAseq | | CCGT |
| | 4 | defined_community.no_P_copri_coloni- | 69,667,767 | ACTTGTTA |
| | | zation.postnatal_day_53.pup.m4.cecal_ | | TCCGCGCC |
| | | contents.microbial_RNAseq | | TAGA |
| | 5 | defined_community.no_P_copri_coloni- | 72,353,465 | CGTGTACC |
| | | zation.postnatal_day_53.pup.m5.cecal_ | | AGAGGATA |
| | | contents.microbial_RNAseq | | AGTT |
| | 6 | defined_community.no_P_copri_coloni- | 70,980,578 | TTAACCTT |
| | | zation.postnatal_day_53.pup.m6.cecal_ | | CGAGGCCA |
| | | contents.microbial_RNAseq | | GACA |
| | 7 | defined_community.no_P_copri_coloni- | 108,058,317 | CATATGCG |
| | | zation.postnatal_day_53.pup.m7.cecal_ | | ATCCTTGA |
| | | contents.microbial_RNAseq | | ACGG |

TABLE 20
Level of gene expression in P. copri PS131.S11 PULs (TPM normalized)

| | | GH Family/ | w/P. copri (Arm 1) | | | | | | | | |
|---|---|---|---|---|---|---|---|---|---|---|---|
| Predicted PUL | Gene locus | Gene | mouse | mouse | mouse | mouse | mouse | mouse | mouse | mouse | mean ± |
| target(s) | tag | annotation* | 1 | 2 | 3 | 4 | 5 | 6 | 7 | 8 | SD |
| O- | NJCFFJJN_00266 | SusD | 63.8 | 73.5 | 99.6 | 35.1 | 39.6 | 13.4 | 121.8 | 56.5 | 62.9 ± 35.3 |
| glycans/mucins | | | | | | | | | | | |
| | NJCFFJJN_00267 | SusC | 73.5 | 76.7 | 118.2 | 38.0 | 42.6 | 12.6 | 130.4 | 63.7 | 69.4 ± 39.9 |
| | NJCFFJJN_00268 | GH16 | 45.9 | 55.4 | 90.8 | 33.7 | 31.3 | 11.6 | 85.7 | 61.0 | 51.9 ± 27.2 |
| | NJCFFJJN_00269 | ROK | 175.9 | 166.1 | 249.6 | 88.1 | 95.3 | 32.7 | 263.4 | 164.4 | 154.5 ± 79.6 |
| | NJCFFJJN_00270 | ROK | 19.0 | 23.8 | 24.1 | 12.9 | 12.2 | 5.0 | 36.5 | 20.8 | 19.3 ± 9.5 |
| | NJCFFJJN_00271 | Est | 21.1 | 24.4 | 29.7 | 16.4 | 14.4 | 7.0 | 49.7 | 23.5 | 23.3 ± 12.8 |
| | NJCFFJJN_00272 | GH20 | 19.2 | 24.6 | 27.5 | 13.0 | 11.9 | 4.9 | 46.8 | 20.4 | 21.0 ± 12.7 |
| | NJCFFJJN_00273 | GH2 [Fc] | 15.4 | 26.6 | 30.3 | 12.7 | 10.8 | 4.4 | 48.0 | 21.9 | 21.3 ± 13.8 |
| | NJCFFJJN_00274 | [unk] | 30.7 | 37.4 | 38.7 | 18.6 | 20.5 | 8.7 | 78.1 | 34.4 | 33.4 ± 20.9 |
| a-L-fucoside + | NJCFFJJN_00375 | [unk] | 75.8 | 54.1 | 87.5 | 26.3 | 28.8 | 9.8 | 85.7 | 34.1 | 50.3 ± 29.9 |
| b-galactoside | | | | | | | | | | | |
| | NJCFFJJN_00376 | GH2 | 84.9 | 63.8 | 91.1 | 32.5 | 34.4 | 10.9 | 111.6 | 39.2 | 58.6 ± 34.8 |
| | NJCFFJJN_00377 | GH29 | 125.5 | 119.0 | 143.6 | 62.7 | 57.6 | 21.4 | 255.2 | 80.4 | 108.2 ± 71.9 |
| | NJCFFJJN_00378 | SusD | 29.6 | 34.7 | 42.2 | 21.0 | 13.4 | 7.2 | 39.7 | 34.4 | 27.8 ± 12.7 |
| | NJCFFJJN_00379 | SusC | 32.1 | 32.7 | 45.3 | 21.3 | 15.1 | 8.2 | 41.4 | 36.1 | 29.0 ± 13.0 |
| pectin | NJCFFJJN_00575 | GH127 | 29.0 | 20.0 | 26.4 | 7.8 | 9.3 | 3.5 | 17.5 | 15.5 | 16.1 ± 9.0 |
| | NJCFFJJN_00576 | GH43_34- | 13.7 | 9.2 | 15.9 | 3.4 | 4.6 | 1.7 | 8.6 | 8.7 | 8.2 ± 4.9 |
| | | CBM32 | | | | | | | | | |
| | NJCFFJJN_00577 | GH97 | 6.3 | 5.2 | 8.7 | 1.9 | 2.4 | 1.0 | 4.7 | 4.4 | 4.3 ± 2.5 |
| | NJCFFJJN_00578 | GH146 | 12.7 | 10.6 | 19.4 | 2.3 | 6.2 | 2.0 | 8.3 | 8.6 | 8.8 ± 5.7 |
| | NJCFFJJN_00579 | SusC | 36.8 | 21.6 | 36.4 | 6.4 | 11.9 | 3.2 | 21.6 | 15.9 | 19.2 ± 12.5 |
| | NJCFFJJN_00580 | SusD | 43.2 | 23.0 | 35.2 | 7.2 | 12.4 | 3.5 | 23.1 | 16.6 | 20.5 ± 13.6 |
| | NJCFFJJN_00581 | [unk] | 30.3 | 20.1 | 29.1 | 6.9 | 11.2 | 2.9 | 20.5 | 17.0 | 17.2 ± 9.9 |
| no CAZyme | NJCFFJJN_01056 | [unk] | 12.7 | 15.7 | 17.5 | 4.5 | 4.7 | 1.5 | 7.8 | 8.2 | 9.1 ± 5.7 |
| | NJCFFJJN_01056 | [unk] | 12.7 | 15.7 | 17.5 | 4.5 | 4.7 | 1.5 | 7.8 | 8.2 | 9.1 ± 5.7 |
| | NJCFFJJN_01057 | [unk] | 14.6 | 16.1 | 20.8 | 5.4 | 4.2 | 1.9 | 7.1 | 10.3 | 10.0 ± 6.6 |
| | NJCFFJJN_01058 | [unk] | 12.0 | 15.0 | 20.2 | 5.2 | 4.5 | 1.6 | 6.5 | 8.8 | 9.2 ± 6.2 |
| | NJCFFJJN_01059 | SusD | 16.4 | 18.5 | 23.0 | 5.6 | 5.6 | 2.3 | 6.8 | 11.8 | 11.2 ± 7.4 |
| | NJCFFJJN_01060 | SusC | 25.5 | 25.3 | 33.4 | 8.6 | 8.4 | 3.2 | 11.6 | 14.6 | 16.3 ± 10.5 |
| b-mannan | NJCFFJJN_01811 | GH130 | 34.7 | 27.8 | 37.5 | 17.3 | 17.8 | 13.7 | 29.7 | 17.3 | 24.5 ± 9.1 |
| | NJCFFJJN_01812 | GH26 | 37.0 | 29.2 | 51.0 | 22.3 | 20.7 | 14.1 | 32.1 | 18.2 | 28.1 ± 12.0 |
| | NJCFFJJN_01813 | CE7 | 39.6 | 31.8 | 49.0 | 21.1 | 21.5 | 15.5 | 27.8 | 19.4 | 28.2 ± 11.4 |
| | NJCFFJJN_01814 | GH26 | 36.2 | 27.8 | 45.8 | 18.2 | 20.0 | 15.2 | 37.9 | 19.4 | 27.6 ± 11.2 |
| | NJCFFJJN_01815 | [unk] | 27.2 | 21.3 | 31.1 | 14.1 | 13.5 | 9.5 | 24.7 | 12.4 | 19.2 ± 7.9 |
| | NJCFFJJN_01816 | SusD | 31.0 | 22.1 | 34.3 | 14.7 | 13.5 | 11.3 | 27.6 | 13.0 | 20.9 ± 9.1 |
| | NJCFFJJN_01817 | SusC | 31.5 | 22.0 | 36.1 | 13.4 | 13.1 | 11.0 | 24.2 | 12.8 | 20.5 ± 9.5 |
| homogalac- | NJCFFJJN_01898 | ECF-s | 67.6 | 61.2 | 92.1 | 30.0 | 27.4 | 12.9 | 60.4 | 40.7 | 49.0 ± 25.9 |
| turonan | | | | | | | | | | | |
| | NJCFFJJN_01899 | GH28-GH105 | 89.9 | 68.2 | 103.0 | 36.5 | 31.8 | 17.4 | 35.6 | 54.2 | 54.6 ± 30.1 |
| | NJCFFJJN_01900 | [unk] | 2.5 | 1.6 | 3.2 | 0.9 | 0.8 | 0.3 | 1.3 | 1.6 | 1.5 ± 0.9 |
| | NJCFFJJN_01901 | [unk] | 27.1 | 33.8 | 47.3 | 39.5 | 18.7 | 13.6 | 35.1 | 51.0 | 33.3 ± 13.1 |
| | NJCFFJJN_01902 | CE8 | 27.2 | 11.5 | 41.7 | 7.1 | 12.6 | 4.9 | 5.5 | 10.2 | 15.1 ± 12.9 |
| | NJCFFJJN_01903 | SusC | 192.9 | 81.6 | 165.5 | 39.7 | 56.7 | 21.1 | 34.7 | 46.0 | 79.8 ± 64.2 |
| | NJCFFJJN_01904 | SusD | 238.4 | 90.9 | 180.8 | 47.4 | 68.1 | 23.6 | 40.7 | 51.1 | 92.6 ± 76.4 |
| | NJCFFJJN_01905 | [unk] | 191.9 | 84.1 | 179.3 | 40.1 | 60.7 | 22.1 | 44.8 | 52.5 | 84.4 ± 65.0 |
| pectin | NJCFFJJN_01907 | SusC | 194.7 | 120.1 | 179.6 | 61.3 | 58.4 | 24.0 | 61.0 | 73.2 | 96.5 ± 62.0 |
| | NJCFFJJN_01908 | SusD | 236.7 | 171.3 | 218.4 | 80.4 | 78.7 | 38.8 | 90.3 | 106.5 | 127.6 ± 72.1 |
| | NJCFFJJN_01909 | GH28 | 50.3 | 26.9 | 84.8 | 18.1 | 20.6 | 9.8 | 17.4 | 28.8 | 32.1 ± 24.5 |
| | NJCFFJJN_01910 | GH43_10 | 101.0 | 59.7 | 147.1 | 34.3 | 38.2 | 20.3 | 36.3 | 54.7 | 61.5 ± 42.4 |
| | NJCFFJJN_01911 | GH57 | 70.8 | 50.1 | 86.9 | 27.1 | 27.8 | 14.2 | 54.5 | 32.9 | 45.5 ± 24.6 |
| | NJCFFJJN_01912 | GT4 | 73.3 | 49.3 | 89.7 | 26.7 | 29.7 | 13.3 | 54.4 | 31.5 | 46.0 ± 25.8 |
| | NJCFFJJN_01913 | GH133 | 61.0 | 41.1 | 70.1 | 22.0 | 23.4 | 12.1 | 42.7 | 23.5 | 37.0 ± 20.5 |
| b-glucan, xylan | NJCFFJJN_02065 | GH3 | 10.0 | 7.5 | 7.9 | 3.1 | 2.7 | 1.5 | 8.3 | 5.3 | 5.8 ± 3.1 |
| | NJCFFJJN_02066 | [unk] | 5.0 | 3.8 | 5.6 | 2.6 | 1.8 | 0.8 | 4.1 | 3.1 | 3.4 ± 1.6 |
| | NJCFFJJN_02067 | SusD | 5.2 | 3.8 | 5.0 | 2.0 | 1.8 | 0.9 | 4.3 | 3.3 | 3.3 ± 1.6 |
| | NJCFFJJN_02068 | SusC | 5.4 | 3.9 | 5.1 | 2.1 | 1.7 | 0.6 | 4.1 | 3.8 | 3.3 ± 1.7 |
| | NJCFFJJN_02069 | GH5_4 | 3.7 | 2.9 | 3.7 | 2.4 | 1.0 | 0.6 | 2.7 | 3.2 | 2.5 ± 1.2 |
| | NJCFFJJN_02070 | HTCS | 9.6 | 7.7 | 10.9 | 4.2 | 2.9 | 1.3 | 9.8 | 7.8 | 6.8 ± 3.5 |
| b-1,6-glucan | NJCFFJJN_02072 | SusR | 5.4 | 4.0 | 7.6 | 3.8 | 2.3 | 1.5 | 7.0 | 5.9 | 4.7 ± 2.2 |
| | NJCFFJJN_02073 | [unk] | 19.5 | 11.4 | 8.2 | 9.9 | 6.9 | 4.4 | 8.8 | 9.1 | 9.8 ± 4.5 |
| | NJCFFJJN_02074 | GH30_3 | 18.9 | 8.6 | 7.8 | 8.0 | 6.2 | 3.0 | 8.2 | 7.0 | 8.5 ± 4.6 |
| | NJCFFJJN_02075 | SusD | 15.5 | 8.2 | 6.9 | 8.6 | 5.5 | 2.8 | 8.0 | 7.5 | 7.9 ± 3.6 |
| | NJCFFJJN_02076 | SusC | 15.6 | 7.7 | 6.9 | 7.2 | 5.0 | 2.1 | 7.4 | 6.4 | 7.3 ± 3.8 |
| no CAZyme | NJCFFJJN_02231 | [unk] | 39.2 | 10.1 | 33.1 | 1.8 | 7.5 | 1.9 | 4.0 | 1.6 | 12.4 ± 15.0 |
| | NJCFFJJN_02232 | SusD | 34.8 | 8.2 | 24.2 | 1.2 | 5.2 | 1.6 | 3.2 | 1.2 | 10.0 ± 12.6 |
| | NJCFFJJN_02233 | SusC | 38.4 | 10.2 | 24.1 | 2.0 | 6.7 | 2.2 | 4.5 | 1.1 | 11.2 ± 13.3 |
| sucrose, inulin, | NJCFFJJN_02248 | GH32 | 6.7 | 3.8 | 5.1 | 3.8 | 2.6 | 1.0 | 5.6 | 4.4 | 4.1 ± 1.8 |
| levan | | | | | | | | | | | |
| | NJCFFJJN_02249 | SusD | 6.6 | 3.5 | 5.6 | 3.0 | 2.2 | 1.1 | 5.9 | 4.8 | 4.1 ± 2.0 |
| | NJCFFJJN_02250 | SusC | 4.9 | 2.7 | 3.7 | 2.3 | 1.7 | 0.8 | 4.4 | 3.2 | 3.0 ± 1.4 |
| xylan | NJCFFJJN_02284 | [unk] | 3.5 | 5.0 | 5.3 | 4.6 | 0.4 | 1.4 | 2.7 | 7.9 | 3.8 ± 2.4 |
| | NJCFFJJN_02285 | GH3 | 2.6 | 3.8 | 3.0 | 3.1 | 0.2 | 0.8 | 2.1 | 4.3 | 2.5 ± 1.4 |
| | NJCFFJJN_02286 | GH31 [Fs] | 0.7 | 0.9 | 0.7 | 0.2 | 0.1 | 0.0 | 0.4 | 0.3 | 0.4 ± 0.3 |
| | NJCFFJJN_02287 | [unk] | 1.3 | 1.3 | 1.3 | 0.4 | 0.1 | 0.1 | 0.6 | 0.4 | 0.7 ± 0.6 |
| | NJCFFJJN_02288 | GH43_7- | 0.6 | 0.7 | 0.5 | 0.2 | 0.1 | 0.0 | 0.3 | 0.3 | 0.3 ± 0.2 |
| | | CBM13 | | | | | | | | | |
| | NJCFFJJN_02289 | [unk] | 0.7 | 0.3 | 0.5 | 0.0 | 0.1 | 0.1 | 0.2 | 0.3 | 0.3 ± 0.2 |
| | NJCFFJJN_02290 | GH43_2- | 0.7 | 0.8 | 0.5 | 0.1 | 0.1 | 0.0 | 0.3 | 0.3 | 0.3 ± 0.3 |
| | | CBM6-GH8 | | | | | | | | | |
| | NJCFFJJN_02291 | SusD | 3.3 | 4.1 | 2.4 | 1.2 | 0.4 | 0.6 | 1.9 | 2.3 | 2.0 ± 1.3 |
| | NJCFFJJN_02292 | SusC | 3.9 | 3.0 | 1.9 | 1.0 | 0.5 | 0.2 | 1.4 | 1.5 | 1.7 ± 1.3 |
| | NJCFFJJN_02293 | [unk] | 0.0 | 0.0 | 0.0 | 0.0 | 0.0 | 0.0 | 0.0 | 0.0 | 0.0 ± 0.0 |
| | NJCFFJJN_02294 | CE6-CBM48 | 0.8 | 1.0 | 1.3 | 1.2 | 0.3 | 0.5 | 0.7 | 1.0 | 0.8 ± 0.4 |
| arabinoxylan | NJCFFJJN_02307 | GH10_{—} | 7.8 | 13.8 | 23.6 | 6.3 | 2.6 | 3.1 | 9.8 | 9.6 | 9.6 ± 6.7 |
| | | CMB4_CH10 | | | | | | | | | |
| | NJCFFJJN_02308 | [unk] | 6.9 | 8.5 | 17.4 | 4.3 | 2.6 | 1.7 | 6.4 | 5.7 | 6.7 ± 4.9 |
| | NJCFFJJN_02309 | SusD | 8.4 | 10.7 | 19.3 | 5.3 | 2.3 | 1.6 | 7.3 | 6.4 | 7.7 ± 5.6 |
| | NJCFFJJN_02310 | SusC | 7.2 | 9.3 | 17.2 | 4.6 | 1.9 | 1.4 | 6.1 | 5.4 | 6.6 ± 5.0 |
| | NJCFFJJN_02311 | SusD | 10.3 | 11.5 | 21.6 | 6.4 | 2.7 | 1.8 | 7.4 | 7.0 | 8.6 ± 6.2 |
| | NJCFFJJN_02312 | SusC | 9.0 | 13.5 | 19.5 | 6.2 | 2.5 | 1.8 | 7.0 | 7.4 | 8.4 ± 5.8 |
| | NJCFFJJN_02313 | GH5_21 | 2.6 | 2.9 | 13.1 | 2.9 | 1.2 | 1.2 | 1.9 | 3.0 | 3.6 ± 3.9 |
| | NJCFFJJN_02314 | HTCS | 31.6 | 35.9 | 38.9 | 22.6 | 4.0 | 6.6 | 31.1 | 32.6 | 25.4 ± 13.3 |
| | NJCFFJJN_02315 | GH43_12 | 46.4 | 44.2 | 53.0 | 25.8 | 6.5 | 9.3 | 29.8 | 32.6 | 30.9 ± 16.9 |
| pectic galactan | NJCFFJJN_02485 | [unk] | 30.8 | 29.1 | 32.4 | 17.3 | 12.5 | 4.8 | 32.8 | 26.6 | 23.3 ± 10.5 |
| | NJCFFJJN_02486 | [unk] | 73.9 | 62.4 | 69.5 | 23.6 | 28.7 | 10.1 | 81.4 | 48.4 | 49.7 ± 26.3 |
| | NJCFFJJN_02487 | [unk] | 71.8 | 50.3 | 61.2 | 18.9 | 27.1 | 8.1 | 55.0 | 30.7 | 40.4 ± 22.4 |
| | NJCFFJJN_02488 | Pept_SB | 61.1 | 50.1 | 54.3 | 17.2 | 24.4 | 6.5 | 54.9 | 29.2 | 37.2 ± 20.4 |
| | NJCFFJJN_02489 | [unk] | 55.2 | 41.7 | 48.8 | 16.2 | 21.1 | 5.8 | 50.5 | 26.4 | 33.2 ± 18.3 |
| | NJCFFJJN_02490 | [unk] | 51.0 | 40.5 | 50.4 | 16.1 | 22.1 | 5.5 | 48.3 | 25.8 | 32.5 ± 17.5 |
| | NJCFFJJN_02491 | [unk] | 46.1 | 36.5 | 45.1 | 15.3 | 20.5 | 5.4 | 42.8 | 24.2 | 29.5 ± 15.3 |
| | NJCFFJJN_02492 | [unk] | 38.5 | 30.4 | 36.3 | 11.9 | 17.2 | 3.2 | 34.7 | 20.0 | 24.0 ± 12.9 |
| | NJCFFJJN_02493 | SusD | 43.3 | 32.0 | 39.2 | 13.7 | 17.4 | 3.9 | 36.9 | 21.6 | 26.0 ± 13.9 |
| | NJCFFJJN_02494 | SusC | 39.8 | 29.8 | 32.1 | 12.0 | 15.3 | 3.8 | 30.1 | 18.6 | 22.7 ± 12.1 |
| | NJCFFJJN_02495 | HTCS | 19.8 | 20.6 | 22.1 | 9.1 | 8.9 | 3.9 | 17.8 | 14.5 | 14.6 ± 6.6 |
| | NJCFFJJN_02496 | GH2 | 412.1 | 380.4 | 369.6 | 192.4 | 141.8 | 67.2 | 259.1 | 239.9 | 257.8 ± 122.9 |
| | NJCFFJJN_02497 | GH53 | 198.5 | 198.6 | 237.0 | 117.7 | 74.6 | 38.5 | 144.7 | 138.9 | 143.6 ± 66.8 |
| | NJCFFJJN_02498 | PL1 | 13.6 | 7.5 | 17.0 | 5.0 | 5.7 | 3.9 | 1.9 | 6.2 | 7.6 ± 5.1 |
| arabinoxylan | NJCFFJJN_02552 | GH43_1 | 16.5 | 8.9 | 5.7 | 5.7 | 2.8 | 1.3 | 6.4 | 4.7 | 6.5 ± 4.6 |
| | NJCFFJJN_02553 | GH10 | 10.2 | 4.2 | 3.6 | 3.0 | 1.6 | 0.5 | 3.7 | 2.5 | 3.7 ± 2.9 |
| | NJCFFJJN_02554 | MFS | 13.1 | 3.7 | 3.9 | 2.4 | 2.5 | 0.6 | 2.9 | 2.7 | 4.0 ± 3.8 |
| | NJCFFJJN_02555 | Est | 11.4 | 4.4 | 4.3 | 2.8 | 2.2 | 0.7 | 2.9 | 3.1 | 4.0 ± 3.2 |
| | NJCFFJJN_02556 | HTCS | 7.4 | 7.4 | 9.5 | 3.5 | 3.5 | 1.2 | 10.4 | 7.9 | 6.4 ± 3.2 |
| | NJCFFJJN_02557 | GH67 | 23.0 | 15.5 | 17.8 | 8.3 | 6.0 | 2.7 | 15.1 | 12.5 | 12.6 ± 6.6 |
| | NJCFFJJN_02558 | GH35 | 19.9 | 15.1 | 18.4 | 8.8 | 6.2 | 2.4 | 15.2 | 13.2 | 12.4 ± 6.1 |
| | NJCFFJJN_02559 | SusC | 29.4 | 13.5 | 12.0 | 8.0 | 4.5 | 1.1 | 12.8 | 7.2 | 11.1 ± 8.5 |
| | NJCFFJJN_02560 | SusD | 33.8 | 17.9 | 12.5 | 9.2 | 5.5 | 1.9 | 16.9 | 9.0 | 13.3 ± 9.9 |
| | NJCFFJJN_02561 | GH10 [Fnc] | 23.7 | 18.0 | 9.4 | 6.6 | 6.0 | 0.5 | 8.4 | 5.7 | 9.8 ± 7.5 |
| no CAZyme | NJCFFJJN_02565 | SusC | 13.1 | 12.0 | 17.0 | 3.5 | 5.1 | 1.7 | 4.0 | 5.6 | 7.8 ± 5.5 |
| | NJCFFJJN_02566 | SusD | 12.7 | 10.8 | 16.8 | 3.3 | 5.3 | 1.4 | 4.4 | 5.4 | 7.5 ± 5.3 |
| | NJCFFJJN_02567 | [unk] | 14.0 | 10.1 | 21.5 | 5.3 | 5.1 | 2.0 | 7.6 | 8.6 | 9.3 ± 6.1 |
| unknown b- | NJCFFJJN_02758 | GH165 | 11.8 | 23.7 | 24.9 | 9.1 | 14.5 | 3.5 | 11.7 | 3.7 | 12.9 ± 8.1 |
| galactoside | | | | | | | | | | | |
| | NJCFFJJN_02759 | [unk] | 18.0 | 31.8 | 36.5 | 13.1 | 19.0 | 4.5 | 18.3 | 6.5 | 18.5 ± 11.1 |
| | NJCFFJJN_02760 | GH165 | 19.2 | 32.6 | 40.1 | 12.8 | 19.6 | 4.4 | 17.7 | 6.5 | 19.1 ± 12.2 |
| | NJCFFJJN_02761 | GH165 | 19.0 | 29.5 | 41.0 | 12.2 | 18.1 | 4.6 | 16.8 | 5.3 | 18.3 ± 12.2 |
| | NJCFFJJN_02762 | SusD | 19.7 | 30.9 | 44.0 | 14.2 | 21.1 | 4.8 | 18.8 | 5.9 | 19.9 ± 12.9 |
| | NJCFFJJN_02763 | SusC | 20.7 | 28.9 | 42.5 | 13.3 | 20.3 | 4.4 | 17.0 | 5.8 | 19.1 ± 12.4 |
| | NJCFFJJN_02764 | [unk] | 21.7 | 25.9 | 40.1 | 15.5 | 19.4 | 4.7 | 15.9 | 6.3 | 18.7 ± 11.3 |
| | NJCFFJJN_02765 | MFS | 25.1 | 34.4 | 41.0 | 17.9 | 23.0 | 5.6 | 15.9 | 7.4 | 21.3 ± 12.3 |
| no CAZyme | NJCFFJJN_02912 | SusC | 5.5 | 3.6 | 6.4 | 1.4 | 1.5 | 0.5 | 2.6 | 2.4 | 3.0 ± 2.1 |
| | NJCFFJJN_02913 | SusD | 6.6 | 3.9 | 7.7 | 1.6 | 2.1 | 0.8 | 2.9 | 2.5 | 3.5 ± 2.4 |
| b-1,2-glucan | NJCFFJJN_02921 | SusC | 55.8 | 32.3 | 20.0 | 12.0 | 8.6 | 2.2 | 25.2 | 32.5 | 23.6 ± 17.0 |
| | NJCFFJJN_02922 | SusD | 67.7 | 40.9 | 24.2 | 15.2 | 9.4 | 3.2 | 31.6 | 40.2 | 29.1 ± 20.8 |
| | NJCFFJJN_02923 | [unk] | 62.5 | 25.1 | 19.2 | 11.2 | 8.7 | 1.9 | 23.7 | 29.8 | 22.8 ± 18.6 |
| | NJCFFJJN_02924 | GH144 | 30.1 | 16.7 | 11.0 | 6.8 | 2.9 | 0.7 | 12.6 | 16.8 | 12.2 ± 9.3 |
| | NJCFFJJN_02925 | GH3 | 25.4 | 15.6 | 8.9 | 5.5 | 3.0 | 0.5 | 11.5 | 14.1 | 10.6 ± 8.0 |
| | NJCFFJJN_02926 | [unk] | 31.0 | 17.6 | 9.4 | 7.2 | 3.9 | 0.6 | 11.0 | 14.6 | 11.9 ± 9.4 |
| no CAZyme | NJCFFJJN_02957 | SusC | 230.1 | 108.2 | 133.1 | 28.4 | 40.0 | 16.2 | 57.4 | 37.1 | 81.3 ± 72.5 |
| | NJCFFJJN_02958 | SusD | 229.2 | 115.4 | 147.7 | 30.4 | 42.2 | 15.7 | 63.8 | 45.5 | 86.3 ± 73.0 |
| N- and O- | NJCFFJJN_02960 | GH2 | 8.9 | 12.7 | 15.7 | 6.0 | 6.8 | 2.3 | 33.1 | 8.1 | 11.7 ± 9.5 |
| glycans | | | | | | | | | | | |
| | NJCFFJJN_02961 | Est | 6.5 | 10.4 | 10.3 | 3.8 | 6.5 | 1.6 | 29.5 | 4.4 | 9.1 ± 8.8 |
| | NJCFFJJN_02962 | [unk] | 7.5 | 13.5 | 16.4 | 4.4 | 7.4 | 2.1 | 37.1 | 5.7 | 11.8 ± 11.3 |
| | NJCFFJJN_02963 | [unk] | 5.6 | 7.7 | 9.6 | 3.6 | 5.7 | 1.3 | 31.9 | 4.5 | 8.7 ± 9.7 |
| | NJCFFJJN_02964 | SusD | 12.5 | 14.8 | 19.8 | 7.4 | 12.0 | 2.3 | 75.6 | 9.6 | 19.3 ± 23.4 |
| | NJCFFJJN_02965 | SusC | 14.1 | 16.2 | 20.4 | 7.5 | 12.2 | 2.3 | 83.4 | 10.9 | 20.9 ± 25.9 |
| | NJCFFJJN_02966 | GH33 | 14.4 | 13.3 | 21.5 | 6.6 | 10.6 | 1.7 | 62.5 | 10.0 | 17.6 ± 19.0 |
| | NJCFFJJN_02967 | EPI | 15.1 | 15.8 | 19.9 | 9.2 | 9.7 | 2.8 | 71.8 | 12.8 | 19.6 ± 21.7 |
| | NJCFFJJN_02968 | [unk] | 17.8 | 22.9 | 29.3 | 12.7 | 13.4 | 3.1 | 101.5 | 17.7 | 27.3 ± 30.9 |
| | NJCFFJJN_02969 | MFS | 16.1 | 15.7 | 21.9 | 11.3 | 10.7 | 3.5 | 85.0 | 16.0 | 22.5 ± 25.8 |
| a-glucoside, a- | NJCFFJJN_03039 | SusR | 8.7 | 9.7 | 13.4 | 6.7 | 3.9 | 2.2 | 19.0 | 5.9 | 8.7 ± 5.4 |
| 1,6-glucan | | | | | | | | | | | |
| (dextran) | | | | | | | | | | | |
| | NJCFFJJN_03040 | GH31 | 1.2 | 0.8 | 2.5 | 0.4 | 0.4 | 0.1 | 2.1 | 0.6 | 1.0 ± 0.9 |
| | NJCFFJJN_03041 | [unk] | 0.6 | 0.6 | 2.1 | 0.3 | 0.3 | 0.2 | 2.4 | 0.4 | 0.9 ± 0.9 |
| | NJCFFJJN_03042 | SusC | 1.4 | 0.7 | 6.1 | 1.2 | 0.5 | 0.3 | 7.7 | 1.7 | 2.4 ± 2.8 |
| | NJCFFJJN_03043 | SusC | 1.3 | 1.0 | 2.7 | 0.5 | 0.4 | 0.2 | 3.4 | 0.7 | 1.3 ± 1.1 |
| | NJCFFJJN_03044 | SusD | 1.1 | 1.0 | 2.2 | 0.4 | 0.4 | 0.2 | 2.9 | 0.4 | 1.1 ± 1.0 |
| | NJCFFJJN_03045 | [unk] | 0.8 | 0.5 | 1.6 | 0.4 | 0.4 | 0.3 | 2.9 | 0.3 | 0.9 ± 0.9 |
| | NJCFFJJN_03046 | [unk] | 1.1 | 0.9 | 1.2 | 0.2 | 0.7 | 0.1 | 2.3 | 0.3 | 0.9 ± 0.7 |
| | NJCFFJJN_03047 | GH27 | 0.9 | 0.7 | 1.8 | 0.4 | 0.6 | 0.1 | 2.2 | 0.4 | 0.9 ± 0.7 |
| | NJCFFJJN_03048 | [unk] | 0.9 | 0.9 | 2.0 | 0.2 | 0.5 | 0.1 | 2.5 | 0.5 | 0.9 ± 0.9 |
| | NJCFFJJN_03049 | GH97 | 0.8 | 0.9 | 2.2 | 0.6 | 0.7 | 0.2 | 3.0 | 0.5 | 1.1 ± 1.0 |
| | NJCFFJJN_03050 | GH66 [Fc] | 0.4 | 0.6 | 1.1 | 0.3 | 0.2 | 0.3 | 1.5 | 0.2 | 0.6 ± 0.5 |
| | NJCFFJJN_03051 | [unk] | 1.4 | 1.9 | 4.3 | 0.5 | 0.3 | 1.6 | 4.7 | 1.4 | 2.0 ± 1.6 |
| Unknown | NJCFFJJN_03087 | HTCS | 11.8 | 10.2 | 18.6 | 5.4 | 4.4 | 2.3 | 8.4 | 17.5 | 9.8 ± 5.9 |
| | NJCFFJJN_03088 | [unk] | 10.2 | 9.0 | 17.6 | 5.5 | 4.2 | 2.6 | 10.6 | 14.5 | 9.3 ± 5.1 |
| | NJCFFJJN_03089 | SusC | 1.5 | 1.0 | 1.3 | 0.4 | 0.5 | 0.1 | 0.8 | 4.9 | 1.3 ± 1.5 |
| | NJCFFJJN_03090 | SusD | 2.3 | 1.1 | 1.5 | 0.5 | 0.5 | 0.1 | 1.2 | 5.2 | 1.5 ± 1.6 |
| | NJCFFJJN_03091 | Est | 1.7 | 1.1 | 1.7 | 0.6 | 0.6 | 0.2 | 1.3 | 5.7 | 1.6 ± 1.7 |
| | NJCFFJJN_03092 | Est | 2.0 | 1.1 | 1.7 | 0.5 | 0.4 | 0.0 | 1.2 | 5.2 | 1.5 ± 1.6 |
| | NJCFFJJN_03093 | GH3 | 2.3 | 1.4 | 2.0 | 0.5 | 0.7 | 0.2 | 1.8 | 6.8 | 2.0 ± 2.1 |
| | NJCFFJJN_03094 | [unk] | 88.9 | 85.3 | 101.1 | 53.9 | 35.5 | 25.6 | 82.4 | 81.8 | 69.3 ± 27.4 |
| | NJCFFJJN_03095 | [unk] | 1.7 | 2.6 | 3.4 | 1.1 | 1.2 | 0.4 | 1.1 | 1.2 | 1.6 ± 1.0 |
| | NJCFFJJN_03096 | GH97 | 1.2 | 1.2 | 1.8 | 0.3 | 0.4 | 0.4 | 0.7 | 0.8 | 0.9 ± 0.5 |
| | NJCFFJJN_03097 | [unk] | 20.1 | 14.8 | 21.8 | 11.3 | 6.9 | 5.0 | 18.0 | 20.1 | 14.7 ± 6.4 |
| | NJCFFJJN_03098 | GH127 | 10.8 | 12.6 | 14.4 | 5.0 | 6.2 | 2.2 | 39.4 | 9.5 | 12.5 ± 11.6 |
| | NJCFFJJN_03099 | ECF-s | 19.1 | 23.1 | 41.3 | 30.0 | 9.6 | 6.7 | 12.5 | 41.6 | 23.0 ± 13.6 |
| | NJCFFJJN_03100 | [unk] | 33.3 | 34.2 | 60.4 | 43.2 | 15.7 | 10.9 | 23.5 | 55.0 | 34.5 ± 17.7 |
| | NJCFFJJN_03101 | [unk] | 33.4 | 35.8 | 63.6 | 40.1 | 16.8 | 10.5 | 28.5 | 55.0 | 35.5 ± 17.8 |
| | NJCFFJJN_03102 | PL33 [Fnc] | 11.7 | 14.9 | 27.3 | 12.3 | 6.3 | 4.1 | 11.0 | 18.1 | 13.2 ± 7.2 |
| no CAZyme | NJCFFJJN_03119 | SusC | 59.2 | 25.6 | 31.7 | 7.1 | 12.7 | 5.0 | 12.1 | 9.0 | 20.3 ± 18.2 |
| | NJCFFJJN_03120 | SusD | 86.7 | 38.0 | 44.3 | 10.6 | 18.5 | 7.0 | 17.4 | 14.0 | 29.6 ± 26.5 |
| | NJCFFJJN_03121 | Pept_MA | 67.7 | 31.2 | 38.8 | 8.1 | 14.1 | 6.0 | 14.7 | 11.2 | 24.0 ± 21.1 |
| | NJCFFJJN_03122 | [unk] | 90.0 | 38.3 | 49.5 | 11.8 | 19.8 | 7.1 | 18.1 | 15.2 | 31.2 ± 27.7 |
| | NJCFFJJN_03123 | [unk] | 78.4 | 35.2 | 40.6 | 9.5 | 17.0 | 6.0 | 19.9 | 15.0 | 27.7 ± 23.7 |
| | NJCFFJJN_03124 | [unk] | 145.9 | 73.1 | 86.9 | 20.7 | 34.6 | 10.9 | 39.2 | 30.9 | 55.3 ± 44.7 |
| | NJCFFJJN_03125 | Pept_MA | 34.1 | 18.1 | 21.7 | 4.9 | 8.3 | 3.4 | 9.5 | 6.0 | 13.2 ± 10.6 |
| type II | NJCFFJJN_03172 | GH43_18- | 91.2 | 32.2 | 33.9 | 11.7 | 6.9 | 4.1 | 6.6 | 17.9 | 25.6 ± 28.9 |
| rhamnogalac- | | GH143- | | | | | | | | | |
| turonan | | GH142 | | | | | | | | | |
| | NJCFFJJN_03173 | GH78 | 51.5 | 23.5 | 18.4 | 7.8 | 4.4 | 2.3 | 5.4 | 10.1 | 15.5 ± 16.3 |
| | NJCFFJJN_03174 | CE19 | 52.5 | 27.8 | 17.5 | 8.6 | 4.9 | 3.2 | 6.4 | 11.9 | 16.6 ± 16.6 |
| | NJCFFJJN_03175 | GH95 | 55.3 | 23.1 | 29.8 | 9.4 | 4.3 | 3.4 | 7.7 | 15.5 | 18.6 ± 17.5 |
| | NJCFFJJN_03176 | GH140 | 103.7 | 42.3 | 48.5 | 16.2 | 9.2 | 6.2 | 12.2 | 24.4 | 32.8 ± 32.5 |
| | NJCFFJJN_03177 | GH78-GH33 | 111.2 | 52.4 | 48.0 | 17.4 | 9.1 | 6.7 | 15.4 | 26.5 | 35.8 ± 34.9 |
| | NJCFFJJN_03178 | [unk] | 7.4 | 0.0 | 1.6 | 0.0 | 0.0 | 2.2 | 0.0 | 3.0 | 1.8 ± 2.6 |
| | NJCFFJJN_03179 | GH138 | 90.2 | 36.8 | 36.8 | 13.6 | 8.4 | 5.4 | 7.3 | 19.9 | 27.3 ± 28.3 |
| | NJCFFJJN_03180 | [unk] | 4.2 | 3.0 | 5.4 | 2.6 | 1.3 | 0.8 | 2.5 | 2.7 | 2.8 ± 1.5 |
| | NJCFFJJN_03181 | [unk] | 6.9 | 5.9 | 5.4 | 3.2 | 1.6 | 1.1 | 3.2 | 4.3 | 3.9 ± 2.0 |
| | NJCFFJJN_03182 | GH137-GH2- | 41.1 | 17.9 | 16.8 | 5.8 | 4.0 | 2.1 | 3.6 | 8.3 | 12.4 ± 13.0 |
| | | CBM57 | | | | | | | | | |
| | NJCFFJJN_03183 | GH2 | 77.4 | 38.9 | 39.5 | 14.1 | 7.3 | 5.3 | 12.9 | 22.6 | 27.2 ± 24.2 |
| | NJCFFJJN_03184 | [unk] | 10.7 | 6.6 | 7.5 | 2.9 | 2.8 | 1.2 | 3.7 | 4.4 | 5.0 ± 3.1 |
| | NJCFFJJN_03185 | GH2 | 26.6 | 14.4 | 13.8 | 4.7 | 4.2 | 2.1 | 7.0 | 9.1 | 10.2 ± 7.9 |
| | NJCFFJJN_03186 | GH106 | 50.3 | 22.3 | 23.2 | 7.1 | 6.6 | 2.6 | 11.2 | 12.8 | 17.0 ± 15.3 |
| | NJCFFJJN_03187 | Est | 56.4 | 31.0 | 28.2 | 9.9 | 6.4 | 2.9 | 15.1 | 14.9 | 20.6 ± 17.5 |
| | NJCFFJJN_03188 | GH2 | 70.7 | 35.7 | 31.1 | 10.3 | 8.5 | 3.7 | 17.1 | 16.7 | 24.2 ± 21.8 |
| | NJCFFJJN_03189 | HTCS | 6.8 | 4.0 | 5.4 | 1.8 | 2.2 | 0.9 | 3.4 | 3.4 | 3.5 ± 1.9 |
| | NJCFFJJN_03190 | SusC | 241.0 | 87.2 | 86.1 | 30.0 | 18.3 | 8.7 | 19.2 | 48.4 | 67.4 ± 76.4 |
| | NJCFFJJN_03191 | SusD | 371.5 | 145.4 | 114.1 | 43.9 | 27.1 | 11.8 | 36.6 | 64.9 | 101.9 ± 118.0 |
| a-glucan | NJCFFJJN_03203 | GH13 | 95.5 | 79.9 | 82.2 | 36.9 | 27.9 | 24.1 | 89.4 | 36.5 | 59.1 ± 30.3 |
| (starch) | | | | | | | | | | | |
| | NJCFFJJN_03204 | Int | 1.8 | 0.3 | 0.9 | 0.7 | 0.4 | 0.1 | 0.3 | 1.5 | 0.7 ± 0.6 |
| | NJCFFJJN_03205 | SusC | 24.4 | 3.2 | 5.9 | 5.8 | 29.7 | 3.2 | 7.1 | 1.2 | 10.1 ± 10.7 |
| | NJCFFJJN_03206 | SusD | 26.0 | 3.8 | 6.6 | 5.9 | 36.6 | 3.9 | 8.9 | 1.5 | 11.6 ± 12.6 |
| | NJCFFJJN_03207 | [unk] | 28.2 | 4.5 | 9.5 | 7.2 | 44.7 | 4.6 | 10.8 | 2.0 | 13.9 ± 14.8 |
| | NJCFFJJN_03208 | [unk] | 24.7 | 4.4 | 9.3 | 7.9 | 41.3 | 5.1 | 12.3 | 2.1 | 13.4 ± 13.3 |
| starch | NJCFFJJN_03225 | [unk] | 385.8 | 300.4 | 286.3 | 98.2 | 71.3 | 76.0 | 426.3 | 117.8 | 220.3 ± 145.9 |
| | NJCFFJJN_03226 | [unk] | 421.0 | 300.9 | 324.6 | 102.8 | 68.0 | 76.6 | 428.0 | 108.2 | 228.8 ± 156.0 |
| | NJCFFJJN_03227 | SusD | 419.3 | 274.8 | 301.4 | 94.4 | 65.6 | 74.1 | 380.1 | 95.6 | 213.2 ± 146.9 |
| | NJCFFJJN_03228 | SusC | 347.6 | 242.6 | 236.1 | 76.2 | 52.3 | 57.6 | 298.5 | 80.4 | 173.9 ± 120.0 |
| | NJCFFJJN_03229 | [unk] | 37.5 | 30.3 | 27.7 | 10.9 | 11.1 | 9.6 | 46.2 | 13.9 | 23.4 ± 14.0 |
| | NJCFFJJN_03230 | MFS | 64.3 | 45.1 | 40.0 | 14.5 | 14.6 | 9.6 | 47.9 | 15.6 | 31.5 ± 20.4 |
| | NJCFFJJN_03231 | CBM20-GH77 | 119.9 | 91.3 | 90.4 | 34.5 | 27.8 | 22.2 | 98.8 | 36.2 | 65.1 ± 38.7 |
| | NJCFFJJN_03232 | GH97 | 76.6 | 62.6 | 60.0 | 23.7 | 22.5 | 16.6 | 75.5 | 26.4 | 45.5 ± 25.6 |
| | NJCFFJJN_03233 | GH13_14 | 62.8 | 50.8 | 57.3 | 19.2 | 18.2 | 12.4 | 64.6 | 19.3 | 38.1 ± 22.7 |
| | NJCFFJJN_03234 | GH13 | 103.8 | 97.3 | 92.1 | 34.3 | 31.3 | 23.7 | 129.1 | 40.2 | 69.0 ± 40.8 |
| arabinogalactan | NJCFFJJN_03238 | HTCS | 31.6 | 33.2 | 35.3 | 15.4 | 12.5 | 6.2 | 36.8 | 25.0 | 24.5 ± 11.7 |
| | NJCFFJJN_03239 | [unk] | 113.0 | 80.8 | 165.6 | 10.7 | 56.8 | 4.3 | 267.4 | 67.6 | 95.8 ± 86.8 |
| | NJCFFJJN_03240 | SusC | 234.7 | 141.5 | 287.0 | 19.1 | 113.3 | 9.6 | 402.3 | 118.2 | 165.7 ± 134.7 |
| | NJCFFJJN_03241 | SusD | 237.3 | 160.9 | 295.3 | 21.9 | 107.3 | 9.5 | 431.7 | 140.8 | 175.6 ± 141.9 |
| | NJCFFJJN_03242 | SusC | 243.1 | 145.4 | 273.8 | 22.9 | 106.0 | 9.8 | 432.9 | 134.4 | 171.0 ± 140.7 |
| | NJCFFJJN_03243 | SusD | 245.3 | 158.0 | 270.7 | 24.8 | 97.7 | 10.2 | 452.2 | 153.2 | 176.5 ± 145.1 |
| | NJCFFJJN_03244 | GH43_4 | 82.5 | 55.1 | 94.5 | 8.3 | 32.6 | 3.7 | 133.9 | 52.5 | 57.9 ± 44.4 |
| | NJCFFJJN_03245 | GH43_5 | 93.7 | 61.5 | 87.8 | 8.7 | 33.2 | 3.7 | 121.2 | 43.2 | 56.6 ± 42.0 |
| no CAZyme | NJCFFJJN_03252 | [unk] | 0.7 | 0.6 | 2.4 | 0.3 | 0.4 | 0.2 | 0.4 | 0.6 | 0.7 ± 0.7 |
| | NJCFFJJN_03253 | SusD | 0.2 | 0.2 | 0.8 | 0.1 | 0.1 | 0.0 | 0.1 | 0.1 | 0.2 ± 0.2 |
| | NJCFFJJN_03254 | SusC | 0.0 | 0.3 | 0.6 | 0.1 | 0.1 | 0.0 | 0.0 | 0.1 | 0.2 ± 0.2 |
| | NJCFFJJN_03255 | [unk] | 0.1 | 0.1 | 0.8 | 0.1 | 0.1 | 0.0 | 0.0 | 0.1 | 0.1 ± 0.3 |
| no CAZyme | NJCFFJJN_03286 | SusC | 16.2 | 8.0 | 13.8 | 1.6 | 3.7 | 0.8 | 2.6 | 1.3 | 6.0 ± 6.0 |
| | NJCFFJJN_03287 | SusD | 2.8 | 3.5 | 8.0 | 0.6 | 1.7 | 0.7 | 0.7 | 0.5 | 2.3 ± 2.5 |
| | NJCFFJJN_03288 | Pept_MA | 2.3 | 4.3 | 9.9 | 1.3 | 1.5 | 0.6 | 0.5 | 0.5 | 2.6 ± 3.2 |
| | NJCFFJJN_03289 | [unk] | 2.8 | 6.4 | 12.2 | 1.0 | 2.2 | 1.3 | 1.3 | 1.4 | 3.6 ± 3.9 |
| b-1,3-glucan | NJCFFJJN_03307 | SusR | 15.2 | 21.6 | 31.6 | 9.9 | 8.8 | 4.1 | 9.5 | 11.3 | 14.0 ± 8.8 |
| | NJCFFJJN_03308 | GH3 | 14.3 | 17.6 | 23.7 | 9.4 | 7.7 | 3.6 | 8.2 | 10.4 | 11.9 ± 6.4 |
| | NJCFFJJN_03309 | SusC | 4.9 | 8.8 | 11.2 | 2.7 | 3.2 | 1.6 | 3.0 | 2.9 | 4.8 ± 3.4 |
| | NJCFFJJN_03310 | SusD | 5.2 | 12.0 | 13.5 | 4.0 | 3.9 | 1.8 | 3.8 | 3.6 | 6.0 ± 4.3 |
| | NJCFFJJN_03311 | [unk] | 5.3 | 11.3 | 13.0 | 3.4 | 3.4 | 2.0 | 3.8 | 4.7 | 5.9 ± 4.0 |
| | NJCFFJJN_03312 | GH16_3 | 5.0 | 6.8 | 10.5 | 3.2 | 3.2 | 1.6 | 3.3 | 3.6 | 4.7 ± 2.8 |
| a-glucan | NJCFFJJN_03339 | SusR | 4.4 | 4.5 | 6.7 | 2.3 | 2.0 | 1.0 | 3.7 | 3.3 | 3.5 ± 1.8 |
| (starch) | | | | | | | | | | | |
| | NJCFFJJN_03340 | SusC | 32.0 | 10.6 | 17.3 | 12.0 | 6.5 | 3.1 | 21.7 | 11.8 | 14.4 ± 9.2 |
| | NJCFFJJN_03341 | SusD | 46.2 | 12.9 | 21.6 | 15.5 | 8.7 | 5.4 | 27.1 | 16.0 | 19.2 ± 12.9 |
| | NJCFFJJN_03342 | GH97 | 58.2 | 20.3 | 30.2 | 21.9 | 12.0 | 7.9 | 37.3 | 23.5 | 26.4 ± 15.9 |
| | NJCFFJJN_03343 | GH13_m52 | 77.4 | 32.8 | 46.5 | 28.7 | 19.0 | 10.9 | 56.8 | 35.5 | 38.4 ± 21.4 |

*[Fcn]: fragment too short at N and C termini; [Fn]: fragment too short at N terminus; [Fc]: fragment too short at C terminus; [Fs]: splicing or gene model problem; [unk]: sequence is not assigned to a CAZyme family

In order to identify pathways that drive separation of samples along PC1, the contribution of each pathway was used in each community member to each singular vector to rank the pathways. Notably, arabinose utilization was consistently among the most upregulated pathways with P. copri colonization (FIG. 11F). Moreover, three of the four bacteria capable of arabinose utilization (Blautia obeum, Bifidobacterium catenulatum, and Mitsuokella multacida) were significantly more abundant in the cecums of mice colonized with P. copri.

Subsequently, differential expression analysis was performed (using edgeR; see Methods) to further assess the effects of P. copri-colonization on the transcriptomic profiles of community members at gene-level resolution. Differentially expressed transcripts associated with complete metabolic pathways are summarized in FIG. 11G. Among the four arabinose-utilizing strains, B. obeum and M. multacida demonstrated significantly higher expression of all of their genes involved in arabinose utilization with P. copri colonization. Both B. obeum and M. multacida also demonstrated statistically significant upregulation of all or most of their genes involved in biosynthesis of glutamine and glutamate, as well as branched-chain amino acids (isoleucine, leucine, and valine). In addition, B. obeum displayed elevated transcription of genes involved in acetate production in the P. copri-colonized mice. Integrating the mass spectrometric and microbial RNA-seq results generated from this defined consortium indicates that P. copri colonization leads to liberation of arabinose from MDCF-2 glycans, which in turn becomes bioavailable to other community members, including positively WLZ-associated members such as B. obeum, resulting in their increased fitness and altered expressed metabolic functions.

### Example 10: SnRNA-Seq of Intestinal Gene Expression

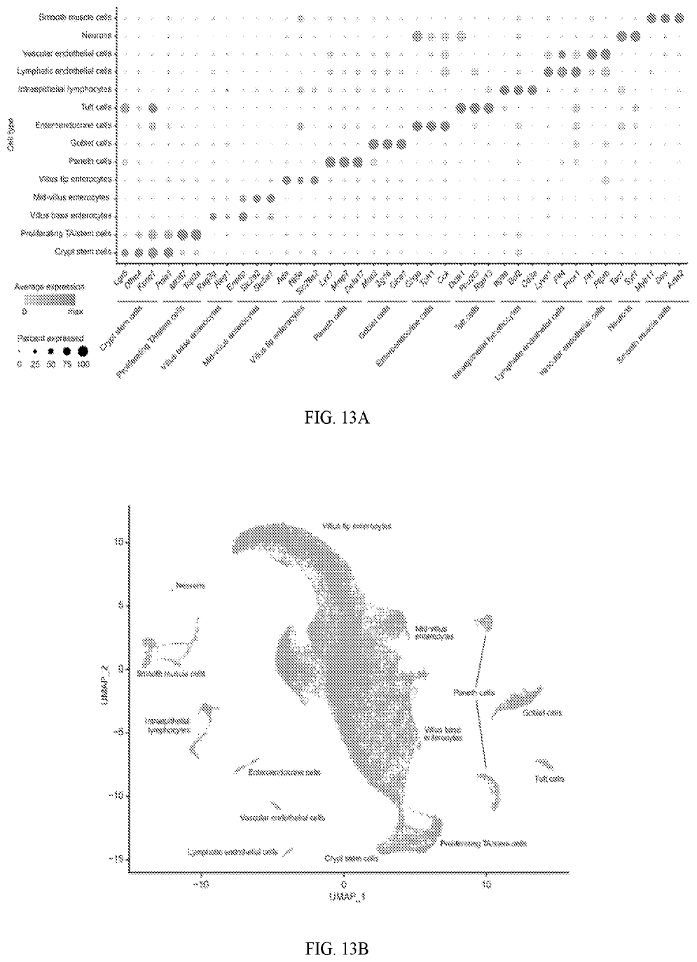

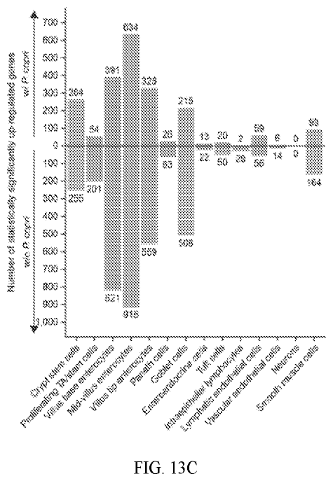

Histomorphometric analysis of villus height and crypt depth in jejunums harvested from mice harboring communities with and without P. copri (n=8 and 7, respectively) disclosed no statistically significant architectural differences between the two treatment groups (P>0.05; Mann-Whitney U test; Table 21). snRNA-Seq was used subsequently, to investigate whether these two colonization states produced differences in expressed functions along the crypt-villus axis in jejunal tissue collected from P53 animals (n=4/treatment arm; FIG. 12A-F; FIG. 13A-C; Tables 22 and 23). A total of 30,717 nuclei passed our quality metrics (see Methods). Marker gene-based annotation disclosed cell clusters that were assigned to the four principal intestinal epithelial cell lineages (enterocytic, goblet, enteroendocrine, and Paneth cell) as well as to vascular endothelial cells, lymphatic endothelial cells, smooth muscle cells and enteric neurons (FIG. 13A-C). Marker gene analysis allowed us to further subdivide the enterocytic lineage into three clusters: ‘villus-base’, ‘mid-villus’ and ‘villus-tip’. Pseudobulk snRNA-seq analysis, which aggregates transcripts for each cell cluster and then uses edgeR to identify differentially expressed genes in each cluster^{18,19}, disclosed that a majority of all statistically significant differentially expressed genes (3,651 of 5,765; 63.3%) were assigned to the three enterocyte clusters (FIG. 13C).

TABLE 21
Quantification of jejunal villus height and crypt depth from
gnotobiotic mice harboring defined bacterial consortia

| | | Villus | Crypt | villus/ |
|---|---|---|---|---|
| | Mouse | height | depth | crypt |
| Treatment | no. | (μm) | (μm) | ratio |
| w/P. copri (Arm 1) | 1 | 430 | 72 | 5.9 |
| | 2 | 516 | 76 | 6.8 |
| | 3 | 434 | 71 | 6.1 |
| | 4 | 488 | 87 | 5.6 |
| | 5 | 451 | 81 | 5.6 |
| | 6 | 573 | 76 | 7.5 |
| | 7 | 524 | 81 | 6.4 |
| | 8 | 603 | 73 | 8.2 |
| w/o P. copri (Arm 3) | 1 | 517 | 88 | 5.9 |
| | 2 | 515 | 80 | 6.4 |
| | 3 | 507 | 77 | 6.6 |
| | 4 | 550 | 64 | 8.6 |
| | 5 | 600 | 96 | 6.3 |
| | 6 | 569 | 84 | 6.8 |
| | 7 | 579 | 78 | 7.4 |

TABLE 22
snRNA-Seq dataset generated from jejunums of gnotobiotic
mice colonized with defined consortia of cultured
bacterial strains, sample metadata

| Treat- | | | | Number | |
|---|---|---|---|---|---|
| ment | Mouse | | | of raw | |
| arm | no. | Sex | Sample ID | reads | Index |
| w/ P. | 4 | male | defined_community.preweaning_ | 3.19E+08 | CGGAGCAC- |
| copri | | | P_copri_colonization.postnatal_ | | GACCTATT- |
| (Arm 1) | | | day_53.pup.m4.jejunum.snRNAseq | | ACTTAGGA- |
| | | | | | TTAGCTCG |
| | 5 | female | defined_community.preweaning | 3.21E+08 | CGTGCAGA- |
| | | | P_copri_colonization.postnatal_ | | AACAAGAT- |
| | | | day_53.pup.m5.jejunum.snRNAseq | | TCGCTTCG- |
| | | | | | GTATGCTC |
| | 7 | male | defined_community.preweaning_ | 3.51E+08 | CATGAACA- |
| | | | P_copri_colonization.postnatal_ | | TCACTCGC- |
| | | | day_53.pup.m7.jejunum.snRNAseq | | AGCTGGAT- |
| | | | | | GTGACTTG |
| | 8 | female | defined_community.preweaning_ | 3.28E+08 | CAAGCTCC- |
| | | | P_copri_colonization.postnatal_ | | GTTCACTG- |
| | | | day_53.pup.m8.jejunum.snRNAseq | | TCGTGAAA- |
| | | | | | AGCATGGT |
| w/o P. | 3 | female | defined_community.no_P_copri_ | 3.05E+08 | GCTTGGCT- |
| copri | | | colonization.postnatal_day_53. | | AAACAAAC- |
| (Arm 3) | | | pup.m3.jejunum.snRNAseq | | CGGGCTTA- |
| | | | | | TTCATCGG |
| | 5 | male | defined_community.no_P_copri_ | 3.11E+08 | GCGAGAGT- |
| | | | colonization.postnatal_day_53. | | TACGTTCA- |
| | | | pup.m5.jejunum.snRNAseq | | AGTCCCAC- |
| | | | | | CTATAGTG |
| | 6 | female | defined_community.no_P_copri_ | 3.29E+08 | TGATGCAT- |
| | | | colonization.postnatal_day_53. | | GCTACTGA- |
| | | | pup.m6.jejunum.snRNAseq | | CACCTGCC- |
| | | | | | ATGGAATG |
| | 7 | male | defined_community.no_P_copri_ | 3.22E+08 | ATGAATCT- |
| | | | colonization.postnatal_day_53. | | GATCTCAG- |
| | | | pup.m7.jejunum.snRNAseq | | CCAGGAGC- |
| | | | | | TGCTCGTA |

TABLE 23
Proportional representation of cell clusters
identified from snRNA-seq dataset

| | Proportional | | | | |
|---|---|---|---|---|---|
| | representation | | scCODA output | | |
| | (mean ± SD) | | | | statistically |
| | w/P. | w/o P. | | Inclusion | credible |
| Cell cluster | copri | copri | Effect | probability | difference |
| Crypt stem cells | 4.34 ± | 1.83 ± | 0.72 | 1.00 | Yes |
| | 1.41% | 0.81% | | | |
| Proliferating | 2.46 ± | 1.05 ± | 0.61 | 0.95 | Yes |
| TA/stem cells | 0.89% | 0.43% | | | |
| Enterocytes villus | 20.63 ± | 18.15 ± | 0 | 0.36 | No |
| base | 1.19% | 1.65% | | | |
| Enterocytes mid | 44.81 ± | 51.96 ± | 0 | 0.90 | No |
| villus | 4.20% | 2.26% | | | |
| Enterocytes villus | 13.70 ± | 13.28 ± | 0 | 0.26 | No |
| tip | 1.41% | 0.78% | | | |
| Paneth cells | 2.75 ± | 2.51 ± | 0 | 0.33 | No |
| | 0.36% | 0.27% | | | |
| Goblet cells | 2.97 ± | 3.16 ± | 0 | 0.34 | No |
| | 0.46% | 0.64% | | | |
| Enteroendocrine | 0.73 ± | 0.79 ± | 0 | 0 | No |
| cells | 0.04% | 0.20% | | | |
| Tuft cells* | 1.24 ± | 0.34 ± | 0.73 | 0.96 | Yes |
| | 0.48% | 0.08% | | | |
| Intraepithelial | 2.29 ± | 1.85 ± | 0 | 0.40 | No |
| lymphocytes | 2.21% | 1.37% | | | |
| Lymphatic | 0.56 ± | 0.72 ± | 0 | 0.46 | No |
| endothelial cells | 0.15% | 0.19% | | | |
| Vascular | 0.51 ± | 0.69 ± | 0 | 0.51 | No |
| endothelial cells | 0.11% | 0.18% | | | |
| Neurons | 0.07 ± | 0.09 ± | 0 | 0.42 | No |
| | 0.03% | 0.06% | | | |
| Smooth muscle | 2.96 ± | 3.58 ± | 0 | 0.50 | No |
| cells | 0.12% | 0.73% | | | |

NicheNet was used initially, to evaluate the effects of the P. copri community on intercellular communications. NicheNet integrates information on signaling and gene regulation from publicly available databases to build a “prior model of ligand-target regulatory potential” and then predicts potential communications between user-defined “sender” and “receiver” cell clusters. After incorporating snRNA-Seq-based expression data from both sender and receiver cells, NicheNet computes a list of potential ligand-receptor interactions between senders and receivers. The ligand-receptor interactions in the resulting list are then ranked based on the effect of the ligand-receptor interactions on downstream genes in their signaling pathway (i.e., more downstream genes are expressed in a ‘high-ranking’ interaction). After this ranking step, an additional filter is applied, with ligand-receptor interactions having firm experimental validation in the literature designated as “bona fide” interactions. Finally, NicheNet uses information generated by Seurat from a snRNA-Seq dataset to identify altered “bona fide” ligand-receptor interactions.

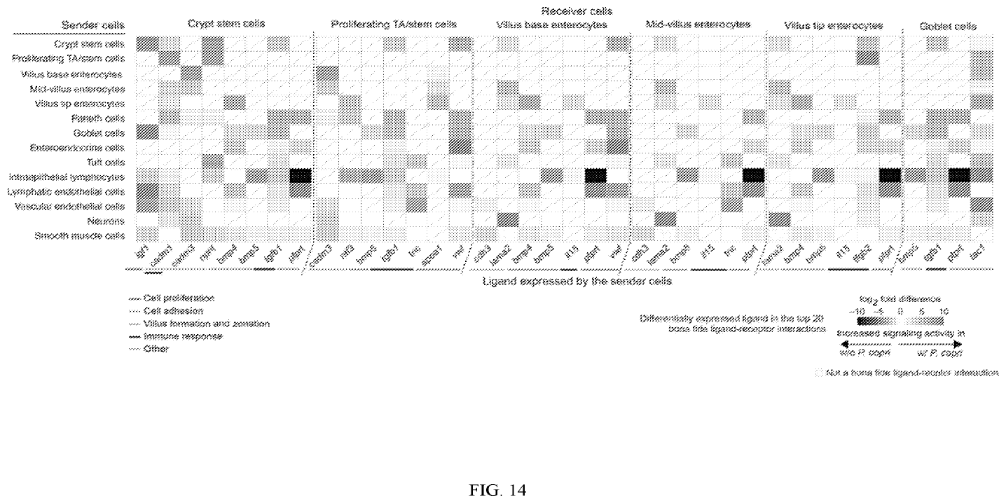

The six epithelial cell clusters (crypt stem cells, proliferating TA/stem cells, villus base, mid-villus, and villus tip enterocytes and goblet cells) were designated as “receiver cells” while all clusters (both epithelial and mesenchymal) were designated “sender cells”. NicheNet analysis was then conducted for each sender-receiver pair. FIG. 14 shows bona fide ligand-receptor interactions that are altered between the two colonization conditions for each receiver cell cluster. Ligands identified include those known to affect cell proliferation (igf-1), cell adhesion (cadm1, cadm3, cdh3, lama2, npnt), zonation of epithelial cell function/differentiation along the length of the villus (bmp4, bmp5), as well as immune responses (cadm1, il15, tgfb1, tnc) (FIG. 14). Among all receiver cell clusters, crypt stem cells exhibited the highest number of altered bona fide ligand-receptor interactions. For example, Igf-1 signaling is known to enhance intestinal epithelial regeneration. The colonization with the P. copri-containing consortium was found associated with markedly elevated expression of igf-1 in goblet cells and lymphatic endothelial cells—an interaction that propagates downstream to activate Igf-1 signal transduction in crypt stem cells.

The Compass algorithm was subsequently applied to our snRNA-Seq datasets to generate in silico predictions of the effects of the consortia containing and lacking P. copri on the metabolic states of (i) stem cell and proliferating TA cell clusters positioned in crypts of Lieberkühn, (ii) the three villus-associated enterocyte clusters, and (iii) the goblet cell cluster. Compass combines snRNA-seq data with the Recon2 database. This database describes 7,440 metabolic reactions grouped into 99 Recon2 subsystems, plus information about reaction stoichiometry, reaction reversibility, and associated enzyme(s). Using snRNA-seq data, Compass computes a score for each metabolic reaction. If the metabolic reaction was reversible, then one score as calculated for the “forward” reaction and another score was calculated for the “reverse” reaction. A ‘metabolic flux difference’ was calculated (see Methods) to quantify the difference in net flux for a given reaction (i.e., the forward and reverse activities) between the two treatment groups.

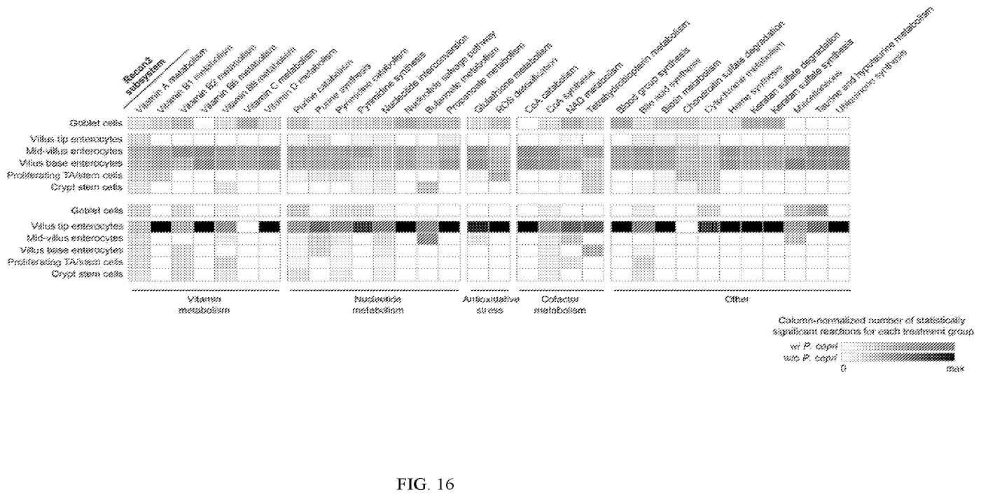

FIG. 15A-F shows the predicted metabolic flux differences for Recon2 reactions in enterocytes distributed along the length of the villus and in goblet cells. In clusters belonging to the enterocyte lineage, the number of statistically significant differences was greatest in villus base enterocytes and decreases towards the villus tip (FIG. 15A). Mice in the w/ P. copri treatment group had the greatest predicted increases (relative to their w/o P. copri counterparts) in activities of subsystems related to energy metabolism, the metabolism of carbohydrates, amino acids and fatty acids, as well as various transporters, in their villus base and mid-villus enterocytes (FIG. 15B, FIG. 16).

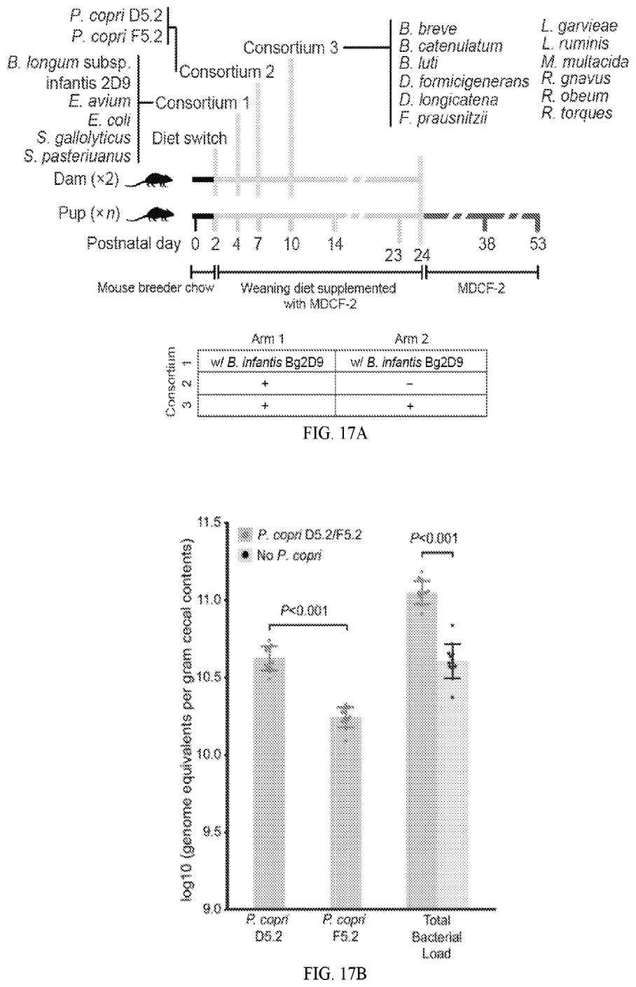

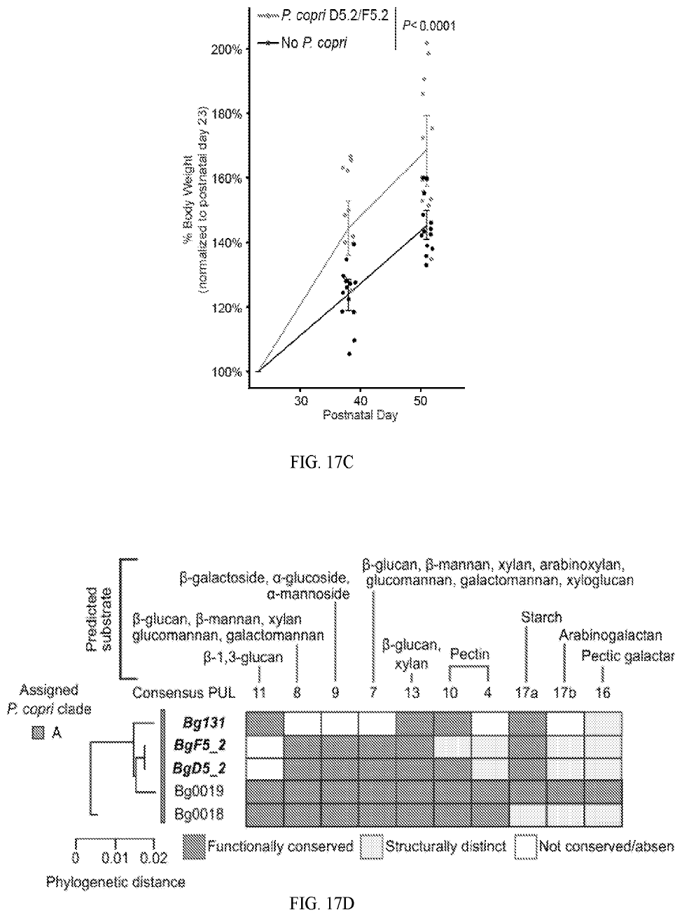

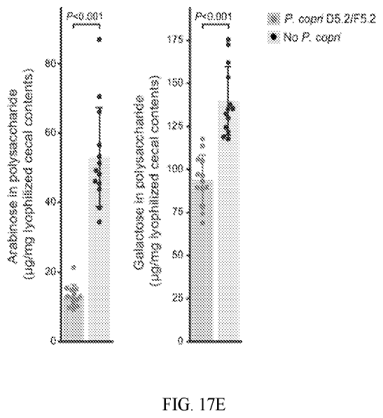

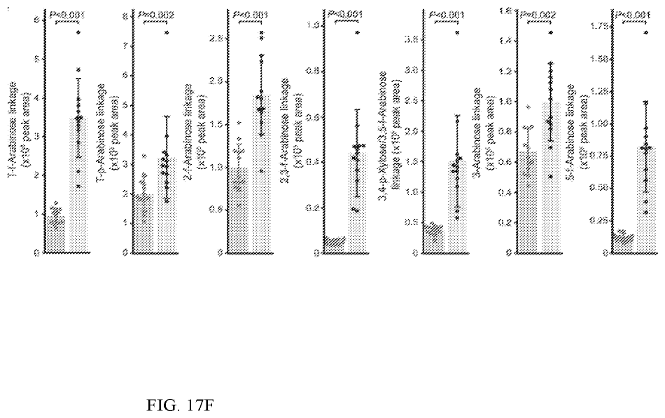

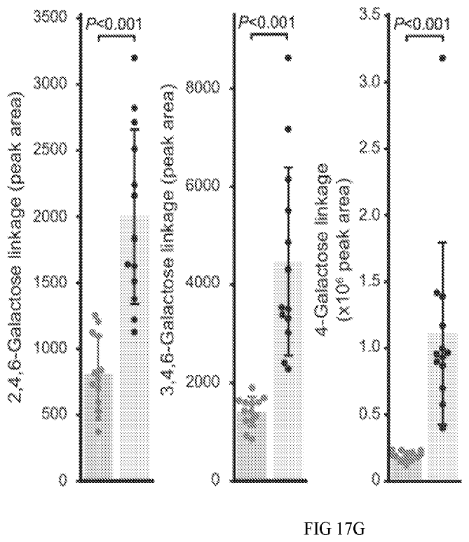

While enterocytes prioritize glutamine as their primary energy source, they were also able to utilize fatty acids and glucose. The Compass-defined increase in reactions related to fatty acid oxidation that occur in the villus enterocytes of mice in the w/ P. copri group extended to their crypts of Lieberkühn (FIG. 17B). Fatty acid oxidation has been linked to intestinal stem cell maintenance and regeneration. Mice colonized with P. copri exhibited ‘statistically credible increases’ in the proportional representation of crypt stem cells and proliferating TA/stem cells but not in their villus-associated enterocytic clusters (FIG. 17C; see Table 23 for results regarding all identified epithelial and mesenchymal cell clusters). [The term ‘statistically credible difference’ was defined by scCODA (see Methods)]. Compared to mice lacking P. copri, those colonized with this organism also had predicted increases in energy metabolism in their goblet cells, as judged by the activities of subsystems involved in glutamate (Glu) metabolism, the urea cycle, fatty acid oxidation and glycolysis (FIG. 17B).

Citrulline is generally poorly represented in human diets; as it is predominantly synthesized via the metabolism of glutamine in small intestinal enterocytes and transported into the circulation. Studies of various enteropathies and short bowel syndrome have demonstrated that citrulline is a quantitative biomarker of metabolically active enterocyte mass and its levels in plasma were indicative of the absorptive capacity of the small intestine. Citrulline was markedly lower in blood from children with severe acute malnutrition compared to levels found in healthy controls from the same community. Low plasma citrulline levels have also been reported in cohorts of children with environmental enteric dysfunction, with higher levels predictive of future weight gain.

Both glutamate and arginine were found important for citrulline production in enterocytes. Glutaminase (Gls) and glutamate dehydrogenase (GluD) in the glutamine pathway provided ammonia for generating carbamoyl phosphate (FIG. 17D). Arginine is a primary precursor for ornithine synthesis: ornithine transcarbamylase (Ots) produces citrulline from carbamoyl phosphate and ornithine. Compass predicted that mice harboring P. copri exhibited statistically significant increases in these reactions in their villus base and mid-villus enterocyte clusters [q<0.05 (adjusted P-value); Wilcoxon Ranked Sum test; FIG. 17D]. Targeted mass spectrometric analysis confirmed that citrulline was significantly increased in jejunal, ileal and colonic tissue segments, as well as in the plasma of mice harboring P. copri (P<0.05; Mann-Whitney U test; FIG. 17E).

The presence of P. copri was also associated with significantly greater predicted activities in Recon2 subsystems involved in transport of nine amino acids (including the essential amino acids leucine, isoleucine, valine, and phenylalanine), dipeptides and monosaccharides (glucose and galactose) in villus base and mid-villus enterocytes (FIG. 17F). This prediction suggested greater absorptive capacity for these important growth-promoting nutrients, which are known to be transported within the jejunum at the base and middle regions of villi.

### Example 11: Additional Assessment of Host Metabolic Effects Produced by P. Copri

To validate some of these Compass-based predictions, the experiment described above was repeated but with just two of its arms (“w/ P. copri” and “w/o P. copri”) and with a larger number of animals (4 dually housed germ-free dams yielding 18-19 viable pups per arm). The same cultured strains, the same sequence of their introduction and the same sequence of diet switches were applied (FIG. 17A). B. infantis strain Bg2D9 was utilized in both arms. Reproducible colonization of consortium members within each arm was confirmed by quantifying their absolute abundances in cecal samples collected at the time of euthanasia (P53; see Table 24). Consistent with the previous experiment, animals in the w/ P. copri arm exhibited significantly greater weight gain between P23 and P53 [P<0.05; linear mixed-effects model (see Methods)] (FIG. 17B).

TABLE 24
Absolute abundances of bacterial strains in dam-pup dyads
colonized with cultured bacterial consortia in the
validation experiment, sample metadata

| | | | | Number | |
|---|---|---|---|---|---|
| Treat- | | Mouse | | ofraw | |
| ment | Sex | ID | Sample ID | reads | Index |
| W/ | male | 1 | defined_community_validation_experiment. | 1,278,473 | TTCTTC |
| P. | | | with_P_copri_colonization.postnatal_day_53. | | TAAACG |
| copri | | | pup_1.male.cecal_contents.CoProSeq | | ATGC |
| | male | 2 | defined_community_validation_experiment. | 1,276,581 | TACCTG |
| | | | with_P_copri_colonization.postnatal_day_53. | | ACAACG |
| | | | pup_2.male.cecal_contents.CoProSeq | | ATGC |
| | male | 3 | defined_community_validation_experiment. | 1,228,696 | AGGACC |
| | | | with_P_copri_colonization.postnatal_day_53. | | GCAACG |
| | | | pup_3.male.cecal_contents.CoProSeq | | ATGC |
| | male | 4 | defined_community_validation_experiment. | 1,232,782 | GTCCGA |
| | | | with_P_copri_colonization.postnatal_day_53. | | TTAACG |
| | | | pup_4.male.cecal contents.CoProSeq | | ATGC |
| | male | 5 | defined_community_validation_experiment. | 1,255,315 | CACGAG |
| | | | with_P_copri_colonization.postnatal_day_53. | | TTAACG |
| | | | pup_5.male.cecal_contents.CoProSeq | | ATGC |
| | male | 6 | defined_community_validation_experiment. | 1,352,032 | CCACGG |
| | | | with_P_copri_colonization.postnatal_day_53. | | CCAACG |
| | | | pup_6.male.cecal_contents.CoProSeq | | ATGC |
| | male | 7 | defined_community_validation_experiment. | 1,321,478 | ACATGT |
| | | | with_P_copri_colonization.postnatal_day_53. | | AAAACG |
| | | | pup_7.male.cecal_contents.CoProSeq | | ATGC |
| | male | 8 | defined_community_validation_experiment. | 1,206,927 | TGTTAA |
| | | | with_P_copri_colonization.postnatal_day_53. | | CTAACG |
| | | | pup_8.male.cecal_contents.CoProSeq | | ATGC |
| | female | 1 | defined_community_validation_experiment. | 1,352,319 | TTCTTC |
| | | | with_P_copri_colonization.postnatal_day_53. | | TAGTCA |
| | | | pup_1.female.cecal_contents.CoProSeq | | ACCT |
| | female | 2 | defined_community_validation_experiment. | 2,021,888 | TACCTG |
| | | | with_P_copri_colonization.postnatal_day_53. | | ACGTCA |
| | | | pup_2.female.cecal_contents.CoProSeq | | ACCT |
| | female | 3 | defined_community_validation_experiment. | 1,277,942 | AGGACC |
| | | | with_P_copri_colonization.postnatal_day_53. | | GCGTCA |
| | | | pup_3.female.cecal_contents.CoProSeq | | ACCT |
| | female | 4 | defined_community_validation_experiment. | 1,268,384 | GTCCGA |
| | | | with_P_copri_colonization.postnatal_day_53. | | TTGTCA |
| | | | pup_4.female.cecal_contents.CoProSeq | | ACCT |
| | female | 6 | defined_community_validation_experiment. | 1,288,500 | CACGAG |
| | | | with_P_copri_colonization.postnatal_day_53. | | TTGTCA |
| | | | pup_6.female.cecal_contents.CoProSeq | | ACCT |
| | female | 7 | defined_community_validation_experiment. | 1,362,292 | CCACGG |
| | | | with_P_copri_colonization.postnatal_day_53. | | CCGTCA |
| | | | pup_7.female.cecal_contents.CoProSeq | | ACCT |
| | female | 9 | defined_community_validation_experiment. | 1,378,099 | ACATGT |
| | | | with_P_copri_colonization.postnatal_day_53. | | AAGTCA |
| | | | pup_9.female.cecal_contents.CoProSeq | | ACCT |
| | female | 10 | defined_community_validation_experiment. | 1,263,105 | TGTTAA |
| | | | with_P_copri_colonization.postnatal_day_53. | | CTGTCA |
| | | | pup_10.female.cecal_contents.CoProSeq | | ACCT |
| | female | 11 | defined_community_validation_experiment. | 1,262,012 | TTCTTC |
| | | | with_P_copri_colonization.postnatal_day_53. | | TACAGT |
| | | | pup_11.female.cecal_contents.CoProSeq | | TTCA |
| | female | 12 | defined_community_validation_experiment. | 1,270,396 | TACCTG |
| | | | with_P_copri_colonization.postnatal_day_53. | | ACCAGT |
| | | | pup_12.female.cecal_contents.CoProSeq | | TTCA |
| w/o | male | 1 | defined_community_validation_experiment. | 1,231,886 | AGGACC |
| P. | | | without_P_copri_colonization.postnatal_ | | GCCAGT |
| copri | | | day_53.pup_1.male.cecal_contents.CoProSeq | | TTCA |
| | male | 2 | defined_community_validation_experiment. | 1,228,042 | GTCCGA |
| | | | without_P_copri_colonization.postnatal_ | | TTCAGT |
| | | | day_53.pup_2.male.cecal_contents.CoProSeq | | TTCA |
| | male | 3 | defined_community_validation_experiment. | 1,229,059 | CACGAG |
| | | | without_P_copri_colonization.postnatal_ | | TTCAGT |
| | | | day_53.pup_3.male.cecal_contents.CoProSeq | | TTCA |
| | male | 4 | defined_community_validation_experiment. | 1,331,457 | CCACGG |
| | | | without_P_copri_colonization.postnatal_ | | CCCAGT |
| | | | day_53.pup_4.male.cecal_contents.CoProSeq | | TTCA |
| | male | 5 | defined_community_validation_experiment. | 1,309,396 | ACATGT |
| | | | without_P_copri_colonization.postnatal_ | | AACAGT |
| | | | day_53.pup_5.male.cecal_contents.CoProSeq | | TTCA |
| | male | 6 | defined_community_validation_experiment. | 1,262,213 | TGTTAA |
| | | | without_P_copri_colonization.postnatal_ | | CTCAGT |
| | | | day_53.pup_6.male.cecal_contents.CoProSeq | | TTCA |
| | male | 7 | defined_community_validation_experiment. | 1,243,030 | TTCTTC |
| | | | without_P_copri_colonization.postnatal_ | | TATGTG |
| | | | day_53.pup_7.male.cecal_contents.CoProSeq | | ATTG |
| | male | 8 | defined_community_validation_experiment. | 1,215,656 | TACCTG |
| | | | without_P_copri_colonization.postnatal_ | | ACTGTG |
| | | | day_53.pup_8.male.cecal_contents.CoProSeq | | ATTG |
| | male | 10 | defined_community_validation_experiment. | 1,305,951 | AGGACC |
| | | | without_P_copri_colonization.postnatal_ | | GCTGTG |
| | | | day_53.pup_10.male.cecal_contents.CoProSeq | | ATTG |
| | female | 1 | defined_community_validation_experiment. | 1,208,348 | GTCCGA |
| | | | without_P_copri_colonization.postnatal_ | | TTTGTG |
| | | | day_53.pup_1.female.cecal_contents.CoProSeq | | ATTG |
| | female | 2 | defined_community_validation_experiment. | 1,250,295 | CACGAG |
| | | | without_P_copri_colonization.postnatal_ | | TTTGTG |
| | | | day_53.pup_2.female.cecal_contents.CoProSeq | | ATTG |
| | female | 3 | defined_community_validation_experiment. | 1,307,996 | CCACGG |
| | | | without_P_copri_colonization.postnatal_ | | CCTGTG |
| | | | day_53.pup_3.female.cecal_contents.CoProSeq | | ATTG |
| | female | 4 | defined_community_validation_experiment. | 1,285,516 | ACATGT |
| | | | without_P_copri_colonization.postnatal_ | | AATGTG |
| | | | day_53.pup_4.female.cecal_contents.CoProSeq | | ATTG |
| | female | 5 | defined_community_validation_experiment. | 1,310,300 | TGTTAA |
| | | | without_P_copri_colonization.postnatal_ | | CTTGTG |
| | | | day_53.pup_5.female.cecal_contents.CoProSeq | | ATTG |
| | female | 6 | defined_community_validation_experiment. | 1,355,158 | TTCTTC |
| | | | without_P_copri_colonization.postnatal_ | | TATTGC |
| | | | day_53.pup_6.female.cecal_contents.CoProSeq | | ATGT |
| | female | 7 | defined_community_validation_experiment. | 1,311,870 | TACCTG |
| | | | without_P_copri_colonization.postnatal_ | | ACTTGC |
| | | | day_53.pup_7.female.cecal_contents.CoProSeq | | ATGT |
| | female | 8 | defined_community_validation_experiment. | 1,254,066 | AGGACC |
| | | | without_P_copri_colonization.postnatal_ | | GCTTGC |
| | | | day_53.pup_8.female.cecal_contents.CoProSeq | | ATGT |
| | female | 9 | defined_community_validation_experiment. | 1,248,274 | GTCCGA |
| | | | without_P_copri_colonization.postnatal_ | | TTTTGC |
| | | | day_53.pup_9.female.cecal_contents.CoProSeq | | ATGT |
| | female | 10 | defined_community_validation_experiment. | 1,309,630 | CACGAG |
| | | | without_P_copri_colonization.postnatal_ | | TTTTGC |
| | | | day_53.pup_10.female.cecal_contents.CoProSeq | | ATGT |

Mass spectrometric analysis of host metabolism—Targeted mass spectrometry was used to quantify levels of 20 amino acids, 19 biogenic amines, and 66 acylcarnitines in the jejunum, colon, gastrocnemius, quadriceps, heart muscle, and liver of the two groups of mice. Additionally, the 66 acylcarnitines were quantified in their plasma (FIG. 15C-E). Consistent with the previous experiment, citrulline, the biomarker for metabolically active enterocyte biomass, was significantly elevated in the jejunums of mice belonging to the w/ P. copri group (P<0.05; Mann-Whitney U test) (FIG. 15C).

Significant elevations of acylcarnitines derived from palmitic acid (C16:0), stearic acid (C18:0), oleic acid (C18:1), linoleic acid (C18:2), and linolenic acid (C18:3) were observed in the jejunums of P. copri-colonized animals (P<0.01; Mann-Whitney U test) (FIG. 15D); and which are the major fatty acids found in soybean oil, a principal source of lipids in MDCF-2. These acylcarnitine chain lengths, were found at higher abundance than all other medium or long-chain acylcarnitine species in the samples, indicating their role as primary dietary lipid energy sources. Elevation of these species suggested an increased transport and ß-oxidation of long-chain dietary lipids in the jejunum.

Analysis of colonic tissue showed significant elevation of C16:0, C18:1, and C18:2 acylcarnitines in P. copri-colonized animals, suggesting that β-oxidation was also elevated in tissue compartments not directly involved in lipid absorption (P<0.01; Mann-Whitney U test) (FIG. 15E). This finding was matched by a significant elevation in plasma levels of non-esterified fatty acids in w/ P. copri animals, suggesting higher circulation of dietary lipids which would support fatty acid β-oxidation in peripheral tissues (P<0.05; Mann-Whitney U test) (FIG. 15F; Table 25). Targeted LC-MS was further conducted in liver, gastrocnemius muscle, quadriceps and heart. The statistically significant difference in levels of acylcarnitines whose chain length corresponded to components of soybean oil was an increase in 18:2 and 18:3 species in the myocardium of w/ P. copri compared to w/o P. copri animals. Additionally, jejunal levels of C3 and C4 acylcarnitines as well as colonic levels of C4 and C5 acylcarnitines known to be derived from branched-chain amino acid catabolism were significantly elevated in the P. copri-colonized animals (P<0.05; Mann-Whitney U test; FIG. 15D, 15E). Together, these results suggested that the presence of P. copri induced differential fuel utilization via fatty acid b-oxidation at sites involved in dietary nutrient absorption.

TABLE 25
Non-esterfied fatty acids in plasma (NEFA; mmol/L)

| Treatment | Sex | Mouse no. | NEFA |
|---|---|---|---|
| w/P. copri | male | 1 | 0.4 |
| | male | 2 | 0.6 |
| | male | 3 | 0.5 |
| | male | 4 | 0.6 |
| | male | 5 | 0.4 |
| | male | 6 | 0.3 |
| | male | 7 | 0.4 |
| | male | 8 | 0.5 |
| | female | 1 | 0.6 |
| | female | 2 | 0.5 |
| | female | 3 | 0.8 |
| | female | 4 | 0.4 |
| | female | 6 | 0.5 |
| | female | 7 | 0.8 |
| | female | 9 | 0.9 |
| | female | 10 | 0.9 |
| | female | 11 | 0.9 |
| | female | 12 | 0.7 |
| | mean ± SD | | 0.6 ± 0.2 |
| w/o P. copri | male | 1 | 0.4 |
| | male | 2 | 0.4 |
| | male | 3 | 0.7 |
| | male | 4 | 0.3 |
| | male | 5 | 0.7 |
| | male | 6 | 0.3 |
| | male | 7 | 0.3 |
| | male | 8 | 0.2 |
| | male | 10 | 0.3 |
| | female | 1 | 0.6 |
| | female | 2 | 0.5 |
| | female | 3 | 0.4 |
| | female | 4 | 0.5 |
| | female | 5 | 0.4 |
| | female | 6 | 0.8 |
| | female | 7 | 0.6 |
| | female | 8 | 0.6 |
| | female | 9 | 0.5 |
| | female | 10 | 0.4 |

| | |
|---|---|
| mean ± SD | 0.5 ± 0.2 |

### Example 11: Evaluation of the Effects of Preweaning P. Copri Colonization

To directly determine whether pre-weaning colonization with P. copri strains resembling MAGs Bg0018 and Bg0019 is sufficient to promote growth and produce the metabolic effects described above, an additional experiment was performed. The design was similar to that described above but there were several important modifications. First, P. stercorea was not included in the second gavage mixture; it only contained P. copri. Second, two strains of P. copri (D5.2 and F5.2) were used, cultured from fecal samples from Bangladeshi children, that displayed greater similarity to Bg0018 and Bg0019 than the PS131 strains quantified by ANI and their content of PULs and mcSEED metabolic pathways. Both P. copri D5.2 and F5.2 shared 102/106 (96%) metabolic pathway completeness annotations with MAG Bg0018 and 101/106 (95%) annotations with MAG Bg0019. Similarly, 9 of the 10 functionally conserved PULs shared by MAGs Bg0018 and Bg0019 were conserved in P. copri D5.2 and F5.2. Third, the fourth gavage was omitted, previously administered at the end of the weaning period, that had included P. copri and P. stercorea. The control group of animals did not receive P. copri (n=2 dams and 13 pups/treatment group).

Shotgun sequencing of DNA isolated from cecal contents collected at the time of euthanasia (P53) confirmed that animals in the experimental group had been colonized with both P. copri isolates as well as all other members of the defined consortia. In animals colonized with both isolates, P. copri D5.2 was present at significantly higher absolute abundance than the F5.2 strain (FIG. 17B); their relative abundances were 37.8±4.4% and 15.5±1.0%, respectively, compared with 31±6.6% and 24±8.0% for P. copri PS131 in the first and second experiments. Colonization of all administered strains was confirmed in the control group. Comparing the experimental and control groups disclosed that carriage of these two isolates was associated with a significantly greater total bacterial load, indicating that their colonization augmented community biomass without displacing other bacteria (FIG. 17B).

A significantly greater increase in body weight was observed between P23 and P53 in mice colonized with P. copri D5.2 and F5.2 compared to those without P. copri [P<0.0001; linear mixed-effects model] (FIG. 17C). The difference in the mean percent increase in postweaning weight between the experimental and control groups in this experiment (24%) was comparable to that document in the two previous experiments (25% in the first and 13% in the second); as in these previous experiments, the weight difference was not attributable to differences in cecal size.

Mass spectrometry confirmed that preweaning colonization with P copri affected intestinal lipid metabolism and was a major determinant of MDCF-2 glycan degradation. Targeted LC-MS of ileal and colonic tissue revealed significant elevation of long-chain acylcarnitines corresponding to soybean oil lipids, consistent with changes observed in the prior experiment (FIG. 15e).

A comparison of the two isolates used in this experiment against our previously used isolate, P. copri PS131, and the 10 functionally conserved PULs of MAGs Bg0018 and Bg0019 disclosed that these isolates contain PULs conserved between Bg0018 and Bg0019 involved in the degradation of substrates including galactose and mannose containing glycans (FIG. 17D). UHPLC-QqQ-MS-based measurement of total monosaccharides in cecal contents indicated that the presence of these two more MAG Bg0018- and Bg0019-like strains resulted in significantly lower levels of arabinose, consistent with previous observations using P. copri PS131, as well as galactose, a finding that was specific to this experiment (FIG. 17E). We simultaneously observed that P. copri D5.2 and F5.2 colonization significantly lower levels of all arabinose-containing linkages measured, as well as three galactose-containing linkages (FIGS. 17F and G). Together, these data suggest that the different PUL content of these new isolates leads to enhanced degradation of dietary glycans by the microbial community.

Targeted UHPLC-QqQ-MS-based measurement of glycosidic linkages in cecal contents indicated that the presence of these two more MAG Bg0018- and Bg0019-like strains resulted in X effects (FIG. 17D). Targeted UPHLC-QqQ-MS measurements of all 20 amino acids and seven B-vitamins revealed that P. copri colonization was associated with significantly higher cecal levels of two essential amino acids (tryptophan, lysine), and seven non-essential amino acids (glutamate, glutamine, aspartate, asparagine, arginine, proline, glycine) and higher levels of pantothenic acid (B5).

It was concluded that pre-weaning colonization with P. copri augments weight gain in the context of the MDCF-2 diet, that the organism is a major determinant/effector of MDCF-2 glycan degradation, and that its presence in the community produces substantial changes in intestinal tissue fatty acid metabolism.

### Example 12: Summary of Results from Examples 7-11

In the disclosed examples, a ‘reverse translation’ strategy was illustrated that can be used to address the mechanisms by which microbiome-targeted nutritional interventions impact the operations of microbial community members and how these changes can alter human physiology at a molecular, cellular and systems level. Gnotobiotic mice were colonized with defined consortia of age- and WLZ-associated bacterial strains cultured from the study population. Dam-to-pup transmission of these communities occurred in the context of a sequence of diets that re-enacted those consumed by children in the clinical study. Microbial RNA-Seq and targeted mass spectrometry of glycosidic linkages present in intestinal contents provided evidence that Prevotella copri, represented by an isolate similar to MAGs identified as WLZ-associated in the clinical trial, was crucial to the metabolism of polysaccharides contained in MDCF-2. snRNA-Seq and targeted mass spectrometry indicated that P. copri increased the uptake and metabolism of lipids, including those fatty acids that are most prominently represented in the soybean oil that comprises the principal lipid component of MDCF-2. Additional effects on uptake and metabolism of amino acids (including essential amino acids) and monosaccharides were predicted. The effects on nutrient processing and energy metabolism involved proliferating epithelial progenitors in the crypts as well as their descendant lineages distributed along the villus. snRNA-Seq revealed discrete spatial features of these effects, with populations of enterocytes positioned at the base-, mid- and tip regions of villi manifesting distinct patterns of differential expression of a number of metabolic functions.

In summary, the above-described examples illustrated an approach for identifying members of a gut microbial community that function as principal metabolizers of MDCF components as well as key effectors of host biological responses. Characterizing their genomic features and expression, can be used for developing microbiome-based diagnostics for stratification of populations of undernourished children who are candidates for treatment with a given MDCF, and for monitoring their treatment responses, including in adaptive clinical trial designs. Further, a knowledge base needed is provided for (i) creation of ‘next generation’ MDCFs composed of (already) identified bioactive components, but from alternative food staples which are more readily available, affordable and culturally acceptable for populations living in different geographic locales; (ii) more informed decisions about the dose of an MDCF for undernourished children as a function of their stage of development and disease severity, and (iii) evolving policies about complementary feeding practices that build upon traditional macro- and micro-nutrient-centric considerations, but now add insights about how food components impact the fitness and expressed beneficial functions of growth-promoting elements of a child's microbiome. Finally, the recovered growth-promoting strains can be used as next-generation probiotics, and/or as components of synbiotics for repairing gut microbial communities that cannot be resuscitated with food-based interventions alone.

### Example 13: Effects of MDCF-2 as Provided in Examples 1-6 Persist Beyond Cessation of the 3-Month Intervention

In order to study if the effect of intervention with administration of MDCF-2 as provided in Examples 1-6, last beyond the 3-month period of intervention, weight-for-length z-score (WLZ), length-for-age z-score (LAZ), weight-for-age z-score (WAZ) between the MDCF-2 and RUSF groups at different time points up to 2 years after cessation of the 3-month intervention in 12-18 month children with primary MAM was calculated. Table 26 provides the baseline characteristics of the children in the primary MAM study.

TABLE 26
Baseline characteristics of children in the primary MAM study

| Variables | MDCF-2 (n = 59) | | RUSF (n = 59) | |
|---|---|---|---|---|
| Age (in months), mean (sd) | 15.37 | (1.91) | 15.61 | (1.93) |
| Child's gender, Male, % (n) | 43.6 | (27) | 41.9 | (26) |
| Child's weight (in kg), mean (sd) | 8.10 | (0.72) | 7.99 | (0.64) |
| Child's height (in cm), mean (sd) | 75.99 | (3.43) | 76.31 | (3.21) |
| Child's MUAC (in cm) | 13.18 | (0.48) | 13.07 | (0.46) |

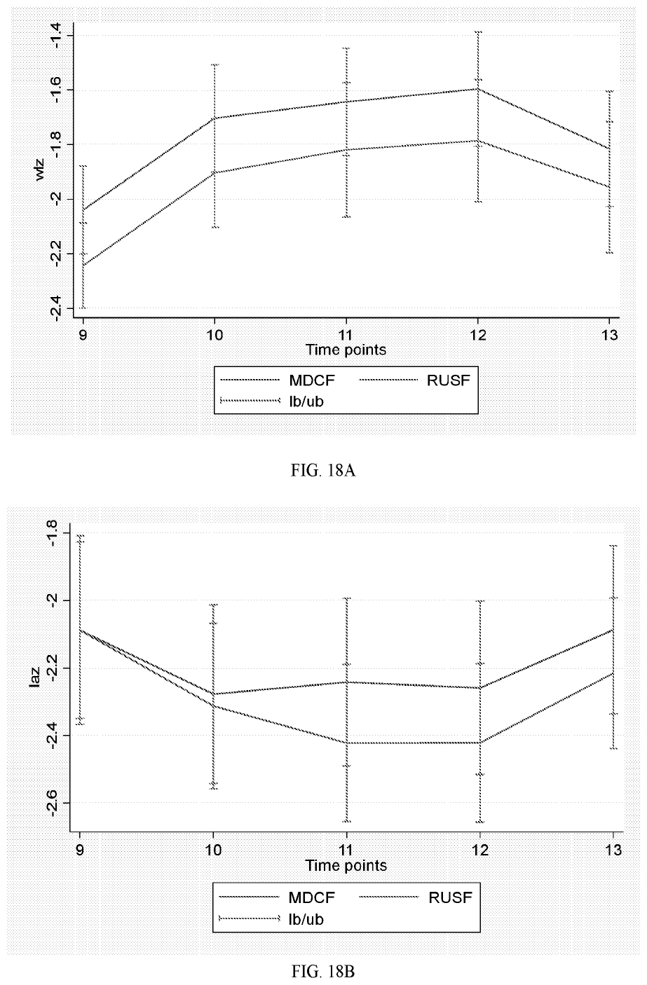

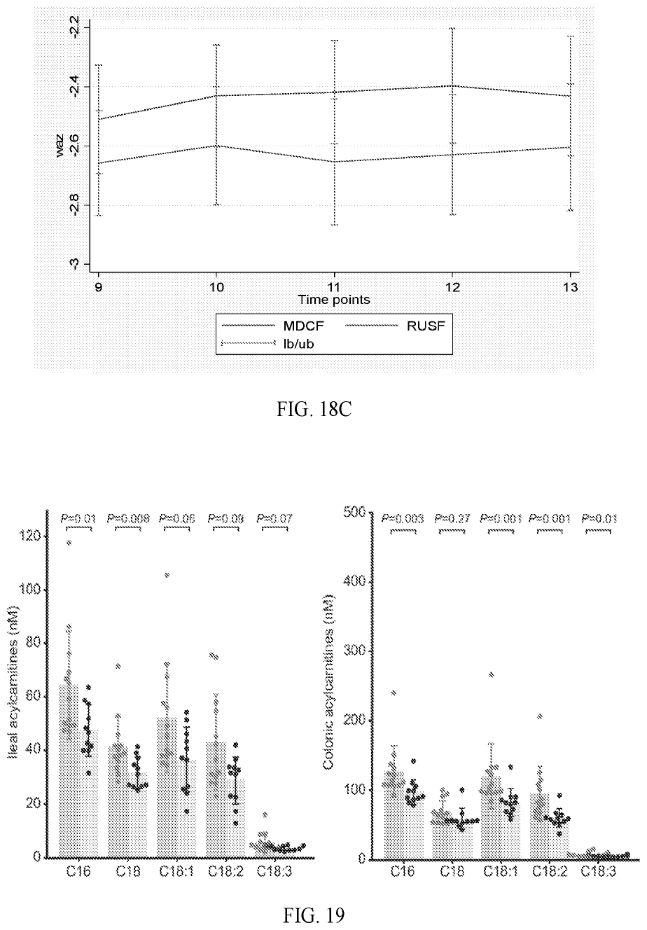

FIGS. 18A-C provide data for the WLZ, LAZ and WAZ comparison corresponding to: 9=One-month follow-up after cessation of intervention; 10=Six-month follow-up after cessation of intervention; 11=12-month follow-up after cessation of intervention; 12=18-month follow-up after cessation of intervention; and 13=24-month follow-up after cessation of intervention.

Table 27 provides a compilation of the results. Mixed effect multiple linear model adjusted for baseline anthropometry (WLZ, LAZ, WAZ score), interventions (RUSF or MDCF-2) child age (in days, continuous), child gender (male and female) and child past seven days morbidity status (yes, no) were developed.

TABLE 27
Results

| | | | |
|---|---|---|---|
| Indicators | Coefficient | 95% CI | p-value |

Weight for-length z-score (WLZ)

| | | | |
|---|---|---|---|
| Types of intervention | | | |
| RUSF (Ref.) | | | |
| MDCF-2 | −.024 | −0.205, 0.158 | 0.799 |
| Length for age z-score (LAZ) | | | |
| Types of intervention | | | |
| RUSF (Ref.) | | | |
| MDCF-2 | 0.1113 | 0.004, 0.222 | 0.042 |
| Weight for age z-score (WAZ) | | | |
| Types of intervention | | | |
| RUSF (Ref.) | | | |
| MDCF-2 | .0444 | −.089, 0.177 | 0.511 |

Results show that the effect of the intervention persist beyond cessation of the 3-month intervention and lead to a delayed but lasting improvement in stunting (up to 2 years after cessation of treatment—FIGS. 18A-C and Table 27). This is a significant finding as there are few if any reported treatments that affect linear growth (LAZ) of stunted children in this way and thus expands the benefits of the MDFIP. copri combination beyond solely ponderal growth (weight gain).

TABLE 28

| | | Genome Type | | | | | | | |
|---|---|---|---|---|---|---|---|---|---|
| | | Isolate | | | | | | MAG | |
| | | Strain ID or MAG ID | | | | | | | |
| | | BgD5_2 | BgF5_2 | BgG5_1 | Bg2C6 | Bg2H3 | Bg131 | MAGBg0019 | MAGBg0018 |
| | | Genus | | | | | | | |
| | | Prevotella | Prevotella | Prevotella | Prevotella | Prevotella | Prevotella | Prevotella | Prevotella |
| | | Species | | | | | | | |
| | | copri | copri | copri | copri | copri | copri | copri | copri |
| Functional | All | 0 | 0 | 0 | 0 | 0 | 0 | 0 | 0 |
| pathways^{b} | (Allose) | | | | | | | | |
| | aAOS | 1 | 1 | 1 | 1 | 1 | 1 | 1 | 1 |
| | Aga | 0 | 0 | 0 | 0 | 0 | 0 | 0 | 0 |
| | Atl | 0 | 0 | 0 | 0 | 0 | 0 | 0 | 0 |
| | Ara | 1 | 1 | 1 | 1 | 1 | 1 | 0 | 0 |
| | bAOS | 0 | 0 | 0 | 0 | 0 | 0 | 0 | 0 |
| | GOS | 1 | 1 | 1 | 1 | 1 | 1 | 1 | 1 |
| | Bgl | 0 | 0 | 0 | 0 | 1 | 1 | 1 | 1 |
| | Chb | 0 | 0 | 0 | 0 | 0 | 0 | 0 | 0 |
| | FOS | 1 | 1 | 1 | 1 | 1 | 1 | 1 | 1 |
| | Fru | 1 | 1 | 1 | 1 | 1 | 1 | 1 | 1 |
| | FruAsn | 0 | 0 | 0 | 0 | 0 | 0 | 0 | 0 |
| | FruLys | 0 | 0 | 0 | 0 | 0 | 0 | 0 | 0 |
| | Fuc | 0 | 0 | 1 | 0 | 0 | 0 | 0 | 0 |
| | Fcs | 0 | 0 | 1 | 0 | 0 | 0 | 0 | 0 |
| | Gal | 1 | 1 | 1 | 1 | 1 | 1 | 1 | 1 |
| | GalA | 1 | 1 | 0 | 1 | 1 | 1 | 1 | 1 |
| | GalAs | 1 | 1 | 0 | 1 | 1 | 1 | 1 | 1 |
| | GalN | 0 | 0 | 0 | 0 | 0 | 0 | 0 | 0 |
| | GalNAc | 0 | 0 | 0 | 0 | 0 | 0 | 0 | 0 |
| | Gnt | 0 | 0 | 0 | 0 | 0 | 0 | 0 | 0 |
| | Glc | 1 | 1 | 1 | 1 | 1 | 1 | 1 | 1 |
| | GlcA | 1 | 1 | 0 | 1 | 1 | 1 | 1 | 1 |
| | GlcAs | 0 | 0 | 0 | 0 | 0 | 0 | 0 | 0 |
| | GlcLys | 0 | 0 | 0 | 0 | 0 | 0 | 0 | 0 |
| | GlcNAc | 0 | 0 | 0 | 0 | 0 | 0 | 0 | 0 |
| | ddGlcA | 0 | 0 | 0 | 0 | 0 | 0 | 0 | 0 |
| | Gtl | 0 | 0 | 0 | 0 | 0 | 0 | 0 | 0 |
| | Hyl | 0 | 0 | 0 | 0 | 0 | 0 | 0 | 0 |
| | Ino | 0 | 0 | 0 | 0 | 0 | 0 | 0 | 0 |
| | Lnb | 0 | 0 | 0 | 0 | 0 | 0 | 0 | 0 |
| | Lac | 1 | 1 | 1 | 1 | 1 | 1 | 1 | 1 |
| | MOS | 1 | 1 | 1 | 1 | 1 | 1 | 1 | 1 |
| | Mal | 1 | 1 | 1 | 1 | 1 | 1 | 1 | 1 |
| | Mtl | 0 | 0 | 0 | 0 | 1 | 0 | 0 | 1 |
| | Man | 0 | 0 | 0 | 0 | 0 | 0 | 0 | 0 |
| | bMnOS | 1 | 1 | 1 | 1 | 1 | 1 | 1 | 1 |
| | Mel | 0 | 0 | 0 | 0 | 0 | 0 | 0 | 0 |
| | ManNAc | 0 | 0 | 0 | 0 | 0 | 0 | 0 | 0 |
| | MurNac | 0 | 0 | 0 | 0 | 0 | 0 | 0 | 0 |
| | NANA | 0 | 0 | 1 | 1 | 0 | 1 | 0 | 0 |
| | PsiLys | 0 | 0 | 0 | 0 | 0 | 0 | 0 | 0 |
| | Raf | 0 | 0 | 0 | 0 | 0 | 0 | 0 | 0 |
| | Rhi | 0 | 0 | 0 | 1 | 0 | 1 | 0 | 0 |
| | Rha | 0 | 0 | 0 | 1 | 0 | 1 | 0 | 0 |
| | Rtl | 0 | 0 | 0 | 0 | 0 | 0 | 0 | 0 |
| | Rbs | 0 | 0 | 0 | 0 | 0 | 0 | 0 | 0 |
| | Srl | 0 | 0 | 0 | 0 | 1 | 0 | 0 | 0 |
| | Scr | 1 | 1 | 1 | 1 | 1 | 1 | 1 | 1 |
| | Tag | 0 | 0 | 0 | 0 | 0 | 0 | 0 | 0 |
| | Tre | 0 | 0 | 0 | 0 | 0 | 0 | 0 | 0 |
| | Xlt | 0 | 0 | 0 | 0 | 0 | 0 | 0 | 0 |
| | XOS | 1 | 1 | 1 | 1 | 1 | 1 | 1 | 1 |
| | aXyl | 1 | 1 | 1 | 1 | 1 | 1 | 1 | 1 |
| | Xyl | 1 | 1 | 1 | 1 | 1 | 1 | 1 | 1 |
| | EA_ut | 0 | 0 | 0 | 0 | 0 | 0 | 0 | 0 |
| | But_ut | 0 | 0 | 0 | 0 | 0 | 0 | 0 | 0 |
| | PD_ut | 0 | 0 | 0 | 0 | 0 | 0 | 0 | 0 |
| | Lac_ut | 1 | 1 | 1 | 1 | 1 | 1 | 0 | 1 |
| | CA_d | 0 | 0 | 0 | 0 | 0 | 0 | 0 | 0 |
| | BA_t | 0 | 0 | 0 | 0 | 0 | 0 | 0 | 0 |
| | Urea_d | 0 | 0 | 0 | 0 | 0 | 0 | 0 | 0 |
| | Pro_d | 0 | 0 | 0 | 0 | 0 | 0 | 0 | 0 |
| | Thr_d | 0 | 0 | 0 | 0 | 0 | 0 | 0 | 0 |
| | Met_d | 0 | 0 | 0 | 0 | 0 | 0 | 0 | 0 |
| | His_d | 0 | 0 | 0 | 0 | 0 | 0 | 0 | 0 |
| | Lys_d | 0 | 0 | 0 | 0 | 0 | 0 | 0 | 0 |
| | Trp_d | 0 | 0 | 0 | 0 | 0 | 0 | 0 | 0 |
| | B1 | 1 | 1 | 1 | 1 | 1 | 1 | 1 | 1 |
| | B2 | 1 | 1 | 1 | 1 | 1 | 1 | 1 | 1 |
| | B3 | 1 | 1 | 1 | 1 | 1 | 1 | 1 | 1 |
| | B5 | 1 | 1 | 1 | 1 | 1 | 1 | 1 | 1 |
| | B6 | 1 | 1 | 1 | 1 | 1 | 1 | 0 | 1 |
| | B7 | 0 | 0 | 0 | 0 | 0 | 0 | 0 | 0 |
| | B9 | 1 | 1 | 1 | 1 | 1 | 1 | 1 | 1 |
| | B12 | 0 | 0 | 0 | 0 | 0 | 0 | 0 | 0 |
| | Q | 0 | 0 | 1 | 0 | 0 | 0 | 0 | 0 |
| | LA | 0 | 0 | 0 | 0 | 0 | 0 | 0 | 0 |
| | MQ | 1 | 1 | 1 | 1 | 1 | 1 | 1 | 1 |
| | Arg | 1 | 1 | 1 | 1 | 1 | 1 | 1 | 1 |
| | Lys | 1 | 1 | 1 | 1 | 1 | 1 | 1 | 1 |
| | His | 1 | 1 | 1 | 1 | 1 | 1 | 1 | 1 |
| | Trp | 1 | 1 | 1 | 1 | 1 | 1 | 1 | 1 |
| | Tyr | 1 | 1 | 1 | 1 | 1 | 1 | 1 | 1 |
| | Phe | 1 | 1 | 1 | 1 | 1 | 1 | 1 | 1 |
| | Chor | 1 | 1 | 1 | 1 | 1 | 1 | 1 | 1 |
| | Ile | 1 | 1 | 1 | 1 | 1 | 1 | 1 | 1 |
| | Val | 1 | 1 | 1 | 1 | 1 | 1 | 1 | 1 |
| | Leu | 1 | 1 | 1 | 1 | 1 | 1 | 1 | 1 |
| | Ser | 1 | 1 | 1 | 1 | 1 | 1 | 1 | 1 |
| | Cys | 1 | 1 | 1 | 1 | 1 | 1 | 1 | 1 |
| | Thr | 1 | 1 | 1 | 1 | 1 | 1 | 0 | 0 |
| | Met | 1 | 1 | 1 | 1 | 1 | 1 | 1 | 1 |
| | Pro | 1 | 1 | 1 | 1 | 1 | 1 | 1 | 1 |
| | Gly | 1 | 1 | 1 | 1 | 1 | 1 | 1 | 1 |
| | Glu | 1 | 1 | 1 | 1 | 1 | 1 | 1 | 1 |
| | Gln | 1 | 1 | 1 | 1 | 1 | 1 | 1 | 1 |
| | Asp | 1 | 1 | 1 | 1 | 1 | 1 | 1 | 1 |
| | Asn | 1 | 1 | 1 | 1 | 1 | 1 | 1 | 1 |
| | Butyrate | 0 | 0 | 0 | 0 | 0 | 0 | 0 | 0 |
| | Propionate | 0 | 0 | 0 | 0 | 0 | 0 | 0 | 0 |
| | L-Lactate | 0 | 0 | 0 | 0 | 0 | 0 | 0 | 0 |
| | D-Lactate | 0 | 0 | 0 | 0 | 0 | 0 | 0 | |
| | Acetate | 1 | 1 | 1 | 1 | 1 | 1 | 1 | 1 |
| | Formate | 1 | 1 | 1 | 1 | 1 | 1 | 1 | 1 |
| | Ethanol | 0 | 0 | 0 | 0 | 0 | 0 | 0 | 0 |

^{b}PUL conservation information for P. copri isolates

TABLE 29

| | | | Consensus PUL | mcSEED Functional annotation | | | | | CAZy Functional |
|---|---|---|---|---|---|---|---|---|---|
| | | | (based on | | | Functional pathway | | Functional pathway | annotation |
| Strain ID | Strain PUL | MAG PUL gene locus | MAGBg0019)^{b} | Gene name | Protein product | abbreviation | Functional pathway | present in MAG | CAZyme |
| BgD5_2 | PUL9 | PFCKPOMF_00840 | Consensus | XynA | Endo-1,4-beta-xylanase | XOS | xylooligosaccharides | + | GH10- |
| | | | PUL3 | | (EC 3.2.1.8) | | utilization | | GH10- |
| | | | | | | | | | CBM4- |
| | | | | | | | | | CBM4 |
| | | PFCKPOMF_00841 | | | | | | | |
| | | PFCKPOMF_00842 | | | | | | | |
| | | PFCKPOMF_00843 | | | | | | | |
| | | PFCKPOMF_00844 | | LacZ | Beta-galactosidase | GOS | galactooligosaccharides | + | GH35 |
| | | | | | (EC 3.2.1.23) | | utilization | | |
| | | PFCKPOMF_00845 | | AguA | Xylan alpha-1,2- | aXyl | alpha-xylosides | + | GH67 |
| | | | | | glucuronosidase | | utilization | | |
| | | | | | (EC 3.2.1.131) | | | | |
| | | PFCKPOMF_00846 | | | | | | | |
| | | PFCKPOMF_00847 | | | | | | | |
| | | PFCKPOMF_00848 | | XynT | Xyloside transporter XynT | XOS | xylooligosaccharides | + | |
| | | | | | | | utilization | | |
| | | PFCKPOMF_00849 | | XynA | Endo-1,4-beta-xylanase | XOS | xylooligosaccharides | + | GH10 |
| | | | | | (EC 3.2.1.8) | | utilization | | |
| | | PFCKPOMF_00850 | | | | | | | GH43_1 |
| | PUL17 | PFCKPOMF_01484 | Consensus | | | | | | GH133 |
| | | PFCKPOMF_01485 | PUL4 | | | | | | GT4 |
| | | PFCKPOMF_01486 | | AmyA | Alpha-amylase | Mal; | maltose utilization; | +; + | GH57 |
| | | | | | (EC 3.2.1.1) | MOS | maltooligosaccharides | | |
| | | | | | | | utilization | | |
| | | PFCKPOMF_01487 | | XynB2 | Xylan 1,4-beta-xylosidase | XOS | xylooligosaccharides | + | GH43_10- |
| | | | | | (EC 3.2.1.37) | | utilization | | CBM91 |
| | | PFCKPOMF_01488 | | | | | | | |
| | | PFCKPOMF_01489 | | | | | | | |
| | | PFCKPOMF_01490 | | | | | | | |
| | | PFCKPOMF_01491 | | | | | | | |
| | | PFCKPOMF_01492 | | PelB | Pectate lyase | GalAs | oligogalacturonate | + | |
| | | | | | (EC 4.2.2.2) | | utilization | | |
| | | PFCKPOMF_01493 | | | | | | | |
| | | PFCKPOMF_01494 | | PelB | Pectate lyase | GalAs | oligogalacturonate | + | PL1_2 |
| | | | | | (EC 4.2.2.2) | | utilization | | |
| | | PFCKPOMF_01495 | | Pgl | Polygalacturonase | GalAs | oligogalacturonate | + | GH28- |
| | | | | | (EC 3.2.1.15) | | utilization | | GH105 |
| | | PFCKPOMF_01496 | | | | | | | |
| | PUL19 | PFCKPOMF_01573 | Consensus | MnnB3 | Mannan endo-1,4-beta- | bMnOS | Beta- | + | GH26 |
| | | | PUL7 | | mannosidase | | mannooligosaccharides | | |
| | | | | | | | utilization | | |
| | | PFCKPOMF_01574 | | | | | | | GH26- |
| | | | | | | | | | GH5_4 |
| | | PFCKPOMF_01575 | | | | | | | |
| | | PFCKPOMF_01576 | | | | | | | |
| | | PFCKPOMF_01577 | | | | | | | |
| | | PFCKPOMF_01578 | | | | | | | |
| | | PFCKPOMF_01579 | | | | | | | |
| | | PFCKPOMF_01580 | | | | | | | |
| | | PFCKPOMF_01581 | | | | | | | |
| | | PFCKPOMF_01582 | | MnbY | Predicted mannan acetyl- | bMnOS | Beta- | + | CE7 |
| | | | | | esterase | | mannooligosaccharides | | |
| | | | | | | | utilization | | |
| | | PFCKPOMF_01583 | | BaMan26A | Extracellular endo-beta-(1- | bMnOS | Beta- | + | GH26 |
| | | | | | 4)-mannanase, GH26 | | mannooligosaccharides | | |
| | | | | | | | utilization | | |
| | | PFCKPOMF_01584 | | BmgP | 4-O-beta-D-mannosyl-D- | bMnOS | Beta- | + | GH130_1 |
| | | | | | glucose phosphorylase | | mannooligosaccharides | | |
| | | | | | (EC 2.4.1.281) | | utilization | | |
| | PUL18 | PFCKPOMF_01565 | Consensus | | | | | | |
| | | PFCKPOMF_01566 | PUL8 | | | | | | |
| | | PFCKPOMF_01567 | | | | | | | |
| | | PFCKPOMF_01568 | | | | | | | |
| | | PFCKPOMF_01569 | | XynA | Endo-1,4-beta-xylanase | XOS | xylooligosaccharides | + | GH5_4 |
| | | | | | (EC 3.2.1.8) | | utilization | | |
| | | PFCKPOMF_01570 | | | | | | | GH5_4 |
| | | PFCKPOMF_01571 | | MnnB2 | Endo-1,4-beta- | bMnOS | Beta- | + | GH5_7 |
| | | | | | mannosidase | | mannooligosaccharides | | |
| | | | | | | | utilization | | |
| | PUL15 | PFCKPOMF_01243 | Consensus | | | | | | |
| | | PFCKPOMF_01244 | PUL9 | | | | | | |
| | | PFCKPOMF_01245 | | | | | | | |
| | | PFCKPOMF_01246 | | | | | | | |
| | | PFCKPOMF_01247 | | LacZ | Beta-galactosidase | GOS | galactooligosaccharides | + | GH2 |
| | | | | | (EC 3.2.1.23) | | utilization | | |
| | | PFCKPOMF_01248 | | | | | | | GH63 |
| | | PFCKPOMF_01249 | | | | | | | |
| | | PFCKPOMF_01250 | | | | | | | |
| | PUL22 | PFCKPOMF_02332 | Consensus | | | | | | GH127 |
| | | PFCKPOMF_02333 | PUL10 | | | | | | GH43_34- |
| | | | | | | | | | CBM32 |
| | | PFCKPOMF_02334 | | AglA | Alpha-glucosidase | Mal; | maltose utilization; | +; + | GH97 |
| | | | | | (EC 3.2.1.20) | MOS | maltooligosaccharides | | |
| | | | | | | | utilization | | |
| | | PFCKPOMF_02335 | | | | | | | GH146 |
| | | PFCKPOMF_02336 | | | | | | | |
| | | PFCKPOMF_02337 | | | | | | | |
| | | PFCKPOMF_02338 | | SerC1 | Phosphoserine | Ser | serine biosynthesis | + | |
| | | | | | aminotransferase | | | | |
| | | | | | (EC 2.6.1.52) | | | | |
| | | PFCKPOMF_02339 | | | | | | | |
| | | PFCKPOMF_02340 | | | | | | | |
| | PUL16 | PFCKPOMF_01326 | Consensus | | | | | | |
| | | PFCKPOMF_01327 | PUL13 | | | | | | GH5_4 |
| | | PFCKPOMF_01328 | | | | | | | |
| | | PFCKPOMF_01329 | | | | | | | |
| | | PFCKPOMF_01330 | | | | | | | |
| | | PFCKPOMF_01331 | | BglA | Beta-glucosidase | Bgl | beta-glucosides | − | GH3 |
| | | | | | (EC 3.2.1.21) | | utilization | | |
| | PUL10 | PFCKPOMF_00908 | Consensus | PelB | Pectate lyase | GalAs | oligogalacturonate | + | PL1- |
| | | | PUL16 | | (EC 4.2.2.2) | | utilization | | CBM77 |
| | | PFCKPOMF_00909 | | Abg | Arabinogalactan endo-1,4- | GOS | galactooligosaccharides | + | GH53 |
| | | | | | beta-galactanase | | utilization | | |
| | | | | | (EC 3.2.1.89) | | | | |
| | | PFCKPOMF_00910 | | LacZ | Beta-galactosidase | GOS | galactooligosaccharides | + | GH2 |
| | | | | | (EC 3.2.1.23) | | utilization | | |
| | | PFCKPOMF_00911 | | | | | | | |
| | | PFCKPOMF_00912 | | | | | | | |
| | | PFCKPOMF_00913 | | SusC_bga | SusC, outer membrane | GOS | galactooligosaccharides | + | |
| | | | | | protein involved in beta- | | utilization | | |
| | | | | | galactoside utilization | | | | |
| | | PFCKPOMF_00914 | | | | | | | |
| | | PFCKPOMF_00915 | | | | | | | |
| | | PFCKPOMF_00916 | | | | | | | |
| | | PFCKPOMF_00917 | | | | | | | |
| | | PFCKPOMF_00918 | | | | | | | |
| | | PFCKPOMF_00919 | | | | | | | |
| | | PFCKPOMF_00920 | | | | | | | |
| | | PFCKPOMF_00921 | | | | | | | |
| | | PFCKPOMF_00922 | | ThiD | Hydroxymethylpyrimidine | B1 | TPP cofactor, de novo | + | |
| | | | | | phosphate kinase ThiD | | synthesis | | |
| | | | | | (EC 2.7.4.7) | | | | |
| | PUL3 | PFCKPOMF_00392 | Consensus | Abf3_GH51 | Exo-alpha-(1->2/1->3)-L- | aAOS | alpha- | + | GH51_2 |
| | | | PUL17a | | arabinofuranosidase | | arabinooligosaccharides | | |
| | | | | | (EC 3.2.1.55), GH51 family | | utilization | | |
| | | PFCKPOMF_00393 | | | | | | | |
| | | PFCKPOMF_00394 | | | | | | | |
| | | PFCKPOMF_00395 | | AbnA | Endo-alpha-(1->5)-L- | aAOS | alpha- | + | GH43_5 |
| | | | | | arabinanase (EC 3.2.1.99), | | arabinooligosaccharides | | |
| | | | | | GH43 family | | utilization | | |
| | | PFCKPOMF_00396 | | AbnA | Endo-alpha-(1->5)-L- | aAOS | alpha- | + | GH43_4 |
| | | | | | arabinanase (EC 3.2.1.99), | | arabinooligosaccharides | | |
| | | | | | GH43 family | | utilization | | |
| | | PFCKPOMF_00397 | | | | | | | |
| | | PFCKPOMF_00398 | | | | | | | |
| | | PFCKPOMF_00399 | | | | | | | |
| | | PFCKPOMF_00400 | | | | | | | |
| | | PFCKPOMF_00401 | | | | | | | |
| | | PFCKPOMF_00402 | | | | | | | |
| | | PFCKPOMF_00403 | | | | | | | |
| | | PFCKPOMF_00404 | | NplT | Neopullulanase | Mal; | maltose utilization; | +; + | GH13_46 |
| | | | | | (EC 3.2.1.135) | MOS | maltooligosaccharides | | |
| | | | | | | | utilization | | |
| | | PFCKPOMF_00405 | | PulA | Pullulanase (EC 3.2.1.41) | Mal; | maltose utilization; | +; + | GH13_14 |
| | | | | | | MOS | maltooligosaccharides | | |
| | | | | | | | utilization | | |
| | | PFCKPOMF_00406 | | SusB2 | Glucan 1,4-alpha- | Mal; | maltose utilization; | +; + | GH97 |
| | | | | | glucosidase (EC 3.2.1.3) | MOS | maltooligosaccharides | | |
| | | | | | | | utilization | | |
| | | PFCKPOMF_00407 | | MalQ | 4-alpha-glucanotransferase | Mal; | maltose utilization; | +; + | CBM20- |
| | | | | | (amylomaltase) | MOS | maltooligosaccharides | | GH77 |
| | | | | | (EC 2.4.1.25) | | utilization | | |
| | | PFCKPOMF_00408 | | MalT | Predicted maltose | Mal; | maltose utilization; | +; + | |
| | | | | | transporter, MFS family | MOS | maltooligosaccharides | | |
| | | | | | | | utilization | | |
| | | PFCKPOMF_00409 | | MalR | Maltose operon | Mal; | maltose utilization; | +; + | |
| | | | | | transcriptional repressor | MOS | maltooligosaccharides | | |
| | | | | | MalR, LacI family | | utilization | | |
| | | PFCKPOMF_00410 | | SusCm | SusC, outer membrane | Mal; | maltose utilization; | +; + | |
| | | | | | protein involved in | MOS | maltooligosaccharides | | |
| | | | | | maltodextrin utilization | | utilization | | |
| | | PFCKPOMF_00411 | | SusDm | SusD, outer membrane | Mal; | maltose utilization; | +; + | |
| | | | | | protein involved in | MOS | maltooligosaccharides | | |
| | | | | | maltodextrin utilization | | utilization | | |
| | | PFCKPOMF_00412 | | | | | | | |
| | | PFCKPOMF_00413 | | | | | | | |
| BgF5_2 | PUL8 | BBPDHENA_01083 | Consensus | XynA | Endo-1,4-beta-xylanase | XOS | xylooligosaccharides | + | GH10- |
| | | | PUL3 | | (EC 3.2.1.8) | | utilization | | GH10- |
| | | | | | | | | | CBM4- |
| | | | | | | | | | CBM4 |
| | | BBPDHENA_01084 | | | | | | | |
| | | BBPDHENA_01085 | | | | | | | |
| | | BBPDHENA_01086 | | | | | | | |
| | | BBPDHENA_01087 | | LacZ | Beta-galactosidase | GOS | galactooligosaccharid | + | GH35 |
| | | | | | (EC 3.2.1.23) | | es utilization | | |
| | | BBPDHENA_01088 | | AguA | Xylan alpha-1,2- | aXyl | alpha-xylosides | + | GH67 |
| | | | | | glucuronosidase | | utilization | | |
| | | | | | (EC 3.2.1.131) | | | | |
| | | BBPDHENA_01089 | | | | | | | |
| | | BBPDHENA_01090 | | | | | | | |
| | | BBPDHENA_01091 | | XynT | Xyloside transporter XynT | XOS | xylooligosaccharides | + | |
| | | | | | | | utilization | | |
| | | BBPDHENA_01092 | | XynA | Endo-1,4-beta-xylanase | XOS | xylooligosaccharides | + | GH10 |
| | | | | | (EC 3.2.1.8) | | utilization | | |
| | | BBPDHENA_01093 | | | | | | | GH43_1 |
| | PUL16 | BBPDHENA_01728 | Consensus | | | | | | GH133 |
| | | BBPDHENA_01729 | PUL4 | | | | | | GT4 |
| | | BBPDHENA_01730 | | AmyA | Alpha-amylase (EC 3.2.1.1) | Mal; | maltose utilization; | +; + | GH57 |
| | | | | | | MOS | maltooligosaccharides | | |
| | | | | | | | utilization | | |
| | | BBPDHENA_01731 | | XynB2 | Xylan 1,4-beta-xylosidase | XOS | xylooligosaccharides | + | GH43_10- |
| | | | | | (EC 3.2.1.37) | | utilization | | CBM91 |
| | | BBPDHENA_01732 | | | | | | | |
| | | BBPDHENA_01733 | | | | | | | |
| | | BBPDHENA_01734 | | | | | | | |
| | | BBPDHENA_01735 | | | | | | | |
| | | BBPDHENA_01736 | | PelB | Pectate lyase (EC 4.2.2.2) | GalAs | oligogalacturonate | + | |
| | | | | | | | utilization | | |
| | | BBPDHENA_01737 | | | | | | | |
| | | BBPDHENA_01738 | | PelB | Pectate lyase (EC 4.2.2.2) | GalAs | oligogalacturonate | + | PL1_2 |
| | | | | | | | utilization | | |
| | | BBPDHENA_01739 | | Pgl | Polygalacturonase | GalAs | oligogalacturonate | + | GH28- |
| | | | | | (EC 3.2.1.15) | | utilization | | GH105 |
| | | BBPDHENA_01740 | | | | | | | |
| | PUL18 | BBPDHENA_01817 | Consensus | MnnB3 | Mannan endo-1,4-beta- | bMnOS | Beta- | + | GH26 |
| | | | PUL7 | | mannosidase | | mannooligosaccharides | | |
| | | | | | | | utilization | | |
| | | BBPDHENA_01818 | | | | | | | GH26- |
| | | | | | | | | | GH5_4 |
| | | BBPDHENA_01819 | | | | | | | |
| | | BBPDHENA_01820 | | | | | | | |
| | | BBPDHENA_01821 | | | | | | | |
| | | BBPDHENA_01822 | | | | | | | |
| | | BBPDHENA_01823 | | | | | | | |
| | | BBPDHENA_01824 | | | | | | | |
| | | BBPDHENA_01825 | | | | | | | |
| | | BBPDHENA_01826 | | MnbY | Predicted mannan acetyl- | bMnOS | Beta- | + | |
| | | | | | esterase | | mannooligosaccharides | | |
| | | | | | | | utilization | | |
| | | BBPDHENA_01827 | | BaMan26A | Extracellular endo-beta-(1- | bMnOS | Beta- | + | GH26 |
| | | | | | 4)-mannanase, GH26 | | mannooligosaccharides | | |
| | | | | | | | utilization | | |
| | | BBPDHENA_01828 | | BmgP | 4-O-beta-D-mannosyl-D- | bMnOS | Beta- | + | GH130_1 |
| | | | | | glucose phosphorylase | | mannooligosaccharides | | |
| | | | | | (EC 2.4.1.281) | | utilization | | |
| | PUL17 | BBPDHENA_01809 | Consensus | | | | | | |
| | | BBPDHENA_01810 | PUL8 | | | | | | |
| | | BBPDHENA_01811 | | | | | | | |
| | | BBPDHENA_01812 | | | | | | | |
| | | BBPDHENA_01813 | | XynA | Endo-1,4-beta-xylanase | XOS | xylooligosaccharides | + | GH5_4 |
| | | | | | (EC 3.2.1.8) | | utilization | | |
| | | BBPDHENA_01814 | | | | | | | GH5_4 |
| | | BBPDHENA_01815 | | MnnB2 | Endo-1,4-beta- | bMnOS | Beta- | + | GH5_7 |
| | | | | | mannosidase | | mannooligosaccharides | | |
| | | | | | | | utilization | | |
| | PUL14 | BBPDHENA_01487 | Consensus | | | | | | |
| | | BBPDHENA_01488 | PUL9 | | | | | | |
| | | BBPDHENA_01489 | | | | | | | |
| | | BBPDHENA_01490 | | | | | | | |
| | | BBPDHENA_01491 | | LacZ | Beta-galactosidase | GOS | galactooligosaccharides | + | GH2 |
| | | | | | (EC 3.2.1.23) | | utilization | | |
| | | BBPDHENA_01492 | | | | | | | GH63 |
| | | BBPDHENA_01493 | | | | | | | |
| | | BBPDHENA_01494 | | | | | | | |
| | PUL20 | BBPDHENA_02103 | Consensus | | | | | | GH127 |
| | | BBPDHENA_02104 | PUL10 | | | | | | GH43_34- |
| | | | | | | | | | CBM32 |
| | | BBPDHENA_02105 | | AglA | Alpha-glucosidase | Mal; | maltose utilization; | +; + | GH97 |
| | | | | | (EC 3.2.1.20) | MOS | maltooligosaccharides | | |
| | | | | | | | utilization | | |
| | | BBPDHENA_02106 | | | | | | | GH146 |
| | | BBPDHENA_02107 | | | | | | | |
| | | BBPDHENA_02108 | | | | | | | |
| | | BBPDHENA_02109 | | SerC1 | Phosphoserine | Ser | serine biosynthesis | + | |
| | | | | | aminotransferase | | | | |
| | | | | | (EC 2.6.1.52) | | | | |
| | | BBPDHENA_02110 | | | | | | | |
| | | BBPDHENA_02111 | | | | | | | |
| | PUL15 | BBPDHENA_01570 | Consensus | | | | | | |
| | | BBPDHENA_01571 | PUL13 | | | | | | GH5_4 |
| | | BBPDHENA_01572 | | | | | | | |
| | | BBPDHENA_01573 | | | | | | | |
| | | BBPDHENA_01574 | | | | | | | |
| | | BBPDHENA_01575 | | BglA | Beta-glucosidase | Bgl | beta-glucosides | − | GH3 |
| | | | | | (EC 3.2.1.21) | | utilization | | |
| | PUL9 | BBPDHENA_01151 | Consensus | PelB | Pectate lyase (EC 4.2.2.2) | GalAs | oligogalacturonate | + | PL1- |
| | | | PUL16 | | | | utilization | | CBM77 |
| | | BBPDHENA_01152 | | Abg | Arabinogalactan endo-1,4- | GOS | galactooligosaccharides | + | GH53 |
| | | | | | beta-galactanase | | utilization | | |
| | | | | | (EC 3.2.1.89) | | | | |
| | | BBPDHENA_01153 | | LacZ | Beta-galactosidase | GOS | galactooligosaccharides | + | GH2 |
| | | | | | (EC 3.2.1.23) | | utilization | | |
| | | BBPDHENA_01154 | | | | | | | |
| | | BBPDHENA_01155 | | | | | | | |
| | | BBPDHENA_01156 | | SusC_bga | SusC, outer membrane | GOS | galactooligosaccharides | + | |
| | | | | | protein involved in beta- | | utilization | | |
| | | | | | galactoside utilization | | | | |
| | | BBPDHENA_01157 | | | | | | | |
| | | BBPDHENA_01158 | | | | | | | |
| | | BBPDHENA_01159 | | | | | | | |
| | | BBPDHENA_01160 | | | | | | | |
| | | BBPDHENA_01161 | | | | | | | |
| | | BBPDHENA_01162 | | | | | | | |
| | | BBPDHENA_01163 | | | | | | | |
| | | BBPDHENA_01164 | | | | | | | |
| | | BBPDHENA_01165 | | ThiD | Hydroxymethylpyrimidine | B1 | TPP cofactor, de novo | + | |
| | | | | | phosphate kinase ThiD | | synthesis | | |
| | | | | | (EC 2.7.4.7) | | | | |
| | PUL2 | BBPDHENA_00634 | Consensus | Abf3_GH51 | Exo-alpha-(1->2/1->3)-L- | aAOS | alpha- | + | GH51_2 |
| | | | PUL17a | | arabinofuranosidase | | arabinooligosaccharides | | |
| | | | | | (EC 3.2.1.55), GH51 family | | utilization | | |
| | | BBPDHENA_00635 | | | | | | | |
| | | BBPDHENA_00636 | | | | | | | |
| | | BBPDHENA_00637 | | AbnA | Endo-alpha-(1->5)-L- | aAOS | alpha- | + | GH43_5 |
| | | | | | arabinanase (EC 3.2.1.99), | | arabinooligosaccharides | | |
| | | | | | GH43 family | | utilization | | |
| | | BBPDHENA_00638 | | AbnA | Endo-alpha-(1->5)-L- | aAOS | alpha- | + | GH43_4 |
| | | | | | arabinanase (EC 3.2.1.99), | | arabinooligosaccharides | | |
| | | | | | GH43 family | | utilization | | |
| | | BBPDHENA_00639 | | | | | | | |
| | | BBPDHENA_00640 | | | | | | | |
| | | BBPDHENA_00641 | | | | | | | |
| | | BBPDHENA_00642 | | | | | | | |
| | | BBPDHENA_00643 | | | | | | | |
| | | BBPDHENA_00644 | | | | | | | |
| | | BBPDHENA_00645 | | | | | | | |
| | | BBPDHENA_00646 | | NplT | Neopullulanase | Mal; | maltose utilization; | +; + | GH13_46 |
| | | | | | (EC 3.2.1.135) | MOS | maltooligosaccharides | | |
| | | | | | | | utilization | | |
| | | BBPDHENA_00647 | | PulA | Pullulanase (EC 3.2.1.41) | Mal; | maltose utilization; | +; + | GH13_14 |
| | | | | | | MOS | maltooligosaccharides | | |
| | | | | | | | utilization | | |
| | | BBPDHENA_00648 | | SusB2 | Glucan 1,4-alpha- | Mal; | maltose utilization; | +; + | GH97 |
| | | | | | glucosidase (EC 3.2.1.3) | MOS | maltooligosaccharides | | |
| | | | | | | | utilization | | |
| | | BBPDHENA_00649 | | MalQ | 4-alpha-glucanotransferase | Mal; | maltose utilization; | +; + | CBM20- |
| | | | | | (amylomaltase) | MOS | maltooligosaccharides | | GH77 |
| | | | | | (EC 2.4.1.25) | | utilization | | |
| | | BBPDHENA_00650 | | MalT | Predicted maltose | Mal; | maltose utilization; | +; + | |
| | | | | | transporter, MFS family | MOS | maltooligosaccharides | | |
| | | | | | | | utilization | | |
| | | BBPDHENA_00651 | | MalR | Maltose operon | Mal; | maltose utilization; | +; + | |
| | | | | | transcriptional repressor | MOS | maltooligosaccharides | | |
| | | | | | MalR, LacI family | | utilization | | |
| | | BBPDHENA_00652 | | SusCm | SusC, outer membrane | Mal; | maltose utilization; | +; + | |
| | | | | | protein involved in | MOS | maltooligosaccharides | | |
| | | | | | maltodextrin utilization | | utilization | | |
| | | BBPDHENA_00653 | | SusDm | SusD, outer membrane | Mal; | maltose utilization; | +; + | |
| | | | | | protein involved in | MOS | maltooligosaccharides | | |
| | | | | | maltodextrin utilization | | utilization | | |
| | | BBPDHENA_00654 | | | | | | | |
| | | BBPDHENA_00655 | | | | | | | |
| Bg131 | PUL15 | NJCFFJJN_02552 | Consensus | | | | | | GH43_1 |
| | | NJCFFJJN_02553 | PUL3 | XynA | Endo-1,4-beta-xylanase | XOS | xylooligosaccharides | + | GH10 |
| | | | | | (EC 3.2.1.8) | | utilization | | |
| | | NJCFFJJN_02554 | | XynT | Xyloside transporter XynT | XOS | xylooligosaccharides | + | |
| | | | | | | | utilization | | |
| | | NJCFFJJN_02555 | | | | | | | |
| | | NJCFFJJN_02556 | | | | | | | |
| | | NJCFFJJN_02557 | | AguA | Xylan alpha-1,2- | aXyl | alpha-xylosides | + | GH67 |
| | | | | | glucuronosidase | | utilization | | |
| | | | | | (EC 3.2.1.131) | | | | |
| | | NJCFFJJN_02558 | | LacZ | Beta-galactosidase | GOS | galactooligosaccharides | + | GH35 |
| | | | | | (EC 3.2.1.23) | | utilization | | |
| | | NJCFFJJN_02559 | | SusCx | SusC, outer membrane | XOS | xylooligosaccharides | + | |
| | | | | | protein involved in XOS | | utilization | | |
| | | | | | utilization | | | | |
| | | NJCFFJJN_02560 | | SusDx | SusD, outer membrane | XOS | xylooligosaccharides | + | |
| | | | | | protein involved in XOS | | utilization | | |
| | | | | | utilization | | | | |
| | | NJCFFJJN_02561 | | | | | | | GH10 |
| | PUL6 | NJCFFJJN_01898 | Consensus | | | | | | |
| | | NJCFFJJN_01899 | PUL4 | Pgl | Polygalacturonase | GalAs | oligogalacturonate | + | GH28- |
| | | | | | (EC 3.2.1.15) | | utilization | | GH105 |
| | | NJCFFJJN_01900 | | | | | | | |
| | | NJCFFJJN_01901 | | | | | | | |
| | | NJCFFJJN_01902 | | PelB | Pectate lyase (EC 4.2.2.2) | GalAs | oligogalacturonate | + | CE8 |
| | | | | | | | utilization | | |
| | | NJCFFJJN_01903 | | | | | | | |
| | | NJCFFJJN_01904 | | | | | | | |
| | | NJCFFJJN_01905 | | | | | | | |
| | PUL7 | NJCFFJJN_01907 | | | | | | | |
| | | NJCFFJJN_01908 | | | | | | | |
| | | NJCFFJJN_01909 | | | | | | | GH28 |
| | | NJCFFJJN_01910 | | XynB2 | Xylan 1,4-beta-xylosidase | XOS | xylooligosaccharides | + | GH43_10 |
| | | | | | (EC 3.2.1.37) | | utilization | | |
| | | NJCFFJJN_01911 | | AmyA | Alpha-amylase (EC 3.2.1.1) | Mal; | maltose utilization; | +; + | GH57 |
| | | | | | | MOS | maltooligosaccharides | | |
| | | | | | | | utilization | | |
| | | NJCFFJJN_01912 | | | | | | | GT4 |
| | | NJCFFJJN_01913 | | | | | | | GH133 |
| | PUL5 | NJCFFJJN_01811 | Consensus | BmgP | 4-O-beta-D-mannosyl-D- | bMnOS | Beta- | + | GH130 |
| | | | PUL7 | | glucose phosphorylase | | mannooligosaccharides | | |
| | | | | | (EC 2.4.1.281) | | utilization | | |
| | | NJCFFJJN_01812 | | BaMan26A | Extracellular endo-beta-(1- | bMnOS | Beta- | + | GH26 |
| | | | | | 4)-mannanase, GH26 | | mannooligosaccharides | | |
| | | | | | | | utilization | | |
| | | NJCFFJJN_01813 | | MnbY | Predicted mannan acetyl- | bMnOS | Beta- | + | CE7 |
| | | | | | esterase | | mannooligosaccharides | | |
| | | | | | | | utilization | | |
| | | NJCFFJJN_01814 | | BaMan26A | Extracellular endo-beta-(1- | bMnOS | Beta- | + | GH26 |
| | | | | | 4)-mannanase, GH26 | | mannooligosaccharides | | |
| | | | | | | | utilization | | |
| | | NJCFFJJN_01815 | | | | | | | |
| | | NJCFFJJN_01816 | | | | | | | |
| | | NJCFFJJN_01817 | | | | | | | |
| | PUL3 | NJCFFJJN_00575 | Consensus | | | | | | GH127 |
| | | NJCFFJJN_00576 | PUL10 | | | | | | GH43_34- |
| | | | | | | | | | CBM32 |
| | | NJCFFJJN_00577 | | AglA | Alpha-glucosidase | Mal; | maltose utilization; | +; + | GH97 |
| | | | | | (EC 3.2.1.20) | MOS | maltooligosaccharides | | |
| | | | | | | | utilization | | |
| | | NJCFFJJN_00578 | | | | | | | GH146 |
| | | NJCFFJJN_00579 | | | | | | | |
| | | NJCFFJJN_00580 | | | | | | | |
| | | NJCFFJJN_00581 | | | | | | | |
| | PUL30 | NJCFFJJN_03307 | Consensus | | | | | | |
| | | NJCFFJJN_03308 | PUL11 | BglA2 | beta-glucosidase | Bgl | beta-glucosides | + | GH3 |
| | | | | | (EC 3.2.1.21) | | utilization | | |
| | | NJCFFJJN_03309 | | SusC_bgl | SusC, outer membrane | Bgl | beta-glucosides | + | |
| | | | | | protein involved in beta- | | utilization | | |
| | | | | | glucoside binding | | | | |
| | | NJCFFJJN_03310 | | SusD_bgl | SusD, outer membrane | Bgl | beta-glucosides | + | |
| | | | | | protein involved in beta- | | utilization | | |
| | | | | | glucoside binding | | | | |
| | | NJCFFJJN_03311 | | | | | | | |
| | | NJCFFJJN_03312 | | LamA | Beta-glucanase precursor | Bgl | beta-glucosides | + | GH16_3 |
| | | | | | (EC 3.2.1.73) | | utilization | | |
| | PUL8 | NJCFFJJN_02065 | Consensus | BglA | Beta-glucosidase | Bgl | beta-glucosides | + | GH3 |
| | | | PUL13 | | (EC 3.2.1.21) | | utilization | | |
| | | NJCFFJJN_02066 | | | | | | | |
| | | NJCFFJJN_02067 | | | | | | | |
| | | NJCFFJJN_02068 | | | | | | | |
| | | NJCFFJJN_02069 | | | | | | | GH5_4 |
| | | NJCFFJJN_02070 | | | | | | | |
| | PUL14 | NJCFFJJN_02485 | Consensus | ThiD | Hydroxymethylpyrimidine | B1 | TPP cofactor, de novo | + | |
| | | | PUL16 | | phosphate kinase ThiD | | synthesis | | |
| | | | | | (EC 2.7.4.7) | | | | |
| | | NJCFFJJN_02486 | | | | | | | |
| | | NJCFFJJN_02487 | | | | | | | |
| | | NJCFFJJN_02488 | | | | | | | |
| | | NJCFFJJN_02489 | | | | | | | |
| | | NJCFFJJN_02490 | | | | | | | |
| | | NJCFFJJN_02491 | | | | | | | |
| | | NJCFFJJN_02492 | | | | | | | |
| | | NJCFFJJN_02493 | | | | | | | |
| | | NJCFFJJN_02494 | | SusC_bga | SusC, outer membrane | GOS | galactooligosaccharides | + | |
| | | | | | protein involved in beta- | | utilization | | |
| | | | | | galactoside utilization | | | | |
| | | NJCFFJJN_02495 | | | | | | | |
| | | NJCFFJJN_02496 | | LacZ | Beta-galactosidase | GOS | galactooligosaccharides | + | GH2 |
| | | | | | (EC 3.2.1.23) | | utilization | | |
| | | NJCFFJJN_02497 | | Abg | Arabinogalactan endo-1,4- | GOS | galactooligosaccharides | + | GH53 |
| | | | | | beta-galactanase | | utilization | | |
| | | | | | (EC 3.2.1.89) | | | | |
| | | NJCFFJJN_02498 | | PelB | Pectate lyase (EC 4.2.2.2) | GalAs | oligogalacturonate | + | PL1 |
| | | | | | | | utilization | | |
| | PUL27 | NJCFFJJN_03225 | Consensus | | | | | | |
| | | NJCFFJJN_03226 | PUL17a/17b | | | | | | |
| | | NJCFFJJN_03227 | | SusDm | SusD, outer membrane | Mal; | maltose utilization; | +; + | |
| | | | | | protein involved in | MOS | maltooligosaccharides | | |
| | | | | | maltodextrin utilization | | utilization | | |
| | | NJCFFJJN_03228 | | SusCm | SusC, outer membrane | Mal; | maltose utilization; | +; + | |
| | | | | | protein involved in | MOS | maltooligosaccharides | | |
| | | | | | maltodextrin utilization | | utilization | | |
| | | NJCFFJJN_03229 | | MalR | Maltose operon | Mal; | maltose utilization; | +; + | |
| | | | | | transcriptional repressor | MOS | maltooligosaccharides | | |
| | | | | | MalR, LacI family | | utilization | | |
| | | NJCFFJJN_03230 | | MalT | Predicted maltose | Mal; | maltose utilization; | +; + | |
| | | | | | transporter, MFS family | MOS | maltooligosaccharides | | |
| | | | | | | | utilization | | |
| | | NJCFFJJN_03231 | | MalQ | 4-alpha-glucanotransferase | Mal; | maltose utilization; | +; + | CBM20- |
| | | | | | (amylomaltase) | MOS | maltooligosaccharides | | GH77 |
| | | | | | (EC 2.4.1.25) | | utilization | | |
| | | NJCFFJJN_03232 | | SusB2 | Glucan 1,4-alpha- | Mal; | maltose utilization; | +; + | GH97 |
| | | | | | glucosidase (EC 3.2.1.3) | MOS | maltooligosaccharides | | |
| | | | | | | | utilization | | |
| | | NJCFFJJN_03233 | | PulA | Pullulanase (EC 3.2.1.41) | Mal; | maltose utilization; | +; + | GH13_14 |
| | | | | | | MOS | maltooligosaccharides | | |
| | | | | | | | utilization | | |
| | | NJCFFJJN_03234 | | NplT | Neopullulanase | Mal; | maltose utilization; | +; + | GH13 |
| | | | | | (EC 3.2.1.135) | MOS | maltooligosaccharides | | |
| | | | | | | | utilization | | |
| | | NJCFFJJN_03235 | | | | | | | |
| | | NJCFFJJN_03236 | | | | | | | |
| | | NJCFFJJN_03237 | | | | | | | |
| | | NJCFFJJN_03238 | | | | | | | |
| | | NJCFFJJN_03239 | | | | | | | |
| | | NJCFFJJN_03240 | | | | | | | |
| | | NJCFFJJN_03241 | | | | | | | |
| | | NJCFFJJN_03242 | | | | | | | |
| | | NJCFFJJN_03243 | | | | | | | |
| | | NJCFFJJN_03244 | | AbnA | Endo-alpha-(1->5)-L- | aAOS | alpha- | + | GH43_4 |
| | | | | | arabinanase (EC 3.2.1.99), | | arabinooligosaccharides | | |
| | | | | | GH43 family | | utilization | | |
| | | NJCFFJJN_03245 | | AbnA | Endo-alpha-(1->5)-L- | aAOS | alpha- | + | GH43_5 |
| | | | | | arabinanase (EC 3.2.1.99), | | arabinooligosaccharides | | |
| | | | | | GH43 family | | utilization | | |
| Bg2C6 | PUL15 | NBFEJGPP_00821 | Consensus | | | | | | GH10[Fnc] |
| | | NBFEJGPP_00822 | PUL3 | SusDx | SusD, outer membrane | XOS | xylooligosaccharides | + | SusD |
| | | | | | protein involved in XOS | | utilization | | |
| | | | | | utilization | | | | |
| | | NBFEJGPP_00823 | | SusCx | SusC, outer membrane | XOS | xylooligosaccharides | + | SusC |
| | | | | | protein involved in XOS | | utilization | | |
| | | | | | utilization | | | | |
| | | NBFEJGPP_00824 | | LacZ | Beta-galactosidase | GOS | galactooligosaccharides | + | GH35 |
| | | | | | (EC 3.2.1.23) | | utilization | | |
| | | NBFEJGPP_00825 | | AguA | Xylan alpha-1,2- | aXyl | alpha-xylosides | + | GH67 |
| | | | | | glucuronosidase | | utilization | | |
| | | | | | (EC 3.2.1.131) | | | | |
| | | NBFEJGPP_00826 | | | | | | | HTCS |
| | | NBFEJGPP_00827 | | | | | | | Est |
| | | NBFEJGPP_00828 | | XynT | Xyloside transporter XynT | XOS | xylooligosaccharides | + | MFS |
| | | | | | | | utilization | | |
| | | NBFEJGPP_00829 | | XynA | Endo-1,4-beta-xylanase | XOS | xylooligosaccharides | + | GH10 |
| | | | | | (EC 3.2.1.8) | | utilization | | |
| | | NBFEJGPP_00830 | | | | | | | GH43_1 |
| | PUL23 | NBFEJGPP_01419 | Consensus | | | | | | GH133 |
| | | NBFEJGPP_01420 | PUL4 | | | | | | GT4 |
| | | NBFEJGPP_01421 | | AmyA | Alpha-amylase (EC 3.2.1.1) | Mal; | maltose utilization; | +; + | GH57 |
| | | | | | | MOS | maltooligosaccharides | | |
| | | | | | | | utilization | | |
| | | NBFEJGPP_01422 | | XynB2 | Xylan 1,4-beta-xylosidase | XOS | xylooligosaccharides | + | GH43_10\| |
| | | | | | (EC 3.2.1.37) | | utilization | | CBM91 |
| | | NBFEJGPP_01423 | | | | | | | GH28 |
| | | NBFEJGPP_01424 | | | | | | | |
| | | NBFEJGPP_01425 | | | | | | | |
| | | NBFEJGPP_01426 | | | | | | | |
| | | NBFEJGPP_01427 | | | | | | | SusD |
| | | NBFEJGPP_01428 | | SusC_bga | SusC, outer membrane | GOS | galactooligosaccharides | + | SusC |
| | | | | | protein involved in beta- | | utilization | | |
| | | | | | galactoside utilization | | | | |
| | | NBFEJGPP_01429 | | | | | | | CE |
| | | NBFEJGPP_01430 | | | | | | | |
| | | NBFEJGPP_01431 | | | | | | | |
| | | NBFEJGPP_01432 | | Pgl | Polygalacturonase | GalAs | oligogalacturonate | + | GH28\|GH105 |
| | | | | | (EC 3.2.1.15) | | utilization | | |
| | | NBFEJGPP_01433 | | | | | | | ECF-sigma |
| | PUL24 | NBFEJGPP_01517 | Consensus | | | | | | |
| | | NBFEJGPP_01518 | PUL7 | | | | | | |
| | | NBFEJGPP_01519 | | | | | | | SusD |
| | | NBFEJGPP_01520 | | | | | | | SusC |
| | | NBFEJGPP_01521 | | MnbY | Predicted mannan acetyl- | bMnOS | Beta- | + | CE7 |
| | | | | | esterase | | mannooligosaccharides | | |
| | | | | | | | utilization | | |
| | | NBFEJGPP_01522 | | BaMan26A | Extracellular endo-beta-(1- | bMnOS | Beta- | + | GH26 |
| | | | | | 4)-mannanase, GH26 | | mannooligosaccharides | | |
| | | | | | | | utilization | | |
| | | NBFEJGPP_01523 | | BmgP | 4-O-beta-D-mannosyl-D- | bMnOS | Beta- | + | GH130 |
| | | | | | glucose phosphorylase | | mannooligosaccharides | | |
| | | | | | (EC 2.4.1.281) | | utilization | | |
| | PUL21 | NBFEJGPP_01201 | Consensus | | | | | | SusC |
| | | NBFEJGPP_01202 | PUL9 | | | | | | SusD |
| | | NBFEJGPP_01203 | | | | | | | |
| | | NBFEJGPP_01204 | | | | | | | |
| | | NBFEJGPP_01205 | | LacZ | Beta-galactosidase | GOS | galactooligosaccharides | + | GH2 |
| | | | | | (EC 3.2.1.23) | | utilization | | |
| | | NBFEJGPP_01206 | | | | | | | GH63 |
| | | NBFEJGPP_01207 | | | | | | | Anti-sigma |
| | | NBFEJGPP_01208 | | | | | | | ECF-sigma |
| | PUL26 | NBFEJGPP_02812 | Consensus | | | | | | |
| | | NBFEJGPP_02813 | PUL10 | | | | | | SusD |
| | | NBFEJGPP_02814 | | | | | | | SusC |
| | | NBFEJGPP_02815 | | | | | | | GH146 |
| | | NBFEJGPP_02816 | | AglA | Alpha-glucosidase | Mal; | maltose utilization; | +; + | GH97 |
| | | | | | (EC 3.2.1.20) | MOS | maltooligosaccharides | | |
| | | | | | | | utilization | | |
| | | NBFEJGPP_02817 | | | | | | | GH43_34\| |
| | | | | | | | | | CBM32 |
| | | NBFEJGPP_02818 | | | | | | | GH127 |
| | PUL4 | NBFEJGPP_00222 | Consensus | SusDm | SusD, outer membrane | Mal; | maltose utilization; | +; + | SusD |
| | | | PUL11 | | protein involved in | MOS | maltooligosaccharides | | |
| | | | | | maltodextrin utilization | | utilization | | |
| | | NBFEJGPP_00223 | | | | | | | SusC |
| | | NBFEJGPP_00224 | | | | | | | |
| | | NBFEJGPP_00225 | | BglA2 | beta-glucosidase | Bgl | beta-glucosides | − | GH3 |
| | | | | | (EC 3.2.1.21) | | utilization | | |
| | | NBFEJGPP_00226 | | | | | | | SusR |
| | PUL22 | NBFEJGPP_01274 | Consensus | | | | | | HTCS |
| | | NBFEJGPP_01275 | PUL13 | | | | | | GH5_4 |
| | | NBFEJGPP_01276 | | | | | | | SusC |
| | | NBFEJGPP_01277 | | | | | | | SusD |
| | | NBFEJGPP_01278 | | | | | | | |
| | | NBFEJGPP_01279 | | BglA | Beta-glucosidase | Bgl | beta-glucosides | − | GH3 |
| | | | | | (EC 3.2.1.21) | | utilization | | |
| | PUL16 | NBFEJGPP_00885 | Consensus | PelB | Pectate lyase (EC 4.2.2.2) | GalAs | oligogalacturonate | + | PL1 |
| | | | PUL16 | | | | utilization | | |
| | | NBFEJGPP_00886 | | Abg | Arabinogalactan endo-1,4- | GOS | galactooligosaccharides | + | GH53 |
| | | | | | beta-galactanase | | utilization | | |
| | | | | | (EC 3.2.1.89) | | | | |
| | | NBFEJGPP_00887 | | LacZ | Beta-galactosidase | GOS | galactooligosaccharides | + | GH2 |
| | | | | | (EC 3.2.1.23) | | utilization | | |
| | | NBFEJGPP_00888 | | | | | | | HTCS |
| | | NBFEJGPP_00889 | | SusC_bga | SusC, outer membrane | GOS | galactooligosaccharides | + | SusC |
| | | | | | protein involved in beta- | | utilization | | |
| | | | | | galactoside utilization | | | | |
| | | NBFEJGPP_00890 | | | | | | | SusD |
| | | NBFEJGPP_00891 | | | | | | | |
| | | NBFEJGPP_00892 | | | | | | | |
| | | NBFEJGPP_00893 | | | | | | | |
| | | NBFEJGPP_00894 | | | | | | | |
| | | NBFEJGPP_00895 | | | | | | | Pept_SB |
| | | NBFEJGPP_00896 | | | | | | | |
| | | NBFEJGPP_00897 | | | | | | | |
| | | NBFEJGPP_00898 | | ThiD | Hydroxymethylpyrimidine | B1 | TPP cofactor, de novo | + | |
| | | | | | phosphate kinase ThiD | | synthesis | | |
| | | | | | (EC 2.7.4.7) | | | | |
| | PUL7 | NBFEJGPP_00278 | Consensus | AbnA | Endo-alpha-(1->5)-L- | aAOS | alpha- | + | GH43_5 |
| | | | PUL17a/17b | | arabinanase (EC 3.2.1.99), | | arabinooligosaccharides | | |
| | | | | | GH43 family | | utilization | | |
| | | NBFEJGPP_00279 | | AbnA | Endo-alpha-(1->5)-L- | aAOS | alpha- | + | GH43_4 |
| | | | | | arabinanase (EC 3.2.1.99), | | arabinooligosaccharides | | |
| | | | | | GH43 family | | utilization | | |
| | | NBFEJGPP_00280 | | | | | | | SusD |
| | | NBFEJGPP_00281 | | | | | | | SusC |
| | | NBFEJGPP_00282 | | | | | | | SusD |
| | | NBFEJGPP_00283 | | | | | | | SusC |
| | | NBFEJGPP_00284 | | | | | | | |
| | | NBFEJGPP_00285 | | | | | | | HTCS |
| | | NBFEJGPP_00286 | | | | | | | |
| | | NBFEJGPP_00287 | | | | | | | |
| | | NBFEJGPP_00288 | | | | | | | |
| | | NBFEJGPP_00289 | | NplT | Neopullulanase | Mal; | maltose utilization; | +; + | GH13_46 |
| | | | | | (EC 3.2.1.135) | MOS | maltooligosaccharides | | |
| | | | | | | | utilization | | |
| | | NBFEJGPP_00290 | | PulA | Pullulanase (EC 3.2.1.41) | Mal; | maltose utilization; | +; + | GH13_14 |
| | | | | | | MOS | maltooligosaccharides | | |
| | | | | | | | utilization | | |
| | | NBFEJGPP_00291 | | SusB2 | Glucan 1,4-alpha- | Mal; | maltose utilization; | +; + | GH97 |
| | | | | | glucosidase (EC 3.2.1.3) | MOS | maltooligosaccharides | | |
| | | | | | | | utilization | | |
| | | NBFEJGPP_00292 | | MalQ | 4-alpha-glucanotransferase | Mal; | maltose utilization; | +; + | CBM20\|GH77 |
| | | | | | (amylomaltase) | MOS | maltooligosaccharides | | |
| | | | | | (EC 2.4.1.25) | | utilization | | |
| | | NBFEJGPP_00293 | | MalT | Predicted maltose | Mal; | maltose utilization; | +; + | MFS |
| | | | | | transporter, MFS family | MOS | maltooligosaccharides | | |
| | | | | | | | utilization | | |
| | | NBFEJGPP_00294 | | MalR | Maltose operon | Mal; | maltose utilization; | +; + | |
| | | | | | transcriptional repressor | MOS | maltooligosaccharides | | |
| | | | | | MalR, LacI family | | utilization | | |
| | | NBFEJGPP_00295 | | SusCm | SusC, outer membrane | Mal; | maltose utilization; | +; + | SusC |
| | | | | | protein involved in | MOS | maltooligosaccharides | | |
| | | | | | maltodextrin utilization | | utilization | | |
| | | NBFEJGPP_00296 | | SusDm | SusD, outer membrane | Mal; | maltose utilization; | +; + | SusD |
| | | | | | protein involved in | MOS | maltooligosaccharides | | |
| | | | | | maltodextrin utilization | | utilization | | |
| | | NBFEJGPP_00297 | | | | | | | |
| | | NBFEJGPP_00298 | | | | | | | |
| BgG5_1 | PUL18 | LACDBDNG_01761 | Consensus | | | | | | SusC |
| | | LACDBDNG_01762 | PUL3 | | | | | | SusC |
| | | LACDBDNG_01763 | | | | | | | SusD |
| | | LACDBDNG_01764 | | | | | | | |
| | | LACDBDNG_01765 | | | | | | | |
| | | LACDBDNG_01766 | | AguA | Xylan alpha-1,2- | aXyl | alpha-xylosides | + | GH67 |
| | | | | | glucuronosidase | | utilization | | |
| | | | | | (EC 3.2.1.131) | | | | |
| | | LACDBDNG_01767 | | | | | | | Est |
| | | LACDBDNG_01768 | | XynT | Xyloside transporter XynT | XOS | xylooligosaccharides | + | MFS |
| | | | | | | | utilization | | |
| | | LACDBDNG_01769 | | XynA | Endo-1,4-beta-xylanase | XOS | xylooligosaccharides | + | GH10 |
| | | | | | (EC 3.2.1.8) | | utilization | | |
| | | LACDBDNG_01770 | | | | | | | GH43_1 |
| | PUL3 | LACDBDNG_00550 | Consensus | SusDm | SusD, outer membrane | Mal; | maltose utilization; | +; + | SusD |
| | | | PUL11 | | protein involved in | MOS | maltooligosaccharides | | |
| | | | | | maltodextrin utilization | | utilization | | |
| | | LACDBDNG_00551 | | | | | | | SusC |
| | | LACDBDNG_00552 | | BglA2 | beta-glucosidase | Bgl | beta-glucosides | − | GH3 |
| | | | | | (EC 3.2.1.21) | | utilization | | |
| | | LACDBDNG_00553 | | | | | | | SusR |
| | PUL4 | LACDBDNG_00554 | Consensus | BglA2 | beta-glucosidase | Bgl | beta-glucosides | − | GH3 |
| | | | PUL13 | | (EC 3.2.1.21) | | utilization | | |
| | | LACDBDNG_00555 | | BglA | Beta-glucosidase | Bgl | beta-glucosides | − | GH3 |
| | | | | | (EC 3.2.1.21) | | utilization | | |
| | | LACDBDNG_00556 | | | | | | | |
| | | LACDBDNG_00557 | | | | | | | SusD |
| | | LACDBDNG_00558 | | | | | | | SusC |
| | | LACDBDNG_00559 | | | | | | | GH5_4 |
| | | LACDBDNG_00560 | | | | | | | HTCS |
| | PUL19 | LACDBDNG_01848 | Consensus | Abg | Arabinogalactan endo-1,4- | GOS | galactooligosaccharides | + | GH53 |
| | | | PUL16 | | beta-galactanase | | utilization | | |
| | | | | | (EC 3.2.1.89) | | | | |
| | | LACDBDNG_01849 | | LacZ | Beta-galactosidase | GOS | galactooligosaccharides | + | GH2 |
| | | | | | (EC 3.2.1.23) | | utilization | | |
| | | LACDBDNG_01850 | | | | | | | HTCS |
| | | LACDBDNG_01851 | | SusC_bga | SusC, outer membrane | GOS | galactooligosaccharides | + | SusC |
| | | | | | protein involved in beta- | | utilization | | |
| | | | | | galactoside utilization | | | | |
| | | LACDBDNG_01852 | | | | | | | SusD |
| | | LACDBDNG_01853 | | | | | | | |
| | | LACDBDNG_01854 | | | | | | | |
| | | LACDBDNG_01855 | | | | | | | |
| | | LACDBDNG_01856 | | | | | | | |
| | | LACDBDNG_01857 | | | | | | | Pept_SB |
| | | LACDBDNG_01858 | | | | | | | |
| | | LACDBDNG_01859 | | | | | | | |
| | PUL12 | LACDBDNG_01142 | Consensus | AbnA | Endo-alpha-(1->5)-L- | aAOS | alpha- | + | GH43_5 |
| | | | PUL17a/17b | | arabinanase (EC 3.2.1.99), | | arabinooligosaccharides | | |
| | | | | | GH43 family | | utilization | | |
| | | LACDBDNG_01143 | | AbnA | Endo-alpha-(1->5)-L- | aAOS | alpha- | + | GH43_4 |
| | | | | | arabinanase (EC 3.2.1.99), | | arabinooligosaccharides | | |
| | | | | | GH43 family | | utilization | | |
| | | LACDBDNG_01144 | | | | | | | |
| | | LACDBDNG_01145 | | | | | | | SusD |
| | | LACDBDNG_01146 | | | | | | | SusC |
| | | LACDBDNG_01147 | | | | | | | HTCS |
| | | LACDBDNG_01148 | | NplT | Neopullulanase | Mal; | maltose utilization; | +; + | GH13_46 |
| | | | | | (EC 3.2.1.135) | MOS | maltooligosaccharides | | |
| | | | | | | | utilization | | |
| | | LACDBDNG_01149 | | PulA | Pullulanase (EC 3.2.1.41) | Mal; | maltose utilization; | +; + | GH13_14 |
| | | | | | | MOS | maltooligosaccharides | | |
| | | | | | | | utilization | | |
| | | LACDBDNG_01150 | | SusB2 | Glucan 1,4-alpha- | Mal; | maltose utilization; | +; + | GH97 |
| | | | | | glucosidase (EC 3.2.1.3) | MOS | maltooligosaccharides | | |
| | | | | | | | utilization | | |
| | | LACDBDNG_01151 | | MalQ | 4-alpha-glucanotransferase | Mal; | maltose utilization; | +; + | CBM20\|GH77 |
| | | | | | (amylomaltase) | MOS | maltooligosaccharides | | |
| | | | | | (EC 2.4.1.25) | | utilization | | |
| | | LACDBDNG_01152 | | MalT | Predicted maltose | Mal; | maltose utilization; | +; + | MFS |
| | | | | | transporter, MFS family | MOS | maltooligosaccharides | | |
| | | | | | | | utilization | | |
| | | LACDBDNG_01153 | | MalR | Maltose operon | Mal; | maltose utilization; | +; + | |
| | | | | | transcriptional repressor | MOS | maltooligosaccharides | | |
| | | | | | MalR, LacI family | | utilization | | |
| | | LACDBDNG_01154 | | SusCm | SusC, outer membrane | Mal; | maltose utilization; | +; + | SusC |
| | | | | | protein involved in | MOS | maltooligosaccharides | | |
| | | | | | maltodextrin utilization | | utilization | | |
| | | LACDBDNG_01155 | | SusDm | SusD, outer membrane | Mal; | maltose utilization; | +; + | SusD |
| | | | | | protein involved in | MOS | maltooligosaccharides | | |
| | | | | | maltodextrin utilization | | utilization | | |
| | | LACDBDNG_01156 | | | | | | | |
| | | LACDBDNG_01157 | | | | | | | |
| | | LACDBDNG_01158 | | | | | | | |
| Bg2H3 | PUL13 | NPHPMIGE_01582 | Consensus | | | | | | GH43_1 |
| | | NPHPMIGE_01583 | PUL3 | XynA | Endo-1,4-beta-xylanase | XOS | xylooligosaccharides | + | GH10 |
| | | | | | (EC 3.2.1.8) | | utilization | | |
| | | NPHPMIGE_01584 | | XynT | Xyloside transporter XynT | XOS | xylooligosaccharides | + | MFS |
| | | | | | | | utilization | | |
| | | NPHPMIGE_01585 | | | | | | | Est |
| | | NPHPMIGE_01586 | | | | | | | HTCS |
| | | NPHPMIGE_01587 | | AguA | Xylan alpha-1,2- | aXyl | alpha-xylosides | + | GH67 |
| | | | | | glucuronosidase | | utilization | | |
| | | | | | (EC 3.2.1.131) | | | | |
| | | NPHPMIGE_01588 | | | | | | | |
| | | NPHPMIGE_01589 | | | | | | | |
| | | NPHPMIGE_01590 | | | | | | | SusC |
| | | NPHPMIGE_01591 | | | | | | | SusD |
| | | NPHPMIGE_01592 | | | | | | | |
| | PUL3 | NPHPMIGE_00840 | Consensus | | | | | | ECF-sigma |
| | | NPHPMIGE_00841 | PUL4 | Pgl | Polygalacturonase | GalAs | oligogalacturonate | + | GH28\|GH105 |
| | | | | | (EC 3.2.1.15) | | utilization | | |
| | | NPHPMIGE_00842 | | | | | | | |
| | | NPHPMIGE_00843 | | | | | | | |
| | | NPHPMIGE_00844 | | | | | | | CE |
| | | NPHPMIGE_00845 | | | | | | | SusC |
| | | NPHPMIGE_00846 | | | | | | | SusD |
| | | NPHPMIGE_00847 | | | | | | | |
| | PUL4 | NPHPMIGE_00849 | | | | | | | SusC |
| | | NPHPMIGE_00850 | | | | | | | SusD |
| | | NPHPMIGE_00851 | | | | | | | GH28 |
| | | NPHPMIGE_00852 | | XynB2 | Xylan 1,4-beta-xylosidase | XOS | xylooligosaccharides | + | GH43_10 |
| | | | | | (EC 3.2.1.37) | | utilization | | |
| | | NPHPMIGE_00853 | | AmyA | Alpha-amylase (EC 3.2.1.1) | Mal; | maltose utilization; | +; + | GH57 |
| | | | | | | MOS | maltooligosaccharides | | |
| | | | | | | | utilization | | |
| | | NPHPMIGE_00854 | | | | | | | GT4 |
| | | NPHPMIGE_00855 | | | | | | | GH133 |
| | PUL1 | NPHPMIGE_00746 | Consensus | BmgP | 4-O-beta-D-mannosyl-D- | bMnOS | Beta- | + | GH130 |
| | | | PUL7 | | glucose phosphorylase | | mannooligosaccharides | | |
| | | | | | (EC 2.4.1.281) | | utilization | | |
| | | NPHPMIGE_00747 | | BaMan26A | Extracellular endo-beta-(1- | bMnOS | Beta- | + | GH26 |
| | | | | | 4)-mannanase, GH26 | | mannooligosaccharides | | |
| | | | | | | | utilization | | |
| | | NPHPMIGE_00748 | | MnbY | Predicted mannan acetyl- | bMnOS | Beta- | + | CE7 |
| | | | | | esterase | | mannooligosaccharides | | |
| | | | | | | | utilization | | |
| | | NPHPMIGE_00749 | | BaMan26A | Extracellular endo-beta-(1- | bMnOS | Beta- | + | GH26 |
| | | | | | 4)-mannanase, GH26 | | mannooligosaccharides | | |
| | | | | | | | utilization | | |
| | | NPHPMIGE_00750 | | | | | | | |
| | | NPHPMIGE_00751 | | | | | | | SusD |
| | | NPHPMIGE_00752 | | | | | | | SusC |
| | PUL2 | NPHPMIGE_00758 | Consensus | MnnB2 | Endo-1,4-beta- | bMnOS | Beta- | + | GH5_7 |
| | | | PUL8 | | mannosidase | | mannooligosaccharides | | |
| | | | | | | | utilization | | |
| | | NPHPMIGE_00759 | | XynA | Endo-1,4-beta-xylanase | XOS | xylooligosaccharides | + | GH5_4[Fc] |
| | | | | | (EC 3.2.1.8) | | utilization | | |
| | | NPHPMIGE_00760 | | XynA | Endo-1,4-beta-xylanase | XOS | xylooligosaccharides | + | GH5_4 |
| | | | | | (EC 3.2.1.8) | | utilization | | |
| | | NPHPMIGE_00761 | | | | | | | |
| | | NPHPMIGE_00762 | | | | | | | |
| | | NPHPMIGE_00763 | | | | | | | SusD |
| | | NPHPMIGE_00764 | | | | | | | SusC |
| | PUL7 | NPHPMIGE_01168 | Consensus | | | | | | ECF-sigma |
| | | NPHPMIGE_01169 | PUL9 | | | | | | Anti-sigma |
| | | NPHPMIGE_01170 | | | | | | | GH63 |
| | | NPHPMIGE_01171 | | LacZ | Beta-galactosidase | GOS | galactooligosaccharides | + | GH2 |
| | | | | | (EC 3.2.1.23) | | utilization | | |
| | | NPHPMIGE_01172 | | | | | | | |
| | | NPHPMIGE_01173 | | | | | | | |
| | | NPHPMIGE_01174 | | | | | | | SusD |
| | | NPHPMIGE_01175 | | | | | | | SusC |
| | PUL30 | NPHPMIGE_02692 | Consensus | | | | | | GH127 |
| | | NPHPMIGE_02693 | PUL10 | | | | | | GH43_34\| |
| | | | | | | | | | CBM32 |
| | | NPHPMIGE_02694 | | AglA | Alpha-glucosidase | Mal; | maltose utilization; | +; + | GH97 |
| | | | | | (EC 3.2.1.20) | MOS | maltooligosaccharides | | |
| | | | | | | | utilization | | |
| | | NPHPMIGE_02695 | | | | | | | GH146 |
| | | NPHPMIGE_02696 | | | | | | | SusC |
| | | NPHPMIGE_02697 | | | | | | | SusD |
| | | NPHPMIGE_02698 | | | | | | | |
| | | NPHPMIGE_02699 | | AgaL | Alpha-galactosidase | Aga | alpha-galactosides | − | GH27 |
| | | | | | (EC 3.2.1.22) | | utilization | | |
| | PUL5 | NPHPMIGE_01099 | Consensus | BglA | Beta-glucosidase | Bgl | beta-glucosides | + | GH3 |
| | | | PUL13 | | (EC 3.2.1.21) | | utilization | | |
| | | NPHPMIGE_01100 | | | | | | | |
| | | NPHPMIGE_01101 | | | | | | | SusD |
| | | NPHPMIGE_01102 | | | | | | | SusC |
| | | NPHPMIGE_01103 | | | | | | | GH5_4 |
| | | NPHPMIGE_01104 | | | | | | | HTCS |
| | PUL12 | NPHPMIGE_01514 | Consensus | ThiD | Hydroxymethylpyrimidine | B1 | TPP cofactor, de novo | + | |
| | | | PUL16 | | phosphate kinase ThiD | | synthesis | | |
| | | | | | (EC 2.7.4.7) | | | | |
| | | NPHPMIGE_01515 | | | | | | | |
| | | NPHPMIGE_01516 | | | | | | | |
| | | NPHPMIGE_01517 | | | | | | | Pept_SB |
| | | NPHPMIGE_01518 | | | | | | | |
| | | NPHPMIGE_01519 | | | | | | | |
| | | NPHPMIGE_01520 | | | | | | | |
| | | NPHPMIGE_01521 | | | | | | | |
| | | NPHPMIGE_01522 | | | | | | | SusD |
| | | NPHPMIGE_01523 | | SusC_bga | SusC, outer membrane | GOS | galactooligosaccharides | + | SusC |
| | | | | | protein involved in beta- | | utilization | | |
| | | | | | galactoside utilization | | | | |
| | | NPHPMIGE_01524 | | | | | | | HTCS |
| | | NPHPMIGE_01525 | | LacZ | Beta-galactosidase | GOS | galactooligosaccharides | + | GH2 |
| | | | | | (EC 3.2.1.23) | | utilization | | |
| | | NPHPMIGE_01526 | | Abg | Arabinogalactan endo-1,4- | GOS | galactooligosaccharides | + | GH53 |
| | | | | | beta-galactanase | | utilization | | |
| | | | | | (EC 3.2.1.89) | | | | |
| | | NPHPMIGE_01527 | | | | | | | |
| | | NPHPMIGE_01528 | | SusD_bga | SusD, outer membrane | GOS | galactooligosaccharides | + | SusD |
| | | | | | protein involved in beta- | | utilization | | |
| | | | | | galactoside utilization | | | | |
| | | NPHPMIGE_01529 | | SusC_bga | SusC, outer membrane | GOS | galactooligosaccharides | + | SusC |
| | | | | | protein involved in beta- | | utilization | | |
| | | | | | galactoside utilization | | | | |
| | | NPHPMIGE_01530 | | PelB | Pectate lyase (EC 4.2.2.2) | (GalA)n | | | PL1\|CBM77 |
| | | NPHPMIGE_01531 | | | | | | | |
| | PUL23 | NPHPMIGE_02141 | Consensus | | | | | | |
| | | NPHPMIGE_02142 | PUL17a/17b | | | | | | |
| | | NPHPMIGE_02143 | | SusDm | SusD, outer membrane | Mal; | maltose utilization; | +; + | SusD |
| | | | | | protein involved in | MOS | maltooligosaccharides | | |
| | | | | | maltodextrin utilization | | utilization | | |
| | | NPHPMIGE_02144 | | SusCm | SusC, outer membrane | Mal; | maltose utilization; | +; + | SusC |
| | | | | | protein involved in | MOS | maltooligosaccharides | | |
| | | | | | maltodextrin utilization | | utilization | | |
| | | NPHPMIGE_02145 | | MalR | Maltose operon | Mal; | maltose utilization; | +; + | |
| | | | | | transcriptional repressor | MOS | maltooligosaccharides | | |
| | | | | | MalR, LacI family | | utilization | | |
| | | NPHPMIGE_02146 | | MalT | Predicted maltose | Mal; | maltose utilization; | +; + | MFS |
| | | | | | transporter, MFS family | MOS | maltooligosaccharides | | |
| | | | | | | | utilization | | |
| | | NPHPMIGE_02147 | | MalQ | 4-alpha-glucanotransferase | Mal; | maltose utilization; | +; + | CBM20\|GH77 |
| | | | | | (amylomaltase) | MOS | maltooligosaccharides | | |
| | | | | | (EC 2.4.1.25) | | utilization | | |
| | | NPHPMIGE_02148 | | SusB | Alpha-glucosidase SusB | Mal; | maltose utilization; | +; + | GH97[Fc] |
| | | | | | (EC 3.2.1.20) | MOS | maltooligosaccharides | | |
| | | | | | | | utilization | | |
| | | NPHPMIGE_02149 | | SusB2 | Glucan 1,4-alpha- | Mal; | maltose utilization; | +; + | GH97[Fn] |
| | | | | | glucosidase (EC 3.2.1.3) | MOS | maltooligosaccharides | | |
| | | | | | | | utilization | | |
| | | NPHPMIGE_02150 | | PulA | Pullulanase (EC 3.2.1.41) | Mal; | maltose utilization; | +; + | GH13_14 |
| | | | | | | MOS | maltooligosaccharides | | |
| | | | | | | | utilization | | |
| | | NPHPMIGE_02151 | | NplT | Neopullulanase | Mal; | maltose utilization; | +; + | GH13_46 |
| | | | | | (EC 3.2.1.135) | MOS | maltooligosaccharides | | |
| | | | | | | | utilization | | |
| | | NPHPMIGE_02152 | | | | | | | |
| | | NPHPMIGE_02153 | | | | | | | |
| | | NPHPMIGE_02154 | | | | | | | |
| | | NPHPMIGE_02155 | | | | | | | HTCS |
| | | NPHPMIGE_02156 | | | | | | | |
| | | NPHPMIGE_02157 | | | | | | | SusC |
| | | NPHPMIGE_02158 | | | | | | | SusD |
| | | NPHPMIGE_02159 | | | | | | | SusC |
| | | NPHPMIGE_02160 | | | | | | | SusD |
| | | NPHPMIGE_02161 | | AbnA | Endo-alpha-(1->5)-L- | aAOS | alpha- | + | GH43 4 |
| | | | | | arabinanase (EC 3.2.1.99), | | arabinooligosaccharides | | |
| | | | | | GH43 family | | utilization | | |
| | | NPHPMIGE_02162 | | | | | | | |
| | | NPHPMIGE_02163 | | AbnA | Endo-alpha-(1->5)-L- | aAOS | alpha- | + | GH43_5 |
| | | | | | arabinanase (EC 3.2.1.99), | | arabinooligosaccharides | | |
| | | | | | GH43 family | | utilization | | |

^{b}Total monosaccharide content (μg monosaccharide/mg of dried therapeutic food formulation or its ingredients).

TABLE 30A

| | | | Monosaccharide | | | | | | | |
|---|---|---|---|---|---|---|---|---|---|---|
| | | | | | | | | | | D-Glu- |
| | | | | | | | | | | curonic |
| | Sample | Technical | | Galac- | | | Arabi- | | Rham- | acid |
| Sample | type | replicate | Glucose | tose | Fructose | Xylose | nose | Fucose | nose | (GlcA) |
| MDCF2 | MDCF-2 | 1 | 208.46 | 13.73 | 12.00 | 1.10 | 5.78 | 1.53 | 0.41 | 0.10 |
| | diet | 2 | 240.76 | 14.47 | 13.03 | 1.73 | 9.69 | 1.51 | 0.84 | 0.22 |
| | | 3 | 240.81 | 11.57 | 17.19 | 1.23 | 6.90 | 1.17 | 0.53 | 0.14 |
| | | 4 | 285.18 | 17.38 | 43.54 | 2.27 | 10.53 | 0.74 | 0.54 | 0.11 |
| chickpea | MDCF-2 | 1 | 385.60 | 34.88 | 2.42 | 2.41 | 31.37 | 0.61 | 1.28 | 0.30 |
| | ingredient | 2 | 386.79 | 35.46 | 2.98 | 1.36 | 20.02 | 0.77 | 1.12 | 0.32 |
| | | 3 | 444.29 | 44.65 | 3.10 | 1.53 | 22.40 | 0.67 | 1.11 | 0.30 |
| | | 4 | 422.55 | 37.67 | 2.69 | 1.82 | 24.43 | 0.87 | 1.29 | 0.28 |
| peanut | MDCF-2 | 1 | 47.92 | 6.41 | 2.65 | 3.58 | 10.53 | 0.64 | 0.99 | 0.01 |
| | ingredient | 2 | 60.94 | 8.52 | 2.43 | 4.46 | 10.90 | 1.08 | 1.22 | 0.08 |
| | | 3 | 63.84 | 8.10 | 2.03 | 4.42 | 12.20 | 1.21 | 1.22 | 0.09 |
| | | 4 | 63.32 | 8.57 | 2.12 | 3.71 | 11.06 | 1.21 | 1.14 | 0.08 |
| soybean | MDCF-2 | 1 | 23.67 | 42.04 | 1.45 | 3.57 | 12.30 | 1.87 | 1.86 | 0.62 |
| | ingredient | 2 | 27.70 | 66.61 | 2.17 | 12.86 | 25.44 | 3.91 | 3.64 | 0.65 |
| | | 3 | 26.12 | 53.20 | 1.62 | 6.18 | 15.38 | 2.68 | 2.55 | 0.57 |
| | | 4 | 26.25 | 43.07 | 1.53 | 3.44 | 11.70 | 1.87 | 2.36 | 0.53 |
| green | MDCF-2 | 1 | 563.81 | 10.45 | 3.89 | 3.90 | 6.02 | 0.36 | 0.26 | 0.02 |
| banana | ingredient | 2 | 667.70 | 8.68 | 4.14 | 3.83 | 6.09 | 0.53 | 0.38 | 0.15 |
| | | 3 | 715.99 | 12.41 | 4.65 | 5.49 | 8.33 | 0.57 | 0.49 | 0.16 |
| | | 4 | 662.34 | 9.59 | 5.64 | 3.86 | 7.58 | 0.49 | 0.39 | 0.10 |
| RUSF | RUSF | 1 | 310.32 | 30.80 | 6.76 | 0.59 | 4.61 | 0.72 | 0.38 | 0.06 |
| | diet | 2 | 380.42 | 38.66 | 21.30 | 0.66 | 4.87 | 0.37 | 0.35 | 0.15 |
| | | 3 | 391.80 | 38.70 | 17.69 | 0.52 | 3.75 | 0.16 | 0.28 | 0.16 |
| | | 4 | 374.29 | 39.24 | 12.08 | 0.47 | 4.58 | 0.60 | 0.43 | 0.10 |
| Rice | RUSF | 1 | 910.02 | 0.87 | 4.21 | 0.61 | 1.99 | 0.15 | 0.04 | 0.03 |
| | ingredient | 2 | 746.76 | 6.12 | 5.60 | 0.60 | 2.55 | 0.17 | 0.05 | 0.09 |
| | | 3 | 742.54 | 5.86 | 4.94 | 0.65 | 2.27 | 0.28 | 0.08 | 0.01 |
| | | 4 | 678.09 | 7.68 | 3.43 | 0.42 | 1.86 | 0.34 | 0.03 | 0.04 |
| lentil | RUSF | 1 | 396.55 | 39.96 | 5.83 | 2.03 | 17.23 | 0.41 | 1.16 | 0.85 |
| | ingredient | 2 | 475.14 | 47.12 | 7.12 | 2.27 | 21.96 | 0.43 | 1.32 | 0.34 |
| | | 3 | 501.13 | 43.48 | 4.83 | 2.11 | 18.52 | 0.56 | 1.52 | 0.26 |
| | | 4 | 509.65 | 36.30 | 4.64 | 1.63 | 16.71 | 0.51 | 1.18 | 0.26 |
| milk | RUSF | 1 | 198.84 | 218.67 | 1.95 | 0.07 | 0.40 | 0.15 | 0.01 | 0.09 |
| powder | ingredient | 2 | 210.61 | 220.57 | 1.65 | 0.17 | 0.64 | 0.23 | 0.00 | 0.02 |
| | | 3 | 220.42 | 205.49 | 1.80 | 0.12 | 0.53 | 0.21 | 0.03 | 0.11 |
| | | 4 | 208.91 | 229.54 | 1.57 | 0.07 | 0.54 | 0.26 | 0.01 | 0.14 |

| | | | Monosaccharide | | | | | |
|---|---|---|---|---|---|---|---|---|
| | | | D-Galact- | | | | | |
| | | | uronic | N-Acetylglu- | N-Acetylgal- | | | |
| | Sample | Technical | acid | cosamine | actosamine | | | |
| Sample | type | replicate | (GalA) | (GlcNAc) | (GalNAc) | Mannose | Allose | Ribose |
| MDCF2 | MDCF-2 | 1 | 0.54 | 0.02 | 0.03 | 1.06 | 0.00 | 0.30 |
| | diet | 2 | 0.79 | 0.03 | 0.00 | 1.47 | 0.00 | 0.35 |
| | | 3 | 0.68 | 0.00 | 0.00 | 1.40 | 0.01 | 0.39 |
| | | 1 | 0.53 | 0.04 | 0.04 | 1.62 | 0.02 | 0.47 |
| chickpea | MDCF-2 | 1 | 1.16 | 0.08 | 0.01 | 0.83 | 0.00 | 0.68 |
| | ingredient | 2 | 1.00 | 0.02 | 0.04 | 0.92 | 0.00 | 0.89 |
| | | 3 | 0.90 | 0.00 | 0.03 | 1.09 | 0.00 | 1.03 |
| | | 4 | 0.95 | 0.03 | 0.01 | 0.84 | 0.00 | 0.83 |
| peanut | MDCF-2 | 1 | 1.22 | 0.01 | 0.00 | 0.77 | 0.00 | 0.35 |
| | ingredient | 2 | 1.94 | 0.02 | 0.01 | 0.88 | 0.00 | 0.34 |
| | | 3 | 1.71 | 0.06 | 0.00 | 0.96 | 0.00 | 0.40 |
| | | 4 | 1.82 | 0.04 | 0.01 | 0.86 | 0.00 | 0.45 |
| soybean | MDCF-2 | 1 | 2.34 | 0.04 | 0.03 | 4.23 | 0.01 | 1.25 |
| | ingredient | 2 | 4.44 | 0.12 | 0.01 | 11.19 | 0.01 | 1.58 |
| | | 3 | 3.30 | 0.06 | 0.00 | 8.52 | 0.00 | 1.36 |
| | | 4 | 2.48 | 0.09 | 0.01 | 5.81 | 0.00 | 1.31 |
| green | MDCF-2 | 1 | 1.75 | 0.02 | 0.00 | 7.17 | 0.00 | 0.32 |
| banana | ingredient | 2 | 3.17 | 0.01 | 0.02 | 7.11 | 0.02 | 0.37 |
| | | 3 | 3.11 | 0.05 | 0.00 | 7.68 | 0.01 | 0.40 |
| | | 4 | 1.14 | 0.04 | 0.04 | 7.28 | 0.01 | 0.46 |
| RUSF | RUSF | 1 | 0.26 | 0.01 | 0.02 | 0.39 | 0.00 | 0.41 |
| | diet | 2 | 0.15 | 0.18 | 0.09 | 0.43 | 0.01 | 0.63 |
| | | 3 | 0.19 | 0.01 | 0.01 | 0.36 | 0.00 | 0.52 |
| | | 4 | 0.31 | 0.12 | 0.01 | 0.46 | 0.00 | 0.50 |
| Rice | RUSF | 1 | 0.04 | 0.04 | 0.03 | 0.48 | 0.00 | 0.36 |
| | ingredient | 2 | 0.06 | 0.05 | 0.01 | 0.37 | 0.00 | 0.31 |
| | | 3 | 0.05 | 0.00 | 0.00 | 0.43 | 0.00 | 0.34 |
| | | 4 | 0.04 | 0.03 | 0.03 | 0.43 | 0.00 | 0.22 |
| lentil | RUSF | 1 | 1.05 | 0.09 | 0.06 | 1.03 | 0.00 | 1.08 |
| | ingredient | 2 | 0.45 | 0.15 | 0.02 | 1.14 | 0.03 | 1.40 |
| | | 3 | 0.56 | 0.07 | 0.03 | 1.03 | 0.00 | 1.25 |
| | | 4 | 0.57 | 0.00 | 0.00 | 1.14 | 0.00 | 1.19 |
| milk | RUSF | 1 | 0.00 | 0.10 | 0.01 | 0.72 | 0.01 | 1.00 |
| powder | ingredient | 2 | 0.00 | 0.07 | 0.01 | 0.90 | 0.00 | 1.39 |
| | | 3 | 0.01 | 0.10 | 0.02 | 0.87 | 0.02 | 1.37 |
| | | 4 | 0.01 | 0.07 | 0.00 | 0.68 | 0.00 | 1.34 |

TABLE 30B(i)
Glycosidic linkage composition (peak area, arbitrary units/ng dried diet or ingredient) - MDCF-2

| | Sample | | | | | | | | | |
|---|---|---|---|---|---|---|---|---|---|---|
| | MDCF2 | | | | Chickpea | | | | Peanut | |
| | Sample type | | | | | | | | | |
| | | | | | | | | | MDCF-2 | |
| | MDCF-2 diet | | | | MDCF-2 ingredient | | | | ingredient | |
| | R# | | | | | | | | | |
| | 1 | 2 | 3 | 4 | 1 | 2 | 3 | 4 | 1 | 2 |
| Glycosidic | 301.74 | 285.58 | 210.33 | 252.94 | 33.05 | 34.44 | 49.01 | 36.20 | 60.82 | 34.57 |
| linkage | | | | | | | | | | |
| 4-Glucose | 176.52 | 166.50 | 113.34 | 153.47 | 188.60 | 180.00 | 212.21 | 181.79 | 82.53 | 101.12 |
| 6-Glucose | 1.86 | 2.65 | 1.78 | 2.74 | 1.09 | 3.22 | 2.00 | 1.31 | 0.43 | 0.23 |
| 3-Glucose/ | 16.23 | 10.60 | 9.14 | 10.74 | 2.42 | 2.18 | 2.60 | 1.36 | 1.75 | 1.73 |
| 3-Galactose | | | | | | | | | | |
| 2-Glucose | 2.67 | 2.00 | 1.52 | 2.31 | 0.81 | 1.06 | 1.56 | 0.87 | 3.78 | 1.85 |
| 4,6-Glucose | 0.68 | 0.47 | 0.32 | 1.23 | 0.65 | 0.63 | 1.05 | 1.35 | 0.33 | 0.45 |
| 3,4-Glucose | 3.15 | 1.03 | 0.76 | 1.88 | 3.53 | 1.62 | 4.38 | 2.54 | 2.98 | 2.78 |
| 2,4-Glucose | 0.22 | 0.36 | 0.27 | 0.17 | 0.26 | 0.73 | 0.97 | 0.61 | 0.20 | 0.18 |
| 3,4,6-Glucose | 0.62 | 0.40 | 0.35 | 0.32 | 0.90 | 1.37 | 0.66 | 0.32 | 0.37 | 0.68 |
| 2,4,6-Glucose | 0.01 | 0.02 | 0.01 | 0.01 | 0.04 | 0.01 | 0.01 | 0.02 | 0.02 | 0.00 |
| T-Galactose | 25.45 | 32.02 | 21.52 | 29.24 | 40.16 | 47.65 | 65.87 | 55.84 | 12.94 | 26.58 |
| 4-Galactose | 10.70 | 13.15 | 7.93 | 9.41 | 3.68 | 5.50 | 4.77 | 3.35 | 0.92 | 1.96 |
| 2-Galactose | 3.37 | 4.52 | 1.96 | 4.28 | 3.68 | 3.35 | 5.33 | 3.83 | 1.87 | 2.50 |
| 4,6-Galactose | 0.76 | 0.45 | 0.30 | 0.38 | 1.06 | 0.41 | 0.95 | 0.90 | 0.51 | 0.39 |
| 3,6-Galactose | 0.19 | 0.22 | 0.14 | 0.33 | 0.18 | 0.03 | 0.05 | 0.24 | 0.02 | 0.07 |
| 3,4-Galactose | 0.32 | 0.15 | 0.08 | 0.09 | 0.29 | 0.17 | 0.16 | 0.12 | 0.11 | 0.12 |
| 3,4,6- | 0.07 | 0.05 | 0.07 | 0.05 | 0.08 | 0.08 | 0.08 | 0.07 | 0.05 | 0.06 |
| Galactose | | | | | | | | | | |
| 2,4,6- | 0.04 | 0.03 | 0.02 | 0.03 | 0.04 | 0.02 | 0.02 | 0.03 | 0.02 | 0.02 |
| Galactose | | | | | | | | | | |
| 2,3,6-Glucose | 0.09 | 0.07 | 0.05 | 0.06 | 0.11 | 0.15 | 0.07 | 0.05 | 0.03 | 0.22 |
| T-Fructose | 0.84 | 1.80 | 1.25 | 2.44 | 0.40 | 0.72 | 0.77 | 0.40 | 0.49 | 0.70 |
| T-P-Xylose | 5.96 | 5.63 | 3.89 | 5.46 | 4.96 | 2.60 | 4.16 | 3.33 | 5.59 | 1.86 |
| 4-P-Xylose | 1.76 | 0.01 | 0.01 | 0.30 | 0.06 | 0.07 | 0.06 | 0.11 | 1.28 | 0.12 |
| 2-P-Xylose | 0.93 | 0.86 | 0.56 | 0.73 | 0.63 | 0.26 | 0.33 | 0.22 | 0.47 | 0.34 |
| 3,4-P-Xylose/ | 0.90 | 0.66 | 0.52 | 0.26 | 1.19 | 1.00 | 1.10 | 0.68 | 1.03 | 0.99 |
| 3,5-Arabinose | | | | | | | | | | |
| 2,4-P-Xylose | 0.13 | 0.08 | 0.10 | 0.17 | 0.10 | 0.10 | 0.12 | 0.06 | 0.35 | 0.14 |
| T-P-Arabinose | 2.52 | 0.52 | 0.53 | 0.32 | 1.88 | 0.12 | 0.22 | 0.37 | 0.45 | 0.12 |
| T-F-Arabinose | 19.64 | 15.22 | 11.14 | 11.45 | 30.47 | 26.80 | 31.51 | 19.43 | 13.05 | 9.86 |
| 5-F-Arabinose | 3.82 | 1.87 | 1.63 | 3.61 | 8.07 | 2.31 | 2.58 | 1.76 | 3.57 | 2.86 |
| 3-Arabinose | 0.56 | 0.46 | 0.28 | 0.36 | 0.41 | 0.30 | 0.35 | 0.21 | 0.28 | 0.30 |
| 2-F-Arabinose | 0.71 | 0.73 | 0.33 | 0.60 | 0.65 | 0.38 | 0.46 | 0.33 | 0.41 | 0.21 |
| 2,5-F- | 0.07 | 0.05 | 0.04 | 0.05 | 0.09 | 0.10 | 0.13 | 0.08 | 0.03 | 0.05 |
| Arabinose | | | | | | | | | | |
| 2,3-F- | 0.40 | 0.40 | 0.27 | 0.12 | 0.65 | 0.58 | 0.58 | 0.38 | 0.27 | 0.27 |
| Arabinose | | | | | | | | | | |
| T-Rhamnose | 1.87 | 1.19 | 0.71 | 0.83 | 1.47 | 1.45 | 2.41 | 1.47 | 0.63 | 0.61 |
| 4-Rhamnose | 0.20 | 0.08 | 0.09 | 0.08 | 0.34 | 0.05 | 0.08 | 0.04 | 0.07 | 0.01 |
| 2-Rhamnose | 0.53 | 0.18 | 0.15 | 0.27 | 0.38 | 0.21 | 0.20 | 0.15 | 0.30 | 0.09 |
| T-Fucose | 3.01 | 1.01 | 0.61 | 0.73 | 1.71 | 0.46 | 0.50 | 0.32 | 0.53 | 0.45 |
| T-GlcA | 0.07 | 0.09 | 0.04 | 0.08 | 0.05 | 0.05 | 0.07 | 0.04 | 0.10 | 0.02 |
| T-GalA | 0.47 | 0.34 | 0.24 | 0.26 | 0.67 | 0.58 | 0.65 | 0.44 | 0.37 | 0.24 |
| T-Mannose | 17.16 | 13.11 | 7.74 | 7.93 | 3.04 | 2.16 | 1.88 | 1.53 | 2.06 | 1.74 |
| 6-Mannose | 0.00 | 0.01 | 0.01 | 0.01 | 0.00 | 0.01 | 0.01 | 0.01 | 0.01 | 0.01 |
| 4-Mannose | 9.24 | 8.07 | 6.93 | 6.62 | 6.64 | 6.96 | 8.70 | 6.39 | 3.45 | 5.03 |
| 3-Mannose | 0.33 | 0.16 | 0.14 | 0.19 | 0.18 | 0.07 | 0.09 | 0.07 | 0.09 | 0.11 |
| 2-Mannose | 0.13 | 0.29 | 0.16 | 0.05 | 0.11 | 0.33 | 0.40 | 0.27 | 0.03 | 0.05 |
| 4,6-Mannose | 0.17 | 0.12 | 0.10 | 0.14 | 0.08 | 0.08 | 0.08 | 0.06 | 0.06 | 0.07 |
| 3,4,6-Mannose | 0.05 | 0.04 | 0.04 | 0.04 | 0.04 | 0.04 | 0.03 | 0.03 | 0.00 | 0.03 |
| X-Hexose | 3.30 | 1.46 | 0.97 | 1.09 | 4.86 | 3.56 | 3.89 | 2.75 | 0.91 | 0.80 |
| 2,X,X- | 0.07 | 0.02 | 0.02 | 0.05 | 0.12 | 0.03 | 0.03 | 0.02 | 0.05 | 0.03 |
| Hexose (I) | | | | | | | | | | |
| 2,X,X- | 0.15 | 0.26 | 0.37 | 0.23 | 0.27 | 0.83 | 0.37 | 0.28 | 0.15 | 0.95 |
| Hexose (II) | | | | | | | | | | |

| | Sample | | | | | | | | | |
|---|---|---|---|---|---|---|---|---|---|---|
| | Peanut | | Soybean | | | | green banana | | | |
| | Sample type | | | | | | | | | |
| | MDCF-2 | | | | | | | | | |
| | ingredient | | MDCF-2 ingredient | | | | MDCF-2 ingredient | | | |
| | R# | | | | | | | | | |
| | 3 | 4 | 1 | 2 | 3 | 4 | 1 | 2 | 3 | 4 |
| Glycosidic | 23.98 | 22.67 | 56.10 | 27.36 | 41.10 | 47.49 | 6.63 | 11.38 | 32.80 | 8.06 |
| linkage | | | | | | | | | | |
| 4-Glucose | 55.31 | 60.49 | 55.90 | 31.41 | 66.02 | 41.54 | 76.79 | 83.94 | 179.60 | 69.27 |
| 6-Glucose | 0.43 | 0.45 | 2.80 | 5.58 | 5.06 | 5.96 | 0.02 | 0.20 | 0.19 | 0.10 |
| 3-Glucose/ | 0.93 | 0.62 | 3.05 | 1.35 | 1.61 | 2.14 | 0.01 | 0.55 | 1.88 | 0.22 |
| 3-Galactose | | | | | | | | | | |
| 2-Glucose | 1.49 | 1.30 | 0.85 | 0.80 | 0.74 | 0.96 | 0.04 | 0.13 | 0.36 | 0.08 |
| 4,6-Glucose | 0.19 | 0.23 | 0.28 | 0.15 | 0.32 | 0.22 | 0.15 | 0.18 | 0.93 | 0.14 |
| 3,4-Glucose | 1.16 | 1.21 | 0.57 | 0.09 | 0.87 | 0.37 | 0.68 | 1.05 | 4.59 | 0.55 |
| 2,4-Glucose | 0.08 | 0.10 | 0.09 | 0.13 | 0.36 | 0.13 | 0.07 | 0.09 | 0.34 | 0.06 |
| 3,4,6-Glucose | 0.25 | 0.22 | 0.14 | 0.11 | 2.40 | 0.10 | 0.13 | 0.24 | 1.40 | 0.07 |
| 2,4,6-Glucose | 0.01 | 0.02 | 0.06 | 0.02 | 0.08 | 0.06 | 0.01 | 0.01 | 0.20 | 0.01 |
| T-Galactose | 18.13 | 15.63 | 46.75 | 41.90 | 44.82 | 48.05 | 0.40 | 12.74 | 38.55 | 5.75 |
| 4-Galactose | 1.04 | 0.88 | 22.76 | 42.10 | 33.78 | 42.38 | 0.06 | 1.07 | 2.24 | 0.62 |
| 2-Galactose | 1.43 | 1.44 | 3.45 | 4.32 | 3.67 | 3.99 | 0.25 | 1.53 | 2.25 | 0.99 |
| 4,6-Galactose | 0.35 | 0.33 | 0.90 | 0.67 | 0.60 | 0.71 | 0.09 | 0.13 | 0.39 | 0.12 |
| 3,6-Galactose | 0.04 | 0.01 | 0.36 | 0.35 | 0.34 | 0.37 | 0.00 | 0.01 | 0.10 | 0.00 |
| 3,4-Galactose | 0.08 | 0.08 | 0.85 | 0.57 | 0.55 | 0.63 | 0.02 | 0.04 | 0.11 | 0.02 |
| 3,4,6- | 0.04 | 0.06 | 0.19 | 0.14 | 0.19 | 0.05 | 0.04 | 0.04 | 0.14 | 0.05 |
| Galactose | | | | | | | | | | |
| 2,4,6- | 0.02 | 0.03 | 0.07 | 0.06 | 0.13 | 0.06 | 0.02 | 0.02 | 0.09 | 0.04 |
| Galactose | | | | | | | | | | |
| 2,3,6-Glucose | 0.03 | 0.03 | 0.02 | 0.03 | 0.23 | 0.03 | 0.02 | 0.03 | 0.38 | 0.02 |
| T-Fructose | 0.26 | 0.18 | 0.73 | 0.74 | 1.02 | 1.38 | 0.06 | 0.19 | 0.68 | 0.11 |
| T-P-Xylose | 1.50 | 2.03 | 5.63 | 1.67 | 5.59 | 5.01 | 1.39 | 1.42 | 2.36 | 0.30 |
| 4-P-Xylose | 0.04 | 0.95 | 4.31 | 2.02 | 0.97 | 1.11 | 0.50 | 0.19 | 0.31 | 0.21 |
| 2-P-Xylose | 0.34 | 0.34 | 2.74 | 2.24 | 2.44 | 2.34 | 0.02 | 0.13 | 0.26 | 0.07 |
| 3,4-P-Xylose/ | 0.77 | 0.88 | 0.86 | 0.86 | 0.97 | 0.90 | 0.02 | 0.13 | 0.19 | 0.09 |
| 3,5-Arabinose | | | | | | | | | | |
| 2,4-P-Xylose | 0.12 | 0.12 | 0.27 | 0.27 | 0.42 | 0.28 | 0.03 | 0.04 | 0.44 | 0.03 |
| T-P-Arabinose | 0.17 | 0.09 | 1.50 | 0.18 | 0.25 | 0.28 | 0.31 | 1.03 | 0.11 | 0.12 |
| T-F-Arabinose | 4.47 | 3.70 | 22.83 | 6.33 | 22.86 | 24.37 | 0.95 | 2.67 | 7.40 | 1.26 |
| 5-F-Arabinose | 2.63 | 2.60 | 3.29 | 4.01 | 2.86 | 4.21 | 0.03 | 0.43 | 1.06 | 0.27 |
| 3-Arabinose | 0.22 | 0.20 | 0.59 | 0.62 | 0.55 | 0.67 | 0.05 | 0.11 | 0.79 | 0.19 |
| 2-F-Arabinose | 0.20 | 0.18 | 1.57 | 1.35 | 1.51 | 1.85 | 0.03 | 0.12 | 0.23 | 0.09 |
| 2,5-F- | 0.03 | 0.03 | 0.08 | 0.07 | 0.14 | 0.08 | 0.01 | 0.02 | 0.13 | 0.02 |
| Arabinose | | | | | | | | | | |
| 2,3-F- | 0.21 | 0.25 | 0.95 | 1.12 | 1.00 | 1.07 | 0.02 | 0.05 | 0.20 | 0.04 |
| Arabinose | | | | | | | | | | |
| T-Rhamnose | 0.26 | 0.18 | 5.96 | 1.31 | 5.76 | 5.58 | 0.04 | 0.17 | 0.54 | 0.08 |
| 4-Rhamnose | 0.04 | 0.06 | 0.45 | 0.07 | 0.08 | 0.07 | 0.03 | 0.06 | 0.15 | 0.14 |
| 2-Rhamnose | 0.10 | 0.12 | 1.13 | 0.22 | 0.24 | 0.68 | 0.61 | 0.17 | 0.43 | 0.08 |
| T-Fucose | 0.37 | 0.28 | 5.48 | 1.22 | 2.57 | 2.58 | 0.05 | 0.15 | 0.50 | 0.08 |
| T-GlcA | 0.02 | 0.02 | 0.05 | 0.03 | 0.09 | 0.08 | 0.00 | 0.01 | 0.02 | 0.01 |
| T-GalA | 0.11 | 0.08 | 0.55 | 0.14 | 0.52 | 0.55 | 0.02 | 0.06 | 0.19 | 0.03 |
| T-Mannose | 1.49 | 1.28 | 7.02 | 3.21 | 3.51 | 4.85 | 0.22 | 0.55 | 1.48 | 0.30 |
| 6-Mannose | 0.00 | 0.01 | 0.01 | 0.01 | 0.01 | 0.01 | 0.00 | 0.01 | 0.01 | 0.01 |
| 4-Mannose | 3.13 | 4.14 | 10.95 | 12.86 | 10.83 | 12.58 | 2.95 | 4.55 | 11.99 | 2.99 |
| 3-Mannose | 0.04 | 0.07 | 0.25 | 0.11 | 0.08 | 0.13 | 0.09 | 0.04 | 0.06 | 0.02 |
| 2-Mannose | 0.02 | 0.01 | 0.27 | 0.57 | 0.38 | 0.84 | 0.02 | 0.01 | 0.02 | 0.02 |
| 4,6-Mannose | 0.05 | 0.05 | 0.43 | 0.54 | 0.41 | 0.50 | 0.02 | 0.03 | 0.11 | 0.02 |
| 3,4,6-Mannose | 0.00 | 0.00 | 0.03 | 0.02 | 0.06 | 0.02 | 0.01 | 0.01 | 0.02 | 0.00 |
| X-Hexose | 0.86 | 0.76 | 4.21 | 2.42 | 2.00 | 2.51 | 0.06 | 0.25 | 0.77 | 0.20 |
| 2,X,X- | 0.01 | 0.02 | 0.04 | 0.02 | 0.04 | 0.21 | 0.02 | 0.02 | 0.07 | 0.02 |
| Hexose (I) | | | | | | | | | | |
| 2,X,X- | 0.06 | 0.07 | 0.08 | 0.06 | 1.24 | 0.08 | 0.11 | 0.20 | 1.47 | 0.12 |
| Hexose (II) | | | | | | | | | | |

TABLE 30B(ii)
Glycosidic linkage composition (peak area, arbitrary units/ng dried diet or ingredient) - RUSF

| | Sample | | | | | | | | |
|---|---|---|---|---|---|---|---|---|---|
| | RUSF | | | | Rice | | | | Lentil |
| | Sample type | | | | | | | | |
| | | | | | | | | | RUSF |
| | RUSF diet | | | | RUSF ingredient | | | | ingredient |
| | R# | | | | | | | | |
| | 1 | 2 | 3 | 4 | 1 | 2 | 3 | 4 | 1 |
| Glycosidic | 102.56 | 209.77 | 167.96 | 150.56 | 22.93 | 15.77 | 17.45 | 12.68 | 41.58 |
| linkage | | | | | | | | | |
| 4-Glucose | 120.80 | 235.54 | 171.87 | 159.18 | 157.16 | 181.60 | 124.91 | 100.28 | 188.31 |
| 6-Glucose | 1.16 | 1.13 | 1.96 | 0.64 | 0.29 | 0.27 | 0.28 | 0.21 | 2.18 |
| 3-Glucose/ | 4.71 | 7.23 | 6.92 | 3.10 | 1.27 | 0.69 | 0.62 | 0.69 | 4.15 |
| 3-Galactose | | | | | | | | | |
| 2-Glucose | 0.71 | 0.78 | 1.12 | 0.72 | 0.25 | 0.17 | 0.03 | 0.11 | 1.28 |
| 4,6-Glucose | 0.33 | 2.45 | 0.44 | 1.25 | 0.63 | 0.73 | 0.23 | 0.70 | 1.07 |
| 3,4-Glucose | 2.01 | 4.91 | 1.32 | 2.91 | 4.38 | 1.76 | 1.02 | 0.96 | 5.57 |
| 2,4-Glucose | 0.15 | 0.60 | 0.42 | 0.37 | 0.64 | 0.57 | 0.07 | 0.18 | 0.41 |
| 3,4,6-Glucose | 0.46 | 1.54 | 0.64 | 0.49 | 0.48 | 2.78 | 0.23 | 0.07 | 1.67 |
| 2,4,6-Glucose | 0.03 | 0.01 | 0.01 | 0.01 | 0.00 | 0.01 | 0.00 | 0.00 | 0.05 |
| T-Galactose | 29.48 | 82.22 | 60.40 | 53.82 | 3.01 | 2.63 | 3.70 | 2.84 | 57.03 |
| 6-Galactose | 0.67 | 4.85 | 3.73 | 2.94 | 0.10 | 0.25 | 0.26 | 0.16 | 17.38 |
| 4-Galactose | 2.46 | 2.21 | 1.54 | 0.61 | 0.32 | 0.13 | 0.14 | 0.12 | 8.24 |
| 2-Galactose | 3.32 | 8.09 | 6.50 | 5.60 | 1.35 | 0.85 | 0.31 | 0.21 | 2.71 |
| 4,6-Galactose | 0.49 | 0.32 | 0.24 | 0.25 | 0.18 | 0.10 | 0.04 | 0.04 | 1.65 |
| 3,6-Galactose | 0.14 | 0.42 | 0.02 | 0.21 | 0.02 | 0.02 | 0.01 | 0.02 | 0.07 |
| 3,4-Galactose | 0.20 | 0.08 | 0.07 | 0.05 | 0.06 | 0.03 | 0.02 | 0.02 | 0.38 |
| 3,4,6- | 0.07 | 0.06 | 0.02 | 0.03 | 0.03 | 0.03 | 0.01 | 0.00 | 0.16 |
| Galactose | | | | | | | | | |
| 2,4,6- | 0.03 | 0.11 | 0.03 | 0.01 | 0.02 | 0.03 | 0.00 | 0.00 | 0.03 |
| Galactose | | | | | | | | | |
| 2,3,6-Glucose | 0.07 | 0.14 | 0.06 | 0.05 | 0.02 | 0.29 | 0.02 | 0.04 | 0.08 |
| T-Fructose | 0.67 | 1.53 | 2.76 | 7.68 | 0.13 | 0.36 | 0.36 | 0.14 | 0.77 |
| T-P-Xylose | 3.48 | 0.83 | 1.12 | 0.96 | 0.85 | 0.23 | 0.64 | 0.15 | 3.89 |
| 4-P-Xylose | 1.21 | 0.29 | 0.03 | 0.10 | 0.72 | 0.12 | 0.20 | 0.12 | 2.87 |
| 2-P-Xylose | 0.41 | 0.14 | 0.10 | 0.07 | 0.06 | 0.02 | 0.05 | 0.02 | 0.82 |
| 3,4-P-Xylose/ | 0.36 | 0.37 | 0.22 | 0.18 | 0.24 | 0.19 | 0.05 | 0.10 | 1.35 |
| 3,5-Arabinose | | | | | | | | | |
| 2,4-P-Xylose | 0.09 | 0.05 | 0.02 | 0.04 | 0.04 | 0.05 | 0.02 | 0.01 | 0.18 |
| T-P-Arabinose | 2.51 | 0.08 | 0.16 | 0.06 | 0.32 | 0.04 | 0.07 | 0.04 | 1.56 |
| T-F-Arabinose | 7.05 | 8.87 | 7.87 | 5.92 | 1.74 | 1.06 | 3.41 | 0.94 | 43.96 |
| 5-F-Arabinose | 1.16 | 0.94 | 0.61 | 1.08 | 0.26 | 0.15 | 0.19 | 0.15 | 2.95 |
| 3-Arabinose | 0.18 | 0.31 | 0.23 | 0.19 | 0.04 | 0.05 | 0.04 | 0.03 | 0.54 |
| 2-F-Arabinose | 0.47 | 0.41 | 0.27 | 0.23 | 0.04 | 0.06 | 0.07 | 0.03 | 0.53 |
| 2,5-F- | 0.04 | 0.05 | 0.02 | 0.02 | 0.01 | 0.01 | 0.01 | 0.01 | 0.09 |
| Arabinose | | | | | | | | | |
| 2,3-F- | 0.15 | 0.31 | 0.20 | 0.14 | 0.01 | 0.02 | 0.02 | 0.01 | 1.12 |
| Arabinose | | | | | | | | | |
| T-Rhamnose | 1.04 | 1.52 | 1.47 | 1.26 | 0.08 | 0.09 | 0.16 | 0.08 | 4.21 |
| 4-Rhamnose | 0.21 | 0.02 | 0.03 | 0.02 | 0.13 | 0.01 | 0.01 | 0.00 | 0.17 |
| 2-Rhamnose | 0.41 | 0.07 | 0.06 | 0.05 | 0.48 | 0.03 | 0.03 | 0.01 | 1.02 |
| T-Fucose | 1.73 | 0.32 | 0.24 | 0.21 | 0.25 | 0.09 | 0.16 | 0.09 | 4.73 |
| T-GlcA | 0.03 | 0.04 | 0.03 | 0.02 | 0.00 | 0.01 | 0.01 | 0.00 | 0.03 |
| T-GalA | 0.15 | 0.19 | 0.16 | 0.13 | 0.05 | 0.02 | 0.07 | 0.02 | 1.10 |
| T-Mannose | 4.14 | 4.90 | 3.84 | 5.84 | 0.78 | 0.39 | 0.53 | 0.40 | 3.92 |
| 6-Mannose | 0.00 | 0.01 | 0.01 | 0.00 | 0.00 | 0.00 | 0.00 | 0.01 | 0.02 |
| 4-Mannose | 5.49 | 7.64 | 6.09 | 5.88 | 4.68 | 3.73 | 3.07 | 2.25 | 8.58 |
| 3-Mannose | 0.21 | 0.14 | 0.06 | 0.09 | 0.13 | 0.04 | 0.07 | 0.02 | 0.18 |
| 2-Mannose | 0.05 | 0.08 | 0.09 | 0.06 | 0.02 | 0.01 | 0.01 | 0.01 | 0.18 |
| 4,6-Mannose | 0.04 | 0.05 | 0.03 | 0.04 | 0.04 | 0.04 | 0.02 | 0.02 | 0.10 |
| 3,4,6-Mannose | 0.06 | 0.02 | 0.02 | 0.01 | 0.01 | 0.02 | 0.00 | 0.00 | 0.05 |
| X-Hexose | 2.40 | 1.63 | 1.38 | 1.08 | 0.55 | 0.16 | 0.10 | 0.13 | 8.14 |
| 2,X,X- | 0.07 | 0.03 | 0.01 | 0.01 | 0.04 | 0.06 | 0.00 | 0.00 | 0.11 |
| Hexose (I) | | | | | | | | | |
| 2,X,X- | 0.30 | 0.44 | 0.52 | 0.29 | 0.21 | 1.84 | 0.07 | 0.11 | 0.10 |
| Hexose (II) | | | | | | | | | |

| | Sample | | | | | | |
|---|---|---|---|---|---|---|---|
| | Lentil | | | Milk powder | | | |
| | Sample type | | | | | | |
| | RUSF | | | | | | |
| | ingredient | | | RUSF ingredient | | | |
| | R# | | | | | | |
| | 2 | 3 | 4 | 1 | 2 | 3 | 4 |
| Glycosidic | 24.42 | 28.49 | 28.58 | 3.04 | 8.84 | 6.70 | 7.95 |
| linkage | | | | | | | |
| 4-Glucose | 163.94 | 147.23 | 115.92 | 53.62 | 106.78 | 76.56 | 103.18 |
| 6-Glucose | 4.72 | 5.30 | 3.88 | 0.06 | 0.09 | 0.18 | 0.28 |
| 3-Glucose/ | 1.08 | 1.60 | 1.05 | 1.00 | 2.57 | 1.26 | 2.96 |
| 3-Galactose | | | | | | | |
| 2-Glucose | 0.41 | 0.51 | 0.36 | 0.18 | 0.17 | 0.10 | 0.23 |
| 4,6-Glucose | 1.78 | 0.58 | 1.32 | 0.21 | 0.57 | 0.11 | 0.79 |
| 3,4-Glucose | 1.40 | 1.73 | 2.90 | 0.25 | 0.68 | 0.38 | 1.38 |
| 2,4-Glucose | 0.57 | 0.49 | 0.32 | 0.16 | 0.11 | 0.07 | 0.29 |
| 3,4,6-Glucose | 0.32 | 1.22 | 0.22 | 0.06 | 0.03 | 0.06 | 0.41 |
| 2,4,6-Glucose | 0.01 | 0.02 | 0.00 | 0.00 | 0.00 | 0.00 | 0.01 |
| T-Galactose | 44.49 | 47.42 | 41.81 | 199.95 | 304.96 | 220.73 | 256.89 |
| 6-Galactose | 12.36 | 13.54 | 11.01 | 0.22 | 1.29 | 1.07 | 1.14 |
| 4-Galactose | 3.29 | 3.95 | 1.85 | 2.03 | 3.40 | 1.32 | 3.19 |
| 2-Galactose | 2.85 | 1.84 | 2.20 | 45.72 | 28.46 | 13.06 | 32.03 |
| 4,6-Galactose | 0.39 | 0.46 | 0.35 | 0.17 | 0.18 | 0.14 | 0.15 |
| 3,6-Galactose | 0.04 | 0.04 | 0.03 | 0.02 | 0.04 | 0.04 | 0.04 |
| 3,4-Galactose | 0.07 | 0.09 | 0.05 | 0.17 | 0.22 | 0.18 | 0.21 |
| 3,4,6- | 0.02 | 0.04 | 0.01 | 0.06 | 0.01 | 0.02 | 0.03 |
| Galactose | | | | | | | |
| 2,4,6- | 0.01 | 0.03 | 0.00 | 0.02 | 0.01 | 0.01 | 0.01 |
| Galactose | | | | | | | |
| 2,3,6-Glucose | 0.03 | 0.13 | 0.02 | 0.02 | 0.02 | 0.01 | 0.20 |
| T-Fructose | 0.26 | 0.50 | 0.50 | 6.90 | 5.55 | 2.62 | 2.05 |
| T-P-Xylose | 1.12 | 1.55 | 1.81 | 0.85 | 0.34 | 0.25 | 0.21 |
| 4-P-Xylose | 0.12 | 0.05 | 0.09 | 1.54 | 0.18 | 0.09 | 0.08 |
| 2-P-Xylose | 0.23 | 0.28 | 0.17 | 0.04 | 0.04 | 0.04 | 0.06 |
| 3,4-P-Xylose/ | 0.24 | 0.59 | 0.30 | 0.10 | 0.05 | 0.04 | 0.03 |
| 3,5-Arabinose | | | | | | | |
| 2,4-P-Xylose | 0.03 | 0.06 | 0.03 | 0.07 | 0.03 | 0.03 | 0.05 |
| T-P-Arabinose | 0.08 | 0.10 | 0.06 | 0.51 | 0.04 | 0.03 | 0.05 |
| T-F-Arabinose | 7.24 | 10.88 | 12.58 | 0.53 | 1.53 | 1.37 | 1.32 |
| 5-F-Arabinose | 1.83 | 1.80 | 2.56 | 0.06 | 0.13 | 0.17 | 0.30 |
| 3-Arabinose | 0.21 | 0.27 | 0.14 | 0.15 | 0.50 | 0.33 | 0.63 |
| 2-F-Arabinose | 0.29 | 0.23 | 0.17 | 0.63 | 1.15 | 0.57 | 0.95 |
| 2,5-F- | 0.05 | 0.06 | 0.03 | 0.01 | 0.01 | 0.01 | 0.02 |
| Arabinose | | | | | | | |
| 2,3-F- | 0.54 | 0.68 | 0.31 | 0.02 | 0.03 | 0.04 | 0.04 |
| Arabinose | | | | | | | |
| T-Rhamnose | 0.96 | 1.83 | 2.78 | 0.06 | 0.19 | 0.20 | 0.19 |
| 4-Rhamnose | 0.05 | 0.05 | 0.08 | 0.05 | 0.00 | 0.00 | 0.00 |
| 2-Rhamnose | 0.04 | 0.04 | 0.07 | 0.58 | 0.02 | 0.03 | 0.02 |
| T-Fucose | 0.22 | 0.33 | 0.25 | 0.20 | 0.27 | 0.24 | 0.24 |
| T-GlcA | 0.03 | 0.02 | 0.04 | 0.01 | 0.01 | 0.00 | 0.01 |
| T-GalA | 0.16 | 0.26 | 0.28 | 0.02 | 0.04 | 0.03 | 0.03 |
| T-Mannose | 1.54 | 2.19 | 1.65 | 1.35 | 1.17 | 0.74 | 1.08 |
| 6-Mannose | 0.01 | 0.01 | 0.01 | 0.00 | 0.00 | 0.01 | 0.02 |
| 4-Mannose | 2.98 | 3.74 | 2.83 | 17.92 | 43.95 | 25.41 | 32.05 |
| 3-Mannose | 0.04 | 0.07 | 0.03 | 0.32 | 0.18 | 0.15 | 0.29 |
| 2-Mannose | 0.07 | 0.16 | 0.09 | 0.17 | 0.07 | 0.08 | 0.35 |
| 4,6-Mannose | 0.04 | 0.04 | 0.03 | 0.06 | 0.11 | 0.06 | 0.08 |
| 3,4,6-Mannose | 0.01 | 0.00 | 0.00 | 0.05 | 0.00 | 0.01 | 0.02 |
| X-Hexose | 2.93 | 3.29 | 2.18 | 3.41 | 5.61 | 3.61 | 4.86 |
| 2,X,X- | 0.01 | 0.02 | 0.01 | 0.01 | 0.00 | 0.00 | 0.02 |
| Hexose (I) | | | | | | | |
| 2,X,X- | 0.15 | 0.65 | 0.08 | 0.08 | 0.04 | 0.02 | 0.78 |
| Hexose (II) | | | | | | | |

TABLE 30C
Polysaccharide composition (FITDOG, μg polysaccharide/mg of dried diet or ingredient)

| | | Technical | Polysaccharide | | | | | |
|---|---|---|---|---|---|---|---|---|
| Sample | Sample type | replicate | Starch | Cellulose | Mannan | Galactan | Arabinan | Xylan |
| MDCF2 | MDCF-2 | 1 | 222.89 | 4.45 | 0.51 | 1.74 | 0.82 | 0.40 |
| | Diet | 2 | 213.09 | 3.72 | 0.44 | 1.53 | 0.72 | 0.59 |
| | | 3 | 212.61 | 4.46 | 0.35 | 1.72 | 0.99 | 0.67 |
| Chickpea | MDCF-2 | 1 | 310.62 | 2.77 | 0.06 | 1.02 | 0.76 | 0.39 |
| | Ingredient | 2 | 327.43 | 2.54 | 0.01 | 0.40 | 0.44 | 0.21 |
| | | 3 | 387.61 | 1.71 | 0.01 | 0.91 | 0.67 | 0.30 |
| Peanut | MDCF-2 | 1 | 190.78 | 1.06 | 0.00 | 0.21 | 0.21 | 0.25 |
| | Ingredient | 2 | 241.82 | 0.84 | 0.01 | 0.10 | 0.47 | 0.23 |
| | | 3 | 250.39 | 1.60 | 0.01 | 0.73 | 0.17 | 0.09 |
| Soybean | MDCF-2 | 1 | 73.88 | 11.00 | 0.24 | 2.31 | 1.47 | 1.20 |
| | Ingredient | 2 | 15.75 | 6.63 | 0.15 | 2.22 | 1.85 | 1.19 |
| | | 3 | 18.95 | 6.70 | 0.07 | 2.29 | 2.53 | 1.16 |
| Green | MDCF-2 | 1 | 353.74 | 15.74 | 0.38 | 2.95 | 0.35 | 0.34 |
| banana | Ingredient | 2 | 335.77 | 14.24 | 0.33 | 3.78 | 0.55 | 0.42 |
| | | 3 | 343.38 | 14.78 | 0.19 | 4.44 | 0.96 | 0.41 |
| RUSF | RUSF Diet | 1 | 369.54 | 6.74 | 0.07 | 0.72 | 0.66 | 0.29 |
| | | 2 | 354.76 | 8.47 | 0.08 | 0.72 | 0.66 | 0.35 |
| | | 3 | 311.75 | 8.42 | 0.05 | 0.59 | 0.60 | 0.40 |
| Rice | RUSF | 1 | 489.61 | 5.86 | 0.06 | 1.18 | 0.79 | 0.35 |
| | Ingredient | 2 | 495.79 | 5.14 | 0.06 | 1.49 | 0.86 | 0.12 |
| | | 3 | 490.83 | 5.46 | 0.05 | 0.89 | 0.38 | 0.10 |
| Lentil | RUSF | 1 | 254.83 | 1.40 | 0.00 | 1.14 | 1.07 | 0.21 |
| | Ingredient | 2 | 302.18 | 1.43 | 0.01 | 1.46 | 0.88 | 0.46 |
| | | 3 | 322.48 | 1.19 | 0.05 | 4.49 | 1.06 | 0.55 |
| Milk | RUSF | 1 | 11.47 | 0.22 | 0.00 | 0.26 | 0.09 | 0.08 |
| powder | Ingredient | 2 | 8.85 | 0.38 | 0.00 | 0.03 | 0.08 | 0.10 |
| | | 3 | 6.97 | 0.68 | 0.00 | 0.01 | 0.20 | 0.21 |

1. A composition comprising a bacterial strain and carrier, wherein the bacterial strain comprises one or more polysaccharide utilization loci (PUL) selected from the group consisting of PUL3a, PUL3b, PUL9, PUL10, PUL15, PUL16, PUL17, PUL18, PUL 19, PUL20, PUL22, and PUL30.

2. The composition of claim 1, wherein the one or more PUL comprises a polynucleotide sequence at least about 90% identical to a PUL from a sequence deposited at the European Nucleotide Archive with accession number ERZ17359655a corresponding to Prevotella copri Bg131, accession number ERZ17359674 corresponding to Prevotella copri BgF5_2 or accession number ERZ17359677 corresponding to Prevotella copri BgD5_2.

3. The composition of claim 2, wherein the bacterial strain comprises a genome sequence at least about 90% identical to a sequence deposited at the European Nucleotide Archive with accession number ERZ17359655a corresponding to Prevotella copri Bg131, accession number ERZ17359674 corresponding to Prevotella copri BgF5_2 or accession number ERZ17359677 corresponding to Prevotella copri BgD5_2.

4. (canceled)

5. The composition of claim 1, wherein the bacterial strain is P. copri.

6. (canceled)

7. (canceled)

8. The composition of claim 1, wherein the composition comprises a microbiome-directed therapeutic food (MDF).

9. The composition of claim 8, wherein the MDF comprises chickpea flour, peanut flour, soy flour, green banana, sugar, at least one oil, optionally an amino acid mix, a micronutrient premix, wherein the micronutrient premix provides at least 60% of the recommended daily allowance of vitamin A, vitamin C, vitamin D, vitamin E, vitamin B, calcium, copper, iron, magnesium, manganese, phosphorus, potassium, and zinc for a child aged 6-24 months.

10. The composition of claim 9, wherein the MDF contains no milk, powdered milk or milk product.

11. (canceled)

12. The composition of claim 8, wherein the MDF is selected from the group consisting of MDCF-1, MDCF-2, MDCF-3, MDCF-2SS, MDSF, and MD-RUTF.

13. The composition of claim 1, comprising an additional probiotic bacterial strain, wherein the additional strain is a probiotic strain.

14. The composition of claim 13, wherein the additional probiotic bacterial strain is a strain of Bifidobacterium longum subspecies infantis.

15. (canceled)

16. The composition of claim 13, wherein the additional probiotic bacterial strain is Bifidobacterium longum subsp. infantis having NRRL deposit #NRRL B-68253.

17.-23. (canceled)

24. The composition of 1, wherein the bacterial strain is an engineered prebiotic bacterial strain.

25. (canceled)

26. The composition of claim 24, wherein the one or more PUL is exogenous to the bacterial genome and is within genome of the bacterial strain or is present as an extrachromosomal element.

27-40. (canceled)

41. A method of treatment, the method comprising administering to a subject in need thereof, a therapeutically effective quantity of a composition of claim 1.

42-67. (canceled)

68. A food formulation selected from the group consisting of MDCF-1, MDCF-2, MDCF-3, MDCF-2SS, MDSF, and MD-RUTF, for the treatment of wasting, below average weight gain, or stunting.

69-71. (canceled)

72. The food formulation of claim 68, wherein the food formulation comprises chickpea flour, peanut flour, soy flour, and raw banana, wherein the chickpea flour, the peanut flour, the soy flour, and the raw banana provide at least 8.5 g of protein per 100 g of the food formulation.

73. The food formulation of claim 72, wherein the food formulation lacks milk of any kind.

74. An engineered bacterium, wherein the engineered bacterium comprises one or more exogenous polysaccharide utilization loci (PUL) selected from the group consisting of PUL3a, PUL3b, PUL9, PUL10, PUL15, PUL16, PUL17, PUL18, PUL 19, PUL20, PUL22, and PUL30, wherein at least one of the exogenous one of more PUL is within the genome of the bacteria or within an extrachromosomal element.

75. The engineered bacterium of claim 74, wherein the exogenous one or more PUL comprises a polynucleotide sequence at least 90% identical to a PUL from a genome sequence deposited at the European Nucleotide Archive with accession number ERZ17359655a corresponding to Prevotella copri Bg131, accession number ERZ17359674 corresponding to Prevotella copri BgF5_2 or accession number ERZ17359677 corresponding to Prevotella copri BgD5_2.

76. The engineered bacterium of claim 75, wherein the engineered bacterium comprises a genome sequence at least about 90% identical to any one of the sequences deposited at the European Nucleotide Archive with accession number ERZ17359655a corresponding to Prevotella copri Bg131, accession number ERZ17359674 corresponding to Prevotella copri BgF5_2 or accession number ERZ17359677 corresponding to Prevotella copri BgD5_2.
